(19) United States
(12) Patent Application Publication
(10) Pub. No.: US 2025/0171495 A1
(43) Pub. Date: May 29, 2025

(54) MUTLI-TARGETED SERINE PROTEASE INHIBITORS FOR TREATMENT OF CANCER AND VIRAL INFECTION

(71) Applicant: Washington University, St. Louis, MO (US)

(72) Inventor: James Janetka, St. Louis, MO (US)

(21) Appl. No.: 18/964,037

(22) Filed: Nov. 29, 2024

Related U.S. Application Data
(60) Provisional application No. 63/706,454, filed on Oct. 11, 2024.
(60) Provisional application No. 63/603,525, filed on Nov. 28, 2023.

(51) Int. Cl. C07K 5/11; A61K 38/00; C07K 5/02; C07K 5/072; C07K 5/09

(52) U.S. Cl. CPC C07K 5/1019; C07K 5/02; C07K 5/06095; C07K 5/0817; A61K 38/00

(57) ABSTRACT

Among the various aspects of the present disclosure are compounds that are useful for inhibiting one or more proteases including various serine proteases such as Hepatocyte Growth Factor Activator (HGFA), and the type II transmembrane serine proteases (TTSPs) including matriptase, hepsin, and TMPRSS2. The present invention also relates to various methods of using the inhibitor compounds to treat and/or prevent infections and their symptoms/complications, including those caused by coronaviruses and influenza viruses, conditions associated with inflammation, various other malignancies, pre-malignant conditions, and/or cancer.

### CROSS REFERENCE TO RELATED APPLICATIONS

This application claims the benefit of U.S. Provisional Application No. 63/706,454, filed Oct. 11, 2024, and U.S. Provisional Application No. 63/603,525, filed Nov. 28, 2023, the contents of which are incorporated by reference herein in their entirety.

### FIELD OF INVENTION

The present disclosure generally relates to compounds that are useful for inhibiting one or more proteases including various serine proteases such as Hepatocyte Growth Factor Activator (HGFA), and the type II transmembrane serine proteases (TTSPs) including matriptase, hepsin, and TMPRSS2 The present invention also relates to various methods of using the inhibitor compounds to treat and/or prevent infections and their symptoms/complications, including those caused by coronaviruses and influenza viruses, conditions associated with inflammation, various other malignancies, pre-malignant conditions, and/or cancer.

### BACKGROUND OF INVENTION

Proteases, also known as proteinases, peptidases, or proteolytic enzymes, are enzymes that process proteins by hydrolyzing peptide bonds between amino acid residues. It is known that proteases regulate numerous physiological processes which enable or stimulate the growth, proliferation, transformation, motility, survival, and metastasis of tumor cells. Metastasis involves the proteolytic degradation of the extracellular matrix proteins (e.g. collagen) surrounding the tumor cells by proteases, which enables the invasion of tumor cells metastasizing from the primary tumors into the surrounding tissue and the lymph system or the blood system. Theses degradation proteases include matrix metalloproteases (MMPs) and cysteine proteases including various subfamily members of the cathepsins. Proteases are also involved in the activation of growth factors, cytokines, and other proteins that stimulate the growth, proliferation, motility, and survival of cancer cells, thus enabling tumors to develop and expand in size. These include multiple members of trypsin-like S1 serine protease such as HGFA, the kallikrein (KLK) family of proteases, such as KLK5, and of the subfamily of transmembrane serine proteases such as matriptase, hepsin, TMPRSS2, TMPRSS11D (HAT, human airway trypsin-like protease), and TMPRSS13 which have been found to be important in tissue homeostasis, infection, other diseases such as cancer, including but not limited to tumor cell signaling, motility, survival, angiogenesis, and transformation, leading to disease progression and metastasis.

Hepatocyte Growth Factor Activator (HGFA) is a member of the S1 trypsin-like serine protease family. Characteristic of coagulation cascade proteases like thrombin and Factor Xa, HGFA circulates in plasma as an inactive form, pro-HGFA at relatively high levels (40 nM). pro-HGFA is produced by the liver (or cancer cells) and is activated by other serine proteases in the plasma including thrombinand kallikrein-related (KLK) peptidases such as KLK5. The normal biological function of HGFA is to proteolytic process and activate the MET and RON receptor tyrosine kinase (RTK) ligands, HGF (hepatocyte growth factor), and MSP (macrophage stimulating protein), respectively primarily in injured tissue. HGFA is overexpressed and its activity is unregulated in multiple cancer types including breast and prostate cancer.

HGF and MSP are members of the plasminogen family of proteins that are secreted as inactive single-chain zymogens, pro-HGF and pro-MSP. The latter are the only two known HGFA substrates and are enzymatically hydrolyzed at the Arg494-Val495 and the Arg483-Val484 peptide bonds, respectively, by either HGFA or the cell-surface serine proteases matriptase and hepsin or TMPRSS2. After enzymatic cleavage, the α-chain N-terminal fragment and β-chain C-terminal fragments of the processed growth factor spontaneously form a two-chain disulfide bridged heterodimer capable of binding to the extracellular domain of its respective receptor tyrosine kinase and causing its activation. The processing of HGF and MSP is thus critical for ligand-dependent cell signaling of the MET and RON pathways. HGF is a key driver of cancer disease progression and metastasis in the tumor microenvironment and its increased production has been demonstrated to be a mechanism for resistance to targeted therapies, immunotherapy, and chemotherapy.

TMPRSS2, matriptase and hepsin are type II transmembrane serine proteases (TTSPs) which are present on epithelial cells in a normal physiological state. Like HGFA, they are members of the S1 family of serine endopeptidases and are upregulated and overexpressed in multiple cancer cell types and invasive tumors. The proteolytic activity of all four proteases, TMPRSS2, matriptase, hepsin, and HGFA, is regulated by the endogenous polypeptide Kunitz Domain type inhibitors HAI-1 and HAI-2 which inhibit all three proteases at a low nM concentration. An imbalance of normal HGFA, matriptase, and hepsin relative to HAI-1 and HAI-2 expression has been demonstrated to lead to invasive phenotypes in breast and other types of cancer. HAI-1 and HAI-2 act in the tumor pericellular microenvironment to block cell signaling by preventing the ligand-based activation of MET and/or RON receptor tyrosine kinases. Thus, potent and selective small molecule inhibitors of each HGF-activating protease can be useful as new therapeutics and chemical tools such as activity-based probes for studying the individual roles of HGFA, matriptase, and hepsin in cancer and other diseases.

Further, TMPRSS2 and matriptase are involved in the pathogenesis of coronavirus and influenza infections. The COVID-19 pandemic, caused by the SARS-CoV-2 coronavirus, has been an eye-opening experience for both the general and scientific community across the world. It has impacted all aspects of human life and the threat of future outbreaks via infections from another virus or bacteria for which currently available drugs will be ineffective, like SARS-CoV-2 at the outset. Incredibly, vaccines as well as several new drug candidates targeting this virus were developed at unprecedented speed. This was made possible by the combined efforts of scientists worldwide who elucidated details about the makeup and pathogenesis of SARS-CoV-2 infection. This work resulted in the identification of several potential therapeutic targets such as two viral proteases MPro and ClPro, as well as several host cell proteases, most importantly TMPRSS2 and matriptase.

The role of TMPRSS2 in SARS-CoV-2 infection and other viral infections from coronaviruses and influenza (and potentially others) is related to proteolytic processing of the RNA virus's outer glycoproteins Spike and Hemagglutinin, respectively. The Spike and HA proteins require partial proteolysis to bind to their host cell receptors, namely ACE2 in SARS-CoV-2. Matriptase has also been shown to be important for HA processing in influenza. Both TMPRSS2 and matriptase are advantageously located on the surface of lung epithelial cells and is highjacked by these respiratory viruses to perform this function and thus is essential for host cell viral binding and entry in the lung. It has been demonstrated that inhibition of TMPRSS2 and/or matriptase with small molecule inhibitors also results in decreased viral replication with multiple advantageous effects on alleviating symptoms and life-threatening complications of infection, including effects on the immune system in reducing inflammation.

TMPRSS2 and matriptase have a trypsin-like S1 serine protease domain and belongs to the family of Type II Transmembrane Serine Protease (TTSP) proteolytic enzymes an dlarger family of membrane-associated serine proteases (MASPs). In addition to SARS-CoV-2, TMPRSS2 has previously been shown to be important in other coronavirus infections caused by SARS-CoV and MERS. Furthermore, TMPRSS2 and matriptase have been demonstrated to proteolytically process the Hemagglutinin (HA) protein on the surface of influenza A viruses, allowing viral cell adhesion and entry as well as replication in these infections. Furthermore, there are reports of TMPRSS11D (HAT), TMPRSS4, and TMPRSS13 also being involved in coronavirus and influenza viral cell entry and pathogenesis. Various inhibitors of TMPRSS2 and matriptase have been developed which have demonstrated antiviral activity against influenza viruses.

### SUMMARY OF INVENTION

Among the various aspects of the present disclosure is the provision of a serine protease inhibitor for the treatment of cancer and viral infections.

Briefly, therefore, the present disclosure is directed to serine protease inhibitor compositions and related therapeutic methods.

The present teachings include compositions for a serine protease inhibitor. In some aspects, the composition can be a peptidomimetic molecule. In another aspect, the composition can be selected from any of the molecular structures described in the present disclosure. In yet another aspect, the composition can target 1, 2 or more serine proteases.

The present teachings also include a method to treat a disease, the method comprising administering a serine protease inhibitor to a subject in need. In some aspects, the disease can be selected from cancer and viral infection. In one aspect, the viral infection can be SARS-CoV-2 or other coronaviruses. In another aspect, the viral infection can be influenza A or other influenza viruses. In yet another aspect, the serine protease inhibitor can be selected from any of the molecular structures described in the present disclosure. In accordance with another aspect, the composition can target/inhibit 1, 2 or more serine proteases.

Other objects and features will be in part apparent and in part pointed out hereinafter.

An aspect of the disclosure is the description of compounds of Formula (IA), (IB), or (IC), a pharmaceutically acceptable salt thereof, or a stereoisomer thereof:

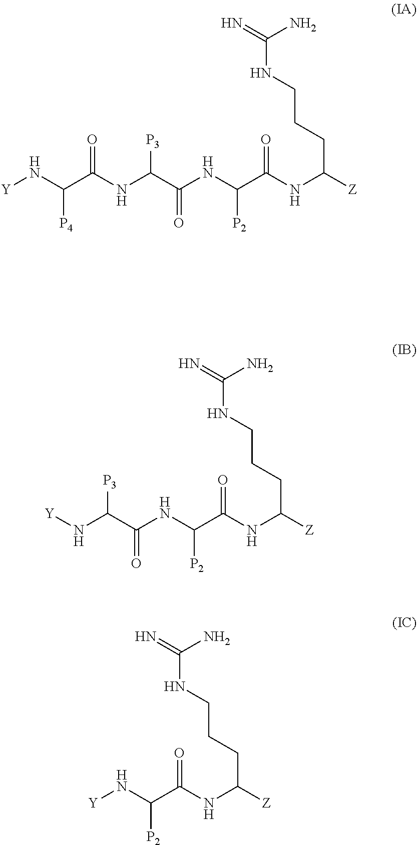

wherein each P₂ is independently a side chain of 1-Nal, 2-Nal, Gly(2-th), D-Ala, L-Ala, L-Ala(2-th), L-Arg, L-Arg(Z)₂, L-Asn, L-Bla, L-Cha, L-Chg, L-Glu(OBzl), L-hArg, L-hCha, L-His(Bzl), L-hLeu, L-hPhe, L-hTyr, L-hTyr(Me), L-Igl, L-Leu, L-Lys, L-Lys(2-ClZ), L-Nle, L-Nle(OBzl), L-NptGly, L-Nva, L-Orn, L-Phe, L-Phe(3,4-F₂), L-Phe(3-Cl), L-Phe(3-F), L-Phe(4-F), L-phg, L-Ser, L-Thr, L-Trp, or L-Val; each P₃ is independently a side chain of 3-AMPA, 4-AMBA, 4-AMPA, D-Arg, D-Gln, D-Lys, D-Phg, D-Ser, D-Trp, L-2-Aoc, L-Abu(Bth), L-Agb, L-Agp, L-Ala(Bth), L-Arg, L-Arg(Z)₂, L-Asp(OCHx), L-Dab, L-Dap, L-Dht, L-Gln, L-Glu, L-Glu(All), L-Glu(Bzl), L-Glu(Obzl), L-Glu(OCHx), L-Glu(OMe), L-hArg, L-hCha, L-His(3-Bom), L-hPhe, L-hTyr, L-Igl, L-Leu, L-Lys, L-Lys(2-ClZ), L-Met, L-Met(O), L-Met(O)₂, L-Nle(OBzl), L-Orn, L-Phe(4-NO₂), L-Phe(F₅), L-Ser, L-Ser(Ac), L-Thr, or L-Trp; each P₄ is independently a side chain of 2-Abz, 3-Abz, 4-Abz, D-Arg, dhAbu, dhLeu, D-Lys, D-Trp, Gly, L-2-Aoc, L-Agb, L-Agp, L-Ala(Bth), L-Arg, L-Arg(NO₂), L-Arg(Z)₂, L-Chg, L-Cys(4-MeOBzl), L-Cys(Bzl), L-Cys(MeBzl), L-DAB(Z), L-Glu(OBzl), L-Glu(OCHx), L-hArg, L-hCha, L-His(3-Bom), L-hLeu, L-hPhe, L-hTyr, L-Hyp, L-Hyp(Bzl), L-Idc, L-Ile, L-Leu, L-Lys, L-Lys(2-Cl-Z), L-Lys(TFA), L-Met, L-Nle, L-Nle(OBzl), L-Nva, L-Oic, L-Orn, L-Phe, L-Phe(4-I), L-Phe(F₅), L-Pro, L-Ser, L-Ser(Bzl), L-Thr(Bzl), L-Trp, or TmbGly; Y is H, acetyl, tert-butyloxycarbonyl (Boc), benzyloxycarbonyl (Cbz), fluorenylmethyloxycarbonyl (Fmoc), benzyl, —C(O)R, —SOOR, —COOR, —C(O)NHR, —COCH(NHC(O)CH₃)—(CH₂)ₓ—NHC(O)—(CH₂)ₓ-p-halo-phenyl, substituted or unsubstituted —(CH₂)ₓaryl, substituted or unsubstituted —(CH₂)ₓheteroaryl, substituted or unsubstituted —(CH₂)ₓcycloalkyl, or substituted or unsubstituted —(CH₂)ₓheterocycle; each x is independently an integer of 0, 1, 2, 3, or 4; each R is independently C₁ to C₆ alkyl, C₃ to C₆ cycloalkyl, heterocycle, alkylheterocycle, aralkyl, or aryl; each Z is independently

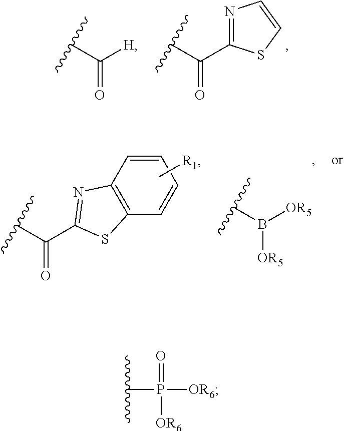

R₁ is hydrogen

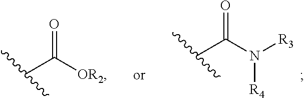

R₂ and R₃ are each independently hydrogen, substituted or unsubstituted alkyl, substituted or unsubstituted cycloalkyl, substituted or unsubstituted aryl, substituted or unsubstituted aralkyl, substituted or unsubstituted heteroaryl, or substituted or unsubstituted heteroarylalkyl; R₄ is hydrogen, substituted or unsubstituted alkyl, or a residue of an amino acid, or R₃ and R₄ can form a ring; each R₅ is independently hydrogen, substituted or unsubstituted alkyl, or the R₅ moieties can form a ring; and each R₆ is substituted or unsubstituted aryl.

Another aspect of the disclosure is a compound of Formula (IA), (IB), or (IC), a pharmaceutically acceptable salt thereof, or a stereoisomer thereof:

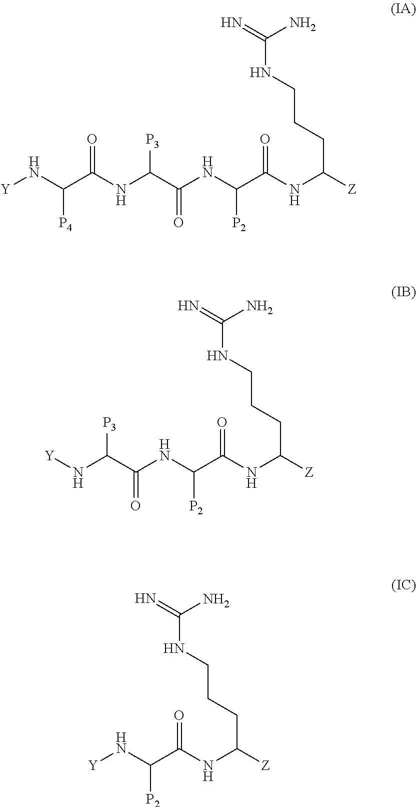

wherein each P₂ is independently a side chain of L-Ala(2-th), L-Arg(Z)₂, L-Asn, L-Bta, L-Cha, L-Chg, L-Glu(OBzl), L-hArg, L-hCha, L-His(Bzl), L-hLeu, L-hPhe, L-hTyr(Me), L-hTyr, L-Igl, L-Leu, L-Lys(2-ClZ), L-Nle(OBzl), L-Nle, L-NptGly, L-Nva, L-Orn, L-Phe(3,4-F2), L-Phe(3-Cl), L-Phe(3-F), L-Phe(4-F), L-Phe, L-Thr, or L-Trp; each P₃ is independently a side chain of D-Arg, D-Gln, D-Lys, D-Trp, L-2-Aoc, L-Agp, L-Ala(Bth), L-Arg(Z)₂, L-Arg, L-Dab, L-Dap, L-Dht, L-Glu(All), L-Glu(OBzl), L-Glu(OCHx), L-Glu(OMe), L-hArg, L-hCha, L-hPhe, L-hTyr, L-Igl, L-Lys, L-Met(O), L-Met(O)₂, L-Nle(OBzl), L-Orn, L-Phe(F₅), or L-Ser(Ac); each P₄ is independently a side chain of 4-Abz, chAbu, D-Arg, dhAbu, dhLeu, L-Agp, L-Ala(Bth), L-Arg(NO₂), L-Arg(Z)₂, L-Arg, L-Chg, L-Cys(Bzl), L-Dab(Z), L-Glu(OBzl), L-hArg, L-His(3-BOM), L-hTyr, L-Hyp, L-Idc, L-Lys(2-ClZ), L-Lys, L-Nle(OBzl), or L-Oic, L-Orn; Y is H, acetyl, tert-butyloxycarbonyl (Boc), benzyloxycarbonyl (Cbz), fluorenylmethyloxycarbonyl (Fmoc), benzyl, —C(O)R, —SOOR, —COOR, —C(O)NHR, —COCH(NHC(O)CH₃)—(CH₂)ₓ—NHC(O)—(CH₂)ₓ-p-halo-phenyl, substituted or unsubstituted —(CH₂)ₓaryl, substituted or unsubstituted —(CH₂)ₓheteroaryl, substituted or unsubstituted —(CH₂)ₓcycloalkyl, or substituted or unsubstituted —(CH₂)ₓheterocycle; each x is independently an integer of 0, 1, 2, 3, or 4; each R is independently C₁ to C₆ alkyl, C₃ to C₆ cycloalkyl, heterocycle, alkylheterocycle, aralkyl, or aryl; each Z is independently

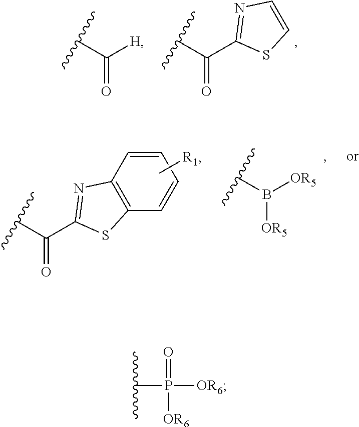

R₁ is hydrogen,

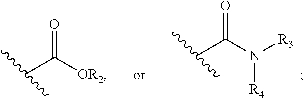

R₂ and R₃ are each independently hydrogen, substituted or unsubstituted alkyl, substituted or unsubstituted cycloalkyl, substituted or unsubstituted aryl, substituted or unsubstituted aralkyl, substituted or unsubstituted heteroaryl, or substituted or unsubstituted heteroarylalkyl; R₄ is hydrogen, substituted or unsubstituted alkyl, or a residue of an amino acid, or R₃ and R₄ can form a ring; each R₅ is independently hydrogen, substituted or unsubstituted alkyl, or the R₅ moieties can form a ring; and each R₆ is substituted or unsubstituted aryl.

Yet another aspect of the disclosure is a compound of Formula II, a pharmaceutically acceptable salt thereof, or a stereoisomer thereof:

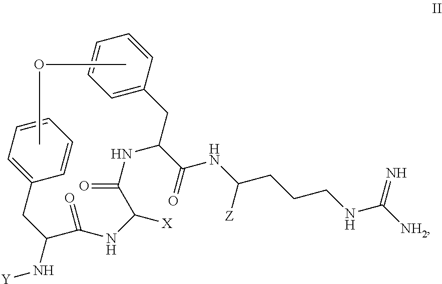

wherein X is a side chain of a natural or unnatural amino acid; Y is H, acetyl, tert-butyloxycarbonyl (Boc), benzyloxycarbonyl (Cbz), fluorenylmethyloxycarbonyl (Fmoc), benzyl, —C(O)R, —SOOR, —COOR, —C(O)NHR, —COCH(NHC(O)CH₃)—(CH₂)ₓ—NHC(O)—(CH₂)ₓ-p-halo-phenyl, substituted or unsubstituted —(CH₂)ₓaryl, substituted or unsubstituted —(CH₂)ₓheteroaryl, substituted or unsubstituted —(CH₂)ₓcycloalkyl, or substituted or unsubstituted —(CH₂)ₓheterocycle; each R is independently C₁ to C₆ alkyl, C₃ to C₆ cycloalkyl, heterocycle, alkylheterocycle, aralkyl, or aryl; and Z is independently

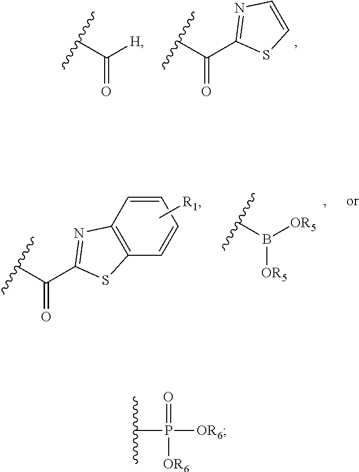

R₁ is hydrogen,

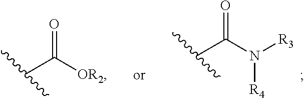

R₂ and R₃ are each independently hydrogen, substituted or unsubstituted alkyl, substituted or unsubstituted cycloalkyl, substituted or unsubstituted aryl, substituted or unsubstituted aralkyl, substituted or unsubstituted heteroaryl, or substituted or unsubstituted heteroarylalkyl; R₄ is hydrogen, substituted or unsubstituted alkyl, or a residue of an amino acid, or R₃ and R₄ can form a ring; R₅ is independently hydrogen, substituted or unsubstituted alkyl, or the R₅ moieties can form a ring; and R₆ is substituted or unsubstituted aryl.

A further aspect of the disclosure is a methods of treatment of various conditions including cancer and viral infections by administering the compounds of Formulae IA, IB, IC, and II to a patient in need thereof.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION OF INVENTION

The present disclosure is based, at least in part, on the discovery of macrocyclic peptides and peptides containing unnatural amino acids as inhibitors of TMPRSS2, matriptase, hepsin, HGFA, TMPRSS13, TMPRSS11D (HAT), and other serine proteases that have potent anticancer and antiviral activity.

As shown herein, the use of PS-SCL and HyCoSuL and unnatural amino acids in the rational design of selective serine protease inhibitors, including the application to HGFA, matriptase and hepsin, is described. Further, the rational design of mechanism-based TMPRSS2 serine protease inhibitors from PS-SCL and HyCoSuL substrate specificity screening is also described. Additionally, macrocyclic inhibitors of serine proteases, and the effect of ring size, orientation, and different cyclic peptide linkers on potency, selectivity, and ADME properties are also described.

One aspect of the present disclosure provides for compositions of serine protease inhibitors and related therapeutic methods.

### Serine Protease Modulation Agents

As described herein, serine protease expression has been implicated in various diseases, disorders, and conditions. As such, modulation of serine proteases (e.g., modulation of a serine protease with peptides) can be used for the treatment of such conditions. A serine protease modulation agent can modulate serine protease response or induce or inhibit a serine protease. Serine protease modulation can comprise modulating the expression of serine proteases in cells, modulating the quantity of cells that express a serine protease, or modulating the quality of the serine protease-expressing cells.

Serine protease modulation agents can be any composition or method that can modulate serine protease expression on cells. For example, a serine protease modulation agent can be an activator, an inhibitor, an agonist, or an antagonist. As another example, serine protease modulation can be the result of gene editing.

A serine protease modulation agent can be an anti-serine protease antibody (e.g., a monoclonal antibody to a serine protease).

A serine protease modulating agent can be an agent that induces or inhibits progenitor cell differentiation into serine protease-expressing cells. For example, peptidomimetic molecules can be used to block HGFA, TMPRSS2, matriptase, or hepsin.

Serine Protease Signal Reduction, Elimination, or Inhibition by Small Molecule Inhibitors, shRNA, siRNA, or ASOs

As described herein, a serine protease modulation agent can be used for use in cancer or viral therapy. A serine protease modulation agent can be used to reduce/eliminate or enhance/increase serine protease signals. For example, a serine protease modulation agent can be a small molecule inhibitor of a serine protease. As another example, a serine protease modulation agent can be a short hairpin RNA (shRNA). As another example, a serine protease modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

### Serine Protease Inhibiting Agent

One aspect of the present disclosure provides for the targeting of a serine protease, its receptor, or its downstream signaling. The present disclosure provides methods of treating or preventing cancer or a viral infection based on the discovery that serine protease inhibitors show efficacy in the treatment of both cancer and viral infection.

As described herein, inhibitors of a serine protease (e.g., antibodies, fusion proteins, small molecules) can reduce or prevent cancer or a viral infection. A serine protease inhibiting agent can be any agent that can inhibit a serine protease, downregulate a serine protease, or knockdown a serine protease.

As an example, a serine protease inhibiting agent can inhibit serine protease signaling.

For example, the serine protease inhibiting agent can be an anti-serine protease antibody. As an example, the anti-serine protease antibody can be an anti-TMPRSS2 antibody, an anti-matriptase antibody, an anti-hepsin antibody, an anti-HGFA antibody, an anti-TMPRSS13 antibody or another serine protease inhibitor or an anti-serine protease antibody with activity against any combination of TMPRSS2, matriptase, hepsin, HGFA, TMPRSS13, TMPRSS11D (HAT), and other serine proteases. Furthermore, the anti-serine protease antibody can be a murine antibody, a humanized murine antibody, or a human antibody.

As another example, the serine protease inhibiting agent can be an anti-serine protease antibody, wherein the anti-serine protease antibody prevents the binding of a serine protease to its receptor or prevents activation of a serine protease and downstream signaling.

As another example, the serine protease inhibiting agent can be a fusion protein. For example, the fusion protein can be a decoy receptor for a serine protease. Furthermore, the fusion protein can comprise a mouse or human Fc antibody domain fused to the ectodomain of a serine protease.

As another example, a serine protease inhibiting agent can be a peptidomimetic molecule, which has been shown to be a potent and specific inhibitor of serine protease signaling.

As another example, a serine protease inhibiting agent can be an inhibitory protein that antagonizes a serine protease. For example, the serine protease inhibiting agent can be a viral protein, which has been shown to antagonize a serine protease.

As another example, a serine protease inhibiting agent can be a short hairpin RNA (shRNA) or a short interfering RNA (siRNA) targeting a serine protease.

As another example, a serine protease inhibiting agent can be an sgRNA targeting a serine protease.

Methods for preparing a serine protease inhibiting agent (e.g., an agent capable of inhibiting serine protease signaling) can comprise the construction of a protein/Ab scaffold containing the natural serine protease receptor as a serine protease neutralizing agent; developing inhibitors of the serine protease receptor “down-stream”; or developing inhibitors of the serine protease production “up-stream”.

Inhibiting a serine protease can be performed by genetically modifying a serine protease in a subject or genetically modifying a subject to reduce or prevent expression of the serine protease gene, such as through the use of CRISPR-Cas9 or analogous technologies, wherein, such modification reduces or prevents cancer or a viral infection.

### Chemical Agent:

Examples of serine protease modulation agents are described herein.

An aspect of the disclosure is the description of compounds of Formula (IA), (IB), or (IC), a pharmaceutically acceptable salt thereof, or a stereoisomer thereof:

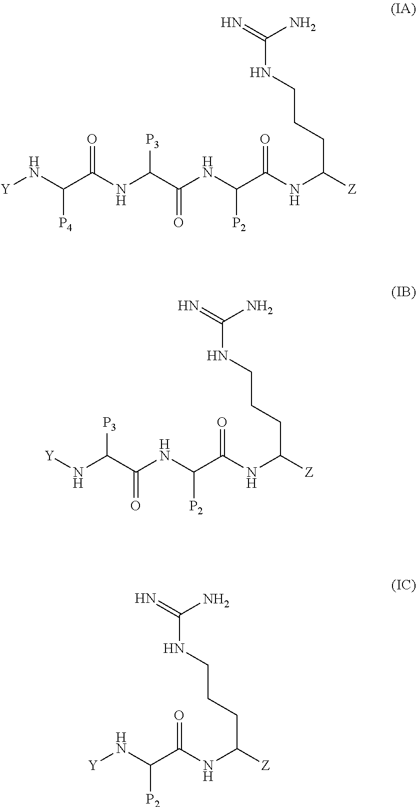

wherein each P₂ is independently a side chain of 1-Nal, 2-Nal, Gly(2-th), D-Ala, L-Ala, L-Ala(2-th), L-Arg, L-Arg(Z)₂, L-Asn, L-Bla, L-Cha, L-Chg, L-Glu(OBzl), L-hArg, L-hCha, L-His(Bzl), L-hLeu, L-hPhe, L-hTyr, L-hTyr(Me), L-Igl, L-Leu, L-Lys, L-Lys(2-ClZ), L-Nle, L-Nle(OBzl), L-NptGly, L-Nva, L-Orn, L-Phe, L-Phe(3,4-F₂), L-Phe(3-Cl), L-Phe(3-F), L-Phe(4-F), L-phg, L-Ser, L-Thr, L-Trp, or L-Val; each P₃ is independently a side chain of 3-AMPA, 4-AMBA, 4-AMPA, D-Arg, D-Gln, D-Lys, D-Phg, D-Ser, D-Trp, L-2-Aoc, L-Abu(Bth), L-Agb, L-Agp, L-Ala(Bth), L-Arg, L-Arg(Z)₂, L-Asp(OCHx), L-Dab, L-Dap, L-Dht, L-Gln, L-Glu, L-Glu(All), L-Glu(Bzl), L-Glu(Obzl), L-Glu(OCHx), L-Glu(OMe), L-hArg, L-hCha, L-His(3-Bom), L-hPhe, L-hTyr, L-Igl, L-Leu, L-Lys, L-Lys(2-ClZ), L-Met, L-Met(O), L-Met(O)₂, L-Nle(OBzl), L-Orn, L-Phe(4-NO₂), L-Phe(F₅), L-Ser, L-Ser(Ac), L-Thr, or L-Trp; each P₄ is independently a side chain of 2-Abz, 3-Abz, 4-Abz, D-Arg, dhAbu, dhLeu, D-Lys, D-Trp, Gly, L-2-Aoc, L-Agb, L-Agp, L-Ala(Bth), L-Arg, L-Arg(NO₂), L-Arg(Z)₂, L-Chg, L-Cys(4-MeOBzl), L-Cys(Bzl), L-Cys(MeBzl), L-DAB(Z), L-Glu(OBzl), L-Glu(OCHx), L-hArg, L-hCha, L-His(3-Bom), L-hLeu, L-hPhe, L-hTyr, L-Hyp, L-Hyp(Bzl), L-Idc, L-Ile, L-Leu, L-Lys, L-Lys(2-Cl-Z), L-Lys(TFA), L-Met, L-Nle, L-Nle(OBzl), L-Nva, L-Oic, L-Orn, L-Phe, L-Phe(4-I), L-Phe(F₅), L-Pro, L-Ser, L-Ser(Bzl), L-Thr(Bzl), L-Trp, or TmbGly; Y is H, acetyl, tert-butyloxycarbonyl (Boc), benzyloxycarbonyl (Cbz), fluorenylmethyloxycarbonyl (Fmoc), benzyl, —C(O)R, —SOOR, —COOR, —C(O)NHR, —COCH(NHC(O)CH₃)—(CH₂)ₓ—NHC(O)—(CH₂)ₓ-p-halo-phenyl, substituted or unsubstituted —(CH₂)ₓaryl, substituted or unsubstituted —(CH₂)ₓheteroaryl, substituted or unsubstituted —(CH₂)ₓcycloalkyl, or substituted or unsubstituted —(CH₂)ₓheterocycle; each x is independently an integer of 0, 1, 2, 3, or 4; each R is independently C₁ to C₆ alkyl, C₃ to C₆ cycloalkyl, heterocycle, alkylheterocycle, aralkyl, or aryl; each Z is independently

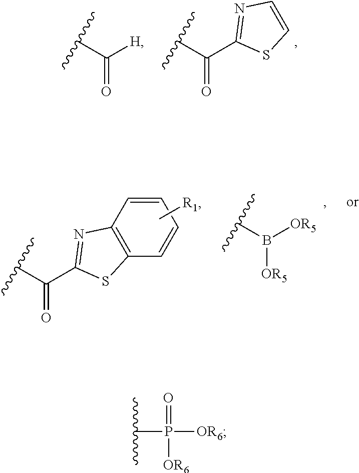

R₁ is hydrogen,

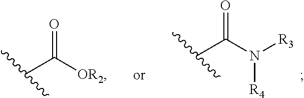

R₂ and R₃ are each independently hydrogen, substituted or unsubstituted alkyl, substituted or unsubstituted cycloalkyl, substituted or unsubstituted aryl, substituted or unsubstituted aralkyl, substituted or unsubstituted heteroaryl, or substituted or unsubstituted heteroarylalkyl; R₄ is hydrogen, substituted or unsubstituted alkyl, or a residue of an amino acid, or R₃ and R₄ can form a ring; each R₅ is independently hydrogen, substituted or unsubstituted alkyl, or the R₅ moieties can form a ring; and each R₆ is substituted or unsubstituted aryl.

For the compounds of Formulae IA, IB, and IC, each P₂ can independently be a side chain of 1-Nal, 2-Nal, Gly(2-th), L-Ala, L-Ala(2-th), L-Bla, L-Cha, L-Glu(OBzl), L-hCha, L-His(Bzl), L-hPhe, L-hTyr, L-Igl, L-Lys(2-ClZ), L-Nle, L-Nle(OBzl), L-NptGly, L-Nva, L-Orn, L-Phe(3,4-F₂), L-Phe(3-Cl), L-Phe(3-F), or L-Phe(4-F).

Additionally, for the compounds of Formulae IA, IB, and IC, each P₂ can independently be a side chain of 1-Nal, 2-Nal, Gly(2-th), L-Ala, L-Ala(2-th), L-Bla, L-Glu(OBzl), L-hCha, L-His(Bzl), L-hPhe, L-hTyr, L-Lys(2-ClZ), L-Nle, L-Nle(OBzl), L-Nva, L-Phe(3-F).

The compounds of Formulae IA, IB, and IC can have each P₃ independently be a side chain of 3-AMPA, 4-AMBA, 4-AMPA, D-Arg, D-Lys, D-Phg, D-Ser, L-2-Aoc, L-Abu(Bth), L-Agb, L-Ala(Bth), L-Arg(Z)₂, L-Asp(OCHx), L-Dab, L-Dap, L-Dht, L-Glu, L-Glu(All), L-Glu(Bzl), L-Glu(Obzl), L-Glu(OCHx), L-Glu(OMe), L-Igl, L-Leu, L-Lys(2-ClZ), L-Met, L-Met(O), L-Met(O)₂, L-Phe(F₅), L-Ser, L-Ser(Ac), or L-Thr.

For the compounds of Formulae IA, IB, and IC, each P₃ can independently be a side chain of 4-AMBA, L-Glu, L-Glu(All), L-Glu(OCHx), L-Met, L-Ser, or L-Thr.

Also, the compounds of Formulae IA, IB, and IC can have each P₄ independently be a side chain of 2-Abz, 3-Abz, 4-Abz, D-Arg, dhAbu, dhLeu, D-Lys, Gly, L-2-Aoc, L-Agp, L-Ala(Bth), L-Chg, L-Cys(4-MeOBzl), L-Cys(Bzl), L-Cys(MeBzl), L-Glu(OBzl), L-Glu(OCHx), L-hCha, L-hLeu, L-hPhe, L-hTyr, L-Hyp, L-Hyp(Bzl), L-Idc, L-Lys(TFA), L-Nle, L-Nva, L-Oic, L-Phe, L-Phe(4-I), L-Phe(F₅), L-Ser(Bzl), L-Thr(Bzl), or TmbGly.

The compounds of Formulae IA, IB, and IC can have each P₄ independently be a side chain of dhLeu, Gly, L-Idc, L-Ile, L-Leu, L-Met, L-Oic, or L-Pro.

The compounds of Formulae IA, IB, and IC can also have each P₄ is independently a side chain of dhLeu, Gly, L-Idc, or L-Oic.

The compounds of Formula IC can have P₂ be a side chain of 1-Nal, 2-Nal, Gly(2-th), D-Ala, L-Ala, L-Ala(2-th), L-Arg, L-Arg(Z)₂, L-Asn, L-Bla, L-Cha, L-Chg, L-Glu(OBzl), L-hArg, L-hCha, L-His(Bzl), L-hLeu, L-hPhe, L-hTyr, L-hTyr(Me), L-Igl, L-Leu, L-Lys, L-Lys(2-ClZ), L-Nle, L-Nle(OBzl), L-NptGly, L-Nva, L-Orn, L-Phe, L-Phe(3,4-F₂), L-Phe(3-Cl), L-Phe(3-F), L-Phe(4-F), L-phg, L-Ser, L-Thr, L-Trp, or L-Val.

Additionally, the compounds of Formula IC can have P₂ be a side chain of 1-Nal, 2-Nal, Gly(2-th), L-Ala, L-Ala(2-th), L-Bla, L-Cha, L-Glu(OBzl), L-hCha, L-His(Bzl), L-hPhe, L-hTyr, L-Igl, L-Lys(2-ClZ), L-Nle, L-Nle(OBzl), L-NptGly, L-Nva, L-Orn, L-Phe(3,4-F₂), L-Phe(3-Cl), L-Phe(3-F), or L-Phe(4-F).

Further, the compounds of Formula IC can have P₂ be a side chain of 1-Nal, 2-Nal, Gly(2-th), L-Ala, L-Ala(2-th), L-Bla, L-Glu(OBzl), L-hCha, L-His(Bzl), L-hPhe, L-hTyr, L-Lys(2-ClZ), L-Nle, L-Nle(OBzl), L-Nva, L-Phe(3-F).

The compounds of Formula IB can have P₂ be a side chain of 1-Nal, 2-Nal, Gly(2-th), D-Ala, L-Ala, L-Ala(2-th), L-Arg, L-Arg(Z)₂, L-Asn, L-Bla, L-Cha, L-Chg, L-Glu(OBzl), L-hArg, L-hCha, L-His(Bzl), L-hLeu, L-hPhe, L-hTyr, L-hTyr(Me), L-Igl, L-Leu, L-Lys, L-Lys(2-ClZ), L-Nle, L-Nle(OBzl), L-NptGly, L-Nva, L-Orn, L-Phe, L-Phe(3,4-F₂), L-Phe(3-Cl), L-Phe(3-F), L-Phe(4-F), L-phg, L-Ser, L-Thr, L-Trp, or L-Val and can have P₃ be a side chain of 3-AMPA, 4-AMBA, 4-AMPA, D-Arg, D-Gln, D-Lys, D-Phg, D-Ser, D-Trp, L-2-Aoc, L-Abu(Bth), L-Agb, L-Agp, L-Ala(Bth), L-Arg, L-Arg(Z)₂, L-Asp(OCHx), L-Dab, L-Dap, L-Dht, L-Gln, L-Glu, L-Glu(All), L-Glu(Bzl), L-Glu(Obzl), L-Glu(OCHx), L-Glu(OMe), L-hArg, L-hCha, L-His(3-Bom), L-hPhe, L-hTyr, L-Igl, L-Leu, L-Lys, L-Lys(2-ClZ), L-Met, L-Met(O), L-Met(O)₂, L-Nle(OBzl), L-Orn, L-Phe(4-NO₂), L-Phe(F₅), L-Ser, L-Ser(Ac), L-Thr, or L-Trp.

Additionally, the compounds of Formula IB can have P₂ be a side chain of 1-Nal, 2-Nal, Gly(2-th), L-Ala, L-Ala(2-th), L-Bla, L-Cha, L-Glu(OBzl), L-hCha, L-His(Bzl), L-hPhe, L-hTyr, L-Igl, L-Lys(2-ClZ), L-Nle, L-Nle(OBzl), L-NptGly, L-Nva, L-Orn, L-Phe(3,4-F₂), L-Phe(3-Cl), L-Phe(3-F), or L-Phe(4-F) and P₃ can be a side chain of 3-AMPA, 4-AMBA, 4-AMPA, D-Arg, D-Lys, D-Phg, D-Ser, L-2-Aoc, L-Abu(Bth), L-Agb, L-Ala(Bth), L-Arg(Z)₂, L-Asp(OCHx), L-Dab, L-Dap, L-Dht, L-Glu, L-Glu(All), L-Glu(Bzl), L-Glu(Obzl), L-Glu(OCHx), L-Glu(OMe), L-Igl, L-Leu, L-Lys(2-ClZ), L-Met, L-Met(O), L-Met(O)₂, L-Phe(F₅), L-Ser, L-Ser(Ac), or L-Thr.

Further, the compounds of Formula IB can have P₂ be a side chain of 1-Nal, 2-Nal, Gly(2-th), L-Ala, L-Ala(2-th), L-Bla, L-Glu(OBzl), L-hCha, L-His(Bzl), L-hPhe, L-hTyr, L-Lys(2-ClZ), L-Nle, L-Nle(OBzl), L-Nva, L-Phe(3-F) and P₃ can be a side chain of 4-AMBA, L-Glu, L-Glu(All), L-Glu(OCHx), L-Met, L-Ser, or L-Thr.

The compounds of Formula IA can have P₂ be a side chain of 1-Nal, 2-Nal, Gly(2-th), D-Ala, L-Ala, L-Ala(2-th), L-Arg, L-Arg(Z)₂, L-Asn, L-Bla, L-Cha, L-Chg, L-Glu(OBzl), L-hArg, L-hCha, L-His(Bzl), L-hLeu, L-hPhe, L-hTyr, L-hTyr(Me), L-Igl, L-Leu, L-Lys, L-Lys(2-ClZ), L-Nle, L-Nle(OBzl), L-NptGly, L-Nva, L-Orn, L-Phe, L-Phe(3,4-F₂), L-Phe(3-Cl), L-Phe(3-F), L-Phe(4-F), L-phg, L-Ser, L-Thr, L-Trp, or L-Val; P₃ can be a side chain of 3-AMPA, 4-AMBA, 4-AMPA, D-Arg, D-Gln, D-Lys, D-Phg, D-Ser, D-Trp, L-2-Aoc, L-Abu(Bth), L-Agb, L-Agp, L-Ala(Bth), L-Arg, L-Arg(Z)₂, L-Asp(OCHx), L-Dab, L-Dap, L-Dht, L-Gln, L-Glu, L-Glu(All), L-Glu(Bzl), L-Glu(Obzl), L-Glu(OCHx), L-Glu(OMe), L-hArg, L-hCha, L-His(3-Bom), L-hPhe, L-hTyr, L-Igl, L-Leu, L-Lys, L-Lys(2-ClZ), L-Met, L-Met(O), L-Met(O)₂, L-Nle(OBzl), L-Orn, L-Phe(4-NO₂), L-Phe(F₅), L-Ser, L-Ser(Ac), L-Thr, or L-Trp; and P₄ can be a side chain of 2-Abz, 3-Abz, 4-Abz, D-Arg, dhAbu, dhLeu, D-Lys, D-Trp, Gly, L-2-Aoc, L-Agb, L-Agp, L-Ala(Bth), L-Arg, L-Arg(NO₂), L-Arg(Z)₂, L-Chg, L-Cys(4-MeOBzl), L-Cys(Bzl), L-Cys(MeBzl), L-DAB(Z), L-Glu(OBzl), L-Glu(OCHx), L-hArg, L-hCha, L-His(3-Bom), L-hLeu, L-hPhe, L-hTyr, L-Hyp, L-Hyp(Bzl), L-Idc, L-Ile, L-Leu, L-Lys, L-Lys(2-Cl-Z), L-Lys(TFA), L-Met, L-Nle, L-Nle(OBzl), L-Nva, L-Oic, L-Orn, L-Phe, L-Phe(4-I), L-Phe(F₅), L-Pro, L-Ser, L-Ser(Bzl), L-Thr(Bzl), L-Trp, or TmbGly.

Additionally, the compounds of Formula IA can have P₂ be a side chain of 1-Nal, 2-Nal, Gly(2-th), L-Ala, L-Ala(2-th), L-Bla, L-Cha, L-Glu(OBzl), L-hCha, L-His(Bzl), L-hPhe, L-hTyr, L-Igl, L-Lys(2-ClZ), L-Nle, L-Nle(OBzl), L-NptGly, L-Nva, L-Orn, L-Phe(3,4-F₂), L-Phe(3-Cl), L-Phe(3-F), or L-Phe(4-F); P₃ can be a side chain of 3-AMPA, 4-AMBA, 4-AMPA, D-Arg, D-Lys, D-Phg, D-Ser, L-2-Aoc, L-Abu(Bth), L-Agb, L-Ala(Bth), L-Arg(Z)₂, L-Asp(OCHx), L-Dab, L-Dap, L-Dht, L-Glu, L-Glu(All), L-Glu(Bzl), L-Glu(Obzl), L-Glu(OCHx), L-Glu(OMe), L-Igl, L-Leu, L-Lys(2-ClZ), L-Met, L-Met(O), L-Met(O)₂, L-Phe(F₅), L-Ser, L-Ser(Ac), or L-Thr; and P₄ can be a side chain of 2-Abz, 3-Abz, 4-Abz, D-Arg, dhAbu, dhLeu, D-Lys, Gly, L-2-Aoc, L-Agp, L-Ala(Bth), L-Chg, L-Cys(4-MeOBzl), L-Cys(Bzl), L-Cys(MeBzl), L-Glu(OBzl), L-Glu(OCHx), L-hCha, L-hLeu, L-hPhe, L-hTyr, L-Hyp, L-Hyp(Bzl), L-Idc, L-Lys(TFA), L-Nle, L-Nva, L-Oic, L-Phe, L-Phe(4-I), L-Phe(F₅), L-Ser(Bzl), L-Thr(Bzl), or TmbGly.

Further, the compounds of Formula IA can have P₂ be a side chain of 1-Nal, 2-Nal, Gly(2-th), L-Ala, L-Ala(2-th), L-Bla, L-Glu(OBzl), L-hCha, L-His(Bzl), L-hPhe, L-hTyr, L-Lys(2-ClZ), L-Nle, L-Nle(OBzl), L-Nva, L-Phe(3-F); P₃ can be a side chain of 4-AMBA, L-Glu, L-Glu(All), L-Glu(OCHx), L-Met, L-Ser, or L-Thr; and P₄ can be a side chain of dhLeu, Gly, L-Idc, L-Ile, L-Leu, L-Met, L-Oic, or L-Pro.

Another aspect of the disclosure is a compound of Formula (IA), (IB), or (IC), a pharmaceutically acceptable salt thereof, or a stereoisomer thereof:

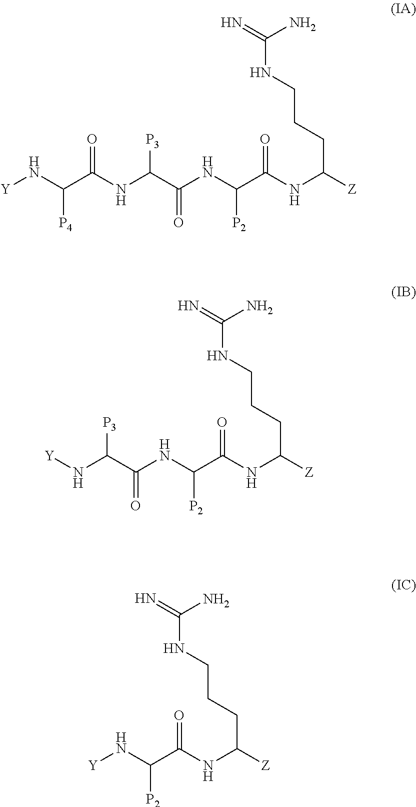

wherein each P₂ is independently a side chain of L-Ala(2-th), L-Arg(Z)₂, L-Asn, L-Bta, L-Cha, L-Chg, L-Glu(OBzl), L-hArg, L-hCha, L-His(Bzl), L-hLeu, L-hPhe, L-hTyr(Me), L-hTyr, L-Igl, L-Leu, L-Lys(2-ClZ), L-Nle(OBzl), L-Nle, L-NptGly, L-Nva, L-Orn, L-Phe(3,4-F2), L-Phe(3-Cl), L-Phe(3-F), L-Phe(4-F), L-Phe, L-Thr, or L-Trp; each P₃ is independently a side chain of D-Arg, D-Gln, D-Lys, D-Trp, L-2-Aoc, L-Agp, L-Ala(Bth), L-Arg(Z)₂, L-Arg, L-Dab, L-Dap, L-Dht, L-Glu(All), L-Glu(OBzl), L-Glu(OCHx), L-Glu(OMe), L-hArg, L-hCha, L-hPhe, L-hTyr, L-Igl, L-Lys, L-Met(O), L-Met(O)₂, L-Nle(OBzl), L-Orn, L-Phe(F₅), or L-Ser(Ac); each P₄ is independently a side chain of 4-Abz, chAbu, D-Arg, dhAbu, dhLeu, L-Agp, L-Ala(Bth), L-Arg(NO₂), L-Arg(Z)₂, L-Arg, L-Chg, L-Cys(Bzl), L-Dab(Z), L-Glu(OBzl), L-hArg, L-His(3-BOM), L-hTyr, L-Hyp, L-Idc, L-Lys(2-ClZ), L-Lys, L-Nle(OBzl), or L-Oic, L-Orn; Y is H, acetyl, tert-butyloxycarbonyl (Boc), benzyloxycarbonyl (Cbz), fluorenylmethyloxycarbonyl (Fmoc), benzyl, —C(O)R, —SOOR, —COOR, —C(O)NHR, —COCH(NHC(O)CH₃)—(CH₂)ₓ—NHC(O)—(CH₂)ₓ-p-halo-phenyl, substituted or unsubstituted —(CH₂)ₓaryl, substituted or unsubstituted —(CH₂)ₓheteroaryl, substituted or unsubstituted —(CH₂)ₓcycloalkyl, or substituted or unsubstituted —(CH₂)ₓheterocycle; each x is independently an integer of 0, 1, 2, 3, or 4; each R is independently C₁ to C₆ alkyl, C₃ to C₆ cycloalkyl, heterocycle, alkylheterocycle, aralkyl, or aryl; each Z is independently

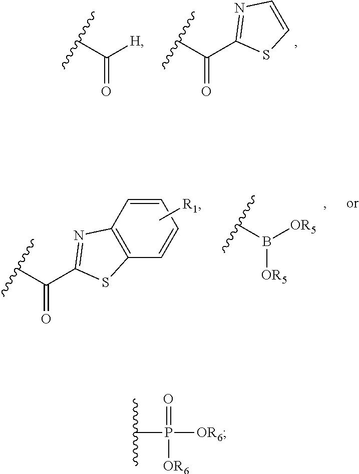

R₁ is hydrogen,

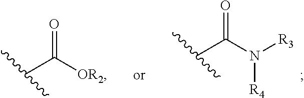

R₂ and R₃ are each independently hydrogen, substituted or unsubstituted alkyl, substituted or unsubstituted cycloalkyl, substituted or unsubstituted aryl, substituted or unsubstituted aralkyl, substituted or unsubstituted heteroaryl, or substituted or unsubstituted heteroarylalkyl; R₄ is hydrogen, substituted or unsubstituted alkyl, or a residue of an amino acid, or R₃ and R₄ can form a ring; each R₅ is independently hydrogen, substituted or unsubstituted alkyl, or the R₅ moieties can form a ring; and each R₆ is substituted or unsubstituted aryl.

The compounds of Formula IC can have P₂ be a side chain of L-Phe(4-F), L-Ph3(3,4-F2), L-Phe, L-Phe(3-Cl), L-His(Bzl), L-Phe(3-F), L-Glu(OBzl), L-Nle(OBzl), L-Ala(2-th), or L-Trp.

The compounds of Formula IC can have P₂ be a side chain of L-Phe(4-F), L-Ph3(3,4-F2), L-Phe, L-Phe(3-Cl), or L-His(Bzl).

The compounds of Formula IB can have P₂ be a side chain of L-Phe(4-F), L-Phe(3,4-F2), L-Phe, L-Phe(3-Cl), L-His(Bzl), L-Phe(3-F), L-Glu(OBzl), L-Nle(OBzl), L-Ala(2-th), or L-Trp; and P₃ be a side chain of L-Glu(OCHx), L-Glu(All), L-Glu(OBzl), L-Met(O)₂, L-hCha, L-Ser(Ac), L-2-Aoc, L-Nle(OBzl), L-Glu(Ome), or L-Ala(Bth).

The compounds of Formula IB can have P₂ be a side chain of L-Phe(4-F), L-Ph3(3,4-F2), L-Phe, L-Phe(3-Cl), or L-His(Bzl) and P₃ be a side chain of L-Glu(OCHx), L-Glu(All), L-Glu(OBzl), L-Met(O)₂, or L-hCha.

The compounds of Formula IA can have P₂ be a side chain of L-Phe(4-F), L-Ph3(3,4-F2), L-Phe, L-Phe(3-Cl), L-His(Bzl), L-Phe(3-F), L-Glu(OBzl), L-Nle(OBzl), L-Ala(2-th), or L-Trp; P₃ be a side chain of L-Glu(OCHx), L-Glu(All), L-Glu(OBzl), L-Met(O)₂, L-hCha, L-Ser(Ac), L-2-Aoc, L-Nle(OBzl), L-Glu(Ome), or L-Ala(Bth); and P₄ be a side chain of L-Oic, dhLeu, L-Chg, L-Lys(2-ClZ), 4-Abz, L-Idc, L-Ala(Bth), dhAbu, L-hTyr, or L-Cys(Bzl).

The compounds of Formula IA can have P₂ be a side chain of L-Phe(4-F), L-Ph3(3,4-F2), L-Phe, L-Phe(3-Cl), or L-His(Bzl); P₃ be a side chain of L-Glu(OCHx), L-Glu(All), L-Glu(OBzl), L-Met(O)₂, or L-hCha; and P₄ be a side chain of L-Oic, dhLeu, L-Chg, L-Lys(2-ClZ), or 4-Abz.

Alternatively, the compounds of Formula IC can have P₂ be a side chain of L-Igl, L-Phe(3,4-F₂), L-Phe(3-Cl), L-Phe(4-F), L-Glu(OBzl), L-Phe(3-F), L-Phe, L-hCha, L-Bta, or L-Trp.

The compounds of Formula IC can have P₂ be a side chain of L-Igl, L-Phe(3,4-F₂), L-Phe(3-Cl), L-Phe(4-F), or L-Glu(OBzl).

The compounds of Formula IB can have P₂ be a side chain of L-Igl, L-Phe(3,4-F₂), L-Phe(3-Cl), L-Phe(4-F), L-Glu(OBzl), L-Phe(3-F), L-Phe, L-hCha, L-Bta, or L-Trp and P₃ be a side chain of L-Agp, L-Lys, L-Nle(OBzl), L-Orn, L-Arg, L-Dab, L-Arg(Z)₂, L-Dap, L-Met(O), or L-Phe(F₅).

The compounds of Formula IB can have P₂ be a side chain of L-Igl, L-Phe(3,4-F₂), L-Phe(3-Cl), L-Phe(4-F), or L-Glu(OBzl) and P₃ be a side chain of L-Agp, L-Lys, L-Nle(OBzl), L-Orn, or L-Arg.

Also, the compounds of Formula IA can have P₂ be a side chain of L-Igl, L-Phe(3,4-F₂), L-Phe(3-Cl), L-Phe(4-F), L-Glu(OBzl), L-Phe(3-F), L-Phe, L-hCha, L-Bta, or L-Trp; P₃ be a side chain of L-Agp, L-Lys, L-Nle(OBzl), L-Orn, L-Arg, L-Dab, L-Arg(Z)₂, L-Dap, L-Met(O), or L-Phe(F₅); and P₄ be a side chain of L-Arg, L-hArg, L-Orn, L-Lys, L-Arg(Z)₂, L-Lys(2-ClZ), dhLeu, L-hTyr, L-Hyp, or L-Agp.

The compounds of Formula IA can have P₂ be a side chain of L-Igl, L-Phe(3,4-F₂), L-Phe(3-Cl), L-Phe(4-F), or L-Glu(OBzl); P₃ be a side chain of L-Agp, L-Lys, L-Nle(OBzl), L-Orn, or L-Arg; and P₄ be a side chain of L-Arg, L-hArg, L-Orn, L-Lys, or L-Arg(Z)₂.

Also, the compounds of Formula IC can have P₂ be a side chain of L-Leu, L-Orn, L-Cha, L-Thr, L-Asn, L-Nle, L-Nva, L-hArg, L-Arg(Z)₂.

Preferably, the compounds of Formula IC can have P₂ be a side chain of L-Leu, L-Orn, L-Cha, L-Thr, or L-Asn.

Also, the compounds of Formula IB can have P₂ be a side chain of L-Leu, L-Orn, L-Cha, L-Thr, L-Asn, L-Nle, L-Nva, L-hArg, L-Arg(Z)₂ and P₃ can be a side chain of D-Gln, L-Agp, L-Nle(OBzl), L-Lys, L-Orn, L-Arg, L-Met(O), D-Arg, L-Glu(OMe), or D-Lys.

Preferably, the compounds of Formula IB can have P₂ be a side chain of L-Leu, L-Orn, L-Cha, L-Thr, or L-Asn and P₃ be a side chain of D-Gln, L-Agp, L-Nle(OBzl), L-Lys, or L-Orn.

The compounds of Formula IA can have P₂ be a side chain of L-Leu, L-Orn, L-Cha, L-Thr, L-Asn, L-Nle, L-Nva, L-hArg, L-Arg(Z)₂; P₃ can be a side chain of D-Gln, L-Agp, L-Nle(OBzl), L-Lys, L-Orn, L-Arg, L-Met(O), D-Arg, L-Glu(OMe), or D-Lys; and P₄ can be a side chain of L-Agp, L-Dab(Z), L-Nle(OBzl), L-Orn, L-Arg(NO₂), L-Arg, L-Lys, L-Arg(Z)₂, L-Glu(OBzl), or L-hArg.

Preferably, the compounds of Formula IA can have P₂ be a side chain of L-Leu, L-Orn, L-Cha, L-Thr, or L-Asn; P₃ be a side chain of D-Gln, L-Agp, L-Nle(OBzl), L-Lys, or L-Orn; and P₄ be a side chain of L-Agp, L-Dab(Z), L-Nle(OBzl), L-Orn, or L-Arg(NO₂).

Further, the compounds of Formula IC can have P₂ be a side chain of L-Leu, L-hLeu, L-NptGly, L-Nle, L-hTyr, L-Nva, L-hPhe, L-Lys(2-ClZ), L-Chg, or L-hTyr(Me).

Preferably, the compounds of Formula IC can have P₂ be a side chain of L-Leu, L-hLeu, L-NptGly, L-Nle, or L-hTyr.

The compounds of Formula IB can have P₂ be a side chain of L-Leu, L-hLeu, L-NptGly, L-Nle, L-hTyr, L-Nva, L-hPhe, L-Lys(2-ClZ), L-Chg, or L-hTyr(Me) and P₃ be a side chain of L-hArg, D-Trp, L-Agp, L-hCha, L-hTyr, L-hPhe, L-Dht, L-Orn, L-Igl, or L-Lys.

Preferably, the compounds of Formula IB can have P₂ be a side chain of L-Leu, L-hLeu, L-NptGly, L-Nle, or L-hTyr and P₃ be a side chain of L-hArg, D-Trp, L-Agp, L-hCha, or L-hTyr.

The compounds of Formula IA can have P₂ be a side chain of L-Leu, L-hLeu, L-NptGly, L-Nle, L-hTyr, L-Nva, L-hPhe, L-Lys(2-ClZ), L-Chg, or L-hTyr(Me); P₃ be a side chain of L-hArg, D-Trp, L-Agp, L-hCha, L-hTyr, L-hPhe, L-Dht, L-Orn, L-Igl, or L-Lys; and P₄ be a side chain of L-His(3-BOM), L-Agp, L-Lys(2-ClZ), dhLeu, L-Idc, L-Chg, chAbu, L-Arg, D-Arg, or L-hArg.

Preferably, the compounds of Formula IA can have P₂ be a side chain of L-Leu, L-hLeu, L-NptGly, L-Nle, or L-hTyr; P₃ be a side chain of L-hArg, D-Trp, L-Agp, L-hCha, or L-hTyr; and P₄ be a side chain of L-His(3-BOM), L-Agp, L-Lys(2-ClZ), dhLeu, or L-Idc.

The compounds of Formulae IA and IB can have Y be acetyl, benzyl-SO₂, tert-butyloxycarbonyl, benzyl, benzyloxycarbonyl, cyclopropyl-SO₂, fluorenylmethyloxycarbonyl, or benzylcarbonyl.

The compounds of Formula IC can have Y be acetyl or benzyloxycarbonyl (Cbz).

The compounds of Formulae IA, IB, and IC can have Y having the structure of

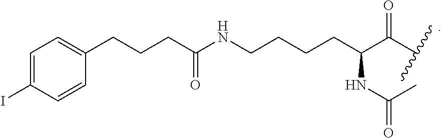 [SMILES: *C(=O)[C@H](CCCCNC(=O)CCCc1ccc(I)cc1)NC(C)=O]

Additionally, the compounds of Formulae IA, IB, and IC can have each x be an integer of 2, 3, or 4.

The compounds of Formulae IA, IB, and IC can have each R independently be methyl, ethyl propyl, cyclopropyl, cyclohexyl, benzyl, or phenyl.

The compounds of Formulae IA, IB, and IC can have Z be

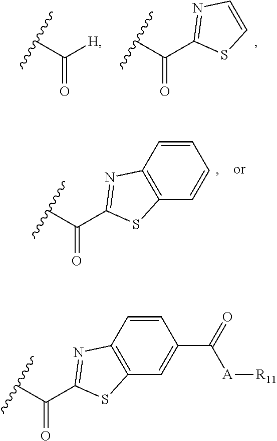

wherein A is —O— or NH, and R₁ is H, methyl, benzyl, optionally substituted alkyl, optionally substituted aryl, heterocycle, or a residue of an α-amino acid.

Preferably, the compounds of Formulae IA, IB, and IC have Z of

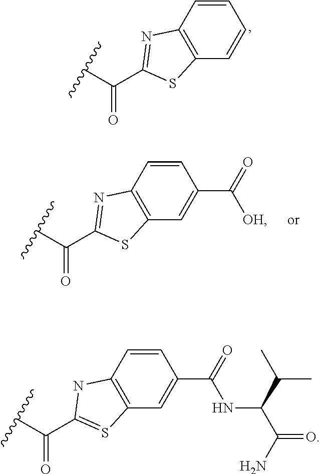

The compounds of Formula IA, IB, or IC having the following structure:

| | |
|---|---|
| PK-1-102A1 | Ac-WFR-kbt |
| (MN1066) | |
| MF1064 (YK) | Ac-OicGlu(OChx)Phe(4-F)Arg-kbt |
| MF1140 | Ac-dhLeu-Glu(OAII)-Phe(4-F)-Arg-kbt |
| MM4009-2 | Ac-ISFR-kbt |
| MF1068 (MPM) | Ac-OicGlu(OAII)Phe(4-F)Arg-kbt |
| ZFH9141 | Cbz-QFR-kbt |
| PK-1-93 (VB) | Ac-dWFR-kbt-COOH |
| PK-1-103 | Ac-WFR-kbt-COOH |
| (MM4095) | |
| CA1043 | Bz-QFR-kbt |
| (MF1017) | |
| CA1018 | PhAc-QFR-kbt |
| MF1105 | BnSO2-QFR-kbt |
| MM3180-2 | Ac-IQVR-kbt |
| MF1101 | PhSO2-QFR-kbt |
| VD5076B | Ac-Cyclo(F(para)LY)-R-kbt |
| MF1125 | Ac-dhLeu-Glu(OChx)-Phe(4-F)-Arg-kbt |
| MF1065 (MLS) | Ac-IdcGlu(OChx)Phe(4-F)Arg-kbt |
| MM3187 | Ac-IQWR-kbt |
| MF1104 | CypSO2-QFR-kbt |
| MM4037-2 | Ac-IEFR-kbt |
| MM3178 | Ac-IQAR-kbt |
| MM4028-2 | Ac-ITFR-kbt |
| MM3130 | Ac-MQFR-kbt |
| MF1142 | Fmoc-Cha-Arg-kbt |
| MM3131 | Ac-LQFR-kbt |
| CA1022-2 | Bn-QFR-kbt |
| CA1033-2 | PhEt-QFR-kbt |
| MM3186 | Ac-IQTR-kbt |
| MM4038 | Ac-IdWFR-kbt |
| MM3177 | Ac-IQSR-kbt |
| MM4027-2 | Ac-IMFR-kbt |
| CA1041-2 | Me2-QFR-kbt |
| PK-1-91 | Ac-dWLR-kbt |
| (MN1063) | |
| AJS4016 | Ac-Ser-4-AMBA-Leu-Arg-kbt |
| MF1163 | Fmoc-hTyr-Arg-kbt |
| MF1067B | Ac-IdcGlu(OAII)Phe(4-F)Arg-kbt |
| (MPM2082B) | |
| MF1177 | Fmoc-Gly(2-th)-Arg-kbt |
| MF1168 | Cbz-Bta-Arg-kbt |
| MM3194A | Ac-PSKR-kbt |
| MF2008 | Cbz-Igl-Arg-kbt |
| MM4037-1 | Ac-IEFdR-kbt |
| MF1184 | Fmoc-Ala(2-th)-Arg-kbt |
| MF1165 | Fmoc-Nle(OBzl)-Arg-kbt |
| PK-1-104 | Ac-WLR-kbt |
| (MN1070) | |
| PK-1-105 | Ac-WLR-kbt-COOH |
| (MM4094) | |
| PK-1-94 | Ac-dWLR-kbt-COOH |
| (MM4123) | |
| MF1134 | Fmoc-Phe(3-Cl)-Arg-kbt |
| CA1046-2 | Pip-QFR-kbt |
| MF1141 | Fmoc-Phe(3-F)-Arg-kbt |
| MF1131 | Fmoc-Phe(4-F)-Arg-kbt |
| MF1143 | Fmoc-Nle-Arg-kbt |
| MF1150 | Fmoc-Nva-Arg-kbt |
| MF1132 | Fmoc-Phe(3,4 di-F)-Arg-kbt |
| MF1158 | Fmoc-His(Bzl)-Arg-kbt |
| MF1169 | Cbz-Glu(OBzl)-Arg-kbt |
| MF2012 | Fmoc-2-NaI-Arg-kbt |
| MF1152 | Fmoc-hPhe-Arg-kbt |
| MF1151 | Fmoc-Lys(2-ClZ)-Arg-kbt |
| MF1164 | Fmoc-hLeu-Arg-kbt |
| MF2009 | Cbz-NptGly-Arg-kbt |
| MF2011 | Fmoc-Orn-Arg-kbt |
| MF1149 | Fmoc-hCha-Arg-kbt |
| MF1144 | Fmoc-1-NaI-Arg-kbt. |

Additionally, the compounds of Formula IA, IB, or IC having the following structure:

| | |
|---|---|
| PK-1-102A1 | Ac-WFR-kbt |
| (MN1066) | |
| MF1064 (YK) | Ac-OicGlu(OChx)Phe(4-F)Arg-kbt |
| MF1140 | Ac-dhLeu-Glu(OAII)-Phe(4-F)-Arg-kbt |
| MM4009-2 | Ac-ISFR-kbt |
| MF1068 (MPM) | Ac-OicGlu(OAll)Phe(4-F)Arg-kbt |
| ZFH9141 | Cbz-QFR-kbt |
| PK-1-93 (VB) | Ac-dWFR-kbt-COOH |
| PK-1-103 | Ac-WFR-kbt-COOH |
| (MM4095) | |
| CA1043 | Bz-QFR-kbt |
| (MF1017) | |
| CA1018 | PhAc-QFR-kbt |
| MF1105 | BnSO2-QFR-kbt |
| MM3180-2 | Ac-IQVR-kbt |
| MF1101 | PhSO2-QFR-kbt |
| MF1125 | Ac-dhLeu-Glu(OChx)-Phe(4-F)-Arg-kbt |
| MF1065 (MLS) | Ac-IdcGlu(OChx)Phe(4-F)Arg-kbt |
| MM3187 | Ac-IQWR-kbt |
| MF1104 | CypSO2-QFR-kbt |
| MM4037-2 | Ac-IEFR-kbt |
| MM3178 | Ac-IQAR-kbt |
| MM4028-2 | Ac-ITFR-kbt |
| MM3130 | Ac-MQFR-kbt |
| MF1142 | Fmoc-Cha-Arg-kbt |
| MM3131 | Ac-LQFR-kbt |
| CA1022-2 | Bn-QFR-kbt |
| CA1033-2 | PhEt-QFR-kbt |
| MM3186 | Ac-IQTR-kbt |
| MM4038 | Ac-IdWFR-kbt |
| MM3177 | Ac-IQSR-kbt |
| MM4027-2 | Ac-IMFR-kbt |
| CA1041-2 | Me2-QFR-kbt |

Additionally, the compounds of Formula IA, IB, or IC having the following structure:

| | |
|---|---|
| PK-1-91 | Ac-dWLR-kbt |
| (MN1063) | |
| AJS4016 | Ac-Ser-4-AMBA-Leu-Arg-kbt |
| MF1163 | Fmoc-hTyr-Arg-kbt |
| MF1067B | Ac-IdcGlu(OAll)Phe(4-F)Arg-kbt |
| (MPM2082B) | |
| MF1177 | Fmoc-Gly(2-th)-Arg-kbt |
| MF1168 | Cbz-Bta-Arg-kbt |
| MM3194A | Ac-PSKR-kbt |
| MF2008 | Cbz-Igl-Arg-kbt |
| MM4037-1 | Ac-IEFdR-kbt |
| MF1184 | Fmoc-Ala(2-th)-Arg-kbt |
| MF1165 | Fmoc-Nle(OBzl)-Arg-kbt |
| PK-1-104 | Ac-WLR-kbt |
| (MN1070) | |
| PK-1-105 | Ac-WLR-kbt-COOH |
| (MM4094) | |
| PK-1-94 | Ac-dWLR-kbt-COOH |
| (MM4123) | |

Yet another aspect of the disclosure is a compound of Formula II, a pharmaceutically acceptable salt thereof, or a stereoisomer thereof:

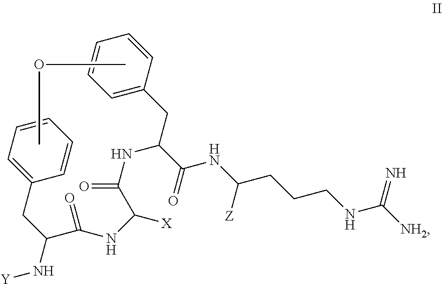

wherein X is a side chain of a natural or unnatural amino acid; Y is H, acetyl, tert-butyloxycarbonyl (Boc), benzyloxycarbonyl (Cbz), fluorenylmethyloxycarbonyl (Fmoc), benzyl, —C(O)R, —SOOR, —COOR, —C(O)NHR, —COCH(NHC(O)CH₃)—(CH₂)ₓ—NHC(O)—(CH₂)ₓ-p-halo-phenyl, substituted or unsubstituted —(CH₂)ₓaryl, substituted or unsubstituted —(CH₂)ₓheteroaryl, substituted or unsubstituted —(CH₂)ₓcycloalkyl, or substituted or unsubstituted —(CH₂)ₓheterocycle; each R is independently C₁ to C₆ alkyl, C₃ to C₆ cycloalkyl, heterocycle, alkylheterocycle, aralkyl, or aryl; and Z is independently

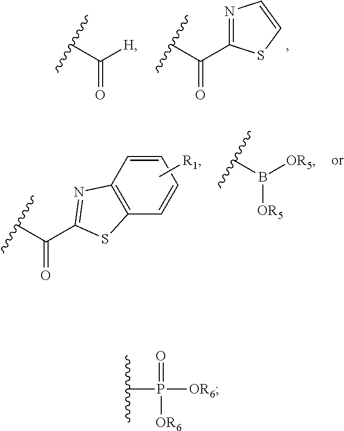

R₁ is hydrogen,

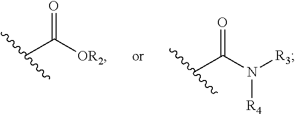

R₂ and R₃ are each independently hydrogen, substituted or unsubstituted alkyl, substituted or unsubstituted cycloalkyl, substituted or unsubstituted aryl, substituted or unsubstituted aralkyl, substituted or unsubstituted heteroaryl, or substituted or unsubstituted heteroarylalkyl; R₄ is hydrogen, substituted or unsubstituted alkyl, or a residue of an amino acid, or R₃ and R₄ can form a ring; R₅ is independently hydrogen, substituted or unsubstituted alkyl, or the R₅ moieties can form a ring; and R₆ is substituted or unsubstituted aryl.

The compounds of Formula II can have Y be hydrogen, acetyl, tert-butyloxycarbonyl (Boc), benzyloxycarbonyl(Cbz), or fluorenylmethyloxycarbonyl (Fmoc). Preferably, Y is hydrogen or acetyl.

Additionally, the compounds of Formula II can have X be a side chain of 2-Abz, 3-Abz, 4-Abz, D-Arg, dhAbu, dhLeu, D-Lys, D-Trp, Gly, L-2-Aoc, L-Agb, L-Agp, L-Ala(Bth), L-Arg, L-Arg(NO₂), L-Arg(Z)₂, L-Chg, L-Cys(4-MeOBzl), L-Cys(Bzl), L-Cys(MeBzl), L-DAB(Z), L-Glu(OBzl), L-Glu(OCHx), L-hArg, L-hCha, L-His(3-BOM), L-hLeu, L-hPhe, L-hTyr, L-Hyp, L-Hyp(Bzl), L-Idc, L-Ile, L-Leu, L-Lys, L-Lys(2-Cl-Z), L-Lys(TFA), L-Met, L-Nle, L-Nle(OBzl), L-Nva, L-Oic, L-Orn, L-Phe, L-Phe(4-I), L-Phe(F), L-Pro, L-Ser, L-Ser(Bzl), L-Thr(Bzl), L-Trp, or TmbGly.

Further, the compounds of Formula II can have X be a side chain of 2-Abz, 3-Abz, 4-Abz, D-Arg, dhAbu, dhLeu, D-Lys, Gly, L-2-Aoc, L-Agp, L-Ala(Bth), L-Chg, L-Cys(4-MeOBzl), L-Cys(Bzl), L-Cys(MeBzl), L-Glu(OBzl), L-Glu(OCHx), L-hCha, L-hLeu, L-hPhe, L-hTyr, L-Hyp, L-Hyp(Bzl), L-Idc, L-Lys(TFA), L-Nle, L-Nva, L-Oic, L-Phe, L-Phe(4-I), L-Phe(F), L-Ser(Bzl), L-Thr(Bzl), or TmbGly.

Preferably, the compounds of Formula II can have X be a side chain of dhLeu, Gly, L-Idc, L-Ile, L-Leu, L-Met, L-Oic, or L-Pro.

Additionally, the compounds of Formula II can have X be a side chain of 4-Abz, chAbu, D-Arg, D-Gln, dhAbu, dhLeu, D-Lys, D-Trp, L-2-Aoc, L-Agp, L-Ala(Bth), L-Arg(NO₂), L-Arg(Z)₂, L-Arg, L-Chg, L-Cys(Bzl), L-Dab(Z), L-Dab, L-Dap, L-Dht, L-Glu(All), L-Glu(OBzl), L-Glu(OCHx), L-Glu(OMe), L-hArg, L-hCha, L-His(3-BOM), L-hPhe, L-hTyr, L-Hyp, L-Idc, L-Igl, L-Lys(2-ClZ), L-Lys, L-Met(O), L-Met(O)₂, L-Nle(OBzl), L-Oic, L-Orn, L-Phe(F₅), or L-Ser(Ac).

Preferably, the compounds of Formula II can have X be a side chain of D-Arg, D-Gln, D-Lys, D-Trp, L-2-Aoc, L-Agp, L-Ala(Bth), L-Arg(Z)₂, L-Arg, L-Dab, L-Dap, L-Dht, L-Glu(All), L-Glu(OBzl), L-Glu(OCHx), L-Glu(OMe), L-hArg, L-hCha, L-hPhe, L-hTyr, L-Igl, L-Lys, L-Met(O), L-Met(O)₂, L-Nle(OBzl), L-Orn, L-Phe(F₅), or L-Ser(Ac).

More preferably, the compounds of Formula II can have X be a side chain of 4-Abz, chAbu, D-Arg, dhAbu, dhLeu, L-Agp, L-Ala(Bth), L-Arg(NO₂), L-Arg(Z)₂, L-Arg, L-Chg, L-Cys(Bzl), L-Dab(Z), L-Glu(OBzl), L-hArg, L-His(3-BOM), L-hTyr, L-Hyp, L-Idc, L-Lys(2-ClZ), L-Lys, L-Nle(OBzl), or L-Oic, L-Orn.

The compounds of Formula II further have R₂ and R₃ each independently be hydrogen, alkyl, cycloalkyl, aryl, aralkyl, heteroaryl, or heteroarylalkyl. Preferably, R₂ and R₃ are each independently hydrogen, C₁-C₆ alkyl, C₁-C₁₀ cycloalkyl, phenyl and benzyl.

The compounds of Formula II can also have R₄ be hydrogen, alkyl, or a residue of an amino acid.

The compounds of Formula II can have R₅ be hydrogen, alkyl, or the R₅ moieties can form a ring.

Preferably, the compounds of Formula II can have R₆ be aryl.

Additionally, the compounds of Formula II have Z of:

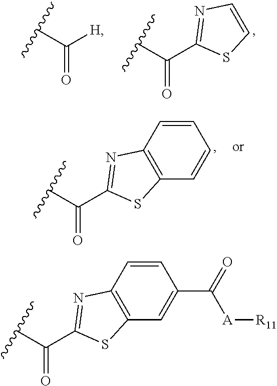

wherein A is —O— or NH, and R₁ is H, methyl, benzyl, optionally substituted alkyl, optionally substituted aryl, heterocycle, or a residue of an α-amino acid. For these groups, A can be NH and A and Rn together form a residue of an α-amino acid.

The compounds of Formula II can have Z be:

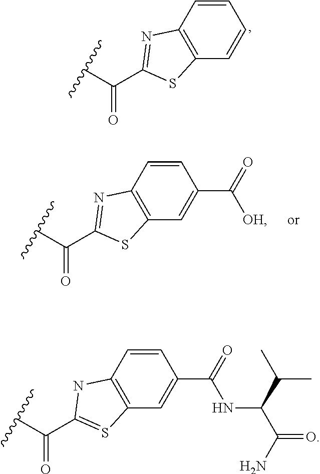

Additionally, the compounds of Formula II can have the following structures:

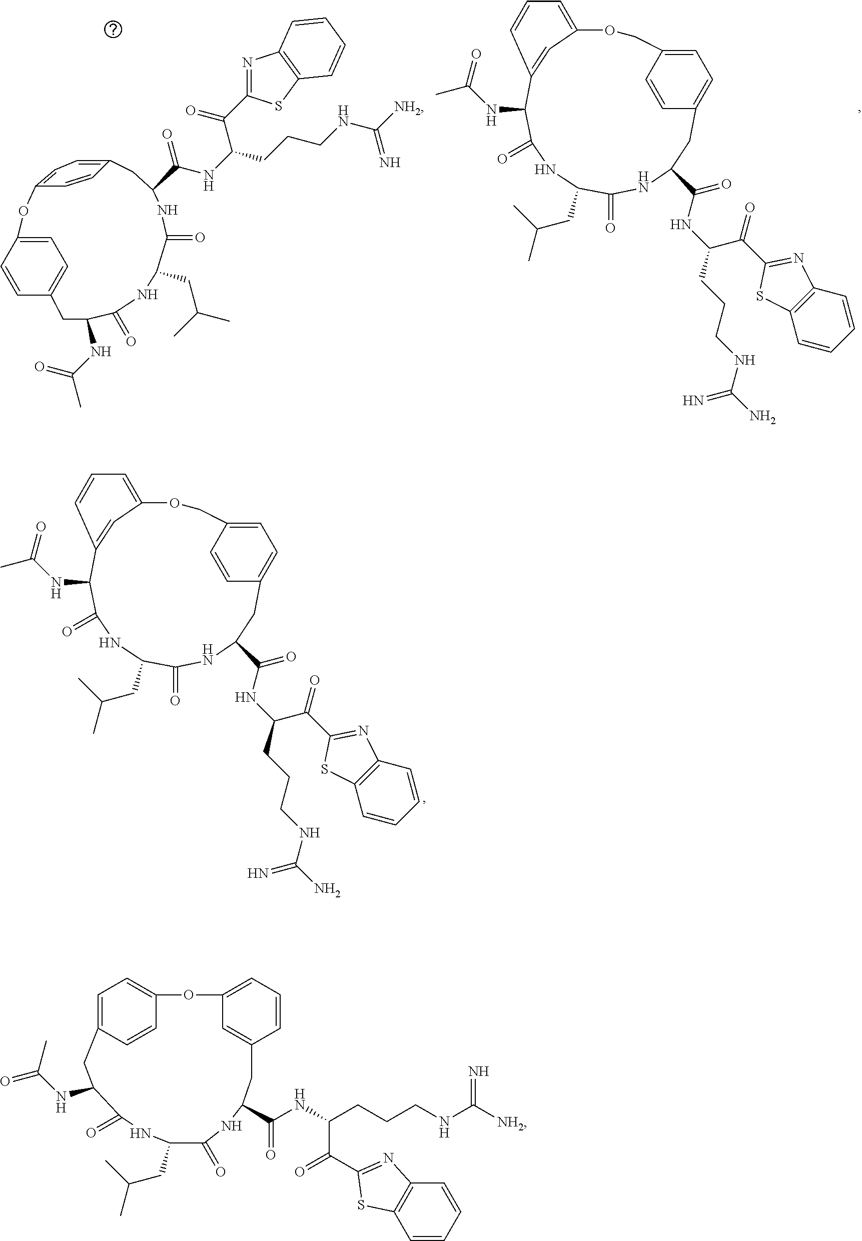

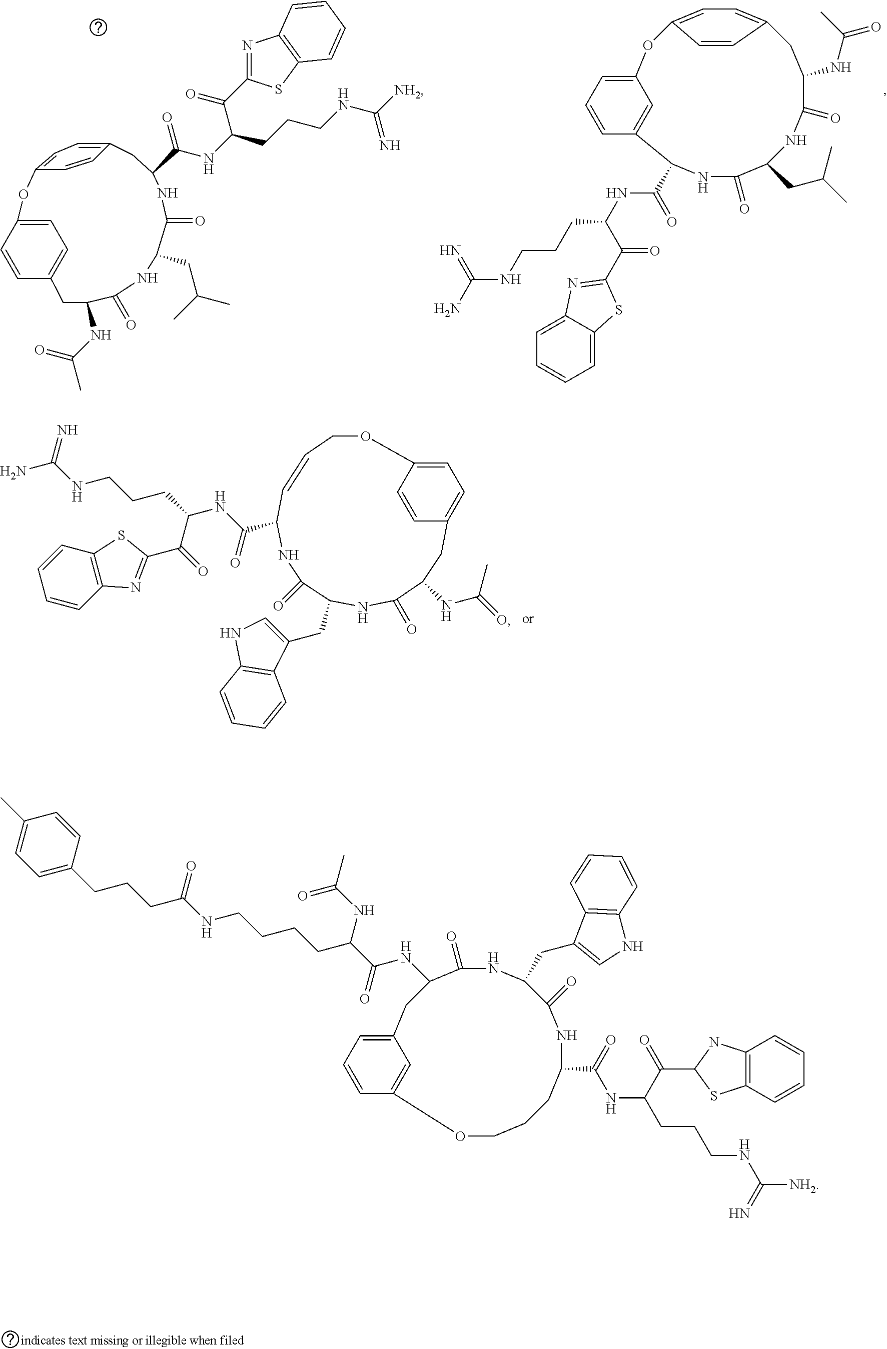

In general, alkyl (e.g, R) groups can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, includes a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O— acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O— cycloheptyl, —O-cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂— cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “hetreocyclic” can be optionally substituted.

The term “indole”, as used herein, is an aromatic heterocyclic organic compound with the formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the instant disclosure in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high-performance liquid chromatography. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the instant disclosure. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the instant disclosure. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the instant disclosure, or a salt thereof, that further can include a stoichiometric or non-stoichiometric amount of water bound by non-covalent intermolecular forces.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

In addition to the active ingredients (e.g., the inhibitor compound), the pharmaceutical composition can contain suitable pharmaceutically acceptable carriers comprising excipients and auxiliaries that facilitate processing of the active compounds into preparations which can be used pharmaceutically. As used herein, the term “pharmaceutically acceptable carrier” means a non-toxic, inert solid, semi-solid or liquid filler, diluent, encapsulating material, or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil; and soybean oil; glycols such as propylene glycol; esters such as ethyl oleate and ethyl laurate; agar; detergents such as TWEEN 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring, and perfuming agents, preservatives and antioxidants can also be present in the composition, according to the judgment of the formulator based on the desired route of administration.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

The determination of a therapeutically effective dose for any one or more of the inhibitor compounds described herein is within the capability of those skilled in the art. A therapeutically effective dose refers to that amount of active ingredient which provides the desired result. The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Typically, the normal dosage amount of the inhibitor can vary from about 0.05 to about 100 mg per kg body weight depending upon the route of administration. Guidance as to particular dosages and methods of delivery is provided in the literature and generally available to practitioners in the art. It will generally be administered so that a daily oral dose in the range, for example, from about 0.1 mg to about 75 mg, from about 0.5 mg to about 50 mg, or from about 1 mg to about 25 mg per kg body weight is given. The active ingredient can be administered in a single dose per day, or alternatively, in divided does (e.g., twice per day, three time a day, four times a day, etc.). In general, lower doses can be administered when a parenteral route is employed. Thus, for example, for intravenous administration, a dose in the range, for example, from about 0.05 mg to about 30 mg, from about 0.1 mg to about 25 mg, or from about 0.1 mg to about 20 mg per kg body weight can be used.

A pharmaceutical composition for oral administration can be formulated using pharmaceutically acceptable carriers known in the art in dosages suitable for oral administration. Such carriers enable the pharmaceutical compositions to be formulated as tablets, pills, dragees, capsules, liquids, gels, syrups, slurries, suspensions, and the like, for ingestion by the subject. In certain embodiments, the composition is formulated for parenteral administration. Further details on techniques for formulation and administration can be found in the latest edition of REMINGTON'S PHARMACEUTICAL SCIENCES (Mack Publishing Co., Easton, Pa., which is incorporated herein by reference). After pharmaceutical compositions have been prepared, they can be placed in an appropriate container and labeled for treatment of an indicated condition. Such labeling would include amount, frequency, and method of administration.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of the agent being metabolized or excreted from the body. The controlled-release of an agent may be stimulated by various inducers, e.g., change in pH, change in temperature, enzymes, water, or other physiological conditions or molecules.

The compounds of the present disclosure can also be used in various nuclear imaging techniques when labeled with a suitable radionuclide. Accordingly, an imaging composition in accordance with the present disclosure comprises a radiolabeled compound of the instant formulas, wherein the labeled compound comprises a radioisotope selected from the group consisting of ¹¹C, ¹³N, ¹⁵O, ¹⁸F, ⁷⁵Br, ¹²⁴I, ¹²⁵I, and ¹³¹I. Methods known in the art for radiolabeling the compounds of the present disclosure may be used.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Another aspect of the disclosure is a method of inhibiting matriptase, hepsin, TMPRSS2, or hepatocyte growth factor activator (HGFA) comprising administering to an organism a composition comprising an effective amount of a compound of Formulae IA, IB, IC, and II.

Further, disclosed are methods of overcoming and preventing resistance to anticancer drugs including targeted therapies, immunotherapy, radiation, and chemotherapy comprising administering to a subject in need thereof a pharmaceutical composition comprising a therapeutically effective amount of a compound of Formulae IA, IB, IC, and II.

Also disclosed are methods of overcoming and preventing resistance to a kinase small molecule or antibody inhibitor including those targeting EGFR and MET by blocking HGF and MSP production or activation comprising administering to a subject in need thereof a pharmaceutical composition comprising a therapeutically effective amount of at least one compound of Formulae IA, IB, IC, and II.

Another method is for overcoming and preventing resistance to a DNA-damaging agent including gemcitabine comprising administering to a subject in need thereof a pharmaceutical composition comprising a therapeutically effective amount of at least one compound of Formulae IA, IB, IC, and II.

Also disclosed are methods of overcoming and preventing resistance to an immunotherapy agent including a PD-1 antagonist comprising administering to a subject in need thereof a pharmaceutical composition comprising a therapeutically effective amount of at least one compound of Formulae IA, IB, IC, and II.

Further disclosed are methods of inhibiting tumor progression and metastasis comprising administering to the subject in need thereof a pharmaceutical composition comprising a therapeutically effective amount of at least one compound of Formulae IA, IB, IC, and II.

Also disclosed are methods of treating or preventing a viral infection in a subject comprising administering to the subject in need thereof a pharmaceutical composition comprising a therapeutically effective amount of a compound of Formulae IA, IB, IC, and II.

In some methods the viral infection is caused by a coronavirus. Preferably, the coronavirus is HCoV-229E, HCoV-NL63, HCoV-OC43, HCoV-HKU1, SARS-CoV, SARS-CoV-2, or MERS-CoV. More preferably, the corona virus is SARS-CoV-2 or MERS-CoV and variants thereof.

The coronavirus infectious disease treated herein is a disease caused by infection with a virus of Coronaviridae, including diseases caused by infected humans or other animals, in particular diseases caused by human coronaviruses that infect humans including, but not limited to, diseases caused by infection with CoV-229E, —OC43, -NL63, —HKU1, SARS-CoV, SARS-CoV-2, and MERS-CoV. In particular, the viral infection treated can be in the subfamily of coronavierinae, and genus, for example, alphacoronavirus (CoV-229E, CoV-NL63), betacoronavirus (CoV-OC43, CoV-HKU1, SARS-CoV, SARS-CoV-2, and MERS-CoV), deltacoronavirus and gammacoronavirus.

Also, the viral infection can be caused by an influenza virus. Preferably, the influenza virus is A(H1N1), A(H3N2), or A(H5N1). More preferably, the influenza virus is A(H1N1) and variants thereof.

Influenza viruses A, B and C are very similar in overall structure. The virus particle is 80-120 nanometres in diameter and usually roughly spherical, although filamentous forms can occur. These filamentous forms are more common in influenza C, which can form cordlike structures up to 500 micrometres long on the surfaces of infected cells. However, despite these varied shapes, the viral particles of all influenza viruses are similar in composition. These are made of a viral envelope containing two main types of glycoproteins, wrapped around a central core. The central core contains the viral RNA genome and other viral proteins that package and protect this RNA. Unusually for a virus, its genome is not a single piece of nucleic acid; instead, it contains seven or eight pieces of segmented negative-sense RNA, each piece of RNA contains either one or two genes. For example, the influenza A genome contains 11 genes on eight pieces of RNA, encoding for 11 proteins: hemagglutinin (HA), neuraminidase (NA), nucleoprotein (NP), M1, M2, NS1, NS2(NEP), PA, PB1, PB1-F2 and PB2.

Hemagglutinin (HA) and neuraminidase (NA) are the two large glycoproteins on the outside of the viral particles. HA is a lectin that mediates binding of the virus to target cells and entry of the viral genome into the target cell, while NA is involved in the release of progeny virus from infected cells, by cleaving sugars that bind the mature viral particles. Thus, these proteins are targets for antiviral drugs. Furthermore, they are antigens to which antibodies can be raised. Influenza A viruses are classified into subtypes based on antibody responses to HA and NA. These different types of HA and NA form the basis of the H and N distinctions in, for example, H5N1. There are 16 H and 9 N subtypes known, but only H 1, 2 and 3, and N 1 and 2 are commonly found in humans.

There are at least 16 different HA antigens. These subtypes are labeled H1 through H16. The last, H16, was discovered only recently on influenza A viruses isolated from black-headed gulls from Sweden and Norway. The first three hemagglutinins, H1, H2, and H3, are found in human influenza viruses.

A highly pathogenic avian flu virus of H5N1 type can infect humans. It is known that single amino acid changes in this avian virus strain's type H5 hemagglutinin found in human patients result in a strain that “can significantly alter receptor specificity of avian H5N1 viruses, providing them with an ability to bind to receptors optimal for human influenza viruses.” The hemagglutinin of the H5N1 virus has been associated with the high pathogenicity of this flu virus strain, apparently due to its ease of conversion to an active form by proteolysis.

Also disclosed are methods of inhibiting TMPRSS2 and/or matriptase in an organism comprising administering to the organism a composition comprising an effective amount of a compound of Formulae IA, IB, IC, and II.

Any of the compounds described herein are useful for inhibiting one or more trypsin-like S1 serine proteases. In particular, compounds of Formulae IA, IB, IC, and II are useful for inhibiting one or more of matriptase, hepsin, and/or HGFA. Accordingly, the present disclosure is also directed to a method of inhibiting a trypsin-like S1 serine protease (e.g., matriptase, hepsin, KLK5, TMPRSS2, TMPRSS11D (HAT, TMPRSS13, and/or HGFA) comprising administering to a subject in need thereof a pharmaceutical composition comprising a therapeutically effective amount of a compound of Formulae IA, IB, IC, and II as described herein. Preferably, the compounds are highly selective for one of TMPRSS2, matriptase, hepsin, or HGFA, or preferably TMPRSS2 and matriptase.

As noted, trypsin-like S1 serine proteases like matriptase, hepsin, HGFA, KLK5, TMPRSS13, and TMPRSS2 are involved in various cancerous disease conditions. Thus, the present disclosure is directed to various methods of using the inhibitor compounds to treat cancer in a subject (e.g., a human). One method includes inhibiting HGF/MET oncogenic signaling by administering to a subject in need thereof a pharmaceutical composition comprising a therapeutically effective amount of at least one compound of Formulae IA, IB, IC, and II as described herein. Another method includes inhibiting MSP/RON oncogene signaling by administering to a subject in need thereof a pharmaceutical composition comprising a therapeutically effective amount of at least one compound of Formulae IA, IB, IC, and II as described herein. Yet another method including reversing resistance to a kinase inhibitor by blocking HGF and/or MPS production and/or activation by administering to a subject in need thereof a pharmaceutical composition comprising a therapeutically effective amount of a compound of Formulae IA, IB, IC, and II as described herein.

Further methods include overcoming and preventing resistance to a DNA-damaging agent including gemcitabine comprising administering to a subject in need thereof a pharmaceutical composition comprising a therapeutically effective amount of a compound of Formulae IA, IB, IC, and II as described herein. Still other methods include overcoming and preventing resistance to an immunotherapy agent including a PD-1 antagonist comprising administering to a subject in need thereof a pharmaceutical composition comprising a therapeutically effective amount of a compound of Formulae IA, IB, IC, and II as described herein.

Another method includes inhibiting carcinoma progression and metastasis comprising administering to the subject in need thereof a pharmaceutical composition comprising a therapeutically effective amount of a compound of Formulae IA, IB, IC, and II as described herein.

A further method includes treating a malignancy, a pre-malignant condition, or cancer in a subject comprising administering to the subject in need thereof a pharmaceutical composition comprising a therapeutically effective amount of a compound of Formulae IA, IB, IC, and II as described herein. The cancer can be selected from the group consisting of breast, ovarian, prostate, endometrial, colon, pancreatic, head and neck, gastric, renal, brain, liver, bladder, kidney, lung, esophageal, leukemia, multiple myeloma, lymphoma, and melanoma. For example, the malignancy and the pre-malignant condition can be a condition of the breast, lung, colon, and/or pancreas. Also, the pre-malignant condition can be selected from the group consisting of a typical ductal hyperplasia of the breast, actinic keratosis, leukoplakia, Barrett's epithelium (columnar metaplasia) of the esophagus, ulcerative colitis, adenomatous colorectal polyps, erythroplasia of Queyrat, Bowen's disease, bowenoid papulosis, vulvar intraepthelial neoplasia, and dysplastic changes to the cervix. In various methods, the cancer can also be metastasized.

As noted, TMPRSS2 and matriptase are type II transmembrane serine proteases (TTSPs) which are essential for host-cell viral entry and replication of SARS-CoV-2 2-4, SARS-CoV and other coronaviruses such as MERS-CoV, and influenza. SARS-CoV-2 cell entry involves binding to the host cell receptor ACE2 which requires proteolytic priming of the Spike protein by TMPRSS2 and/or matriptase, such that TMPRSS2, matriptase or dual TMPRSS2/matriptase inhibitors offer promise as effective therapeutics for diseases caused by coronavirus infections like COVID-19. Proteolytic priming of the HA protein by TMPRSS2 and/or matriptase is essential for viral pathogenesis, such that TMPRSS2, matriptase or dual TMPRSS2/matriptase inhibitors offer promise as effective therapeutics for influenza A infections. Also provided is a process of treating, preventing, or reversing cancer or a viral infection in a subject in need of administration of a therapeutically effective amount of a serine protease inhibitor, so as to inhibit a serine protease to treat cancer or a viral infection.

In virus classification the influenza virus is an RNA virus of three of the five genera of the family Orthomyxoviridae: Influenzavirus A, Influenzavirus B, Influenzavirus C. These viruses are only distantly related to the human parainfluenza viruses, which are RNA viruses belonging to the paramyxovirus family that are a common cause of respiratory infections in children such as croup, but can also cause a disease similar to influenza in adults.

Methods described herein are generally performed on a subject in need thereof. A subject in need of the therapeutic methods described herein can be a subject having, diagnosed with, suspected of having, or at risk for developing cancer or a viral infection. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a serine protease inhibitor is, for example, an amount that would cause the desired therapeutic effect in a subject while minimizing undesired side effects. In various embodiments, an effective amount of a serine protease inhibitor described herein can substantially inhibit cancer or a viral infection, slow the progress of cancer or a viral infection, or limit the development of cancer or a viral infection.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a serine protease inhibitor can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat cancer or a viral infection.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a serine protease inhibitor can occur as a single event or over a time course of treatment. For example, a serine protease inhibitor can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for cancer or a viral infection.

A serine protease inhibitor can be administered simultaneously or sequentially with another agent, such as an antibiotic, an anti-inflammatory, or another agent. For example, a serine protease inhibitor can be administered simultaneously with another agent, such as an antibiotic or an anti-inflammatory. Simultaneous administration can occur through the administration of separate compositions, each containing one or more of a serine protease inhibitor, an antibiotic, an anti-inflammatory, or another agent. Simultaneous administration can occur through the administration of one composition containing two or more of a serine protease inhibitor, an antibiotic, an anti-inflammatory, or another agent. A serine protease inhibitor can be administered sequentially with an antibiotic, an anti-inflammatory, or another agent. For example, a serine protease inhibitor can be administered before or after the administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to a serine protease inhibitor, solubilizers, and solvents. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

The methods and algorithms of the invention may be enclosed in a controller or processor. Furthermore, methods and algorithms of the present invention can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

### Amino Acid Abbreviations

As used herein, the abbreviations of the naturally occurring amino acids are as follows:

TABLE 4
Amino Acid Abbreviations

| | Three | One |
|---|---|---|
| | letter | letter |
| Amino acid | code | code |
| alanine | Ala | A |
| arginine | Arg | R |
| asparagine | Asn | N |
| aspartic acid | Asp | D |
| cysteine | Cys | C |
| glutamic acid | Glu | E |
| glutamine | Gln | Q |
| glycine | Gly | G |
| histidine | His | H |
| isoleucine | Ile | I |
| leucine | Leu | L |
| lysine | Lys | K |
| methionine | Met | M |
| phenylalanine | Phe | F |
| proline | Pro | P |
| serine | Ser | S |
| threonine | Thr | T |
| tryptophan | Trp | W |
| tyrosine | Tyr | Y |
| valine | Val | V |

The naturally occurring amino acids described herein are the L-isomer unless denoted as a D-isomer.

Unless otherwise specified, the unnatural amino acids can be selected from the group listed in the tables below. The unnatural amino acids can be the D and/or L-isomers.

TABLE 5
Unnatural Amino Acids

| No | Structure + code |
|---|---|
| 1 | 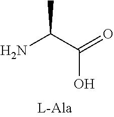 |
| 2 | 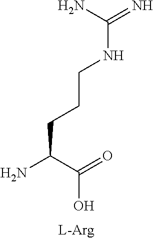 [SMILES: N=C(N)NCCC[C@H](N)C(=O)O] |
| 3 | 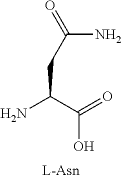 |
| 4 | 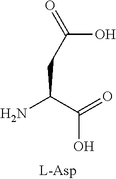 |
| 5 | 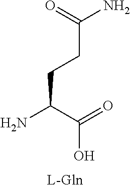 |
| 6 | 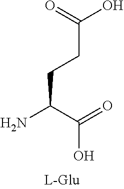 |
| 7 | 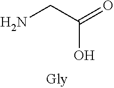 |
| 8 | 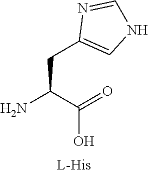 [SMILES: N[C@@H](Cc1c[nH]cn1)C(=O)O] |
| 9 | 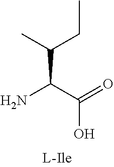 |
| 10 | 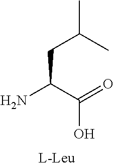 |
| 11 | 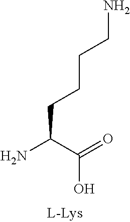 |
| 12 | 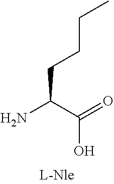 |
| 13 | 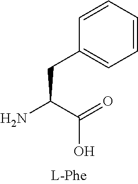 [SMILES: N[C@@H](Cc1ccccc1)C(=O)O] |
| 14 | 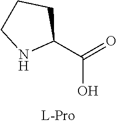 |
| 15 | 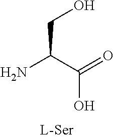 |
| 16 | 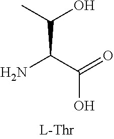 |
| 17 | 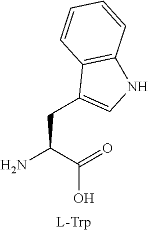 [SMILES: N[C@@H](Cc1c[nH]c2ccccc12)C(=O)O] |
| 18 | 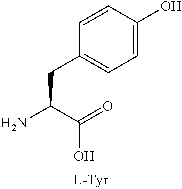 [SMILES: N[C@@H](Cc1ccc(O)cc1)C(=O)O] |
| 19 | 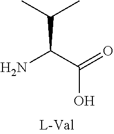 |
| 20 | 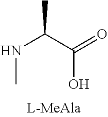 |
| 21 | 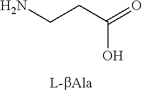 |
| 22 | 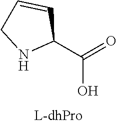 |
| 23 | 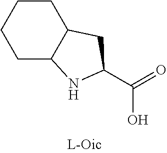 |
| 24 | 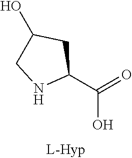 |
| 25 | 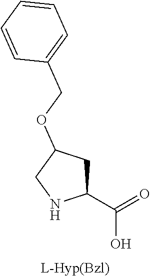 |
| 26 | 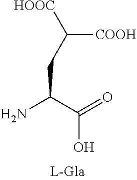 [SMILES: N[C@@H](CC(C(=O)O)C(=O)O)C(=O)O] |
| 27 | 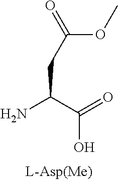 |
| 28 | 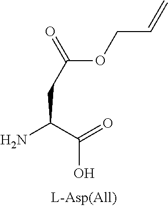 |
| 29 | 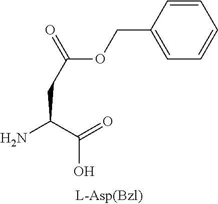 [SMILES: N[C@@H](CC(=O)OCc1ccccc1)C(=O)O] |
| 30 | 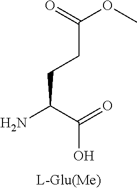 |
| 31 | 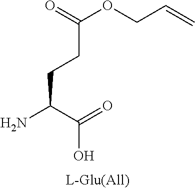 |
| 32 | 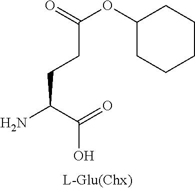 [SMILES: N[C@@H](CCC(=O)OC1CCCCC1)C(=O)O] |
| 33 | 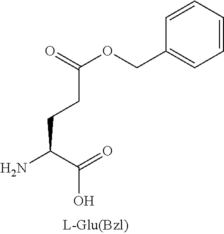 [SMILES: N[C@@H](CCC(=O)OCc1ccccc1)C(=O)O] |
| 34 | 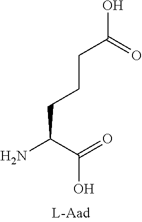 [SMILES: N[C@@H](CCCC(=O)O)C(=O)O] |
| 35 | 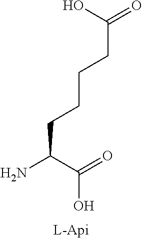 [SMILES: N[C@@H](CCCCC(=O)O)C(=O)O] |
| 36 | 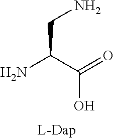 |
| 37 | 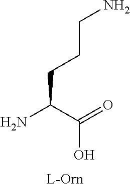 |
| 38 | 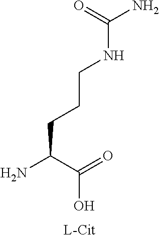 [SMILES: NC(=O)NCCC[C@H](N)C(=O)O] |
| 39 | 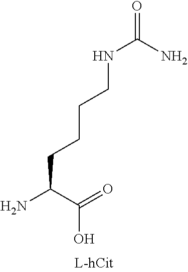 [SMILES: NC(=O)NCCCC[C@H](N)C(=O)O] |
| 40 | 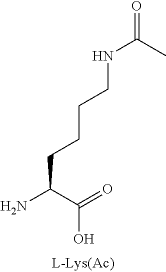 |
| 41 | 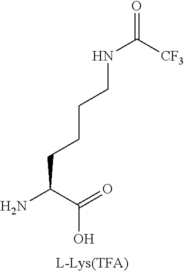 [SMILES: N[C@@H](CCCCNC(=O)C(F)(F)F)C(=O)O] |
| 42 | 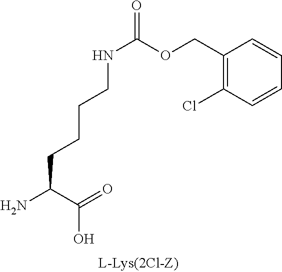 [SMILES: N[C@@H](CCCCNC(=O)OCc1ccccc1Cl)C(=O)O] |
| 43 | 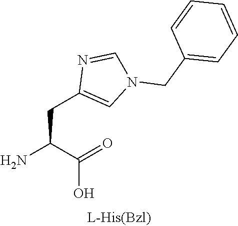 [SMILES: N[C@@H](Cc1cn(Cc2ccccc2)cn1)C(=O)O] |
| 44 | 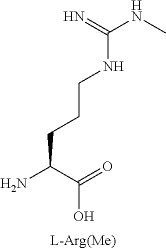 |
| 45 | 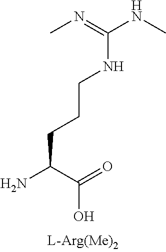 |
| 46 | 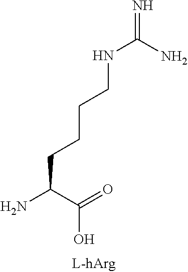 [SMILES: N=C(N)NCCCC[C@H](N)C(=O)O] |
| 47 | 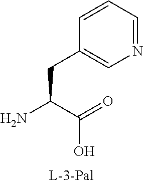 [SMILES: N[C@@H](Cc1cccnc1)C(=O)O] |
| 48 | 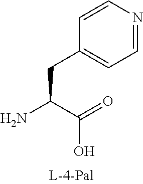 [SMILES: N[C@@H](Cc1ccncc1)C(=O)O] |
| 49 | 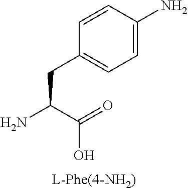 [SMILES: Nc1ccc(C[C@H](N)C(=O)O)cc1] |
| 50 | 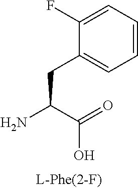 [SMILES: N[C@@H](Cc1ccccc1F)C(=O)O] |
| 51 | 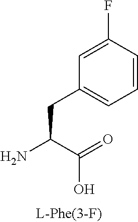 [SMILES: N[C@@H](Cc1cccc(F)c1)C(=O)O] |
| 52 | 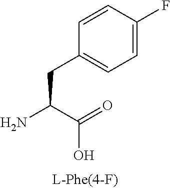 [SMILES: N[C@@H](Cc1ccc(F)cc1)C(=O)O] |
| 53 | 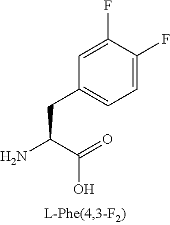 [SMILES: N[C@@H](Cc1ccc(F)c(F)c1)C(=O)O] |
| 54 | 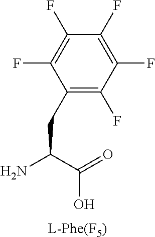 |
| 55 | 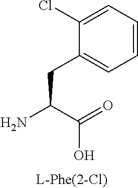 [SMILES: N[C@@H](Cc1ccccc1Cl)C(=O)O] |
| 56 | 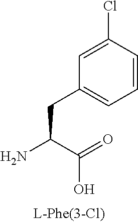 [SMILES: N[C@@H](Cc1cccc(Cl)c1)C(=O)O] |
| 57 | 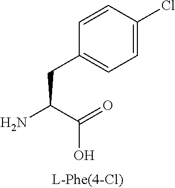 [SMILES: N[C@@H](Cc1ccc(Cl)cc1)C(=O)O] |
| 58 | 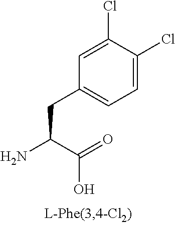 [SMILES: N[C@@H](Cc1ccc(Cl)c(Cl)c1)C(=O)O] |
| 59 | 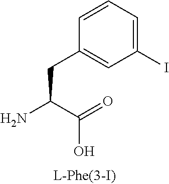 [SMILES: N[C@@H](Cc1cccc(I)c1)C(=O)O] |
| 60 | 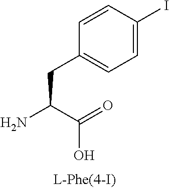 [SMILES: N[C@@H](Cc1ccc(I)cc1)C(=O)O] |
| 61 | 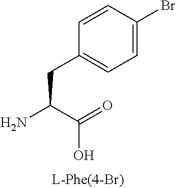 [SMILES: N[C@@H](Cc1ccc(Br)cc1)C(=O)O] |
| 62 | 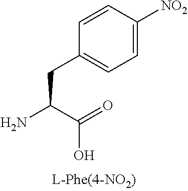 [SMILES: N[C@@H](Cc1ccc([N+](=O)[O-])cc1)C(=O)O] |
| 63 | 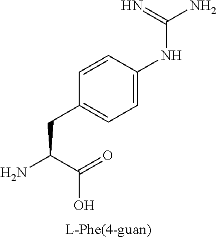 [SMILES: N=C(N)Nc1ccc(C[C@H](N)C(=O)O)cc1] |
| 64 | 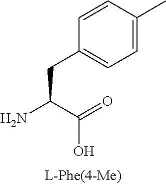 |
| 65 | 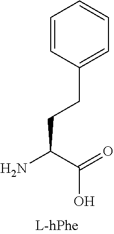 [SMILES: N[C@@H](CCc1ccccc1)C(=O)O] |
| 66 | 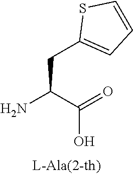 [SMILES: N[C@@H](Cc1cccs1)C(=O)O] |
| 67 | 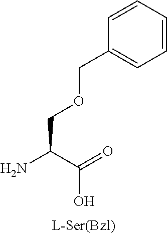 [SMILES: N[C@@H](COCc1ccccc1)C(=O)O] |
| 68 | 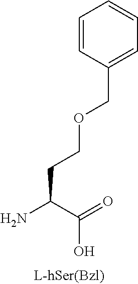 [SMILES: N[C@@H](CCOCc1ccccc1)C(=O)O] |
| 69 | 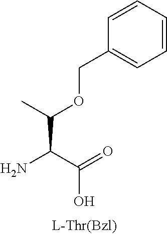 |
| 70 | 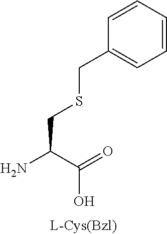 [SMILES: N[C@@H](CSCc1ccccc1)C(=O)O] |
| 71 | 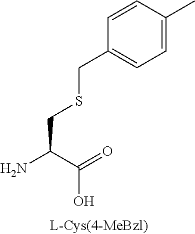 |
| 72 | 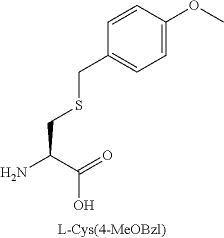 [SMILES: COc1ccc(CSC[C@H](N)C(=O)O)cc1] |
| 73 | 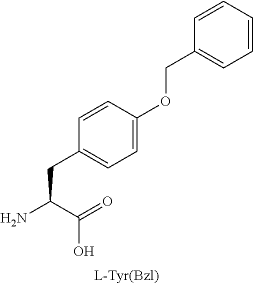 [SMILES: N[C@@H](Cc1ccc(OCc2ccccc2)cc1)C(=O)O] |
| 74 | 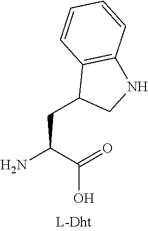 [SMILES: N[C@@H](CC1CNc2ccccc21)C(=O)O] |
| 75 | 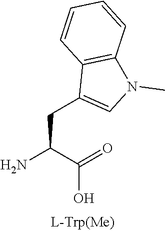 [SMILES: Cn1cc(C[C@H](N)C(=O)O)c2ccccc21] |
| 76 | 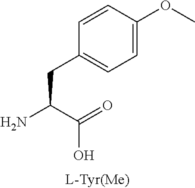 |
| 77 | 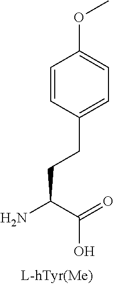 |
| 78 | 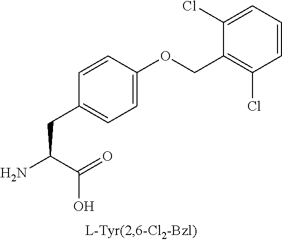 [SMILES: N[C@@H](Cc1ccc(OCc2c(Cl)cccc2Cl)cc1)C(=O)O] |
| 79 | 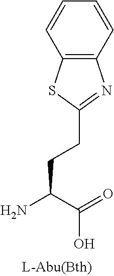 [SMILES: N[C@@H](CCc1nc2ccccc2s1)C(=O)O] |
| 80 | 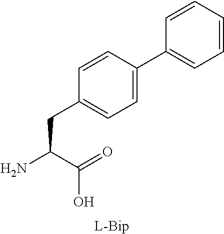 |
| 81 | 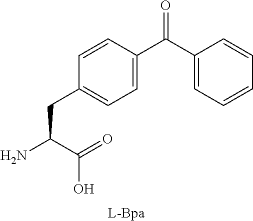 |
| 82 | 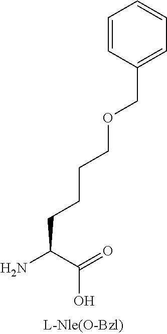 [SMILES: N[C@@H](CCCCOCc1ccccc1)C(=O)O] |
| 83 | 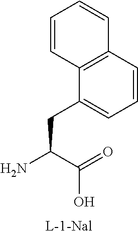 |
| 84 | 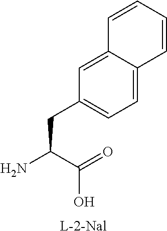 |
| 85 | 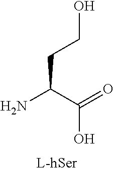 |
| 86 | 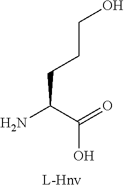 |
| 87 | 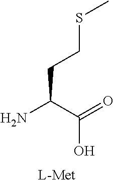 |
| 88 | 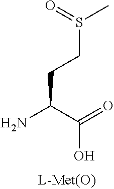 |
| 89 | 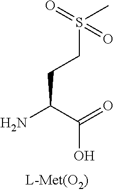 |
| 90 | 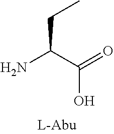 |
| 91 | 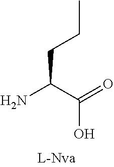 |
| 92 | 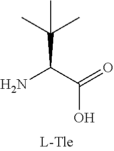 |
| 93 | 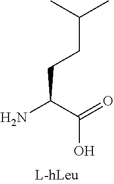 |
| 94 | 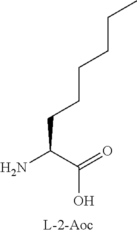 |
| 95 | 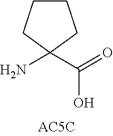 |
| 96 | 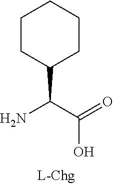 [SMILES: N[C@H](C(=O)O)C1CCCCC1] |
| 97 | 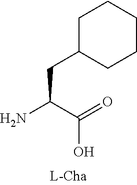 [SMILES: N[C@@H](CC1CCCCC1)C(=O)O] |
| 98 | 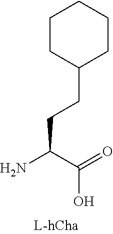 [SMILES: N[C@@H](CCC1CCCCC1)C(=O)O] |
| 99 | 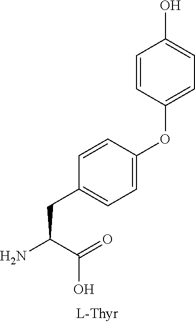 [SMILES: N[C@@H](Cc1ccc(Oc2ccc(O)cc2)cc1)C(=O)O] |
| 100 | 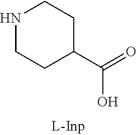 |
| 101 | 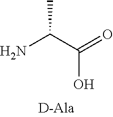 |
| 102 | 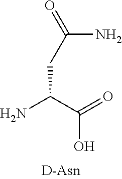 |
| 103 | 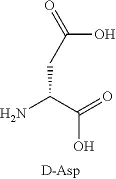 |
| 104 | 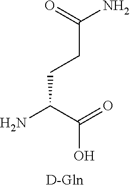 |
| 105 | 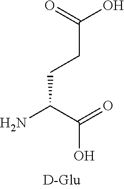 |
| 106 | 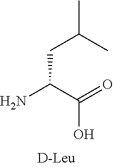 |
| 107 | 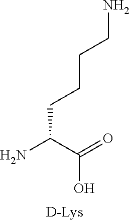 |
| 108 | 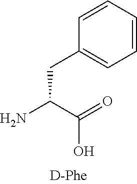 [SMILES: N[C@H](Cc1ccccc1)C(=O)O] |
| 109 | 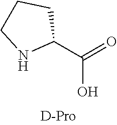 |
| 110 | 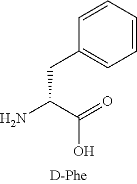 [SMILES: N[C@H](Cc1ccccc1)C(=O)O] |
| 111 | 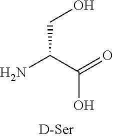 |
| 112 | 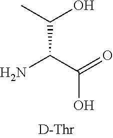 |
| 113 | 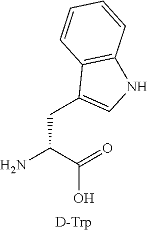 [SMILES: N[C@H](Cc1c[nH]c2ccccc12)C(=O)O] |
| 114 | 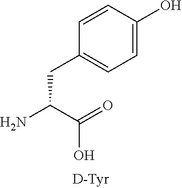 [SMILES: N[C@H](Cc1ccc(O)cc1)C(=O)O] |
| 115 | 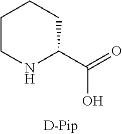 |
| 116 | 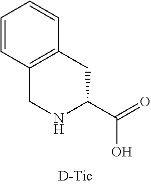 [SMILES: O=C(O)[C@H]1Cc2ccccc2CN1] |
| 117 | 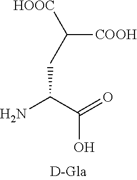 [SMILES: N[C@H](CC(C(=O)O)C(=O)O)C(=O)O] |
| 118 | 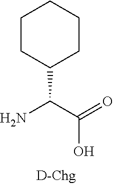 [SMILES: N[C@@H](C(=O)O)C1CCCCC1] |
| 119 | 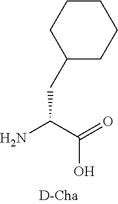 [SMILES: N[C@H](CC1CCCCC1)C(=O)O] |
| 120 | 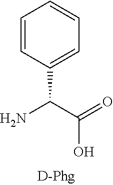 [SMILES: N[C@@H](C(=O)O)c1ccccc1] |
| 121 | 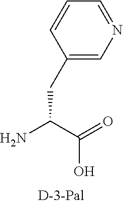 [SMILES: N[C@H](Cc1cccnc1)C(=O)O] |
| 123 | 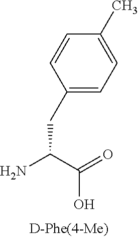 |
| 124 | 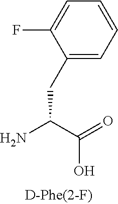 [SMILES: N[C@H](Cc1ccccc1F)C(=O)O] |
| 125 | 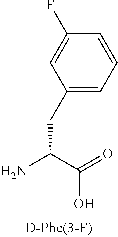 [SMILES: N[C@H](Cc1cccc(F)c1)C(=O)O] |
| 126 | 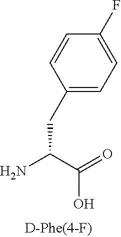 [SMILES: N[C@H](Cc1ccc(F)cc1)C(=O)O] |
| 127 | 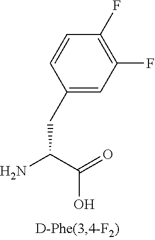 [SMILES: N[C@H](Cc1ccc(F)c(F)c1)C(=O)O] |
| 128 | 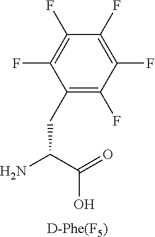 [SMILES: N[C@H](Cc1c(F)c(F)c(F)c(F)c1F)C(=O)O ; "D-Phe(F5)"] |
| 129 | 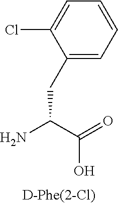 [SMILES: N[C@H](Cc1ccccc1Cl)C(=O)O ; "D-Phe(2-Cl)"] |
| 130 | 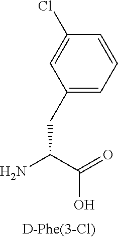 [SMILES: N[C@H](Cc1cccc(Cl)c1)C(=O)O ; "D-Phe(3-Cl)"] |
| 131 | 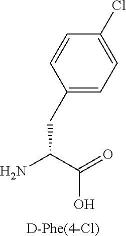 [SMILES: N[C@H](Cc1ccc(Cl)cc1)C(=O)O ; "D-Phe(4-Cl)"] |
| 132 | 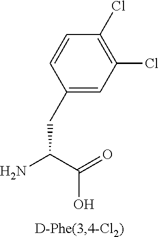 |
| 133 | 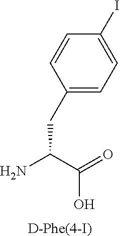 [SMILES: N[C@H](Cc1ccc(I)cc1)C(=O)O ; "D-Phe(4-I)"] |
| 134 | 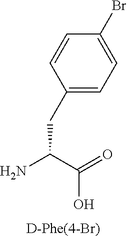 [SMILES: N[C@H](Cc1ccc(Br)cc1)C(=O)O ; "D-Phe(4-Br)"] |
| 135 | 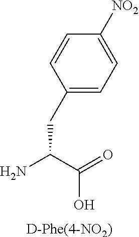 [SMILES: N[C@H](Cc1ccc([N+](=O)[O-])cc1)C(=O)O ; "D-Phe(4-NO2)"] |
| 136 | 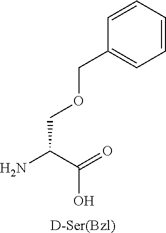 [SMILES: N[C@H](COCc1ccccc1)C(=O)O ; "D-Ser(Bzl)"] |
| 137 | 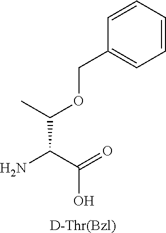 |
| 138 | 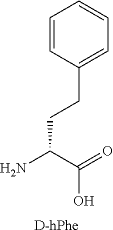 |
| 139 | 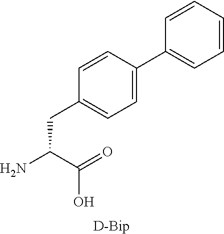 |
| 140 | 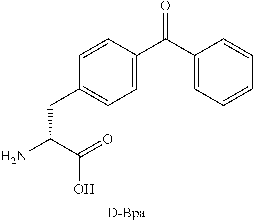 |
| 141 | 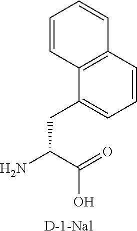 |
| 142 | 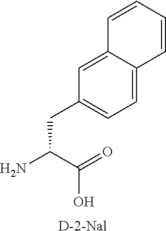 |

TABLE 6
Additional Unnatural Amino Acids

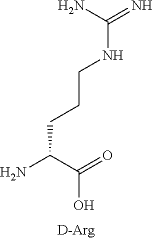 [SMILES: N=C(N)NCCC[C@@H](N)C(=O)O]

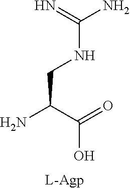

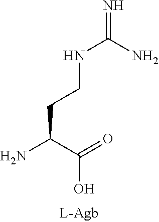 [SMILES: N=C(N)NCC[C@H](N)C(=O)O]

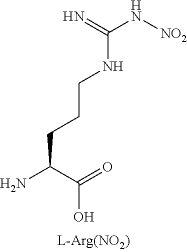 [SMILES: N=C(NCCC[C@H](N)C(=O)O)N[N+](=O)[O-]]

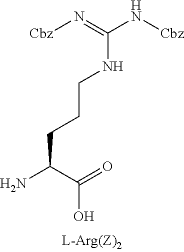 [SMILES: N[C@@H](CCCN/C(=N\C(=O)OCc1ccccc1)NC(=O)OCc1ccccc1)C(=O)O]

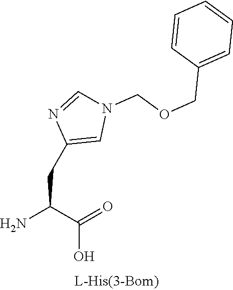 [SMILES: N[C@@H](Cc1cn(COCc2ccccc2)cn1)C(=O)O]

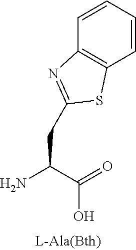 [SMILES: N[C@@H](Cc1nc2ccccc2s1)C(=O)O]

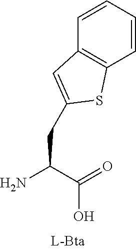 [SMILES: N[C@@H](Cc1cc2ccccc2s1)C(=O)O]

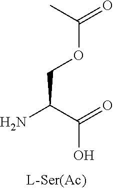

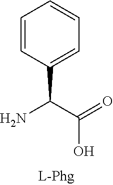 [SMILES: N[C@H](C(=O)O)c1ccccc1]

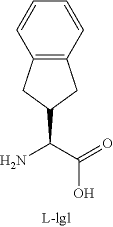 [SMILES: N[C@H](C(=O)O)C1Cc2ccccc2C1]

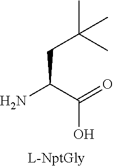

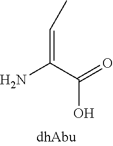

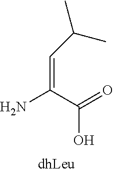

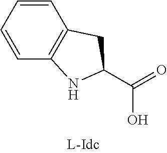 [SMILES: O=C(O)[C@@H]1Cc2ccccc2N1]

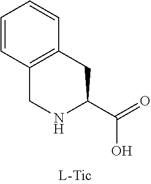 [SMILES: O=C(O)[C@@H]1Cc2ccccc2CN1]

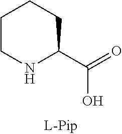

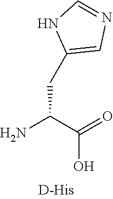 [SMILES: N[C@H](Cc1cnc[nH]1)C(=O)O]

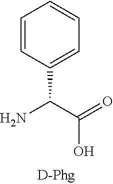 [SMILES: N[C@@H](C(=O)O)c1ccccc1]

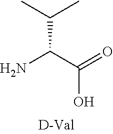

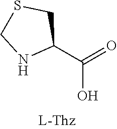

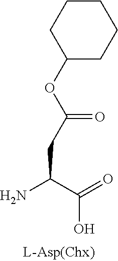 [SMILES: N[C@@H](CC(=O)OC1CCCCC1)C(=O)O]

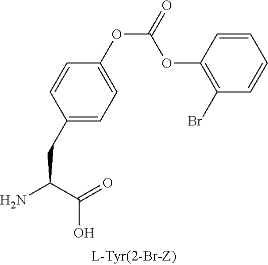 [SMILES: N[C@@H](Cc1ccc(OC(=O)Oc2ccccc2Br)cc1)C(=O)O]

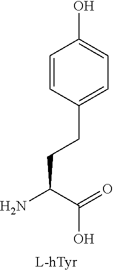 [SMILES: N[C@@H](CCc1ccc(O)cc1)C(=O)O]

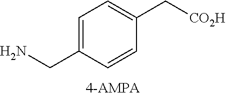 [SMILES: NCc1ccc(CC(=O)O)cc1]

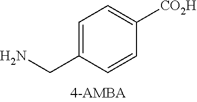 [SMILES: NCc1ccc(C(=O)O)cc1]

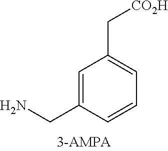 [SMILES: NCc1cccc(CC(=O)O)c1]

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

As used in this application, including the appended claims, the singular forms “a,” “an,” and “the” include plural references unless the content clearly dictates otherwise, and are used interchangeably with “at least one” and “one or more.”

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

The invention will be further described in the following examples, which do not limit the scope of the invention described in the claims.

### EXAMPLES

It is to be understood that while the invention has been described in conjunction with the detailed description thereof, the preceding description is intended to illustrate and not limit the scope of the invention, which is defined by the scope of the appended claims. Other aspects, advantages, and modifications are within the scope of the following claims.

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

For all of the examples, N.D. indicates “not determined.”

For all of the synthesis examples, the compound numbering system restarts for each example but is consistent within individual examples.

### Example 1: Exemplary Compounds

TABLE 7
Exemplary Compounds

| Synonyms | Structure | IUPAC |
|---|---|---|
| Fmoc-2-Nal- Arg-kbt, MF2012 | 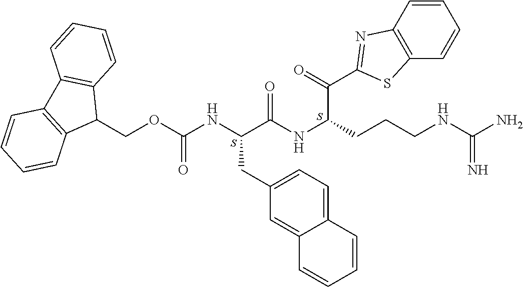 | (S)-2-(2-naphthyl)-1- [N-(S)-1-[(1,3- benzothiazol-2- yl)carbonyl]-4- guanidinobutylcarbamoyl] ethyl (9H-fluoren-9- yl)methanecarbamate |
| Fmoc-Orn- Arg-kbt, MF2011 | 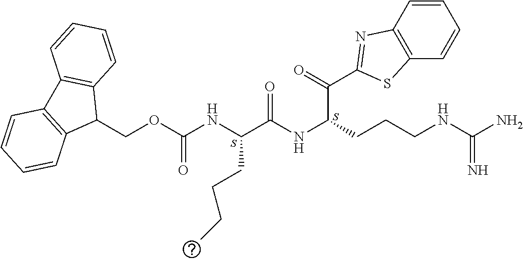 | (S)-4-amino-1-[N- (S)-1-[(1,3- benzothiazol-2- yl)carbonyl]-4- guanidinobutylcarbamoyl] butyl (9H-fluoren-9- yl)methanecarbamate |
| Cbz-NptGly- Arg-kbt, MF2009 | 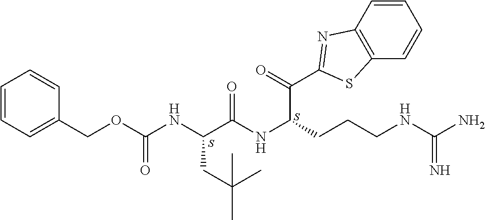 | (S)-3,3-dimethyl-1- [N-(S)-1-[(1,3- benzothiazol-2- yl)carbonyl]-4- guanidinobutylcarbamoyl] butyl- phenylmethanecarbamate |
| Cbz-Igl-Arg- kbt, MF2008 | 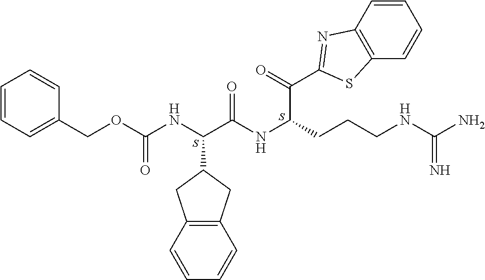 | (S)-(2-indanyl)[N- (S)-1-[(1,3- benzothiazol-2- yl)carbonyl]-4- guanidinobutylcarbamoyl] methyl- phenylmethanecarbamate |
| Fmoc-Ala(2- th)-Arg-kbt, MF1184 | 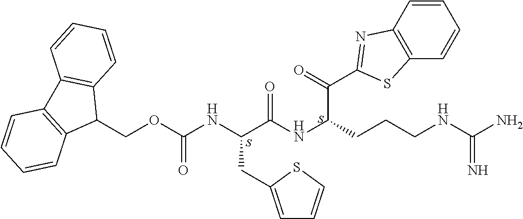 | (S)-1-[N-(S)-1-[(1,3- benzothiazol-2- yl)carbonyl]-4- guanidinobutylcarbamoyl]- 2-(2-thienyl)ethyl (9H- fluoren-9- yl)methanecarbamate |
| Cbz-Gly(2- th)-Arg-kbt, MF1177 | 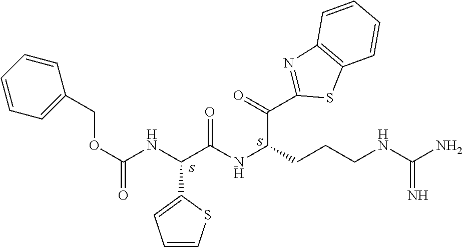 | (R)-[N-(S)-1-[(1,3- benzothiazol-2- yl)carbonyl]-4- guanidinobutylcarbamoyl] (2-thienyl)methyl- phenylmethanecarbamate |
| AJS4016, Ac- Ser-4- AMBA-Leu- Arg-kbt | 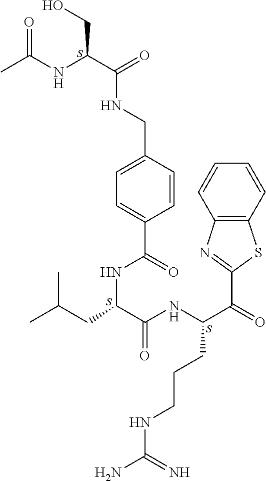 | N-[(S)-1-[(1,3- benzothiazol-2- yl)carbonyl]-4- guanidinobutyl](S)-2- (p-{[(S)-2- acetylamino-3- hydroxypropionylamino] methyl}benzylamino)-4- methylvaleramide |
| Cbz-QFR- kbt, ZFH9141 | 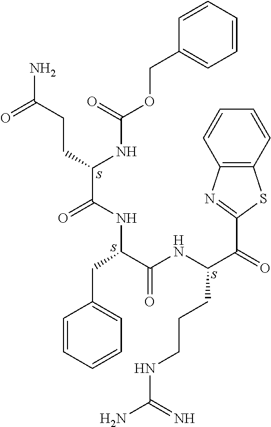 | (S)-1,3-bis[N-(S)-1- [N-(S)-1-[(1,3- benzothiazol-2- yl)carbonyl]-4- guanidinobutylcarbamoyl]- 2- phenylethylcarbamoyl]propyl- phenylmethanecarbamate |
| Cbz- Glu(OBzl)- Arg-kbt, MF1169 | 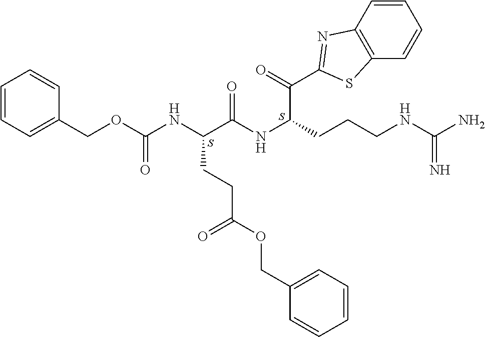 | benzyl (S)-4- [benzyl(oxycarbonylamino)]- 4-[N-(S)-1- [(1,3-benzothiazol-2- yl)carbonyl]-4- guanidinobutylcarbamoyl] butyrate |
| Cbz-Bta- Arg-kbt, MF1168 | 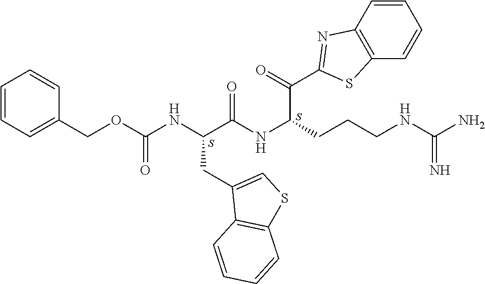 | (S)-2-(1- benzothiophen-3-yl)- 1-[N-(S)-1-[(1,3- benzothiazol-2- yl)carbonyl]-4- guanidinobutylcarbamoyl] ethyl- phenylmethanecarbamate |
| Fmoc-hPhe- Arg-kbt, MF1152 | 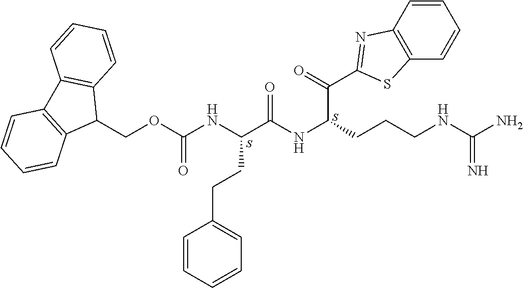 | (S)-1-[N-(S)-1-[(1,3- benzothiazol-2- yl)carbonyl]-4- guanidinobutylcarbamoyl]- 3-phenylpropyl (9H- fluoren-9- yl)methanecarbamate |
| Fmoc-Lys(2- CIZ)-Arg- kbt, MF1151 | 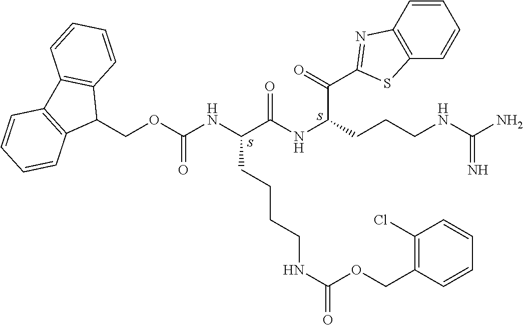 | (S)-5-{[(o- chlorophenyl)methyl] (oxycarbonylamino)}- 1-[N-(S)-1-[(1,3- benzothiazol-2- yl)carbonyl]-4- guanidinobutylcarbamoyl] pentyl (9H-fluoren-9- yl)methanecarbamate |
| Fmoc-Nva- Arg-kbt, MF1150 | 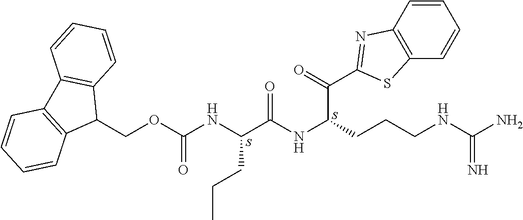 | (S)-1-[N-(S)-1-[(1,3- benzothiazol-2- yl)carbonyl]-4- guanidinobutylcarbamoyl] butyl (9H-fluoren-9- yl)methanecarbamate |
| Fmoc-hCha- Arg-kbt, MF1149 | 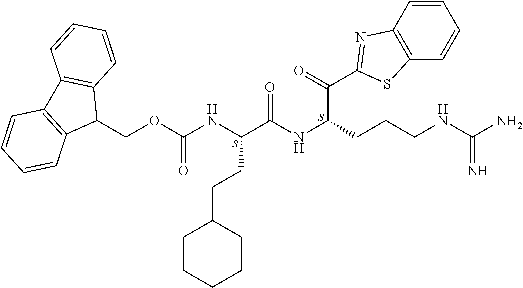 | (S)-3-cyclohexyl-1- [N-(S)-1-[(1,3- benzothiazol-2- yl)carbonyl]-4- guanidinobutylcarbamoyl] propyl (9H-fluoren-9- yl)methanecarbamate |
| Fmoc- Nle(OBzl)- Arg-kbt, MF1165 | 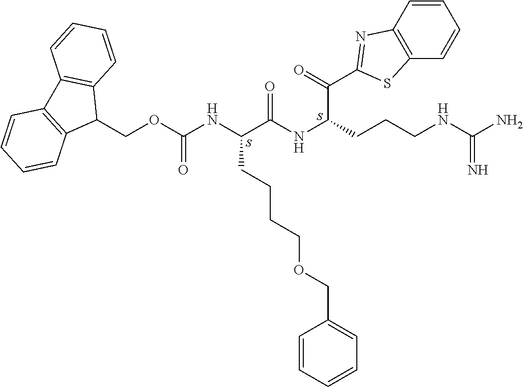 | (S)-5-(benzyloxy)-1- [N-(S)-1-[(1,3- benzothiazol-2- yl)carbonyl]-4- guanidinobutylcarbamoyl] pentyl (9H-fluoren-9- yl)methanecarbamate |
| Fmoc-hLeu- Arg-kbt, MF1164 | 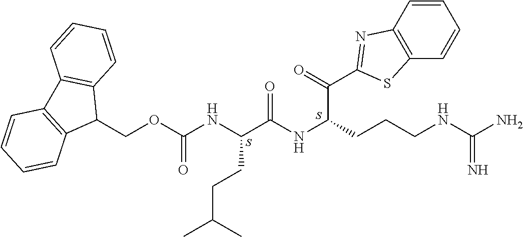 | (S)-4-methyl-1-[N- (S)-1-[(1,3- benzothiazol-2- yl)carbonyl]-4- guanidinobutylcarbamoyl] pentyl (9H-fluoren-9- yl)methanecarbamate |
| Fmoc-hTyr- Arg-kbt, MF1163 | 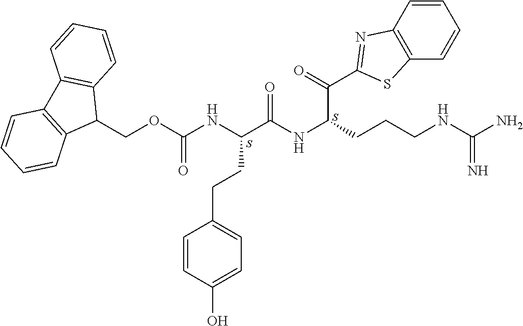 | (S)-3-(p- hydroxyphenyl)-1- [N-(S)-1-[(1,3- benzothiazol-2- yl)carbonyl]-4- guanidinobutylcarbamoyl] propyl (9H-fluoren-9- yl)methanecarbamate |
| Fmoc- His(Bzl)- Arg-kbt, MF1158 | 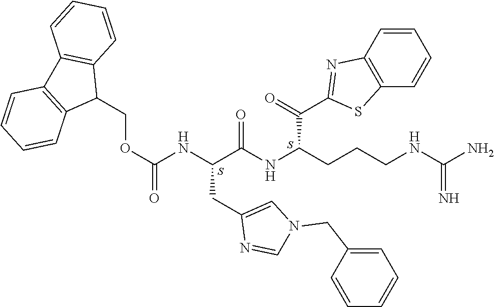 | (S)-2-(1-benzyl-4- imidazolyl)-1-[N-(S)- 1-[(1,3-benzothiazol- 2-yl)carbonyl]-4- guanidinobutylcarbamoyl] ethyl (9H-fluoren-9- yl)methanecarbamate |
| Fmoc-Phe(3- Cl)-Arg-kbt, MF1134 | 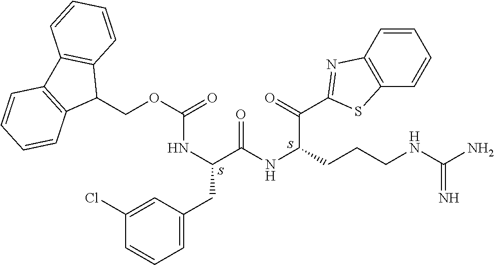 | (S)-2-(m- chlorophenyl)-1-[N- (S)-1-[(1,3- benzothiazol-2- yl)carbonyl]-4- guanidinobutylcarbamoyl] ethyl (9H-fluoren-9- yl)methanecarbamate |
| Fmoc-1-Nal- Arg-kbt, MF1144 | 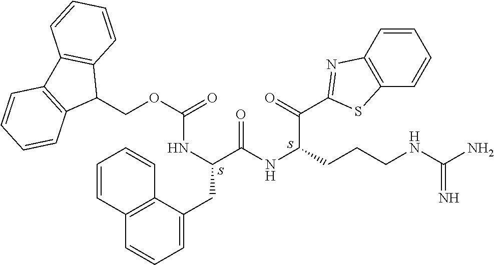 | (S)-2-(1-naphthyl)-1- [N-(S)-1-[(1,3- benzothiazol-2- yl)carbonyl]-4- guanidinobutylcarbamoyl] ethyl (9H-fluoren-9- yl)methanecarbamate |
| Fmoc-Nle- Arg-kbt, MF1143 | 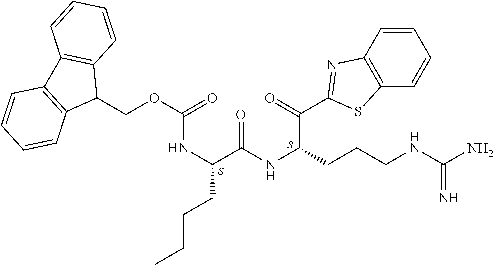 | (S)-1-[N-(S)-1-[(1,3- benzothiazol-2- yl)carbonyl]-4- guanidinobutylcarbamoyl] pentyl (9H-fluoren-9- yl)methanecarbamate |
| Fmoc-Cha- Arg-kbt, MF1142 | 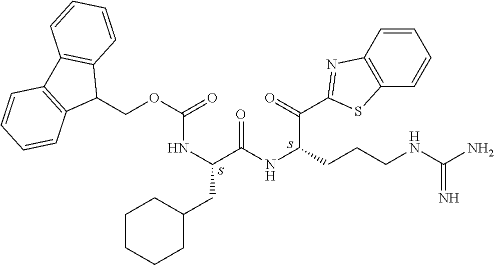 | (S)-2-cyclohexyl-1- [N-(S)-1-[(1,3- benzothiazol-2- yl)carbonyl]-4- guanidinobutylcarbamoyl] ethyl (9H-fluoren-9- yl)methanecarbamate |
| Ac-dhLeu- Glu(OAII)- Phe(4-F)- Arg-kbt, MF1140 | 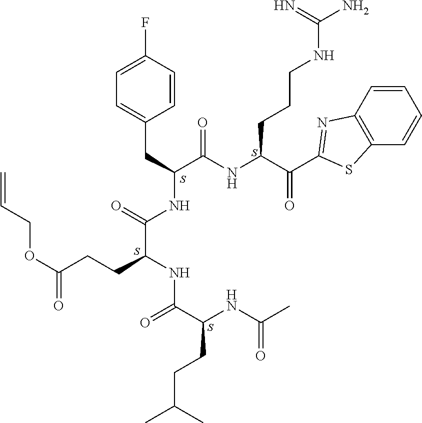 | allyl (S)-4-[(S)-1- acetylamino-4- methylpentylcarbonylamino]- 4-[N-(S)-2- (p-fluorophenyl)-1- [N-(S)-1-[(1,3- benzothiazol-2- yl)carbonyl]-4- guanidinobutylcarbamoyl] ethylcarbamoyl]butyrate |
| Ac-dhLeu- Glu(OChx)- Phe(4-F)- Arg-kbt, MF1125 | 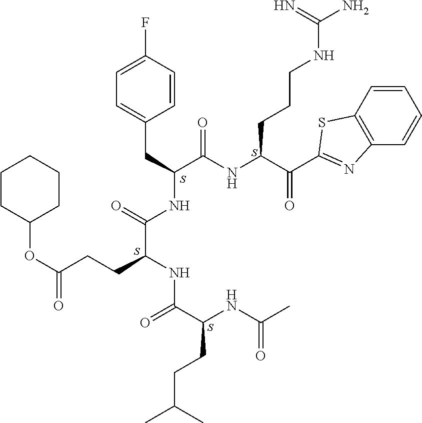 | cyclohexyl (S)-4- [(S)-1-acetylamino-4- methylpentylcarbonylamino]- 4-[N-(S)-2- (p-fluorophenyl)-1- [N-(S)-1-[(1,3- benzothiazol-2- yl)carbonyl]-4- guanidinobutylcarbamoyl] ethylcarbamoyl]butyrate |
| Fmoc-Phe(3- F)-Arg-kbt, MF1141 | 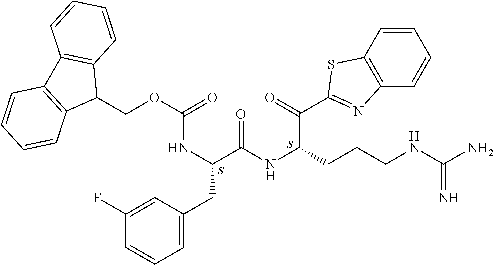 | (S)-2-(m- fluorophenyl)-1-[N- (S)-1-[(1,3- benzothiazol-2- yl)carbonyl]-4- guanidinobutylcarbamoyl] ethyl (9H-fluoren-9- yl)methanecarbamate |
| Fmoc-Phe(3, 4 di-F)-Arg- kbt, MF1132 | 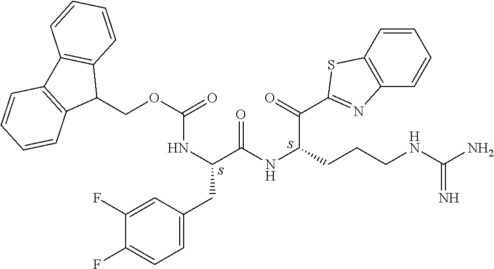 | (S)-2-(3,4- difluorophenyl)-1-[N- (S)-1-[(1,3- benzothiazol-2- yl)carbonyl]-4- guanidinobutylcarbamoyl]ethyl (9H-fluoren-9- yl)methanecarbamate |
| Fmoc-Phe(4- F)-Arg-kbt, MF1131 | 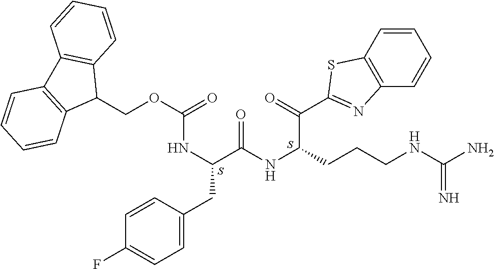 | (S)-2-(p- fluorophenyl)-1-[N- (S)-1-[(1,3- benzothiazol-2- yl)carbonyl]-4- guanidinobutylcarbamoyl]ethyl (9H-fluoren-9- yl)methanecarbamate |
| MF1101, PhSO2-QFR- kbt | 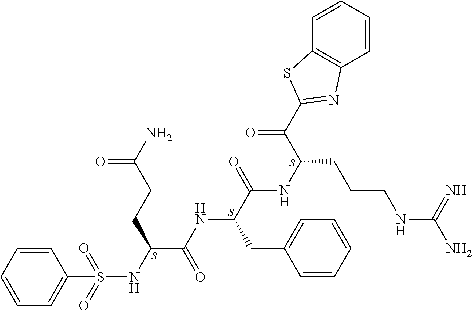 | N-[(S)-1-[N-(S)-1- [(1,3-benzothiazol-2- yl)carbonyl]-4- guanidinobutylcarbamoyl]-2- phenylethyl](S)-2- (phenylsulfonylamino) glutaramide |
| MF1099, MeSO2- QFR-kbt, N- 0385 (competitor patent and paper) | 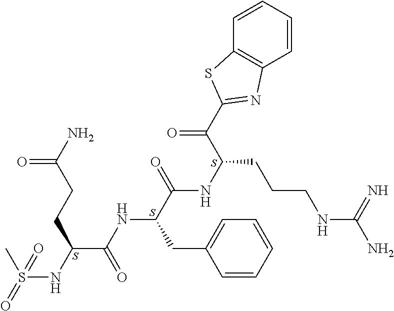 | N-[(S)-1-[N-(S)-1- [(1,3-benzothiazol-2- yl)carbonyl]-4- guanidinobutylcarbamoyl]- 2-phenylethyl](S)-2- (mesylamino)glutaramide |
| BnSO2- QFR-kbt, MF1105 | 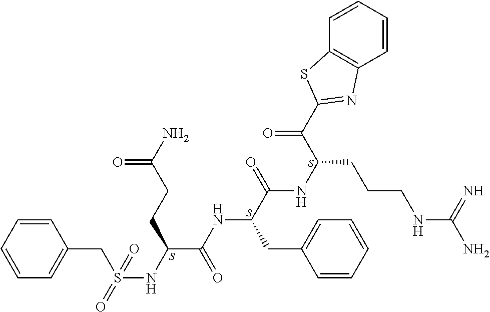 | N-[(S)-1-[N-(S)-1- [(1,3-benzothiazol-2- yl)carbonyl]-4- guanidinobutylcarbamoyl]-2- phenylethyl](S)-2- (benzylsulfonylamino) glutaramide |
| CypSO2- QFR-kbt, MF1104 | 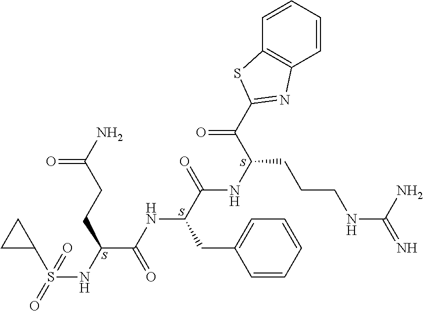 | N-[(S)-1-[N-(S)-1- [(1,3-benzothiazol-2- yl)carbonyl]-4- guanidinobutylcarbamoyl]-2- phenylethyl](S)-2- (cyclopropylsulfonylamino) glutaramide |
| Ac- OicGlu(OAll) Phe(4- F)Arg-kbt, MF1068 | 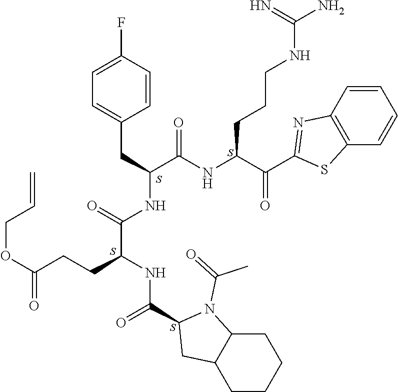 | allyl (S)-4-[(2S)-1- acetyloctahydro-2H- indol-2- ylcarbonylamino]-4- [N-(S)-2-(p- fluorophenyl)-1-[N- (S)-1-[(1,3- benzothiazol-2- yl)carbonyl]-4- guanidinobutylcarbamoyl] ethylcarbamoyl]butyrate |
| Ac- IdcGlu(OChx) Phe(4- F)Arg-kbt, MF1065 | 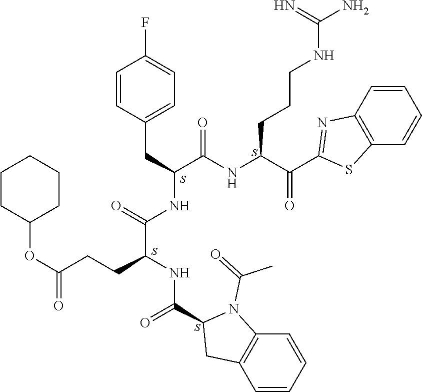 | cyclohexyl (S)-4- [(S)-1-acetyl-2- indolinylcarbonylamino]- 4-[N-(S)-2-(p- fluorophenyl)-1-[N- (S)-1-[(1,3- benzothiazol-2- yl)carbonyl]-4- guanidinobutylcarbamoyl] ethylcarbamoyl]butyrate |
| Ac- OicGlu(OChx) Phe(4- F)Arg-kbt, MF1064 | 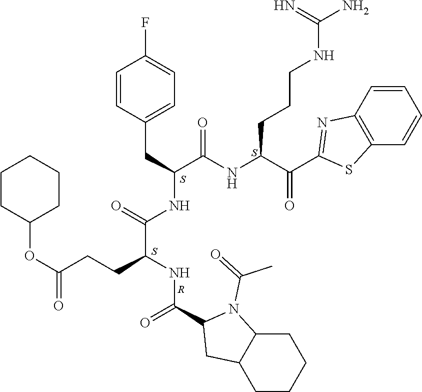 | cyclohexyl (S)-4-[(2R)-1- acetyloctahydro-2H- indol-2- ylcarbonylamino]-4- [N-(S)-2-(p- fluorophenyl)-1-[N- (S)-1-[(1,3-benzothiazol-2- yl)carbonyl]-4- guanidinobutylcarbamoyl] ethylcarbamoyl]butyrate |
| MM4146, VD4162- albumin | 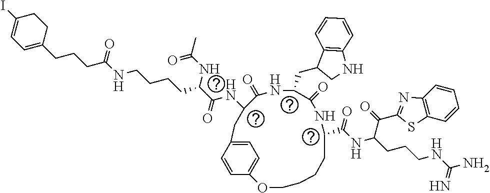 [SMILES: CC(=O)N[C@@H](CCCCNC(=O)CCCC1=CC=C(I)CC1)C(=O)NC1Cc2ccc(cc2)OCCCC[C@@H](C(=O)NC(CCCNC(=N)N)C(=O)c2nc3ccccc3s2)NC(=O)[C@@H](CC2CNc3ccccc32)NC1=O] | (7S,10R,13S)-10- ((1H-indol-3- yl)methyl)-13-((S)-2- acetamido-6-(4-(4- iodophenyl)butanamido) hexanamido)-N- (1-(benzo[d]thiazol- 2-yl)-5-guanidino-1- oxopentan-2-yl)- 9,12-dioxo-2-oxa- 8,11-diaza-1(1,4)- benzenacyclotetradecaphane- 7-carboxamide |
| CA1046-1, Pip-QFdR- kbt | 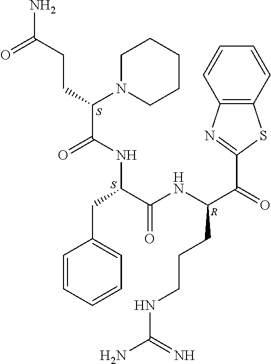 | N-[(S)-1-[N-(R)-1- [(1,3-benzothiazol-2- yl)carbonyl]-4- guanidinobutylcarbamoyl]-2- phenylethyl](S)-2- piperidinoglutaramide |
| CA1046-2, Pip-QFR-kbt | 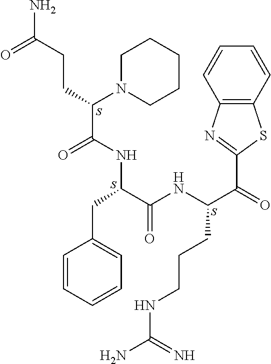 | N-[(S)-1-[N-(S)-1- [(1,3-benzothiazol-2- yl)carbonyl]-4- guanidinobutylcarbamoyl]-2- phenylethyl](S)-2- piperidinoglutaramide |
| CA1041-2, Me2-QFR- kbt | 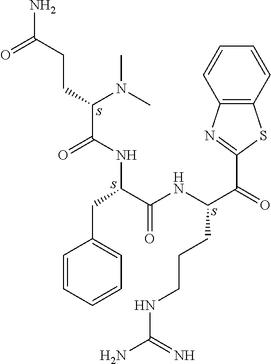 | N-[(S)-1-[N-(S)-1- [(1,3-benzothiazol-2- yl)carbonyl]-4- guanidinobutylcarbamoyl]-2- phenylethyl](S)-2- (dimethylamino)glutaramide |
| CA1041-1, Me2-QFdR- kbt | 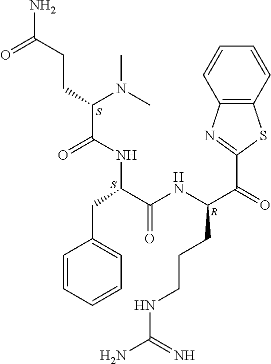 | N-[(S)-1-[N-(R)-1- [(1,3-benzothiazol-2- yl)carbonyl]-4- guanidinobutylcarbamoyl]-2- phenylethyl](S)-2- (dimethylamino)glutaramide |
| CA1033-1, PhEt-QFdR- kbt | 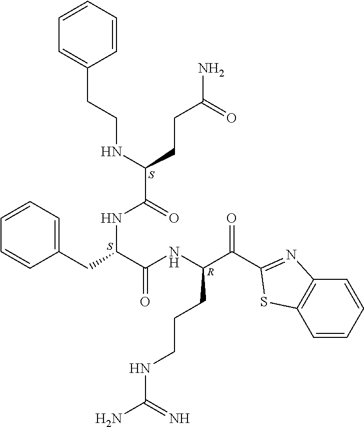 | N-[(S)-1-[N-(R)-1- [(1,3-benzothiazol-2- yl)carbonyl]-4- guanidinobutylcarbamoyl]-2- phenylethyl](S)-2- (phenethylamino)glutaramide |
| Bn-QFdR- kbt, CA1022- 1 | 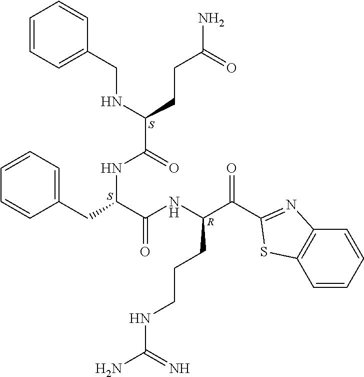 | N-[(S)-1-[N-(R)-1- [(1,3-benzothiazol-2- yl)carbonyl]-4- guanidinobutylcarbamoyl]-2- phenylethyl](S)-2- benzylaminoglutaramide |
| Bz-QFR-kbt, CA1043, MF1017, MF2001, ZFH9131 | 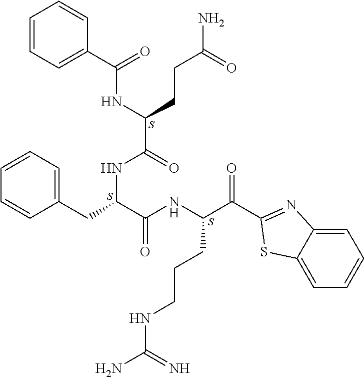 | N-[(S)-1-[N-(S)-1- [(1,3-benzothiazol-2- yl)carbonyl]-4- guanidinobutylcarbamoyl]-2- phenylethyl](S)-2- benzylaminoglutaramide |
| CA1033-2, PhEt-QFR- kbt | 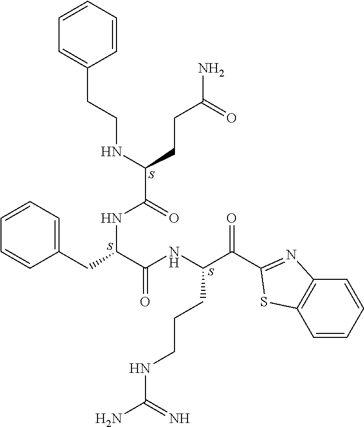 | N-[(S)-1-[N-(S)-1- [(1,3-benzothiazol-2- yl)carbonyl]-4- guanidinobutylcarbamoyl]-2- phenylethyl](S)-2- (phenethylamino)glutaramide |
| Bn-QFR-kbt, CA1022-2 | 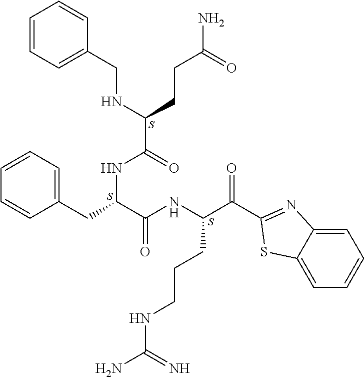 | N-[(S)-1-[N-(S)-1- [(1,3-benzothiazol-2- yl)carbonyl]-4- guanidinobutylcarbamoyl]-2- phenylethyl](S)-2- benzylaminoglutaramide |
| CA1018, PhCH2(CO)- QFR-kbt | 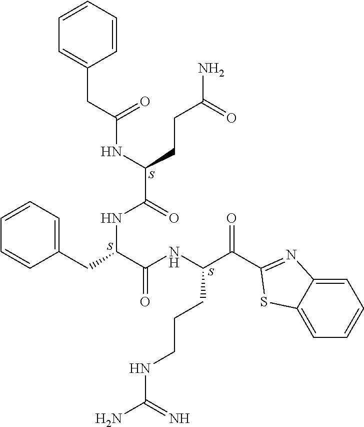 | N-[(S)-1-[N-(S)-1- [(1,3-benzothiazol-2- yl)carbonyl]-4- guanidinobutylcarbamoyl]-2- phenylethyl](S)-2- (benzylcarbonylamino) glutaramide |
| Ac-GQFdR- kbt MM3122-1 | 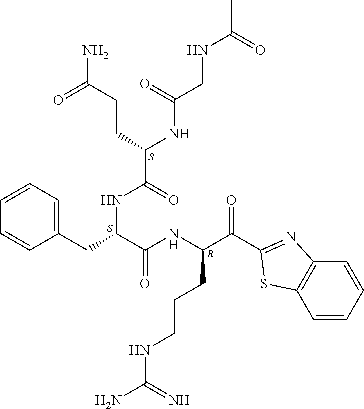 | N-[(S)-1-[N-(R)-1- [(1,3-benzothiazol-2- yl)carbonyl]-4- guanidinobutylcarbamoyl]-2- phenylethyl](S)-2- [(acetylaminomethyl) carbonylamino]glutaramide |
| Ac-IMFR- kbt, MM4027-2 | 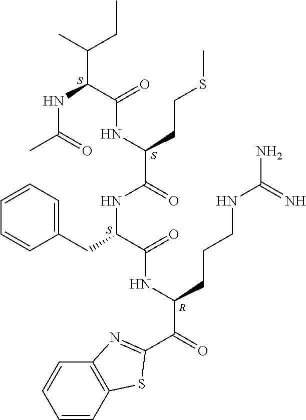 | N-[(S)-3-(methylthio)-1-[N- (S)-1-[N-(S)-1-[(1,3- benzothiazol-2- yl)carbonyl]-4- guanidinobutylcarbamoyl]-2- phenylethylcarbamoyl] propyl](2S)-2-acetylamino-3- methylvaleramide |
| VD5123B | 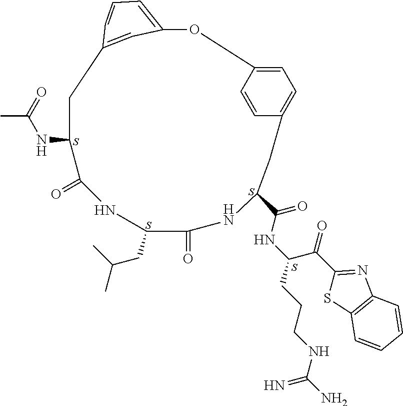 | N-[(S)-1-[(1,3-benzothiazol- 2-yl)carbonyl]-4- guanidinobutyl]- (9S,12S,15S)-9- acetylamino-12- isobutyl-10,13-dioxo- 2-oxa-11,14- diazatricyclo[15.2.2.1³,⁷] docosa-1(19),3,5,7(22), 17,20-hexaene-15- carboxamide |
| VD5123A | 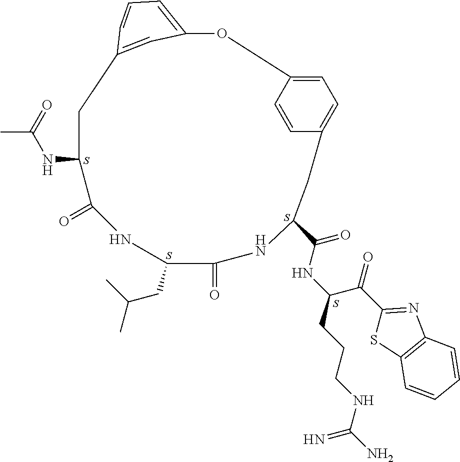 | N-{(R)-1-[(1,3- benzothiazol-2-yl)carbonyl]- 4-guanidinobutyl}- (9S,12S,15S)-9-acetylamino- 12-isobutyl-10,13-dioxo-2- oxa-11,14-diazatricyclo [15.2.2.1³,⁷]docosa- 1(19),3(22),4,6,17,20- hexaene-15-carboxamide |
| Ac-IdWFR- kbt, MM4038 | 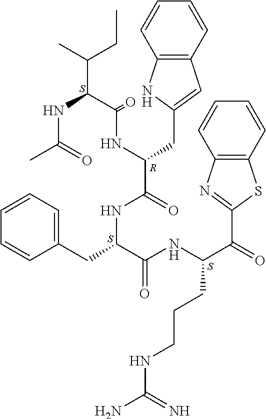 | N-[(R)-2-(2-indolyl)- 1-[N-(S)-1-[N-(S)-1- [(1,3-benzothiazol-2- yl)carbonyl]-4- guanidinobutylcarbamoyl]-2- phenylethylcarbamoy1] ethyl](2S)-2- acetylamino-3- methylvaleramide |
| Ac-IEFdR- kbt, MM4037-1 | 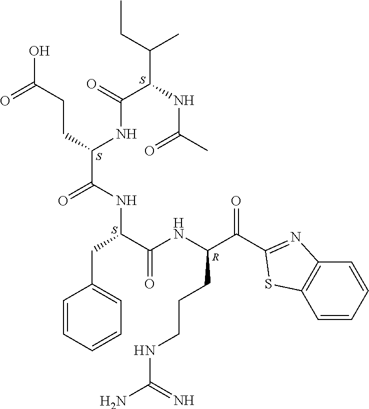 | (S)-4-[(2S)-2-acetylamino-3- methylvalerylamino]- 4-[N-(S)-1-[N-(R)-1- [(1,3-benzothiazol-2- yl)carbonyl]-4- guanidinobutylcarbamoyl]-2- phenylethylcarbamoyl]butyric acid |
| Ac-IEFR- kbt, MM4037-2 | 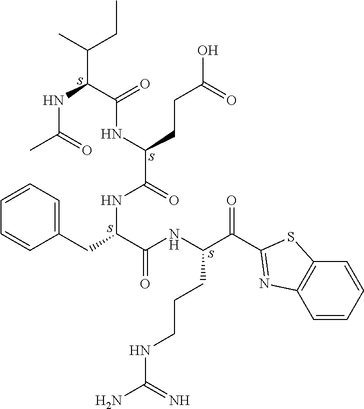 | (S)-4-[(2S)-2-acetylamino-3- methylvalerylamino]- 4-[N-(S)-1-[N-(S)-1- [(1,3-benzothiazol-2- yl)carbonyl]-4- guanidinobutylcarbamoyl]-2- phenylethylcarbamoyl]butyric acid |
| Ac-ITFR- kbt, MM4028-2 | 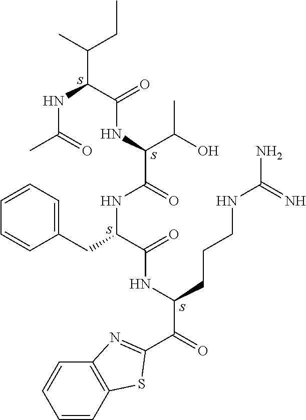 | N-[(1S)-2-hydroxy-1- [N-(S)-1-[N-(S)-1- [(1,3-benzothiazol-2- yl)carbonyl]-4- guanidinobutylcarbamoyl]-2- phenylethylcarbamoyl] propyl](2S)-2- acetylamino-3- methylvaleramide |
| Ac-ITFdR- kbt, MM4028-1 | 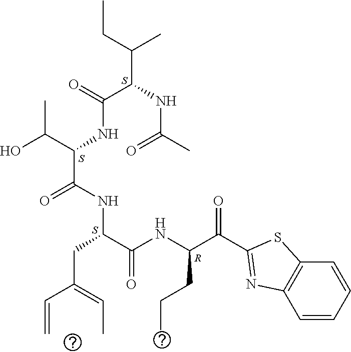 | N-[(1S)-2-hydroxy-1- [N-(S)-1-[N-(R)-1- [(1,3-benzothiazol-2- yl)carbonyl]-4- guanidinobutylcarbamoyl]-2- phenylethylcarbamoyl] propyl](2S)-2- acetylamino-3- methylvaleramide |
| Ac-IMFdR- kbt, MM4027-1 | 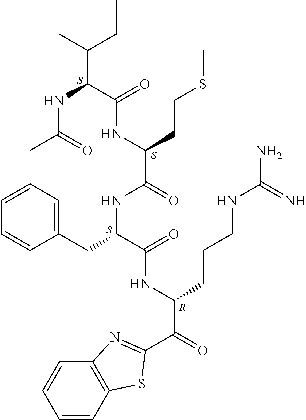 | N-[(S)-3-(methylthio)-1-[N- (S)-1-[N-(R)-1-[(1,3- benzothiazol-2- yl)carbonyl]-4- guanidinobutylcarbamoyl]-2- phenylethylcarbamoyl] propyl](2S)-2- acetylamino-3- methylvaleramide |
| Ac-ISFdR- kbt, MM4009-1 | 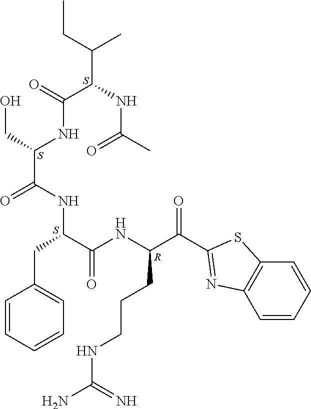 | N-[(S)-2-hydroxy-1- [N-(S)-1-[N-(R)-1- [(1,3-benzothiazol-2- yl)carbonyl]-4- guanidinobutylcarbamoyl]-2- phenylethylcarbamoyl]ethyl](2S)- 2-acetylamino-3- methylvaleramide |
| Ac-ISFR-kbt, MM4009-2 | 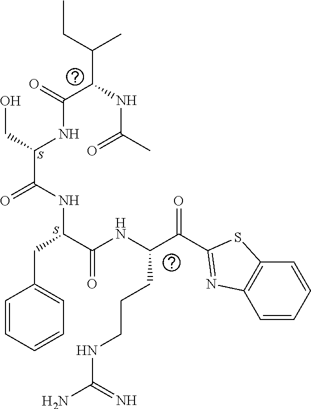 | N-[(S)-2-hydroxy-1- [N-(S)-1-[N-(S)-1- [(1,3-benzothiazol-2- yl)carbonyl]-4- guanidinobutylcarbamoyl]-2- phenylethylcarbamoyl]ethyl] (2S)-2-acetylamino-3- methylvaleramide |
| Ac-Ser- His(BOM)- Leu-Arg-kbt, MM1123-2, MM4036-2, JH1132 | 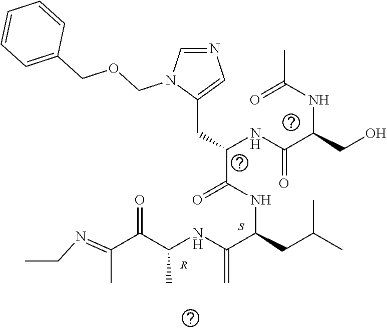 | N-[(S)-1-[(1,3-benzothiazol-2- yl)carbonyl]-4- guanidinobutyl](S)-2- [(S)-2-[(S)-2-acetylamino-3- hydroxypropionylamino]-3-{1- [(benzyloxy)methyl]-5- imidazolyl}propionyl amino]-4-methylvaleramide |
| Ac-Ser- His(BOM)- Leu-dArg- kbt, MM1123-1, MM4036-1 | 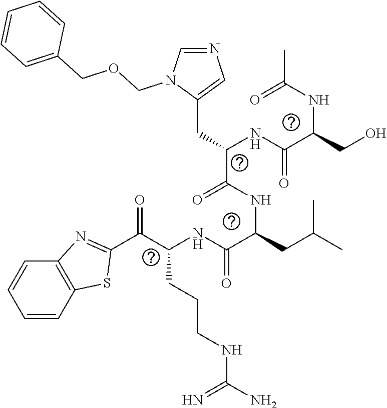 [SMILES: CC(=O)N[C@@H](CO)C(=O)N[C@@H](Cc1cncn1COCc1ccccc1)C(=O)N[C@@H](CC(C)C)C(=O)N[C@H](CCCNC(=N)N)C(=O)c1nc2ccccc2s1] | N-[(R)-1-[(1,3-benzothiazol-2- yl)carbonyl]-4- guanidinobutyl](S)-2- [(S)-2-[(S)-2-acetylamino-3- hydroxypropionylamino]-3-{1- [(benzyloxy)methyl]-5- imidazolyl}propionylamino]-4- methylvaleramide |
| Ac- His(BOM)- hARg-hLeu- dArg-kbt, JH1143-1, MM4032-1 | 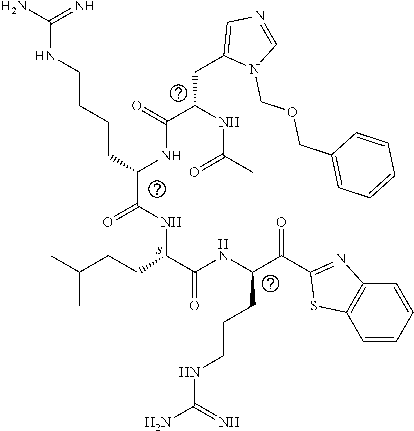 | N-[(R)-1-[(1,3-benzothiazol-2- yl)carbonyl]-4- guanidinobutyl](S)-2- [(S)-1-[(S)-2- acetylamino-3-{1- [(benzyloxy)methyl]-5- imidazolyl}propionylamino]-5- guanidinopentylcarbonylamino]- 5-methylhexanamide |
| Ac-hArg- His(BOM)- hLeu-dArg- kbt, JH1144, MM4031-1 | 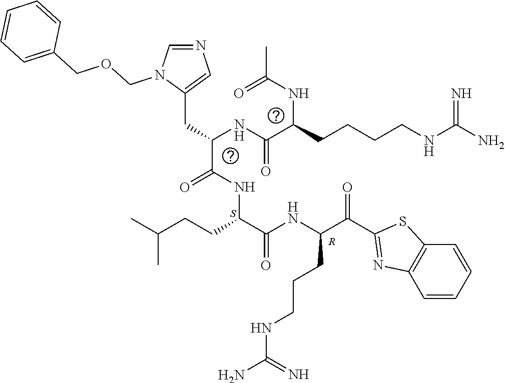 | N-[(R)-1-[(1,3-benzothiazol-2- yl)carbonyl]-4-guanidinobutyl](S)- 2-[(S)-2-[(S)-1-acetylamino-5- guanidinopentylcarbonylamino]- 3-{1-[(benzyloxy)methyl]- 5-imidazolyl}propionylamino]-5- methylhexanamide |
| Ac-hArg- His(BOM)- hLeu-Arg- kbt, JH1144- 2, MM4031-2 | 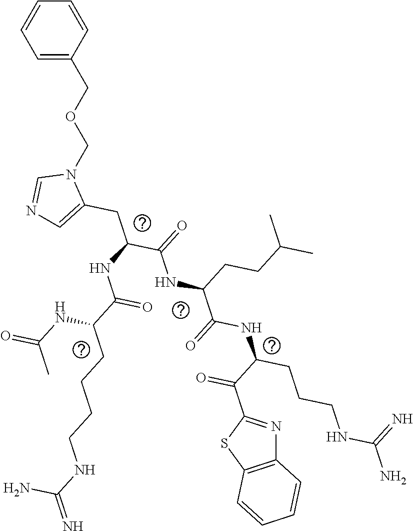 [SMILES: CC(=O)N[C@@H](CCCCNC(=N)N)C(=O)N[C@@H](Cc1cncn1COCc1ccccc1)C(=O)N[C@@H](CCC(C)C)C(=O)N[C@@H](CCCNC(=N)N)C(=O)c1nc2ccccc2s1] | N-[(S)-1-[(1,3-benzothiazol-2- yl)carbonyl]-4- guanidinobutyl](S)-2- [(S)-2-[(S)-1- acetylamino-5- guanidinopentylcarbonylamino]- 3-{1-[(benzyloxy)methyl]- 5-imidazolyl}propionyl amino]-5- methylhexanamide |
| VD5076A | 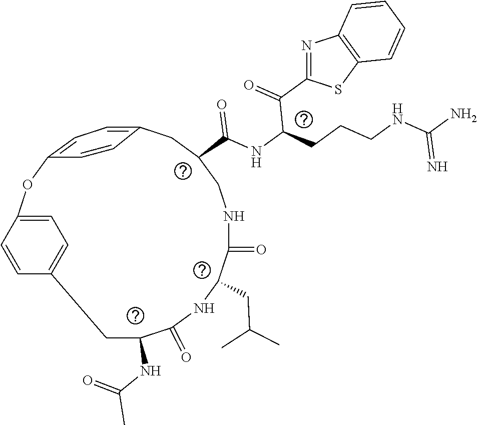 [SMILES: CC(=O)N[C@H]1Cc2ccc(cc2)Oc2ccc(cc2)C[C@@H](C(=O)N[C@H](CCCNC(=N)N)C(=O)c2nc3ccccc3s2)CNC(=O)[C@H](CC(C)C)NC1=O] | N-[(R)-1-[(1,3-benzothiazol-2- yl)carbonyl]-4-guanidinobutyl]- (8S,11S,14S)-14- acetylamino-11- isobutyl-10,13-dioxo- 2-oxa-9,12- diazatricyclo[14.2.2.2³,⁶]docosa- 1(18),3,5,16,19,21- hexaene-8-carboxamide |
| VD5076B | 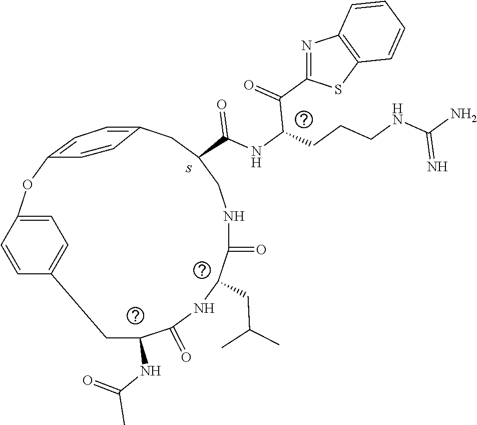 | N-[(S)-1-[(1,3-benzothiazol-2- yl)carbonyl]-4-guanidinobutyl]- (8S,11S,14S)-14- acetylamino-11- isobutyl-10,13-dioxo- 2-oxa-9,12- diazatricyclo[14.2.2.2³,⁶]docosa- 1(18),3,5,16,19,21- hexaene-8-carboxamide |
| VD5027 | 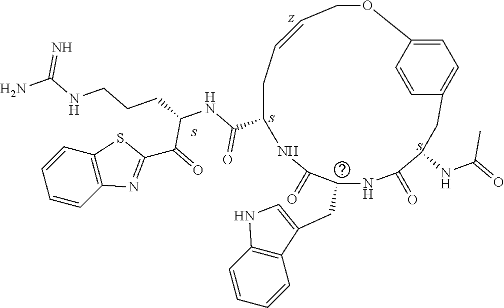 | N-[(S)-1-[(1,3-benzothiazol-2- yl)carbonyl]-4-guanidinobutyl]- (4Z,7S,10R,13S)-13- acetylamino-10-[(3- indolyl)methyl]-9,12- dioxo-2-oxa-8,11- diazabicyclo[13.2.2]nonadeca- 1(17),4,15,18-tetraene-7- carboxamide |
| Ac-PSKdR- kbt, MM3194C | 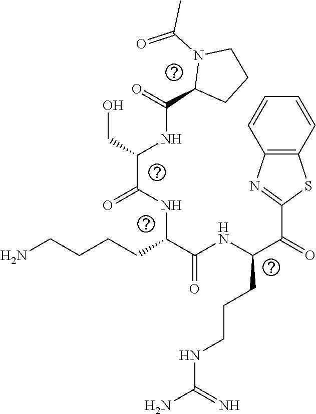 [SMILES: CC(=O)N1CCC[C@H]1C(=O)N[C@@H](CO)C(=O)N[C@@H](CCCCN)C(=O)N[C@H](CCCNC(=N)N)C(=O)c1nc2ccccc2s1] | N-[(S)-2-hydroxy-1-[N-(S)-5- amino-1-[N-(R)-1-[(1,3- benzothiazol-2- yl)carbonyl]-4- guanidinobutylcarbamoyl] pentylcarbamoy1]ethyl]- (S)-1-acetyl-2- pyrrolidinecarboxamide |
| Ac-PSKR- kbt, MM3194A | 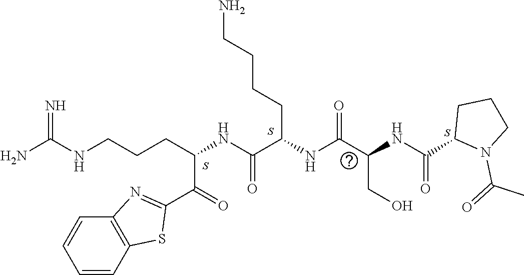 | N-[(S)-2-hydroxy-1- [N-(S)-5-amino-1-[N- (S)-1-[(1,3- benzothiazol-2- yl)carbonyl]-4- guanidinobutylcarbamoyl] pentylcarbamoy1]ethyl]- (S)-1-acetyl-2- pyrrolidinecarboxamide |
| VD5064A | 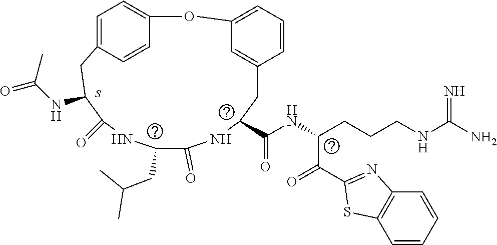 | N-{(R)-1-[(1,3-benzothiazol-2- yl)carbonyl]-4-guanidinobutyl}- (9S,12S,15S)-15- acetylamino-12- isobutyl-11,14-dioxo- 2-oxa-10,13- diazatricyclo[15.2.2.1³,⁷]docosa- 1(19),3,5,7(22),17,20-hexaene- 9-carboxamide |
| VD5064B | 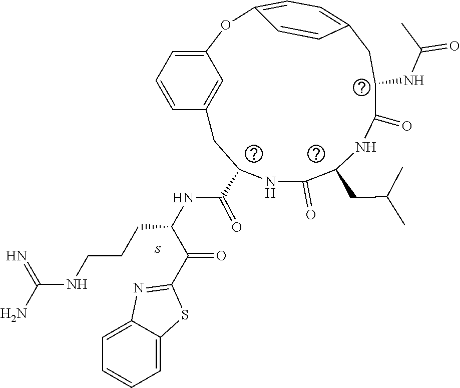 | N-[(S)-1-[(1,3-benzothiazol-2- yl)carbonyl]-4-guanidinobutyl]- (9S,12S,15S)-15- acetylamino-12- isobutyl-11,14-dioxo- 2-oxa-10,13- diazatricyclo[15.2.2.1³,⁷]docosa- 1(19),3,5,7(22),17,20- hexaene-9-carboxamide |
| Ac-IQTR- kbt, MM3186 | 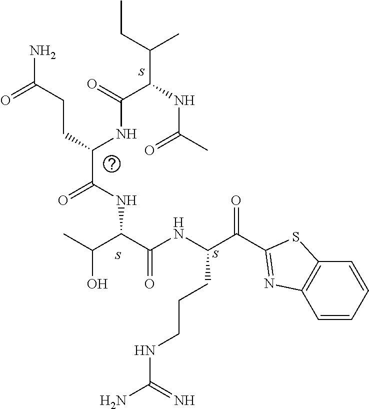 | N-[(1S)-2-hydroxy-1- [N-(S)-1-[(1,3-benzothiazol-2- yl)carbonyl]-4- guanidinobutylcarbamoyl] propyl](S)-2- [(2S)-2-acetylamino-3- methylvalerylamino]glutaramide |
| Ac-IQWR- kbt, MM3187 | 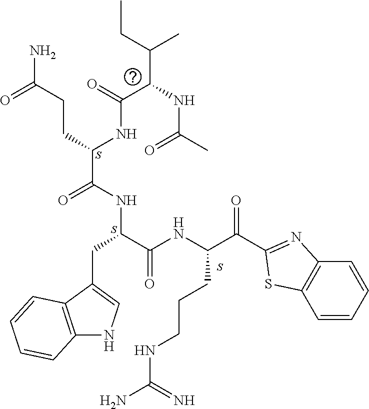 | N-[(S)-2-(3-indolyl)-1-[N-(S)- 1-[(1,3-benzothiazol-2- yl)carbonyl]-4- guanidinobutylcarbamoyl] ethyl](S)-2-[(2S)-2- acetylamino-3- methylvalerylamino]glutaramide |
| Ac-IQVR- kbt, MM3180-2 | 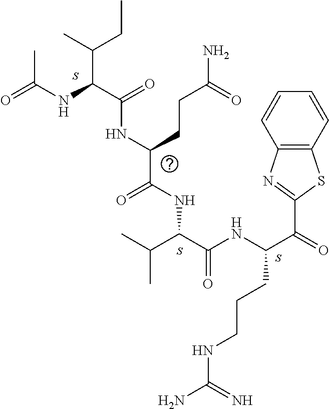 | N-[(S)-2-methyl-1-[N-(S)-1- [(1,3-benzothiazol-2- yl)carbonyl]-4- guanidinobutylcarbamoyl] propyl](S)-2- [(2S)-2-acetylamino-3- methylvalerylamino]glutaramide |
| Ac-IQVdR- kbt, MM3180-1 | 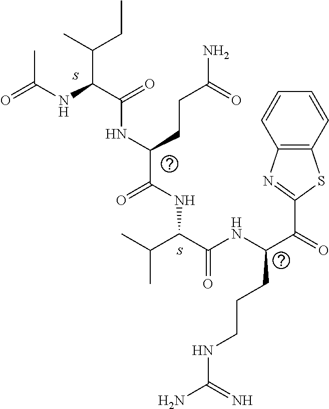 | N-[(S)-2-methyl-1-[N-(R)-1-[(1,3- benzothiazol-2-yl)carbonyl]-4- guanidinobutylcarbamoyl] propyl](S)-2- [(2S)-2-acetylamino-3- methylvalerylamino]glutaramide |
| Ac-IQAR- kbt, MM3178 | 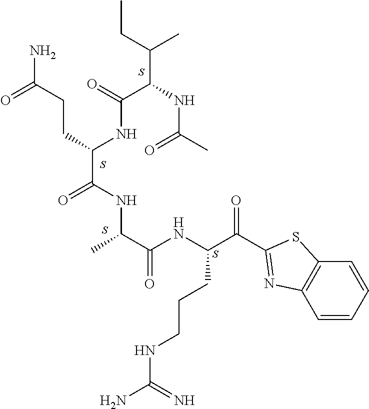 | N-[(S)-1-[N-(S)-1- [(1,3-benzothiazol-2- yl)carbonyl]-4- guanidinobutylcarbamoyl] ethyl](S)-2- [(2S)-2-acetylamino-3- methylvalerylamino]glutaramide |
| Ac-IQSR- kbt, MM3177 | 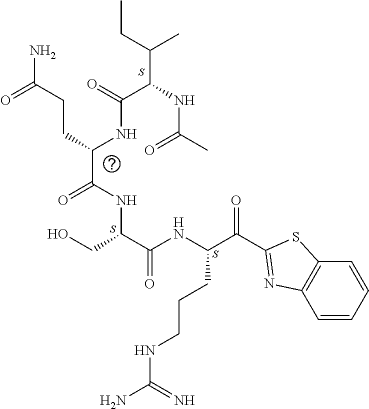 | N-[(S)-2-hydroxy-1-[N-(S)- 1-[(1,3-benzothiazol-2- yl)carbonyl]-4- guanidinobutylcarbamoyl] ethyl](S)-2-[(2S)-2-acetylamino- 3-methylvalerylamino]glutaramide |
| Ac-QFR-kbt, MM3144, N- 0386 | 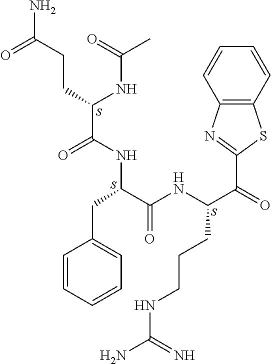 | N-[(S)-1-[N-(S)-1- [(1,3-benzothiazol-2- yl)carbonyl]-4- guanidinobutylcarbamoyl]-2- phenylethyl](S)-2- acetylaminoglutaramide |
| Ac-LQFR- kbt, MM3131 | 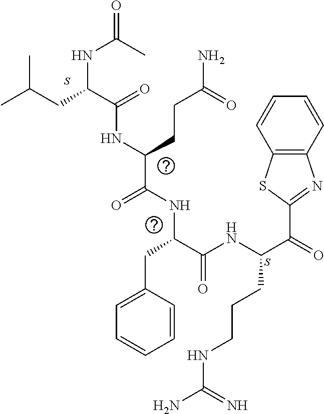 | N-[(S)-1-[N-(S)-1- [(1,3-benzothiazol-2- yl)carbonyl]-4- guanidinobutylcarbamoyl]-2- phenylethyl](S)-2- [(S)-2-acetylamino-4- methylvalerylamino]glutaramide |
| Ac-MQFR- kbt, MM3130 | 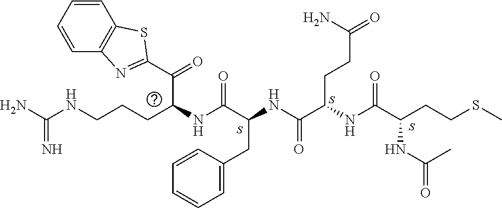 | N-[(S)-1-[N-(S)-1- [(1,3-benzothiazol-2- yl)carbonyl]-4- guanidinobutylcarbamoyl]-2- phenylethyl](S)-2- [(S)-2-acetylamino-4- (methylthio)butyrylamino] glutaramide |
| Ac-PQFR- kbt, MF1003, MF1016, MF2003, MM3123 | 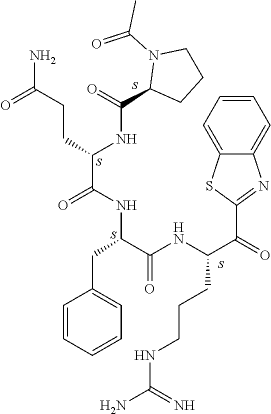 | N-[(S)-1-[N-(S)-1- [(1,3-benzothiazol-2- yl)carbonyl]-4- guanidinobutylcarbamoyl]-2- phenylethyl](S)-2- [(S)-1-acetyl-2- pyrrolidinylcarbonylamino] glutaramide |
| Ac-GQFR- kbt, MM3122-2 | 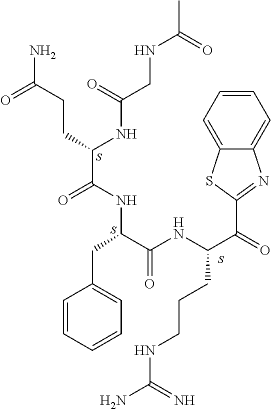 | N-[(S)-1-[N-(S)-1- [(1,3-benzothiazol-2- yl)carbonyl]-4- guanidinobutylcarbamoyl]-2- phenylethyl](S)-2- [(acetylaminomethyl) carbonylamino]glutaramide |
| Ac-IQFR- kbt, MM3116 | 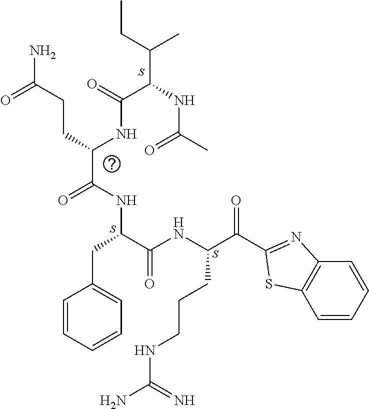 | N-[(S)-1-[N-(S)-1- [(1,3-benzothiazol-2- yl)carbonyl]-4- guanidinobutylcarbamoyl]-2- phenylethyl](S)-2- [(2S)-2-acetylamino-3- methylvalerylamino]glutaramide |
| VD4162A | 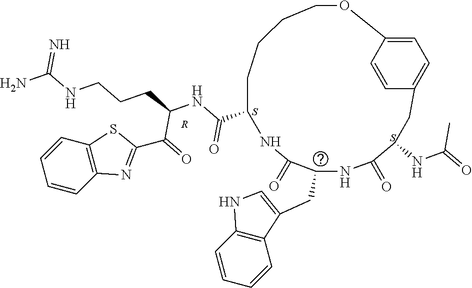 | N-[(R)-1-[(1,3-benzothiazol-2- yl)carbonyl]-4- guanidinobutyl]- (7S,10R,13S)-13- acetylamino-10-[(3- indolyl)methyl]-9,12- dioxo-2-oxa-8,11- diazabicyclo[13.2.2]nonadeca- 1(17),15,18- triene-7-carboxamide |
| VD4162B | 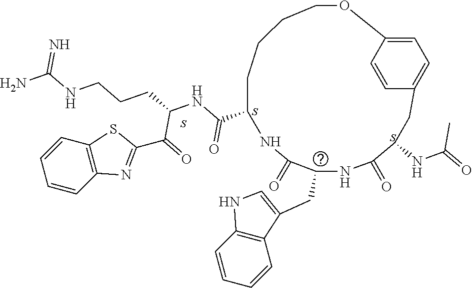 | N-[(S)-1-[(1,3-benzothiazol-2- yl)carbonyl]-4- guanidinobutyl]- (7S,10R,13S)-13- acetylamino-10-[(3- indolyl)methyl]-9,12- dioxo-2-oxa-8,11- diazabicyclo[13.2.2]nonadeca- 1(17),15,18- triene-7-carboxamide |
| VD4158 | 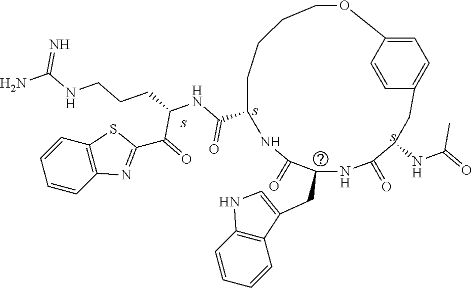 | N-[(S)-1-[(1,3-benzothiazol-2- yl)carbonyl]-4- guanidinobutyl]- (7S,10S,13S)-13- acetylamino-10-[(3- indolyl)methyl]-9,12- dioxo-2-oxa-8,11- diazabicyclo[13.2.2]nonadeca- 1(17),15,18- triene-7-carboxamide |
| VD4118A | 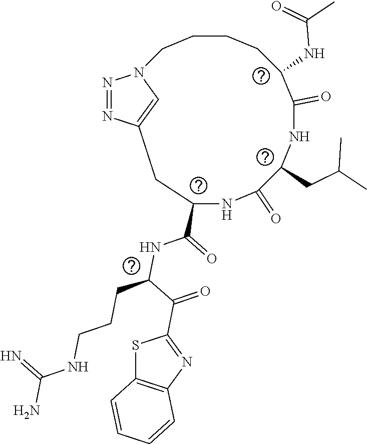 [SMILES: CC(=O)N[C@H]1CCCCn2cc(nn2)C[C@H](C(=O)N[C@H](CCCNC(=N)N)C(=O)c2nc3ccccc3s2)NC(=O)[C@H](CC(C)C)NC1=O] | N-[(R)-1-[(1,3-benzothiazol-2- yl)carbonyl]-4-guanidinobutyl]- (6S,9S,12R)-6-acetylamino-9- isobutyl-7,10-dioxo- 1,8,11,15,16- pentaazabicyclo[12.2.1]heptadeca- 14(17),15-diene-12- carboxamide |
| VD4111A | 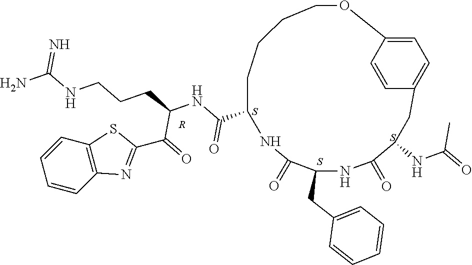 | N-[(R)-1-[(1,3-benzothiazol-2- yl)carbonyl]-4- guanidinobutyl]- (7S,10S,13S)-13- acetylamino-10- benzyl-9,12-dioxo-2-oxa-8,11- diazabicyclo[13.2.2]nonadeca- 1(17),15,18-triene-7-carboxamide |
| VD4118B | 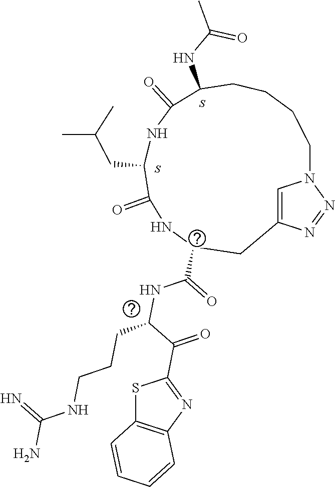 | N-[(S)-1-[(1,3-benzothiazol-2- yl)carbonyl]-4- guanidinobutyl]-(6S,9S,12R)-6- acetylamino-9-isobutyl-7,10- dioxo-1,8,11,15,16- pentaazabicyclo[12.2.1]heptadeca- 14(17),15-diene-12-carboxamide |
| VD4111B | 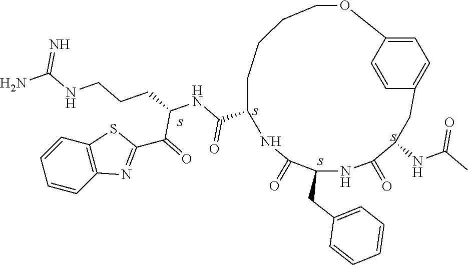 | N-[(S)-1-[(1,3-benzothiazol-2- yl)carbonyl]-4-guanidinobutyl]- (7S,10S,13S)-13-acetylamino- 10-benzyl-9,12-dioxo-2- oxa-8,11- diazabicyclo[13.2.2]nonadeca- 1(17),15,18- triene-7-carboxamide |
| VD4090 | 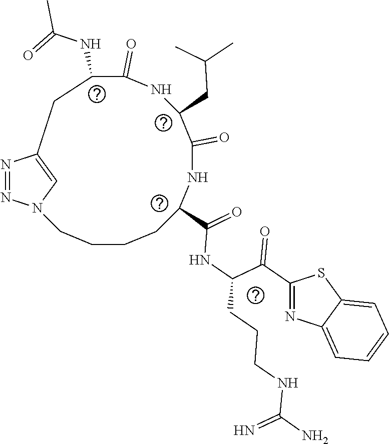 [SMILES: CC(=O)N[C@H]1Cc2cn(nn2)CCCC[C@H](C(=O)N[C@@H](CCCNC(=N)N)C(=O)c2nc3ccccc3s2)NC(=O)[C@H](CC(C)C)NC1=O] | N-[(S)-1-[(1,3-benzothiazol-2- yl)carbonyl]-4-guanidinobutyl]- (6R,9S,12S)-12-acetylamino-9- isobutyl-8,11-dioxo-1,7,10,15,16- pentaazabicyclo[12.2.1]heptadeca- 14(17),15-diene-6-carboxamide |
| VD4072 | 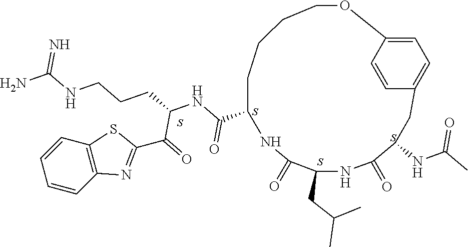 | N-[(S)-1-[(1,3-benzothiazol-2- yl)carbonyl]-4-guanidinobutyl]- (7S,10S,13S)-13-acetylamino-10- isobutyl-9,12-dioxo-2-oxa-8,11- diazabicyclo[13.2.2]nonadeca- 1(17),15,18-triene-7-carboxamide |
| VD4054 | 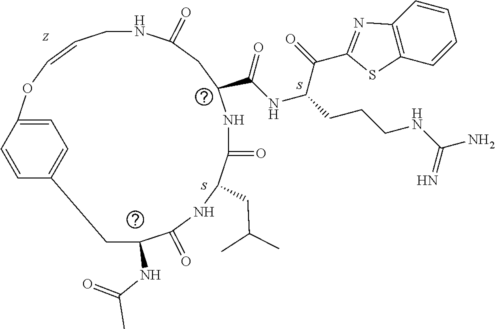 | N-[(S)-1-[(1,3-benzothiazol-2- yl)carbonyl]-4-guanidinobutyl]- (4Z,10S,13S,16S)- 16-acetylamino-13- isobutyl-8,12,15- trioxo-2-oxa-7,11,14- triazabicyclo[16.2.2]docosa- 1(20),4,18,21-tetraene-10- carboxamide |
| VD4051 | 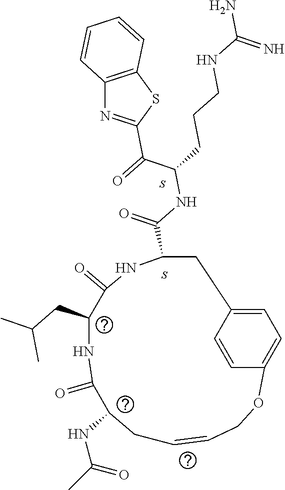 | N-[(S)-1-[(1,3-benzothiazol-2- yl)carbonyl]-4-guanidinobutyl]- (4Z,7S,10S,13S)-7-acetylamino- 10-isobutyl-8,11-dioxo- 2-oxa-9,12- diazabicyclo[13.2.2]nonadeca- 1(17),4,15,18-tetraene-13- carboxamide |
| VD4018 | 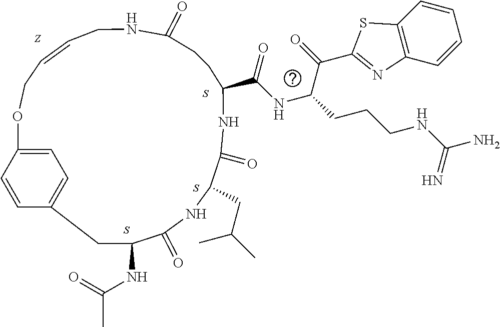 | N-[(S)-1-[(1,3-benzothiazol-2- yl)carbonyl]-4-guanidinobutyl]- (4Z,11S,14S,17S)- 17-acetylamino-14-isobutyl-8,13,16- trioxo-2-oxa-7,12,15- triazabicyclo[17.2.2]tricosa- 1(21),4,19,22-tetraene-11- carboxamide |
| VD4010 | 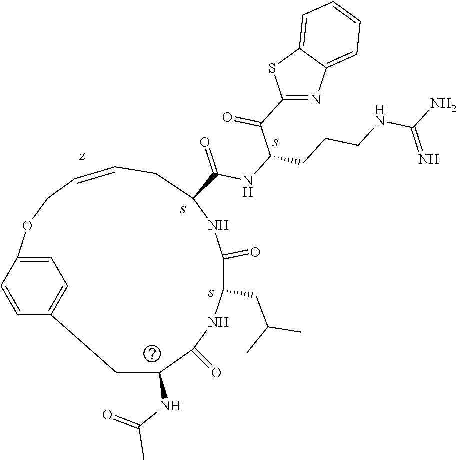 | N-[(S)-1-[(1,3-benzothiazol-2- yl)carbonyl]-4-guanidinobutyl]- (4Z,7S,10S,13S)-13- acetylamino-10-isobutyl-9,12- dioxo-2-oxa-8,11- diazabicyclo[13.2.2]nonadeca- 1(17),4,15,18-tetraene-7- carboxamide |
| VD3173 | 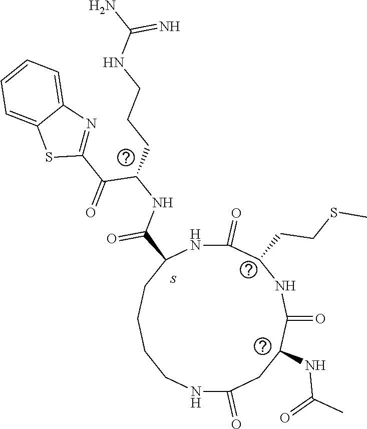 | N-[(S)-1-[(1,3-benzothiazol-2- yl)carbonyl]-4-guanidinobutyl]- (3S,6S,14S)-6-acetylamino-3-[2- (methylthio)ethyl]-2,5,8-trioxo- 1,4,9-triaza-14- cyclotetradecanecarboxamide |
| VD3152 | 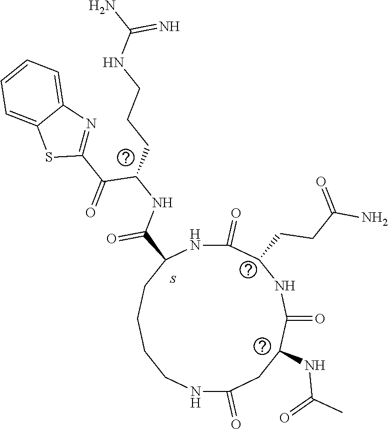 | N-[(S)-1-[(1,3- benzothiazol-2- yl)carbonyl]-4- guanidinobutyl]- (3S,6S,14S)-6- acetylamino-3-(2- carbamoylethyl)- 2,5,8-trioxo-1,4,9- triaza-14- cyclotetradecane- carboxamide |
| PK-1-45A1, dWFR-kbt- COOH | 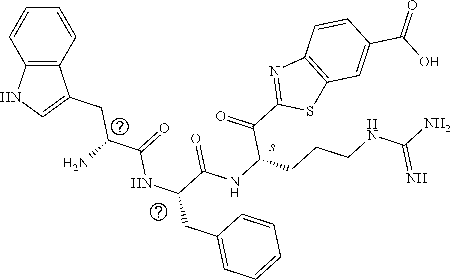 | 2-[(S)-2-[(S)-2-[(R)- 2-amino-3-(3- indolyl)propionylamino]-3- phenylpropionylamino]-5- guanidinovaleryl]- 1,3-benzothiazole-6- carboxylic acid |
| Ac-dWFR- kbt, MM3158, PK-1-89A1 | 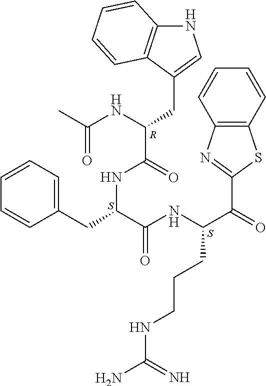 | N-[(S)-1-[(1,3-benzothiazol-2- yl)carbonyl]-4- guanidinobutyl](S)-2- [(R)-2-acetylamino-3-(3- indolyl)propionylamino]-3- phenylpropionamide |
| Ac-dWLR- kbt-COOH, MM4123, PK-1-94A1 | 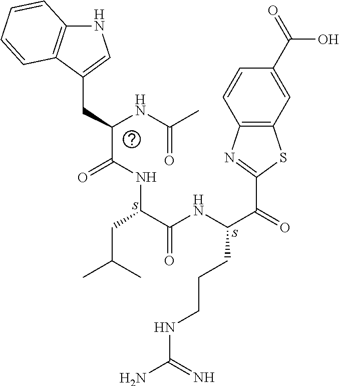 | 2-[(S)-2-[(S)-2-[(R)- 2-acetylamino-3-(3- indolyl)propionylamino]-4- methylvalerylamino]- 5-guanidinovaleryl]- 1,3-benzothiazole-6- carboxylic acid |
| Ac-WLR- kbt-COOH, MM4094, PK-1-105A1 | 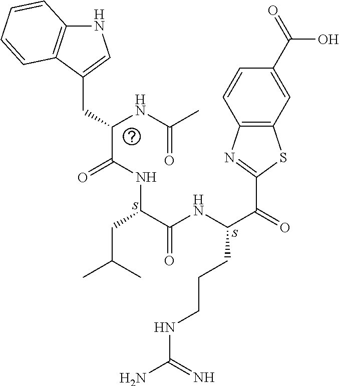 | 2-[(S)-2-[(S)-2-[(S)- 2-acetylamino-3-(3- indolyl)propionylamino]-4- methylvalerylamino]- 5-guanidinovaleryl]- 1,3-benzothiazole-6- carboxylic acid |
| Ac-dWFR- kbt-COOH, MM4058, PK-1-93A1 | 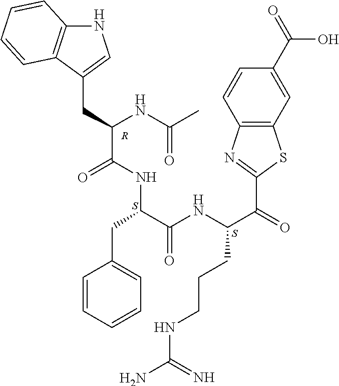 | 2-[(S)-2-[(S)-2-[(R)- 2-acetylamino-3-(3- indolyl)propionylamino]-3- phenylpropionylamino]-5- guanidinovaleryl]- 1,3-benzothiazole-6- carboxylic acid |
| Ac-WFR- kbt-COOH, MM4095, PK-1-103A1 | 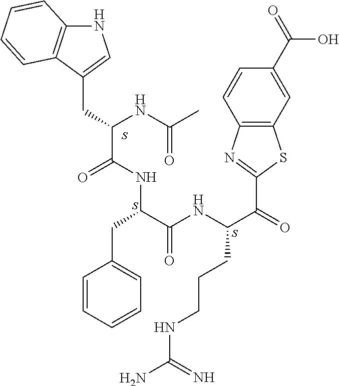 | 2-[(S)-2-[(S)-2-[(S)- 2-acetylamino-3-(3- indolyl)propionylamino]-3- phenylpropionylamino]-5- guanidinovaleryl]- 1,3-benzothiazole-6- carboxylic acid |
| Ac-WFR- kbt, MN1066, PK-1-102, PK-1-102A1 | 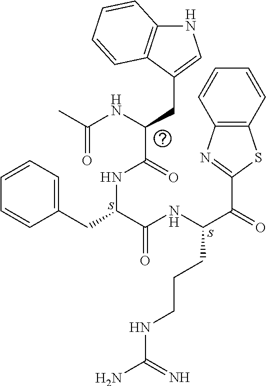 | N-[(S)-1-[(1,3-benzothiazol-2- yl)carbonyl]-4- guanidinobutyl](S)-2- [(S)-2-acetylamino-3-(3- indolyl)propionylamino]-3- phenylpropionamide |
| Ac-WLR- kbt, MN1070, PK-1-104, PK-1-104A1 | 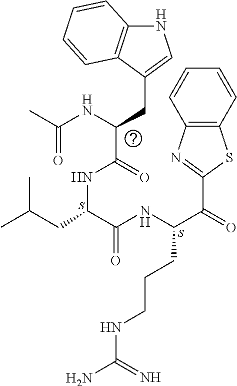 | N-[(S)-1-[(1,3-benzothiazol-2- yl)carbonyl]-4- guanidinobutyl](S)-2- [(S)-2-acetylamino-3-(3- indolyl)propionylamino]-4- methylvaleramide |
| Ac-dWLR- kbt, MN1063, PK-1-91, PK- 1-91A1 | 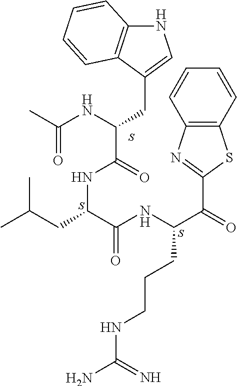 | N-[(S)-1-[(1,3-benzothiazol-2- yl)carbonyl]-4- guanidinobutyl](S)-2- [(R)-2-acetylamino-3-(3- indolyl)propionylamino]-4- methylvaleramide |
| JH1143-2, JH1190, MM4032-2 | 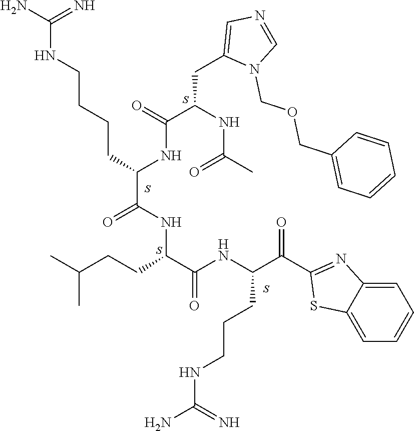 | N-[(S)-1-[(1,3-benzothiazol-2- yl)carbonyl]-4- guanidinobutyl](S)-2- [(S)-1-[(S)-2- acetylamino-3-{1- [(benzyloxy)methyl]-5- imidazolyl}propionyl amino]-5- guanidinopentylcarbonylamino]- 5-methylhexanamide |
| VD2173 | 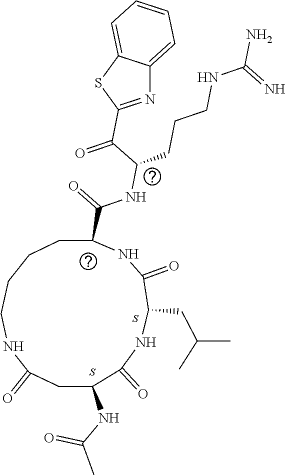 | N-[(S)-1-[(1,3-benzothiazol-2- yl)carbonyl]-4-guanidinobutyl]- (3S,6S,14S)-6-acetylamino-3- isobutyl-2,5,8-trioxo- 1,4,9-triaza-14- cyclotetradecanecarboxamide |
| AJS3200-2-1, Ac-Ser-4- AMPA-Leu- Arg-kbt, JH1125-2, JH1196 | 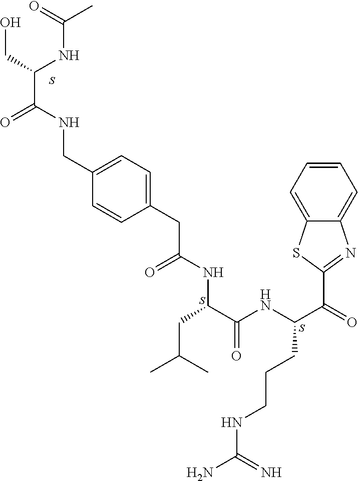 | N-[(S)-1-[(1,3-benzothiazol-2- yl)carbonyl]-4- guanidinobutyl](S)-2- {[(p-{[(S)-2-acetylamino-3- hydroxypropionylamino]methyl} phenyl)methyl]carbonylamino}- 4-methylvaleramide |
| PK-1-18A1, dWFR kbt | 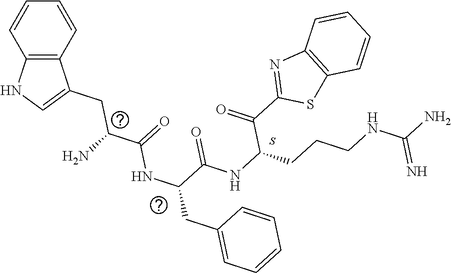 | N-[(S)-1-[(1,3-benzothiazol-2- yl)carbonyl]-4- guanidinobutyl](S)-2- [(R)-2-amino-3-(3- indolyl)propionylamino]-3- phenylpropionamide |
| Ac-SKFdR- kt, ZFH6095- iso | 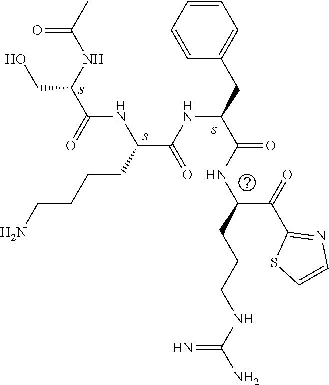 | N-[(S)-1-[N-(R)-4-guanidino-1-[(1,3- thiazol-2- yl)carbonyl]butylcarbamoyl]-2- phenylethyl](S)-2- [(S)-2-acetylamino-3- hydroxypropionylamino]-6- aminohexanamide |
| Ac-SKFR-kt, ZFH6095 | 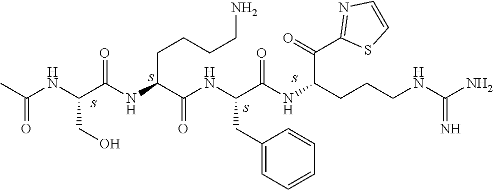 | N-[(S)-1-[N-(S)-4-guanidino-1- [(1,3-thiazol-2- yl)carbonyl]butylcarbamoyl]-2- phenylethyl](S)-2- [(S)-2-acetylamino-3- hydroxypropionylamino]-6- aminohexanamide |
| Ac-KQFdR- kt, ZFH6101- iso | 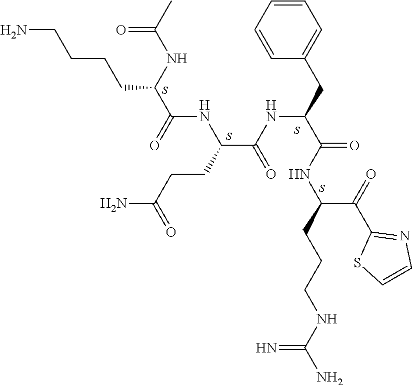 | N-[(S)-1-[N-(R)-4-guanidino-1- [(1,3-thiazol-2- yl)carbonyl]butylcarbamoyl]-2- phenylethyl](S)-2- [(S)-1-acetylamino-5- aminopentylcarbonylamino] glutaramide |
| Ac-KQFR- kt, ZFH6101 | 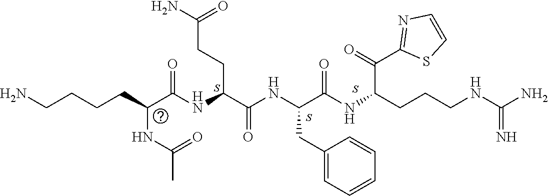 | N-[(S)-1-[N-(S)-4-guanidino-1- [(1,3-thiazol-2- yl)carbonyl]butylcarbamoyl]-2- phenylethyl](S)-2- [(S)-1-acetylamino-5- aminopentylcarbonylamino] glutaramide |
| Ac-SQLR-kt, ZFH6138 | 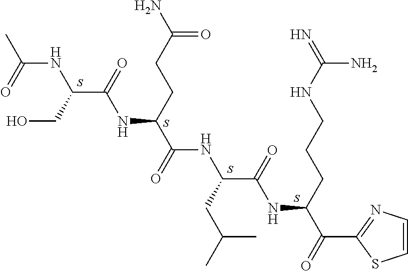 | N-[(S)-3-methyl-1-[N-(S)-4- guanidino-1-[(1,3-thiazol-2- yl)carbonyl]butylcarbamoyl] butyl](S)-2- [(S)-2-acetylamino-3- hydroxypropionylamino]glutaramide |
| Ac-LLR-kt, ZFH6201-1 | 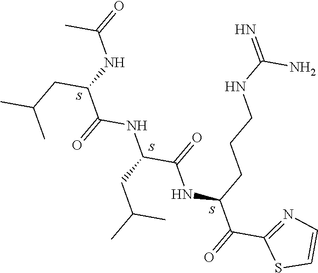 | N-[(S)-4-guanidino-1-[(1,3-thiazol- 2-yl)carbonyl]butyl](S)-2-[(S)-2- acetylamino-4- methylvalerylamino]- 4-methylvaleramide |
| Ac-KQLR- kbt, ZFH7006 | 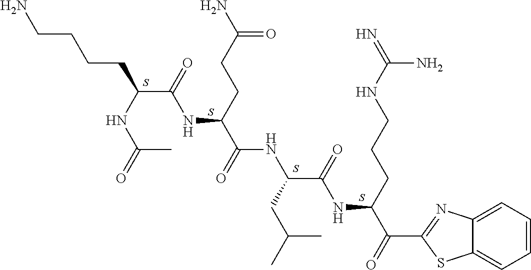 | N-[(S)-3-methyl-1-[N-(S)-1-[(1,3- benzothiazol-2- yl)carbonyl]-4- guanidinobutylcarbamoyl] butyl](S)-2- [(S)-1-acetylamino-5- aminopentylcarbonyl- amino]glutaramide |
| Ac-SKLR kbt, ZFH7053 | 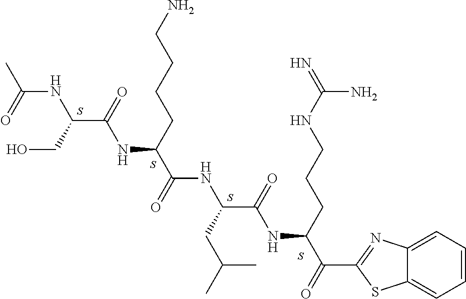 | N-[(S)-3-methyl-1- [N-(S)-1-[(1,3-benzothiazol-2- yl)carbonyl]-4- guanidinobutylcarbamoyl] butyl](S)-2- [(S)-2-acetylamino-3- hydroxypropionylamino]-6- aminohexanamide |
| Ac-FLFR- kbt, ZFH7063 | 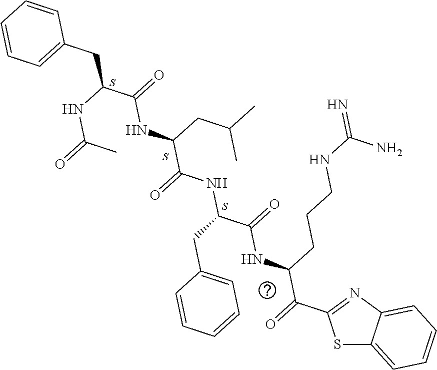 | N-[(S)-1-[N-(S)-1- [(1,3-benzothiazol-2- yl)carbonyl]-4- guanidinobutylcarbamoyl]-2- phenylethyl](S)-2- [(S)-2-acetylamino-3- phenylpropionylamino]-4- methylvaleramide |
| Ac-WLFR- kbt, ZFH7064 | 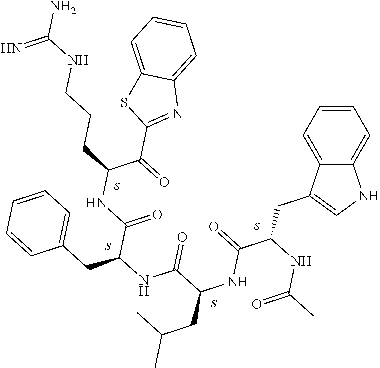 | N-[(S)-1-[N-(S)-1- [(1,3-benzothiazol-2- yl)carbonyl]-4- guanidinobutylcarbamoyl]-2- phenylethyl](S)-2- [(S)-2-acetylamino-3-(3- indolyl)propionylamino]-4- methylvaleramide |
| Ac-SKLR kbt V-amide, VD2156, VD3040, VD3182, VD4132, ZFH7116 | 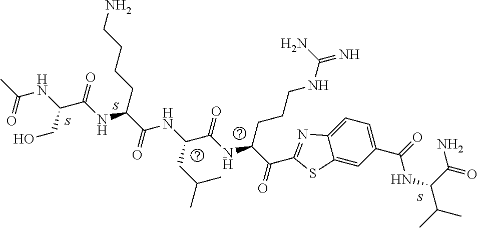 | N-[(S)-1-carbamoyl- 2-methylpropyl]-2- [(S)-2-[(S)-2-[(S)-1- [(S)-2-acetylamino-3- hydroxypropionylamino]-5- aminopentylcarbonyl- amino]-4- methylvalerylamino]- 5-guanidinovaleryl]- 1,3-benzothiazole-6- carboxamide |
| Fmoc-FR- kbt, MF1133, ZFH7187-14 | 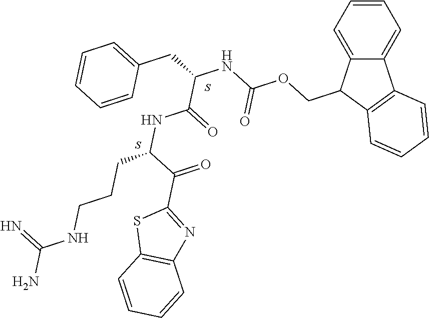 | (S)-1-[N-(S)-1-[(1,3- benzothiazol-2- yl)carbonyl]-4- guanidinobutylcarbamoyl]- 2-phenylethyl(9H-fluoren-9- yl)methanecarbamate |
| Ac- IdcGlu(OAll) [symbol] F)Arg-kbt, M [symbol] MPM2082 | 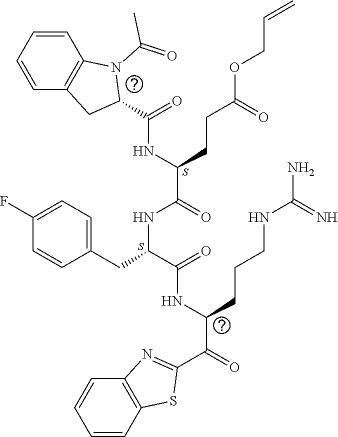 | allyl (S)-4-[(S)-1-acetyl-2- indolinylcarbonylamino]- 4-[N-(S)-2-(p- fluorophenyl)-1-[N- (S)-1-[(1,3-benzothiazol-2- yl)carbonyl]-4- guanidinobutylcarbamoyl] ethylcarbamoyl]butyrate |
| Ac- IdcGlu(OAll) Phe(4- F)dArg-kbt, MF1067, MPM2082A | 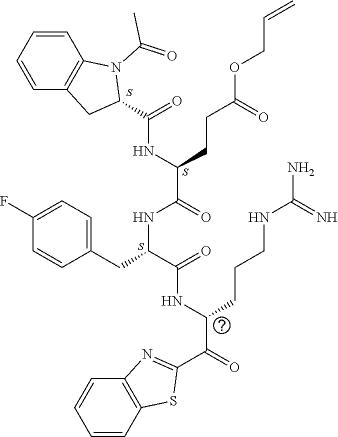 | allyl (S)-4-[(S)-1-acetyl-2- indolinylcarbonylamino]-4- [N-(S)-2-(p- fluorophenyl)-1-[N- (R)-1-[(1,3-benzothiazol-2- yl)carbonyl]-4- guanidinobutylcarbamoyl] ethylcarbamoyl]butyrate |
| Ac-Ser-3- AMPA-Leu- dArg-kbt, MM1132-1, MM4035-1 | 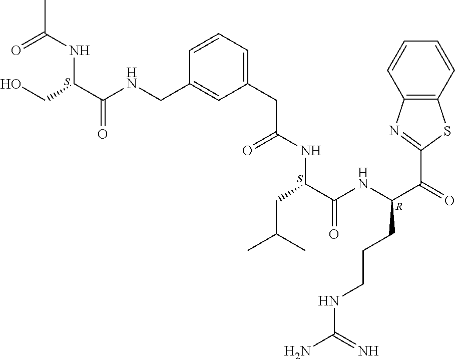 | N-[(R)-1-[(1,3-benzothiazol-2- yl)carbonyl]-4- guanidinobutyl](S)-2- {[(m-{[(S)-2-acetylamino-3- hydroxypropionylamino]methyl} phenyl)methyl]carbonylamino}- 4-methylvaleramide |
| JH1169, JH1189, Ac- Ser-D-Trp- Leu-Arg-kbt | 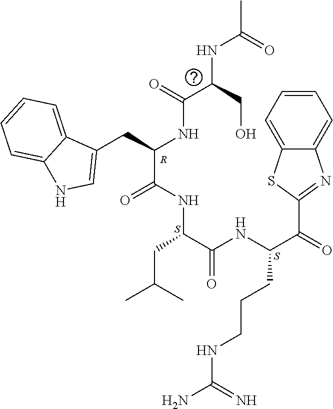 | N-[(S)-1-[(1,3-benzothiazol-2- yl)carbonyl]-4- guanidinobutyl](S)-2- [(R)-2-[(S)-2-acetylamino-3- hydroxypropionylamino]-3-(3- indolyl)propionylamino]-4- methylvaleramide |
| JH1142-2, Ac- His(BOM)- hLeu-Arg- kbt | 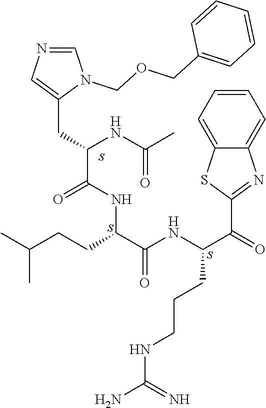 | N-[(S)-1-[(1,3-benzothiazol-2- yl)carbonyl]-4- guanidinobutyl](S)-2- [(S)-2-acetylamino-3-{1- [(benzyloxy)methyl]-5- imidazolyl}propionylamino]-5- methylhexanamide |
| JH1141-2, Ac-hArg- hLeu-Arg- kbt | 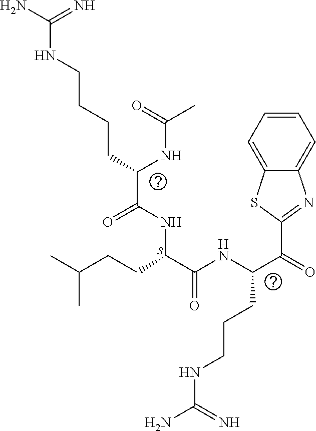 | N-[(S)-1-[(1,3-benzothiazol-2- yl)carbonyl]-4- guanidinobutyl](S)-2- [(S)-1-acetylamino-5- guanidinopentylcarbonylamino]- 5-methylhexanamide |
| JH1140-2, Ac-D-Trp- hArg-hLeu- Arg-kbt | 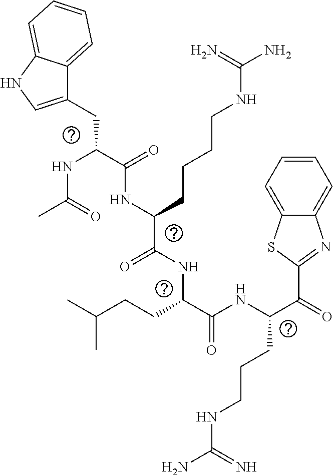 [SMILES: CC(=O)N[C@H](Cc1c[nH]c2ccccc12)C(=O)N[C@@H](CCCCNC(=N)N)C(=O)N[C@@H](CCC(C)C)C(=O)N[C@@H](CCCNC(=N)N)C(=O)c1nc2ccccc2s1] | N-[(S)-1-[(1,3-benzothiazol-2- yl)carbonyl]-4- guanidinobutyl](S)-2- [(S)-1-[(R)-2-acetylamino-3-(3- indolyl)propionylamino]-5- guanidinopentylcarbonylamino]- 5-methylhexanamide |
| Ac-Ser-3- AMPA-Leu- Arg-kbt, JH1126-2, MM1132-2, MM4035-2 | 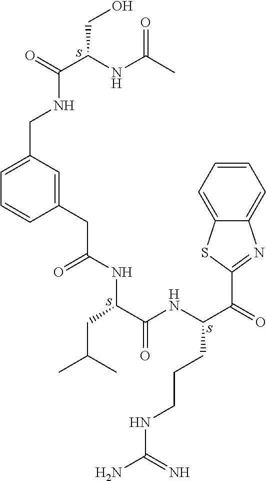 | N-[(S)-1-[(1,3-benzothiazol-2- yl)carbonyl]-4- guanidinobutyl](S)-2- {[(m-{[(S)-2-acetylamino-3- hydroxypropionylamino]methyl} phenyl)methyl]carbonylamino}- 4-methylvaleramide |
| MM1180, Ac-Ser- Phe(p-NO2)- Leu-Arg-kbt V amide | 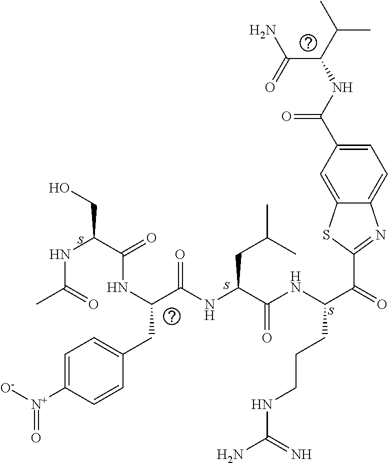 | N-[(S)-1-carbamoyl- 2-methylpropyl]-2- [(S)-2-[(S)-2-[(S)-2- [(S)-2-acetylamino-3- hydroxypropionylamino]-3-(p- nitrophenyl)propionylamino]-4- methylvalerylamino]- 5-guanidinovaleryl]- 1,3-benzothiazole-6- carboxamide |
| JH1114, MM1189, Ac-Ser-D- Trp-Leu- Arg-kbt V amide | 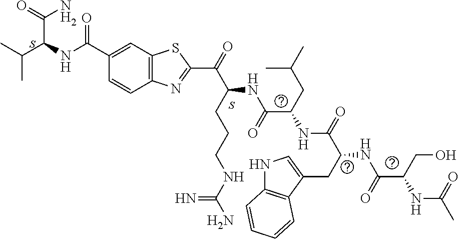 | N-[(S)-1-carbamoyl-2- methylpropyl]-2- [(S)-2-[(S)-2-[(R)-2- [(S)-2-acetylamino-3- hydroxypropionylamino]-3-(3- indolyl)propionylamino]-4- methylvalerylamino]- 5-guanidinovaleryl]- 1,3-benzothiazole-6- carboxamide |

[symbol]

indicates data missing or illegible when filed

### Example 2: Compound NMR and Mass Spectrometry Data Summary

TABLE 8
Compound NMR and Mass Spectrometry Data

| | | LCMS |
|---|---|---|
| | | (ESI, |
| Synonyms | 1H NMR | M + H+) |
| Fmoc-2-Nal- | ¹H NMR (400 MHz, cd3od) δ 8.22 − 8.13 (m, 1H), 8.07 | 711.798 |
| Arg-kbt, | (t, J = 7.3 Hz, 1H), 7.82 − 7.28 (m, 20H), 7.18 (qd, J = | |
| MF2012 | 15.6, 7.9 Hz, 3H), 5.63 (ddd, J = 19.5, 9.2, 4.0 Hz, 1H), | |
| | 4.58 (dt, J = 18.8, 7.5 Hz, 1H), 4.30 − 4.19 (m, 1H), 4.18 − | |
| | 4.01 (m, 2H), 3.21 (hept, J = 6.9 Hz, 2H), 3.08 (dt, J = | |
| | 13.6, 9.3 Hz, 1H), 2.96 (t, J = 7.1 Hz, 1H), 2.14 − 1.92 | |
| | (m, 1H), 1.82 − 1.54 (m, 2H), 1.32 (dd, J = 15.2, 8.0 Hz, | |
| | 2H). | |
| Fmoc-Orn- | ¹H NMR (600 MHz, MeOD) δ 8.28 − 8.15 (m, 1H), | 628.3 |
| Arg-kbt, | 8.14 − 8.07 (m, 1H), 7.82 − 7.77 (m, 3H), 7.70 − 7.57 (m, 5H), | |
| MF2011 | 7.43 − 7.35 (m, 3H), 7.35 − 7.26 (m, 3H), 5.81 − 5.60 (m, | |
| | 1H), 4.40 − 4.31 (m, 2H), 4.27 − 4.13 (m, 3H), 3.25 (t, J = | |
| | 6.8 Hz, 2H), 3.00 − 2.87 (m, 3H), 2.29 − 2.13 (m, 1H), | |
| | 1.92 − 1.64 (m, 9H). | |
| Cbz- | 1H NMR (600 MHz, MeOD) δ 8.24 − 8.18 (m, 1H), 8.16 − | 553.3 |
| NptGly- | 8.10 (m, 1H), 7.69 − 7.57 (m, 2H), 7.41 − 7.25 (m, 5H), | |
| Arg-kbt, | 5.72 − 5.56 (m, 1H), 5.21 − 4.99 (m, 2H), 4.31 − 4.17 (m, | |
| MF2009 | 1H), 3.46 − 3.37 (m, 1H), 3.29 − 3.18 (m, 2H), 2.25 − | |
| | 2.13 (m, 2H), 2.13 − 2.02 (m, 1H), 1.90 − 1.61 (m, 3H), | |
| | 1.58 − 1.38 (m, 3H), 1.37 − 1.25 (m, 1H), 1.07 − 0.77 (m, | |
| | 10H). | |
| Cbz-Igl- | 1H NMR (600 MHz, MeOD) δ 8.25 − 8.18 (m, 1H), 8.18 − | 599.3 |
| Arg-kbt, | 8.11 (m, 1H), 7.70 − 7.59 (m, 2H), 7.38 − 7.26 (m, 4H), | |
| MF2008 | 7.13 − 6.96 (m, 4H), 5.77 − 5.55 (m, 1H), 5.13 − 5.03 (m, | |
| | 2H), 4.26 − 4.16 (m, 1H), 3.44 − 3.33 (m, 0H), 3.30 − | |
| | 3.22 (m, 2H), 3.07 − 2.92 (m, 1H), 2.88 − 2.72 (m, 4H), | |
| | 2.25 − 2.14 (m, 1H), 1.93 − 1.67 (m, 3H), 1.53 − 1.26 (m, | |
| | 1H). | |
| Fmoc-Ala(2- | 1H NMR (600 MHz, MeOD) δ 8.26 − 8.17 (m, 1H), 8.17 − | 667.2 |
| th)-Arg-kbt, | 8.03 (m, 1H), 7.80 (t, J = 7.2 Hz, 2H), 7.69 − 7.56 (m, | |
| MF1184 | 4H), 7.42 − 7.33 (m, 2H), 7.35 − 7.24 (m, 2H), 6.99 − | |
| | 6.77 (m, 2H), 5.76 − 5.59 (m, 1H), 4.54 − 4.40 (m, 1H), | |
| | 4.40 − 4.29 (m, 1H), 4.25 − 4.10 (m, 2H), 3.25 − 3.11 (m, | |
| | 5H), 2.24 − 2.00 (m, 2H), 1.90 − 1.66 (m, 2H), 1.66 − | |
| | 1.53 (m, 1H), 1.40 − 1.22 (m, 5H). | |
| Cbz-Gly(2- | 1H NMR (600 MHz, MeOD) δ 8.22 − 8.15 (m, 1H), 8.15 − | 565.2 |
| th)-Arg-kbt, | 8.08 (m, 1H), 7.66 − 7.57 (m, 2H), 7.41 − 7.23 (m, 6H), | |
| MF1177 | 7.16 − 7.08 (m, 1H), 6.96 − 6.88 (m, 1H), 6.86 (dd, J = | |
| | 5.2, 3.5 Hz, 1H), 5.70 (ddd, J = 29.5, 9.6, 4.4 Hz, 2H), | |
| | 5.21 − 4.97 (m, 3H), 3.30 − 3.24 (m, 1H), 3.23 − 3.16 (m, | |
| | 1H), 2.26 − 2.11 (m, 1H), 1.93 − 1.73 (m, 3H), 1.70 − | |
| | 1.54 (m, 1H). | |
| AJS4016, | 1H NMR (600 MHz, MeOD) δ 8.57 (t, J = 6.1 Hz, 1H), | 667.3 |
| Ac-Ser-4- | 8.19 − 8.15 (m, 1H), 8.13 − 8.11 (m, 1H), 7.80 − 7.77 (m, | |
| AMBA-Leu- | 2H), 7.66 − 7.59 (m, 2H), 7.41 (s, 2H), 5.70 (dd, J = 9.5, | |
| Arg-kbt | 4.1 Hz, 1H), 4.68 − 4.59 (m, 2H), 4.48 (d, J = 5.2 Hz, | |
| | 3H), 4.43 (t, J = 5.3 Hz, 2H), 3.82 (qd, J = 11.0, 5.4 Hz, | |
| | 3H), 2.66 (s, 1H), 2.27 − 2.18 (m, 1H), 2.03 (s, 3H), 1.92 − | |
| | 1.77 (m, 4H), 1.74 − 1.62 (m, 4H), 0.94 (dd, J = 17.2, | |
| | 6.2 Hz, 9H). | |
| Cbz-QFR- | 1H NMR (399 MHz, cd3od) δ 8.22 (d, J = 7.5 Hz, 1H), | 701.3 |
| kbt, | 8.16 − 8.10 (m, 1H), 7.73 − 7.59 (m, 2H), 7.41 − 7.02 (m, | |
| ZFH9141 | 10H), 5.74 − 5.66 (m, 1H), 5.12 − 5.06 (m, 2H), 4.77 − | |
| | 4.58 (m, 1H), 4.10 − 3.95 (m, 1H), 3.26 − 3.17 (m, 2H), | |
| | 3.09 (dd, J = 13.9, 6.7 Hz, 1H), 3.00 − 2.89 (m, 1H), 2.23 | |
| | (q, J = 6.9 Hz, 1H), 2.18 − 2.05 (m, 2H), 2.01 − 1.89 (m, | |
| | 1H), 1.89 − 1.59 (m, 2H). | |
| Cbz- | 1H NMR (400 MHz, CD3OD) δ 8.21 − 8.18 (m, 1H), | 645.98 |
| Glu(OBzl)- | 8.13 − 8.09 (m, 1H), 7.63 − 7.59 (m, 2H), | |
| Arg-kbt, | 7.32 (p, J = 2.7 Hz, 10H), 5.71 − 5.66 (m, 1H), 5.09 − | |
| MF1169 | 5.04 (m, 5H), 4.22 (dd, J = 9.1, 4.9 Hz, | |
| | 1H), 3.27 − 3.21 (m, 2H), 2.50 − 2.45 (m, 2H), 2.11 (dt, J = | |
| | 14.1, 6.7 Hz, 2H), 1.99 − 1.90 (m, | |
| | 1H), 1.79 (q, J = 9.0 Hz, 2H), 1.70 (t, J = 7.3 Hz, 1H). | |
| Cbz-Bta- | 1H NMR (400 MHz, CD3OD) δ 8.19 (dd, J = 7.7, 1.8 | 629.92 |
| Arg-kbt, | Hz, 1H), 8.13 (td, J = 6.1, 1.8 Hz, 1H), 7.85 | |
| MF1168 | (d, J = 8.0 Hz, 1H), 7.76 (dd, J = 12.9, 8.2 Hz, 1H), 7.63 | |
| | (td, J = 7.4, 3.5 Hz, 2H), 7.39 − 7.32 (m, | |
| | 3H), 7.32 − 7.24 (m, 6H), 5.66 − 5.58 (m, 1H), 5.04 (d, J = | |
| | 8.7 Hz, 2H), 4.57 (dt, J = 20.5, 7.4 | |
| | Hz, 1H), 4.41 − 4.15 (m, 1H), 3.75 − 3.62 (m, 1H), 3.41 − | |
| | 3.33 (m, 1H), 3.19 (dt, J = 14.7, 7.3 | |
| | Hz, 3H), 3.10 (t, J = 7.3 Hz, 1H), 2.06 (d, J = 26.0 Hz, | |
| | 2H), 1.79 − 1.63 (m, 2H), 1.42 (d, J = 8.6 | |
| | Hz, 1H). | |
| Fmoc-hPhe- | 1H NMR (400 MHz, CD3OD) δ 8.88 (s, 1H), 8.23 − 8.16 | 675.9 |
| Arg-kbt, | (m, 1H), 8.14 − 8.05 (m, 1H), 7.78 (t, J = | |
| MF1152 | 7.3 Hz, 2H), 7.67 − 7.52 (m, 4H), 7.39 (dd, J = 8.3, 5.5 | |
| | Hz, 2H), 7.33 − 7.23 (m, 8H), 5.65 (td, | |
| | J = 9.6, 4.5 Hz, 1H), 5.32 − 5.21 (m, 2H), 4.68 − 4.59 (m, | |
| | 1H), 4.32 − 4.16 (m, 2H), 4.14 − 4.02 | |
| | (m, 2H), 3.22 (q, J = 5.6 Hz, 2H), 3.05 (dd, J = 15.0, 8.9 | |
| | Hz, 1H), 2.16 (d, J = 12.5 Hz, 1H), 1.80 | |
| | (dt, J = 23.4, 7.3 Hz, 2H). | |
| Fmoc-Lys(2- | 1H NMR (400 MHz, CD3OD) δ 8.18 (td, J = 7.5, 1.3 Hz, | 810.3 |
| CIZ)-Arg- | 1H), 8.09 − 8.05 (m, 1H), 7.77 (dd, J = | |
| kbt, | 7.6, 4.6 Hz, 2H), 7.63 (t, J = 6.9 Hz, 2H), 7.60 − 7.55 (m, | |
| MF1151 | 2H), 7.42 (q, J = 5.2 Hz, 1H), 7.37 (d, J = | |
| | 7.6 Hz, 2H), 7.33 (d, J = 11.4 Hz, 1H), 7.30 − 7.25 (m, | |
| | 4H), 5.71 − 5.61 (m, 1H), 5.15 (d, J = | |
| | 2.5 Hz, 2H), 4.40 − 4.24 (m, 2H), 4.19 − 4.10 (m, 2H), | |
| | 3.23 (d, J = 6.8 Hz, 2H), 3.09 (t, J = 6.8 | |
| | Hz, 2H), 2.17 (d, J = 8.5 Hz, 1H), 1.87 − 1.58 (m, 6H), | |
| | 1.49 (t, J = 6.9 Hz, 3H), 1.42 − 1.33 (m, | |
| | 2H). | |
| Fmoc-Nva- | 1H NMR (400 MHz, CD3OD) δ 8.19 (ddt, J = 8.9, 7.2, | 613.3 |
| Arg-kbt, | 1.1 Hz, 1H), 8.12 − 8.07 (m, 1H), 7.79 | |
| MF1150 | (dd, J = 7.5, 3.6 Hz, 2H), 7.67 − 7.63 (m, 2H), 7.61 − | |
| | 7.56 (m, 2H), 7.38 (q, J = 7.2 Hz, 2H), 7.31 | |
| | (dq, J = 8.8, 2.6 Hz, 2H), 5.71 − 5.65 (m, 1H), 4.40 − | |
| | 4.23 (m, 2H), 4.20 (t, J = 5.4 Hz, 1H), 4.13 | |
| | (dd, J = 8.8, 5.6 Hz, 1H), 3.26 − 3.19 (m, 2H), 2.23 − | |
| | 2.13 (m, 1H), 1.79 (q, J = 7.1 Hz, 2H), 1.71 | |
| | (q, J = 7.9 Hz, 2H), 1.59 (ddt, J = 13.8, 8.8, 4.4 Hz, 1H), | |
| | 1.43 − 1.30 (m, 2H), 0.90 (t, J = 7.3 Hz, | |
| | 3H). | |
| Fmoc-hCha- | 1H NMR (400 MHz, CD3OD) δ 8.21 − 8.16 (m, 1H), | 681.3 |
| Arg-kbt, | 8.12 − 8.06 (m, 1H), 7.80 − 7.76 (m, 2H), | |
| MF1149 | 7.65 (t, J = 6.1 Hz, 2H), 7.62 − 7.56 (m, 2H), 7.38 (q, J = | |
| | 7.2 Hz, 2H), 7.33 − 7.26 (m, 2H), 5.65 | |
| | (dd, J = 9.4, 4.2 Hz, 1H), 4.36 (dd, J = 10.2, 7.1 Hz, 1H), | |
| | 4.31 − 4.24 (m, 1H), 4.19 (q, J = 6.1 | |
| | Hz, 1H), 4.07 (q, J = 6.6 Hz, 1H), 3.23 (d, J = 6.5 Hz, | |
| | 2H), 2.18 (q, J = 4.6 Hz, 1H), 1.90 − 1.66 | |
| | (m, 6H), 1.68 − 1.53 (m, 7H), 1.26 − 1.08 (m, 7H), 0.84 − | |
| | 0.71 (m, 2H). | |
| Fmoc- | ¹H NMR (600 MHz, MeOD) δ 8.25 − 8.16 (m, 1H), 8.15 − | 733.3 |
| Nle(OBzl)- | 8.06 (m, 1H), 7.79 (t, J = 6.7 Hz, 2H), 7.71 − 7.51 (m, | |
| Arg-kbt, | 5H), 7.43 − 7.21 (m, 9H), 5.76 − 5.59 (m, 1H), 4.46 (d, J = | |
| MF1165 | 9.2 Hz, 2H), 4.41 − 4.33 (m, 1H), 4.33 − 4.25 (m, 1H), | |
| | 4.20 (q, J = 6.9 Hz, 1H), 4.13 (t, J = 7.3 Hz, 1H), 3.48 − | |
| | 3.40 (m, 2H), 3.26 − 3.17 (m, 3H), 2.24 − 2.11 (m, 1H), | |
| | 1.87 − 1.26 (m, 13H). | |
| Fmoc-hLeu- | 1H NMR (400 MHz, CD3OD) δ 8.19 (tt, J = 8.2, 1.3 Hz, | 641.3 |
| Arg-kbt, | 1H), 8.10 (t, J = 7.9 Hz, 1H), 7.79 (dd, J = | |
| MF1164 | 7.8, 3.3 Hz, 2H), 7.65 (t, J = 7.0 Hz, 2H), 7.62 − 7.56 | |
| | (m, 2H), 7.38 (q, J = 7.1 Hz, 2H), 7.33 − | |
| | 7.27 (m, 2H), 5.68 (dd, J = 9.1, 4.2 Hz, 1H), 4.36 (dd, J = | |
| | 10.0, 7.2 Hz, 1H), 4.31 − 4.25 (m, 1H), | |
| | 4.20 (dt, J = 10.5, 5.2 Hz, 1H), 4.08 (dd, J = 8.3, 5.6 Hz, | |
| | 1H), 3.27 − 3.21 (m, 2H), 2.17 (q, J = | |
| | 6.3 Hz, 1H), 1.87 − 1.69 (m, 4H), 1.60 (dq, J = 13.5, 4.7 | |
| | Hz, 1H), 1.50 (dq, J = 13.0, 6.9 Hz, 1H), | |
| | 1.21 (ddt, J = 17.8, 12.2, 6.0 Hz, 2H), 0.88 − 0.81 (m, | |
| | 6H). | |
| Fmoc-hTyr- | 1H NMR (400 MHz, CD3OD) δ 8.20 (tt, J = 8.2, 1.3 Hz, | 691.3 |
| Arg-kbt, | 1H), 8.10 (t, J = 7.9 Hz, 1H), 7.80 (dd, J = | |
| MF1163 | 7.8, 3.3 Hz, 2H), 7.69 − 7.51 (m, 4H), 7.39 (q, J = 7.1 | |
| | Hz, 2H), 7.34 − 7.25 (m, 2H), 5.65 (ddd, | |
| | J = 25.1, 9.1, 4.1 Hz, 1H), 4.43 − 4.24 (m, 2H), 4.21 (dt, J = | |
| | 10.5, 5.2 Hz, 1H), 4.09 (dd, J = 8.3, | |
| | 5.6 Hz, 1H), 3.27 − 3.18 (m, 2H), 2.18 (q, J = 6.3 Hz, | |
| | 1H), 1.79 (ddt, J = 32.3, 17.9, 8.3 Hz, 4H), | |
| | 1.60 (dq, J = 13.5, 4.7 Hz, 1H), 1.50 (dq, J = 13.0, 6.9 | |
| | Hz, 1H), 1.23 (dtq, J = 24.3, 12.8, 6.2 Hz, | |
| | 2H), 0.93 − 0.80 (m, 6H). | |
| Fmoc- | 1H NMR (400 MHz, CD3OD) δ 8.18 − 8.12 (m, 1H), | 742.09 |
| His(Bzl)- | 8.09 (t, J = 6.3 Hz, 1H), 7.80 (d, J = 7.2 Hz, | |
| Arg-kbt, | 2H), 7.70 − 7.64 (m, 2H), 7.58 (ddd, J = 9.4, 5.8, 2.0 Hz, | |
| MF1158 | 2H), 7.38 (q, J = 7.5 Hz, 2H), 7.32 (dt, J = | |
| | 8.8, 4.5 Hz, 2H), 6.94 (d, J = 8.1 Hz, 2H), 6.67 (dd, J = | |
| | 8.3, 4.4 Hz, 2H), 5.66 (dd, J = 9.1, 4.1 | |
| | Hz, 1H), 4.44 − 4.35 (m, 1H), 4.31 (dt, J = 10.6, 5.3 Hz, | |
| | 1H), 4.22 (d, J = 7.1 Hz, 1H), 4.11 (q, J = | |
| | 7.2 Hz, 1H), 3.23 (q, J = 7.2 Hz, 2H), 2.61 − 2.45 (m, | |
| | 2H), 2.18 (dd, J = 13.7, 7.6 Hz, 1H), 1.97 | |
| | (q, J = 7.0 Hz, 1H), 1.78 (ddt, J = 39.0, 24.5, 7.5 Hz, 4H). | |
| Fmoc- | 1H NMR (400 MHz, CD3OD) δ 8.23 − 8.16 (m, 1H), | 695.93 |
| Phe(3-Cl)- | 8.13 − 8.06 (m, 1H), 7.78 (t, J = 6.5 Hz, 2H), | |
| Arg-kbt, | 7.67 − 7.53 (m, 4H), 7.41 − 7.19 (m, 7H), 7.16 (s, 1H), | |
| MF1134 | 5.70 − 5.57 (m, 1H), 4.50 − 4.40 (m, 1H), | |
| | 4.36 − 4.25 (m, 1H), 4.15 (dq, J = 11.8, 6.8 Hz, 2H), | |
| | 3.27 − 3.17 (m, 2H), 3.08 (dt, J = 13.1, 6.2 | |
| | Hz, 1H), 2.93 − 2.84 (m, 1H), 2.22 − 2.06 (m, 1H), 1.81 − | |
| | 1.69 (m, 2H), 1.57 (q, J = 7.5 Hz, 1H). | |
| Fmoc-1-Nal- | 1H NMR (400 MHz, CD3OD) δ 8.19 − 8.15 (m, 1H), | 711.3 |
| Arg-kbt, | 8.09 (dd, J = 8.1, 2.4 Hz, 1H), 7.78 (t, J = | |
| MF1144 | 7.0 Hz, 2H), 7.74 − 7.70 (m, 1H), 7.64 − 7.53 (m, 5H), | |
| | 7.48 (dd, J = 14.3, 7.4 Hz, 1H), 7.40 − | |
| | 7.26 (m, 6H), 7.25 − 7.20 (m, 1H), 5.58 − 5.52 (m, 1H), | |
| | 4.56 (t, J = 7.5 Hz, 1H), 4.28 (ddd, J = | |
| | 16.8, 10.3, 6.9 Hz, 1H), 4.22 − 4.08 (m, 2H), 3.57 (ddd, J = | |
| | 29.0, 14.0, 7.3 Hz, 1H), 3.36 (dd, J = | |
| | 14.4, 8.1 Hz, 1H), 3.19 (q, J = 6.5 Hz, 1H), 3.07 (t, J = | |
| | 7.2 Hz, 1H), 2.07 − 1.93 (m, 1H), 1.69 (tt, | |
| | J = 15.5, 7.5 Hz, 2H), 1.32 (p, J = 7.5 Hz, 1H). | |
| Fmoc-Nle- | 1H NMR (400 MHz, CD3OD) δ 8.19 (td, J = 8.2, 1.8 Hz, | 627.3 |
| Arg-kbt, | 1H), 8.09 (t, J = 7.9 Hz, 1H), 7.79 (dd, J = | |
| MF1143 | 7.7, 3.4 Hz, 2H), 7.68 − 7.55 (m, 4H), 7.38 (q, J = 7.1 | |
| | Hz, 2H), 7.30 (tt, J = 9.9, 5.0 Hz, 2H), | |
| | 5.6868 − 5.62 (m, 1H), 4.43 − 4.24 (m, 2H), 4.19 (d, J = | |
| | 4.0 Hz, 1H), 4.11 (t, J = 7.3 Hz, 1H), | |
| | 3.27 − 3.17 (m, 2H), 2.19 (dd, J = 13.3, 6.9 Hz, 1H), 1.80 | |
| | (ddq, J = 37.4, 23.5, 7.4 Hz, 4H), 1.62 | |
| | (t, J = 6.9 Hz, 1H), 1.30 (s, 4H), 0.86 (t, J = 6.6 Hz, 3H). | |
| Fmoc-Cha- | 1H NMR (400 MHz, CD3OD) δ 8.22 − 8.16 (m, 1H), | 667.3 |
| Arg-kbt, | 8.12 − 8.06 (m, 1H), 7.79 (dd, J = 7.9, 3.1 | |
| MF1142 | Hz, 2H), 7.65 (t, J = 6.1 Hz, 2H), 7.61 − 7.56 (m, 2H), | |
| | 7.38 (q, J = 7.1 Hz, 2H), 7.33 − 7.27 (m, | |
| | 2H), 5.67 (dd, J = 9.3, 4.1 Hz, 1H), 4.37 (td, J = 6.8, 3.0 | |
| | Hz, 1H), 4.30 − 4.18 (m, 3H), 3.23 (dq, J = | |
| | 7.3, 3.6 Hz, 2H), 1.87 − 1.61 (m, 10H), 1.53 (q, J = 7.3 | |
| | Hz, 2H), 1.25 − 1.11 (m, 3H), 0.91 (s, | |
| | 2H). | |
| Ac-dhLeu- | 1H NMR (400 MHz, CD3)D) δ 8.31 (t, J = 6.5 Hz, 1H), | 796.13 |
| Glu(OAll)- | 8.15 − 8.12 (m, 1H), 7.68 − 7.60 (m, 2H), | |
| Phe(4-F)- | 7.38 (dd, J = 8.5, 5.4 Hz, 1H), 7.25 (dd, J = 8.5, 5.4 Hz, | |
| Arg-kbt, | 1H), 7.00 (t, J = 8.7 Hz, 1H), 6.89 (dd, J = | |
| MF1140 | 9.7, 7.7 Hz, 1H), 5.99 − 5.86 (m, 1H), 5.67 (dd, J = 9.4, | |
| | 3.7 Hz, 1H), 5.33 − 5.25 (m, 1H), 5.21 | |
| | (ddd, J = 10.4, 3.2, 1.5 Hz, 1H), 4.57 (ddt, J = 7.3, 5.7, | |
| | 1.5 Hz, 3H), 4.31 − 4.25 (m, 1H), 4.14 (t, J = | |
| | 7.4 Hz, 1H), 3.27 − 2.99 (m, 4H), 2.36 − 2.26 (m, 2H), | |
| | 2.13 (s, 2H), 2.01 (s, 2H), 1.87 − 1.67 (m, | |
| | 6H), 1.58 (pd, J = 6.7, 3.5 Hz, 1H), 1.34 − 1.27 (m, 1H), | |
| | 1.19 (dd, J = 11.6, 5.7 Hz, 1H), 0.95 − | |
| | 0.82 (m, 7H). | |
| Ac-dhLeu- | 1H NMR (400 MHz, CD3OD) δ 8.24 − 8.19 (m, 1H), | 838.14 |
| Glu(OChx)- | 8.15 − 8.11 (m, 1H), 7.68 − 7.60 (m, 2H), | |
| Phe(4-F)- | 7.41 − 7.35 (m, 1H), 7.25 (dd, J = 8.5, 5.5 Hz, 1H), | |
| Arg-kbt, | 7.02 − 6.97 (m, 1H), 6.92 − 6.86 (m, 1H), 5.68 | |
| MF1125 | (dt, J = 9.6, 3.4 Hz, 1H), 4.71 (dq, J = 8.8, 4.6 Hz, 1H), | |
| | 4.57 (ddd, J = 9.5, 7.8, 5.6 Hz, 1H), 4.52 − 4.46 | |
| | (m, 1H), 4.14 (t, J = 7.3 Hz, 1H), 4.06 (t, J = 7.5 | |
| | Hz, 1H), 3.27 − 3.11 (m, 3H), 3.04 (dd, J = | |
| | 14.0, 9.7 Hz, 1H), 2.26 (td, J = 8.5, 5.7 Hz, 2H), 2.13 | |
| | (s, 2H), 1.99 (d, J = 10.9 Hz, 2H), 1.82 (q, | |
| | J = 9.1 Hz, 5H), 1.77 − 1.69 (m, 5H), 1.57 (ddt, J = 9.9, | |
| | 6.5, 3.3 Hz, 2H), 1.45 − 1.29 (m, 6H), 0.95 − 0.89 (m, 7H). | |
| Fmoc- | 1H NMR (400 MHz, CD3OD) δ 8.19 (ddd, J = 12.0, 7.6, | 679.99 |
| Phe(3-F)- | 4.0 Hz, 1H), 8.09 (dd, J = 13.0, 7.9 Hz, | |
| Arg-kbt, | 1H), 7.77 (t, J = 6.5 Hz, 2H), 7.67 − 7.60 (m, 1H), 7.61 − | |
| MF1141 | 7.52 (m, 3H), 7.37 (q, J = 8.2 Hz, 2H), | |
| | 7.31 − 7.23 (m, 3H), 7.18 (q, J = 7.5 Hz, 1H), 7.05 (dt, | |
| | J = 16.7, 8.3 Hz, 2H), 6.90 (dt, J = 25.1, | |
| | 8.7 Hz, 1H), 5.68 − 5.63 (m, 1H), 4.50 − 4.42 (m, 1H), | |
| | 4.35 − 4.23 (m, 1H), 4.13 (dt, J = 16.2, 8.1 | |
| | Hz, 2H), 3.20 (dd, J = 18.1, 7.4 Hz, 2H), 3.14 − 3.06 (m, | |
| | 1H), 2.95 − 2.86 (m, 1H), 2.18 − 2.06 | |
| | (m, 1H), 1.78 (dq, J = 20.2, 7.6 Hz, 2H), 1.56 (t, J = 7.6 | |
| | Hz, 1H). | |
| Fmoc-Phe(3, | 1H NMR (400 MHz, CD3OD) δ 8.19 (ddd, J = 12.2, 7.2, | 697.943 |
| 4 di-F)-Arg- | 2.0 Hz, 1H), 8.10 (ddd, J = 13.6, 7.0, 1.9 | |
| kbt, | Hz, 1H), 7.78 (t, J = 6.8 Hz, 2H), 7.66 − 7.52 (m, 4H), | |
| MF1132 | 7.37 (q, J = 8.1 Hz, 2H), 7.28 (q, J = 7.4 | |
| | Hz, 2H), 7.16 (dt, J = 18.5, 9.4 Hz, 1H), 7.06 − 6.96 (m, | |
| | 2H), 5.76 − 5.66 (m, 1H), 5.63 (dd, J = | |
| | 9.4, 4.2 Hz, 1H, H1), 4.48 − 4.38 (m, 1H), 4.32 (ddd, J = | |
| | 20.6, 10.4, 6.8 Hz, 1H), 4.17 (ddt, J = | |
| | 27.7, 13.6, 6.8 Hz, 2H), 3.27 − 3.16 (m, 2H), 3.07 (ddd, | |
| | J = 19.7, 14.0, 6.3 Hz, 1H), 2.86 (dd, J = | |
| | 14.0, 9.3 Hz, 1H), 2.16 (d, J = 16.5 Hz, 1H), 1.86 − 1.69 | |
| | (m, 2H), 1.61 (d, J = 8.6 Hz, 1H). | |
| Fmoc- | 1H NMR (400 MHz, CD3OD) δ 8.24 − 8.15 (m, 1H), | 679.99 |
| Phe(4-F)- | 8.14 − 8.06 (m, 1H), 7.78 (t, J = 6.7 Hz, 2H), | |
| Arg-kbt, | 7.68 − 7.53 (m, 4H), 7.42 − 7.33 (m, 2H), 7.25 (tt, J = | |
| MF1131 | 16.5, 7.7 Hz, 4H), 6.97 (t, J = 8.6 Hz, 1H), | |
| | 6.87 (t, J = 8.6 Hz, 1H), 5.62 (dd, J = 9.4, 4.2 Hz, 1H), | |
| | 4.55 − 4.38 (m, 1H), 4.37 − 4.25 (m, 1H), | |
| | 4.16 (dt, J = 21.5, 8.5 Hz, 2H), 3.26 − 3.15 (m, 2H), 3.07 | |
| | (td, J = 13.7, 6.3 Hz, 1H), 2.87 (dd, J = | |
| | 13.5, 8.9 Hz, 1H), 2.13 (ddt, J = 19.6, 13.5, 6.3 Hz, 1H), | |
| | 1.76 (tt, J = 14.3, 7.2 Hz, 2H), 1.56 (d, J = | |
| | 7.4 Hz, 1H). | |
| MF1101, | 1H NMR (399 MHz, cd3od) δ 8.38 (dd, J = 11.2, 7.8 Hz, | 708.33 |
| PhSO2- | 1H), 8.23 − 8.19 (m, 1H), 8.15 − 8.11 (m, 1H), 7.82 − | |
| QFR-kbt | 7.78 (m, 2H), 7.67 − 7.58 (m, 3H), 7.58 − 7.46 (m, 3H), | |
| | 7.30 − 7.07 (m, 5H), 5.71 − 5.55 (m, 1H), 4.63 − 4.42 (m, | |
| | 1H), 3.69 − 3.59 (m, 1H), 3.27 − 3.21 (m, 1H), 3.20 − | |
| | 3.14 (m, 1H), 3.12 − 3.03 (m, 1H), 2.86 − 2.78 (m, 1H), | |
| | 2.12 − 2.05 (m, 2H), 1.98 (t, J = 7.4 Hz, 1H), 1.83 − 1.64 | |
| | (m, 5H), 1.61 − 1.50 (m, 1H). | |
| MF1099, | 1H NMR (399 MHz, cd3od) δ 8.24 − 8.20 (m, 1H), | 646.73 |
| MeSO2- | 8.15 − 8.12 (m, 1H), 7.69 − 7.60 (m, 2H), 7.32 − 7.09 (m, 7H), | |
| QFR-kbt, N- | 5.72 (dd, J = 9.6, 3.8 Hz, 1H), 4.77 (dt, J = 10.4, 5.3 Hz, | |
| 0385 | 1H), 3.85 (ddd, J = 8.4, 6.0, 2.6 Hz, 1H), 3.80 − 3.75 (m, | |
| (competitor | 1H), 3.20 (ddd, J = 19.1, 14.0, 6.0 Hz, 3H), 2.98 − 2.87 | |
| patent and | (m, 2H), 2.70 (d, J = 3.1 Hz, 1H), 2.39 − 2.11 (m, 4H), | |
| paper) | 1.79 (td, J = 15.7, 8.7 Hz, 4H). | |
| BnSO2- | ¹H NMR (399 MHz, cd30d) δ 8.48 − 8.34 (m, 1H), 8.24 − | 722.31 |
| QFR-kbt, | 8.20 (m, 1H), 8.13 (td, J = 6.9, 1.8 Hz, 1H), 7.67 − 7.61 | |
| MF1105 | (m, 2H), 7.35 − 7.31 (m, 6H), 7.26 − 7.21 (m, 3H), 7.14 | |
| | (t, J = 7.4 Hz, 2H), 7.04 (t, J = 7.4 Hz, 1H), 5.73 − 5.56 | |
| | (m, 1H), 4.78 − 4.72 (m, 1H), 4.17 (s, 1H), 4.09 (s, 1H), | |
| | 3.85 − 3.78 (m, 1H), 3.26 − 3.13 (m, 4H), 2.97 − 2.88 (m, | |
| | 1H), 2.25 − 2.07 (m, 4H), 1.86 − 1.73 (m, 5H), 1.68 − | |
| | 1.54 (m, 1H). | |
| CypSO2- | 1H NMR (399 MHz, cd3od) δ 8.24 − 8.20 (m, 1H), | 672.24 |
| QFR-kbt, | 8.16 − 8.11 (m, 1H), 7.68 − 7.60 (m, 2H), 7.32 − 7.05 (m, 6H), | |
| MF1104 | 5.77 − 5.57 (m, 1H), 4.81 − 4.70 (m, 1H), 3.93 − 3.84 (m, | |
| | 1H), 3.23 − 3.14 (m, 2H), 2.98 − 2.88 (m, 1H), 2.37 − | |
| | 2.10 (m, 5H), 2.00 − 1.69 (m, 5H), 1.61 (p, J = 7.4 Hz, | |
| | 1H), 0.98 − 0.68 (m, 5H). | |
| Ac- | 1H NMR (400 MHz, DMSO): δ 8.70 − 8.57 (m, 1H), | 819.4 |
| OicGlu(OAll)Phe(4- | 8.34 − 8.21 (m, 2H), 8.06 − 7.92 (m, 1H), 7.90 − 7.80 (m, | |
| F)Arg-kbt, | 1H), 7.73 − 7.61 (m, 2H), 7.52 − 7.44 (m, 1H), 7.29 − | |
| MF1068 | 7.17 (m, 2H), 7.09 − 6.92 (m, 2H), 5.94 − 5.79 (m, 1H), | |
| | 5.54 − 5.40 (m, 1H), 5.31 − 5.20 (m, 1H), 5.19 − 5.08 (m, | |
| | 1H), 4.70 − 4.52 (m, 1H), 4.53 − 4.44 (m, 2H), 4.27 − | |
| | 4.06 (m, 2H), 3.78 − 3.65 (m, 1H), 3.19 − 3.05 (m, 2H), | |
| | 3.04 − 2.90 (m, 1H), 2.83 − 2.70 (m, 1H), 2.40 − 2.28 (m, | |
| | 1H), 2.27 − 2.12 (m, 2H), 1.97 − 1.88 (m, 3H), 1.88 − | |
| | 1.66 (m, 6H), 1.62 − 1.39 (m, 6H), 1.35 − 0.97 (m, 4H). | |
| Ac- | 1H NMR (400 MHz, DMSO) δ 8.79 − 8.61 (m, 1H), 8.59 − | 856.4 |
| IdcGlu(OCh | 8.36 (m, 2H), 8.32 − 8.20 (m, 2H), 8.11 − 7.94 (m, 1H), | |
| x)Phe(4- | 7.73 − 7.51 (m, 3H), 7.28 − 7.18 (m, 2H), 7.15 − 6.78 (m, | |
| F)Arg-kbt, | 4H), 5.52 − 5.34 (m, 1H), 5.01 (dd, J = 11.5, 3.0 Hz, 1H), | |
| MF1065 | 4.71 − 4.52 (m, 2H), 4.28 − 4.09 (m, 1H), 3.59 − 3.40 (m, | |
| | 1H), 3.17 − 2.85 (m, 4H), 2.83 − 2.57 (m, 1H), 2.34 (s, | |
| | 1H), 2.26 − 2.08 (m, 1H), 1.94 (s, 3H), 1.91 − 1.82 (m, | |
| | 2H), 1.82 − 1.38 (m, 12H), 1.38 − 1.09 (m, 6H). | |
| Ac- | ¹H NMR (600 MHz, DMSO) δ 8.74 (d, J = 7.1 Hz, | 861.4 |
| OicGlu(OC | 0.3H), 8.61 (d, J = 7.1 Hz, 0.7H), 8.32 − 8.24 (m, 2H), | |
| hx)Phe(4- | 8.20 (d, J = 7.9 Hz, 0.3H), 8.01 (d, J = 7.7 Hz, 0.7H), | |
| F)Arg-kbt, | 7.96 (d, J = 8.3 Hz, 0.3H), 7.83 (d, J = 8.4 Hz, 0.7H), | |
| MF1064 | 7.72 − 7.64 (m, 2H), 7.43 (q, J = 6.2 Hz, 1H), 7.25 (tt, J = | |
| | 5.6, 2.9 Hz, 2H), 7.05 (td, J = 8.8, 1.8 Hz, 2H), 5.48 | |
| | (ddd, J = 9.2, 7.1, 4.5 Hz, 1H), 4.74 − 4.56 (m, 2H), 4.36 − | |
| | 4.31 (m, 0.3H), 4.28 − 4.12 (m, 2H), 3.90 (dt, J = 12.1, | |
| | 6.3 Hz, 0.2H), 3.73 (dt, J = 12.1, 6.3 Hz, 0.7H), 3.11 (q, J = | |
| | 7.0 Hz, 2H), 2.98 (dd, J = 13.8, 5.3 Hz, 1H), 2.80 (ddd, | |
| | J = 15.2, 9.0, 6.1 Hz, 1H), 2.33 − 2.08 (m, 3H), 2.08 − | |
| | 1.40 (m, 23H), 1.39 − 0.97 (m, 10H). | |
| MM4146, | ¹H NMR (600 MHz, MeOD) δ 8.37 − 8.08 (m, 2H), 7.73 − | N.D. |
| VD4162- | 7.49 (m, 4H), 7.03 − 6.92 (m, 3H), 6.87 − 6.73 (m, 2H), | |
| albumin | 4.67 − 4.02 (m, 6H), 3.28 − 3.02 (m, 9H), 3.01 − 2.78 (m, | |
| | 3H), 2.63 − 2.49 (m, 3H), 2.26 − 2.07 (m, 4H), 2.07 − | |
| | 1.94 (m, 4H), 1.92 − 1.79 (m, 4H), 1.75 − 1.56 (m, 5H), | |
| | 1.56 − 1.24 (m, 14H), 1.21 − 0.93 (m, 3H). | |
| CA1046-1, | N.D. | 635.3 |
| Pip-QFdR- | | |
| kbt | | |
| CA1046-2, | 1H NMR (399 MHz, cd3od) δ 8.27 − 8.19 (m, 1H), 8.19 − | 635.3 |
| Pip-QFR- | 8.11 (m, 1H), 7.71 − 7.59 (m, 2H), 7.38 − 7.23 (m, 5H), | |
| kbt | 7.20 (m, 1H), 5.71 (td, J = 9.7, 4.3 Hz, 1H), 5.02 (dd, J = | |
| | 11.3, 4.7 Hz, 1H), 4.79 (dd, J = 10.9, 5.1 Hz, OH), 3.84 | |
| | (dd, J = 9.3, 4.3 Hz, OH), 3.71 − 3.35 (m, 3H), 3.28 (m, | |
| | 2H), 3.11 − 2.77 (m, 2H), 2.77 − 2.39 (m, 1H), 2.39 − | |
| | 2.12 (m, 3H), 2.12 − 1.25 (m, 14H). | |
| CA1041-2, | ¹H NMR (399 MHz, cd30d) δ 8.27 − 8.19 (m, 1H), 8.19 − | 595.91 |
| Me2-QFR- | 8.12 (m, 1H), 7.75 − 7.59 (m, 2H), 7.35 − 7.11 (m, 6H), | |
| kbt | 5.76 − 5.66 (m, 1H), 4.96 (dd, J = 10.9, 4.7 Hz, 1H), 3.62 | |
| | (dd, J = 9.9, 3.7 Hz, 1H), 3.30 − 3.24 (m, 3H), 3.19 (s, | |
| | 1H), 2.92 − 2.80 (m, 2H), 2.79 − 2.35 (m, 3H), 2.34 − | |
| | 2.14 (m, 4H), 2.06 − 1.72 (m, 5H). | |
| CA1041-1, | N.D. | 595.91 |
| Me2-QFdR- | | |
| kbt | | |
| CA1033-1, | N.D. | 671.3 |
| PhEt- | | |
| QFdR-kbt | | |
| Bn-QFdR- | N.D. | 657.3 |
| kbt, | | |
| CA1022-1 | | |
| Bz-QFR- | 1H NMR (399 MHz, dmso) δ 8.68 − 8.52 (m, 2H), 8.28 − | 671.931 |
| kbt, | 8.20 (m, 2H), 8.00 (t, J = 8.7 Hz, 1H), 7.84 (t, J = 7.6 Hz, | |
| CA1043, | 2H), 7.70 − 7.60 (m, 2H), 7.50 (d, J = 7.3 Hz, 1H), 7.43 | |
| MF1017, | (t, J = 7.3 Hz, 3H), 7.29 (s, 1H), 7.18 − 7.13 (m, 3H), | |
| MF2001, | 7.11 − 7.07 (m, 2H), 6.83 (s, 1H), 5.49 − 5.43 (m, 1H), | |
| ZFH9131 | 4.62 − 4.54 (m, 1H), 4.32 − 4.26 (m, 1H), 3.14 − 3.03 (m, | |
| | 2H), 3.01 − 2.90 (m, 1H), 2.82 − 2.72 (m, 1H), 2.15 − | |
| | 1.99 (m, 2H), 1.99 − 1.82 (m, 2H), 1.81 − 1.69 (m, 2H), | |
| | 1.65 − 1.42 (m, 2H). | |
| CA1033-2, | 1H NMR (399 MHz, cd3od) δ 8.23 − 8.20 (m, 1H), 8.14 − | 671.3 |
| PhEt-QFR- | 8.11 (m, 1H), 7.68 − 7.60 (m, 2H), 7.38 − 7.33 (m, 2H), | |
| kbt | 7.33 − 7.21 (m, 6H), 7.20 − 7.16 (m, 2H), 7.13 − 7.08 (m, | |
| | 2H), 6.90 (t, J = 7.2 Hz, 1H), 5.63 (dd, J = 9.6, 4.1 Hz, | |
| | 1H), 3.78 − 3.72 (m, 1H), 3.25 − 3.14 (m, 4H), 2.91 − | |
| | 2.80 (m, 4H), 2.63 − 2.56 (m, 1H), 2.42 − 2.33 (m, 2H), | |
| | 2.20 − 2.11 (m, 1H), 2.08 − 1.97 (m, 3H), 1.86 − 1.73 (m, | |
| | 2H), 1.66 − 1.57 (m, 2H). | |
| Bn-QFR- | 1H NMR (399 MHz, dmso) δ 9.03 − 8.94 (m, 2H), 8.33 − | 657.3 |
| kbt, | 8.23 (m, 2H), 7.74 − 7.61 (m, 3H), 7.54 (s, 1H), 7.41 − | |
| CA1022-2 | 7.15 (m, 12H), 7.14 − 7.06 (m, 3H), 5.57 − 5.45 (m, 1H), | |
| | 4.96 − 4.85 (m, 1H), 3.80 − 3.64 (m, 1H), 3.60 − 3.55 (m, | |
| | 1H), 3.44 − 3.28 (m, 2H), 2.69 (t, J = 12.7 Hz, 1H), 2.19 | |
| | (t, J = 7.2 Hz, 2H), 2.10 − 1.89 (m, 1H), 1.90 − 1.75 (m, | |
| | 2H), 1.66 − 1.61 (m, 1H). | |
| CA1018, | 1H NMR (399 MHz, dmso) δ 8.60 (dd, J = 18.4, 6.7 Hz, | 685.78 |
| PhCH2(CO) | 1H), 8.25 − 8.21 (m, 2H), 8.22 − 8.14 (m, 1H), 8.00 (t, J = | |
| -QFR-kbt | 8.4 Hz, 1H), 7.68 − 7.61 (m, 2H), 7.47 − 7.36 (m, 1H), | |
| | 7.27 − 7.05 (m, 13H), 6.78 − 6.71 (m, 1H), 5.51 − 5.37 | |
| | (m, 1H), 4.63 − 4.51 (m, 1H), 4.18 − 4.08 (m, 1H), 3.13 − | |
| | 3.00 (m, 2H), 2.99 − 2.87 (m, 1H), 2.79 − 2.62 (m, 1H), | |
| | 2.02 − 1.93 (m, 2H), 1.79 − 1.67 (m, 2H), 1.63 − 1.50 (m, | |
| | 3H), 1.45 − 1.36 (m, 1H). | |
| Ac-GQFdR- | N.D. | 666.5 |
| kbt | | |
| MM3122-1 | | |
| Ac-IMFR- | 1H NMR (399 MHz, dmso) δ 8.61 (d, J = 7.0 Hz, 1H), | 725.6 |
| kbt, | 8.32 − 8.23 (m, 2H), 8.01 (d, J = 7.8 Hz, 1H), 7.94 (d, J = | |
| MM4027-2 | 8.2 Hz, 1H), 7.88 (d, J = 8.6 Hz, 1H), 7.73 − 7.62 (m, | |
| | 2H), 7.48 (t, J = 5.8 Hz, 1H), 7.29 − 7.14 (m, 6H), 5.48 − | |
| | 5.39 (m, 1H), 4.66 (q, J = 8.4 Hz, 1H), 4.28 (td, J = 8.6, | |
| | 5.1 Hz, 1H), 4.08 (t, J = 7.8 Hz, 1H), 3.07 (q, J = 6.6 Hz, | |
| | 2H), 2.94 (dd, J = 13.8, 6.0 Hz, 1H), 2.81 (dd, J = 13.6, | |
| | 8.6 Hz, 1H), 2.32 (hept, J = 7.0 Hz, 2H), 2.03 − 1.56 (m, | |
| | 12H), 1.54 − 1.31 (m, 3H), 1.04 (dt, J = 16.0, 8.0 Hz, | |
| | 1H), 0.74 (t, J = 7.2 Hz, 6H). | |
| VD5123B | 1H NMR (400 MHz, DMSO) δ 8.86 (d, J = 6.0 Hz, 1 H), | 755.5 |
| | 8.26 (ddd, J = 12.1, 7.1, 2.5 Hz, 2 H), 8.13 (d, J = 10.0 | |
| | Hz, 1 H), 7.78 (d, J = 9.4 Hz, 1 H), 7.67 (ddd, J = 6.8, | |
| | 4.3, 1.8 Hz, 2 H), 7.39 (d, J = 8.3 Hz, 1 H), 7.27 − 7.16 | |
| | (m, 4 H), 7.00 (dd, J = 8.1, 2.5 Hz, 1 H), 6.97 − 6.91 (m, 1 | |
| | H), 6.67 (dd, J = 8.4, 2.5 Hz, 1 H), 6.54 (d, J = 7.5 Hz, 1 | |
| | H), 5.83 (s, 1 H), 5.45 (s, 1 H), 4.67 (s, 1 H), 4.51 (s, 1 | |
| | H), 4.37 (d, J = 8.3 Hz, 1 H), 3.21 − 2.96 (m, 6 H), 2.63 (s, | |
| | 1 H), 2.59 (s, 1 H), 1.99 (s, 2 H), 1.80 (s, 3 H), 1.63 (s, 2 | |
| | H), 1.38 (s, 1 H), 1.27 (s, 2 H), 0.79 (d, J = 6,4 Hz, 3 H), | |
| | 0.75 (d, J = 6.5 Hz, 3 H). | |
| VD5123A | N.D. | 755.5 |
| Ac-IdWFR- | ¹H NMR (399 MHz, dmso) δ 10.72 (s, 1H), 8.70 (dd, J = | 780.6 |
| kbt, | 18.5, 6.8 Hz, 1H), 8.42 (t, J = 9.5 Hz, 1H), 8.33 − 8.24 | |
| MM4038 | (m, 2H), 8.12 − 8.04 (m, 1H), 7.78 (d, J = 8.6 Hz, 1H), | |
| | 7.74 − 7.63 (m, 2H), 7.59 (d, J = 8.2 Hz, 1H), 7.53 − 7.43 | |
| | (m, 1H), 7.31 − 7.09 (m, 7H), 7.06 − 6.98 (m, 2H), 6.95 | |
| | (t, J = 7.0 Hz, 1H), 5.54 (t, J = 10.3 Hz, 1H), 4.77 − 4.64 | |
| | (m, 1H), 4.52 − 4.41 (m, 1H), 4.10 (t, J = 8.0 Hz, 1H), | |
| | 3.20 − 3.14 (m, 1H), 3.14 − 3.07 (m, 1H), 3.07 − 2.95 (m, | |
| | 1H), 2.82 − 2.68 (m, 2H), 2.65 − 2.53 (m, 1H), 2.08 − | |
| | 1.81 (m, 2H), 1.78 (d, J = 7.0 Hz, 3H), 1.70 − 1.62 (m, | |
| | 1H), 1.58 − 1.45 (m, 1H), 1.46 − 1.35 (m, 1H), 1.15 − | |
| | 1.00 (m, 1H), 0.82 − 0.75 (m, 1H), 0.62 − 0.54 (m, 3H), | |
| | 0.40 (dd, J = 7.0, 3.5 Hz, 3H). | |
| Ac-IEFdR- | N.D. | 723.6 |
| kbt, | | |
| MM4037-1 | | |
| Ac-IEFR- | ¹H NMR (399 MHz, dmso) δ 12.09 (s, 1H), 8.62 (d, J = | 723.6 |
| kbt, | 6.6 Hz, 1H), 8.33 − 8.23 (m, 2H), 7.96 (dd, J = 12.7, 8.0 | |
| MM4037-2 | Hz, 2H), 7.89 (d, J = 7.8 Hz, 1H), 7.68 (dt, J = 6.6, 2.7 | |
| | Hz, 2H), 7.48 (t, J = 5.6 Hz, 1H), 7.23 − 7.11 (m, 5H), | |
| | 5.49 (q, J = 6.9 Hz, 1H), 4.58 (td, J = 9.0, 4.5 Hz, 1H), | |
| | 4.18 (q, J = 8.4 Hz, 1H), 4.08 (t, J = 7.8 Hz, 1H), 3.14 (q, | |
| | J = 7.2 Hz, 2H), 3.00 (dd, J = 14.2, 4.5 Hz, 1H), 2.76 | |
| | (dd, J = 14.2, 9.5 Hz, 1H), 2.21 − 2.09 (m, 2H), 1.97 (d, J = | |
| | 6.6 Hz, 1H), 1.85 (s, 3H), 1.78 (s, 2H), 1.71 − 1.53 (m, | |
| | 4H), 1.46 − 1.31 (m, 1H), 1.14 − 0.98 (m, 1H), 0.81 − | |
| | 0.72 (m, 6H). | |
| Ac-ITFR- | 1H NMR (399 MHz, dmso) δ 8.62 (d, J = 6.6 Hz, 1H), | 695.6 |
| kbt, | 8.28 (ddd, J = 12.5, 6.4, 3.1 Hz, 2H), 7.97 (d, J = 8.6 Hz, | |
| MM4028-2 | 1H), 7.85 (d, J = 8.2 Hz, 1H), 7.74 − 7.62 (m, 3H), 7.51 − | |
| | 7.43 (m, 1H), 7.23 − 7.08 (m, 5H), 5.48 (q, J = 5.6 Hz, | |
| | 1H), 4.89 (d, J = 5.4 Hz, 1H), 4.61 (h, J = 4.7 Hz, 1H), | |
| | 4.21 − 4.13 (m, 2H), 3.97 − 3.84 (m, 1H), 3.14 (q, J = 6.6 | |
| | Hz, 2H), 3.02 (dd, J = 14.4, 4.7 Hz, 1H), 2.81 (dd, J = | |
| | 14.2, 9.1 Hz, 1H), 1.95 (s, 1H), 1.85 (s, 3H), 1.82 − 1.64 | |
| | (m, 2H), 1.58 (d, J = 16.3 Hz, 2H), 1.39 (s, 1H), 1.07 (dt, | |
| | J = 16.0, 7.6 Hz, 1H), 0.95 (d, J = 6.2 Hz, 3H), 0.83 − | |
| | 0.71 (m, 6H). | |
| Ac-ITFdR- | N.D. | 695.6 |
| kbt, | | |
| MM4028-1 | | |
| Ac-IMFdR- | N.D. | 725.6 |
| kbt, | | |
| MM4027-1 | | |
| Ac-ISFdR- | N.D. | 681.5 |
| kbt, | | |
| MM4009-1 | | |
| Ac-ISFR- | ¹H NMR (399 MHz, dmso) δ 8.55 (d, J = 7.0 Hz, 1H), | 681.5 |
| kbt, | 8.33 − 8.23 (m, 2H), 7.98 − 7.88 (m, 3H), 7.69 (td, J = | |
| MM4009-2 | 7.2, 3.9 Hz, 2H), 7.48 (t, J = 5.8 Hz, 1H), 7.21 − 7.10 (m, | |
| | 5H), 5.54 − 5.44 (m, 1H), 4.95 (t, J = 5.6 Hz, 1H), 4.57 | |
| | (dq, J = 9.0, 4.3 Hz, 1H), 4.26 (q, J = 6.0 Hz, 1H), 4.15 | |
| | (t, J = 7.8 Hz, 1H), 3.50 (q, J = 5.1 Hz, 2H), 3.14 (q, J = | |
| | 7.2 Hz, 2H), 3.03 (dd, J = 14.0, 4.7 Hz, 1H), 2.79 (dd, J = | |
| | 14.4, 9.3 Hz, 1H), 1.96 (d, J = 6.2 Hz, 1H), 1.85 (s, | |
| | 3H), 1.82 − 1.51 (m, 4H), 1.38 (ddd, J = 10.9, 7.2, 3.7 | |
| | Hz, 1H), 1.13 − 0.98 (m, 1H), 0.78 (t, J = 7.4 Hz, 6H). | |
| Ac-Ser- | ¹H NMR (400 MHz, DMSO-d6) δ ppm 0.77 − 0.92 (m, 6 | 791.6 |
| His(BOM)- | H) 1.23 (dd, J = 3.33, 1.76 Hz, 1 H) 1.32 − 1.49 (m, 1 H) | |
| Leu-Arg- | 1.82 − 1.85 (m, 2 H) 1.85 − 1.97 (m, 6 H) 2.08 (d, J = 2.74 | |
| kbt, | Hz, 2 H) 2.52 − 2.58 (m, 4 H) 2.75 (d, J = 2.35 Hz, 1 H) | |
| MM1123-2, | 2.92 (t, J = 7.24 Hz, 8 H) 2.99 (d, J = 2.35 Hz, 1 H) 3.23 (q, | |
| MM4036-2, | J = 6.52 Hz, 8 H) 3.52 (dd, J = 5.87, 2.74 Hz, 1 H) 4.50 − | |
| JH1132 | 4.59 (m, 2 H) 5.60 − 5.80 (m, 1 H) 7.21-7.33 (m, 5 H) | |
| | 7.33-7.42 (m, 5 H) 7.49 (t, J = 7.63 Hz, 4 H) 7.58 (br. s., | |
| | 1 H) 7.63 (t, J = 4.89 Hz, 4 H) 7.65-7.71 (m, 2 H) 7.91 | |
| | (d, J = 8.22 Hz, 4 H) 8.02 (d, J = 7.43 Hz, 1 H) 8.03-8.11 | |
| | (m, 4 H) 8.16-8.31 (m, 2 H) 8.35 (d, J = 10.56 Hz, 1 H) | |
| | 9.18 (s, 1 H). | |
| Ac-Ser- | N.D. | N.D. |
| His(BOM)- | | |
| Leu-dArg- | | |
| kbt, | | |
| MM1123-1, | | |
| MM4036-1 | | |
| Ac- | 1H NMR (399 MHz, DMSO-d6) δ ppm 0.69-0.87 (m, 6 | 888.8 |
| His(BOM)- | H) 0.98-1.14 (m, 3 H) 1.23 (br. s., 3 H) 1.34-1.55 (m, | |
| hARg-hLeu- | 6 H) 1.60 (d, J = 6.23 Hz, 3 H) 1.66 (br. s., 1 H) 1.71 (d, | |
| dArg-kbt, | J = 10.51 Hz, 1 H) 1.82 (s, 3 H) 1.96 (br. s., 2 H) 2.88- | |
| JH1143-1, | 3.07 (m, 4 H) 3.07-3.23 (m, 4 H) 4.13 (dd, J = 13.04, | |
| MM4032-1 | 8.37 Hz, 2 H) 4.30-4.46 (m, 2 H) 4.47-4.57 (m, 2 H) | |
| | 4.59-4.74 (m, 1 H) 5.45 (d, J = 10.51 Hz, 2 H) 5.66 (s, 2 | |
| | H) 6.84 (s, 2 H) 7.02 (s, 2 H) 7.15 (br. s., 1 H) 7.18-7.42 | |
| | (m, 8 H) 7.58-7.78 (m, 4 H) 7.97 (d, J = 8.17 Hz, 1 H) | |
| | 8.06 (d, J = 7.40 Hz, 1 H) 8.16-8.30 (m, 2 H) 8.34 (d, | |
| | J = 8.56 Hz, 1 H) 8.79 (d, J = 6.62 Hz, 1 H) 9.11 (br. s., 2 | |
| | H). | |
| Ac-hArg- | N.D. | 888.8 |
| His(BOM)- | | |
| hLeu-dArg- | | |
| kbt, | | |
| JH1144, | | |
| MM4031-1 | | |
| Ac-hArg- | ¹H NMR (400 MHz, DMSO-d6) δ ppm 0.77 (d, J = 6.62 | 888.8 |
| His(BOM)- | Hz, 6 H) 1.08 (d, J = 6.62 Hz, 3 H) 1.23 (br. s., 3 H) 1.33-1.52 | |
| hLeu-Arg- | (m, 5 H) 1.52-1.67 (m, 4 H) 1.67-1.77 (m, 1 H) | |
| kbt, | 1.77-1.87 (m, 3 H) 1.87-2.03 (m, 2 H) 2.92 (dd, | |
| JH1144-2, | J = 15.38, 8.76 Hz, 2 H) 3.02 (d, J = 6.23 Hz, 2 H) | |
| MM4031-2 | 3.07-3.22 (m, 3 H) 4.16-4.31 (m, 2 H) 4.31-4.42 (m, 1 H) | |
| | 4.59-4.76 (m, 2 H) 5.30-5.51 (m, 2 H) 5.68 (s, 2 H) | |
| | 7.01 (br. s., 4 H) 7.14 (br. s., 2 H) 7.22-7.44 (m, 8 H) | |
| | 7.59 (t, J = 5.26 Hz, 2 H) 7.63-7.75 (m, 3 H) 7.94 (d, | |
| | J = 8.56 Hz, 1 H) 8.11-8.36 (m, 4 H) 8.72 (d, J = 6.62 Hz, | |
| | 1 H) 9.13 (br. s., 1 H). | |
| VD5076A | | 755.6 |
| VD5076B | 1H NMR (400 MHz, DMSO-d6) δ = 8.93-8.86 (m, 1 H), | 755.6 |
| | 8.32-8.24 (m, 2 H), 8.17-8.13 (m, 2 H), 7.83-7.76 (m, 2 | |
| | H), 7.69 (br s, 1 H), 7.43-7.38 (m, 2 H), 7.32-7.16 (m, 2 | |
| | H), 7.07-6.90 (m, 1 H), 6.72-6.65 (m, 1 H), 6.60-6.52 | |
| | (m, 1 H), 5.88-5.82 (m, 1 H), 5.53-5,43 (m, 1 H), 4.73-4.63 | |
| | (m, 1 H), 4.57-4.48 (m, 1 H), 4.43-4.36 (m, 1 H), | |
| | 3.22-3.13 (m, 1 H), 2.70-2.60 (m, 1 H), 1.82 (s, 3 H), | |
| | 1.71-1.61 (m, 1 H), 1.34-1.22 (m, 2 H), 0.79 (dd, J = | |
| | 6.6, 15.2 Hz, 6 H) | |
| VD5027 | 1H NMR (600 MHz, DMSO-d6) δ = 1.28-1.42 (m, 1 H), | 792.3 |
| | 1.42-1.48 (m, 1 H), 1.52-1.65 (m, 1 H), 1.65-1.73 (m, 1 | |
| | H), 1.73-1.82 (m, 1 H), 1.82-1.87 (m, 2 H), 1.87-1.92 | |
| | (m, 1 H), 1.92-2.04 (m, 1 H), 2.28 (br s, 1 H), 2.32-2.38 | |
| | (m, 1 H), 2.51-2.55 (m, 2 H), 2.57-2.63 (m, 1 H), 2.72-2.86 | |
| | (m, 1 H), 2.86-2.92 (m, 1 H), 2.96-3.06 (m, 2 H), | |
| | 3.08-3.20 (m, 1 H), 4.12 (q, J = 7.16 Hz, 1 H), 4.39-4.49 | |
| | (m, 2 H), 4.49-4.57 (m, 1 H), 4.58-4.74 (m, 1 H), 5.23- | |
| | 5.34 (m, 1 H), 5.36-5.44 (m, 1 H), 5.44-5.51 (m, 1 H), | |
| | 6.45-6.54 (m, 3 H), 6.55 (d, J = 7.45 Hz, 1 H), 6.64-6.78 | |
| | (m, 1 H), 6.86-6.99 (m, 2 H), 6.99-7.07 (m, 2 H), 7.21 | |
| | (d, J = 7.53 Hz, 1 H), 7.28 (d, J = 8.04 Hz, 1 H), 7.43 | |
| | (d, J = 6.58 Hz, 1 H), 7.45-7.49 (m, 1 H), 7.49-7.54 (m, | |
| | 1 H), 7.57-7.64 (m, 1 H), 7.64-7.74 (m, 2 H), 8.19-8.33 | |
| | (m, 2 H), 8.57 (d, J = 6.14 Hz, 1 H), 10.75-10.86 (m, 1 | |
| | H). | |
| Ac-PSKdR- | N.D. | 646.5 |
| kbt, | | |
| MM3194C | | |
| Ac-PSKR- | ¹H NMR (600 MHz, DMSO) δ 8.37 (d, J = 6.5 Hz, 1H), | 646.5 |
| kbt, | 8.27 (m, 2H), 8.03 − 7.90 (m, 1H), 7.80 (d, J = 8.1 Hz, | |
| MM3194A | 1H), 7.74 − 7.62 (m, 5H), 7.58 − 7.48 (m, 1H), 5.51 − 5.43 | |
| | (m, 1H), 5.25 − 4.91 (m, 1H), 4.48 − 4.15 (m, 3H), | |
| | 3.72 − 3.43 (m, 4H), 3.21 − 3.04 (m, 2H), 2.78 − 2.68 (m, | |
| | 2H), 2.25 − 1.80 (m, 8H), 1.80 − 1.42 (m, 8H), 1.40 − | |
| | 1.15 (m, 3H). | |
| VD5064A | N.D. | 755.5 |
| VD5064B | 1H NMR (400 MHz, DMSO-d6) δ 8.69 (d, J = 6.2 Hz, 1 | 755.5 |
| | H), 8.40 (d, J = 8.2 Hz, 1 H), 8.32 − 8.23 (m, 2 H), 8.18 | |
| | (d, J = 7.4 Hz, 1 H), 7.74 − 7.64 (m, 2 H), 7.31 (dd, J = | |
| | 8.2, 17.5 Hz, 2 H), 7.09 − 6.97 (m, 2 H), 6.89 (dd, J = 8.0, | |
| | 17.3 Hz, 1 H), 6.64 (dd, J = 2.3, 8.2 Hz, 1 H), 6.51 (br s, | |
| | 1 H), 5.38 (br s, 1 H), 4.64 − 4.46 (m, 1 H), 4.10 − 4.00 (m, | |
| | 1 H), 3.11 (d, J = 5.1 Hz, 2 H), 2.92 (br s, 2 H), 2.61 | |
| | (t, J = 12.1 Hz, 1 H), 1.92 (d, J = 10.9 Hz, 1 H), 1.84 (s, | |
| | 3 H), 1.71 (d, J = 9.3 Hz, 1 H), 1,58 (br s, 2 H), 1.43 | |
| | (dd, J = 6.6, 12.8 Hz, 1 H), 1.33 − 1.13 (m, 2 H), 0.82 − | |
| | 0.70 (m, 6 H). | |
| Ac-IQTR- | 1H NMR (399 MHz, dmso) δ 8.43 − 8.33 (m, 1H), 8.32 − | 676.6 |
| kbt, | 8.23 (m, 2H), 8.18 (t, J = 8.8 Hz, 1H), 7.98 − 7.88 (m, | |
| MM3186 | 1H), 7.72 − 7.56 (m, 3H), 7.47 − 7.37 (m, 1H), 7.30 − | |
| | 7.19 (m, 1H), 6.86 − 6.73 (m, 1H), 5.61 − 5.46 (m, 1H), | |
| | 4.93 − 4.77 (m, 1H), 4.37 − 4.19 (m, 2H), 4.13 (t, J = 7.8 | |
| | Hz, 1H), 4.02 − 3.87 (m, 1H), 3.17 − 3.11 (m, 2H), 2.16 − | |
| | 2.07 (m, 2H), 2.04 − 1.88 (m, 2H), 1.86 (s, 3H), 1.78 − | |
| | 1.64 (m, 3H), 1.64 − 1.52 (m, 2H), 1.51 − 1.34 (m, 1H), | |
| | 1.16 − 0.95 (m, 4H), 0.85 − 0.73 (m, 6H). | |
| Ac-IQWR- | 1H NMR (399 MHz, dmso) δ 10.77 (s, 1H), 8.59 − 8.47 | 761.6 |
| kbt, | (m, 1H), 8.32 − 8.23 (m, 2H), 7.99 − 7.80 (m, 2H), 7.73 − | |
| MM3187 | 7.62 (m, 2H), 7.55 − 7.39 (m, 2H), 7.35 − 7.18 (m, 2H), | |
| | 7.11 (dd, J = 10.5, 2.3 Hz, 1H), 7.08 − 6.86 (m, 2H), | |
| | 6.84 − 6.71 (m, 1H), 5.56 − 5.37 (m, 1H), 4.59 (p, J = 7.8 Hz, | |
| | 1H), 4.24 − 4.13 (m, 1H), 4.13 − 4.04 (m, 1H), 3.18 − | |
| | 3.02 (m, 3H), 2.94 (td, J = 15.2, 8.0 Hz, 1H), 1.14 − 1.00 | |
| | (m, 1H), 0.82 − 0.71 (m, 6H), 8.23 − 8.08 (m, 1H), 2.14 − | |
| | 2.01 (m, 2H), 1.95 (s, 1H), 1.86 (s, 3H), 1.86 − 1.53 (m, | |
| | 6H), 1.52 − 1.32 (m, 2H). | |
| Ac-IQVR- | 1H NMR (399 MHz, dmso) δ 8.61 (d, J = 6.2 Hz, 1H), | 674.6 |
| kbt, | 8.26 (ddd, J = 12.5, 6.6, 3.1 Hz, 2H), 8.11 (d, J = 7.8 Hz, | |
| MM3180-2 | 1H), 7.90 (d, J = 8.6 Hz, 1H), 7.74 − 7.60 (m, 3H), 7.48 | |
| | (t, J = 5.8 Hz, 1H), 7.26 (s, 1H), 6.79 (s, 1H), 5.52 − 5.43 | |
| | (m, 1H), 4.29 − 4.19 (m, 2H), 4.14 (t, J = 8.0 Hz, 1H), | |
| | 3.14 (q, J = 6.6 Hz, 2H), 2.18 − 2.03 (m, 2H), 2.00 − 1.89 | |
| | (m, 2H), 1.85 (s, 3H), 1.83 − 1.53 (m, 6H), 1.46 − 1.35 | |
| | (m, 1H), 1.13 − 1.01 (m, 1H), 0.90 − 0.74 (m, 13H). | |
| Ac-IQVdR- | N.D. | 674.6 |
| kbt, | | |
| MM3180-1 | | |
| Ac-IQAR- | ¹H NMR (399 MHz, dmso) δ 8.55 − 8.46 (m, 1H), 8.32 − | 646.5 |
| kbt, | 8.22 (m, 2H), 8.16 − 8.04 (m, 1H), 7.98 − 7.77 (m, 2H), | |
| MM3178 | 7.73 − 7.62 (m, 2H), 7.49 (q, J = 6.2 Hz, 1H), 7.29 − 7.23 | |
| | (m, 1H), 6.78 (s, 1H), 5.51 − 5.42 (m, 1H), 4.39 − 4.29 | |
| | (m, 1H), 4.19 (q, J = 8.0 Hz, 1H), 4.11 (q, J = 7.6 Hz, | |
| | 1H), 3.14 (p, J = 6.6 Hz, 2H), 2.16 − 2.05 (m, 2H), 2.03 − | |
| | 1.91 (m, 1H), 1.86 (s, 4H), 1.80 − 1.53 (m, 5H), 1.48 − | |
| | 1.34 (m, 1H), 1.26 − 1.15 (m, 3H), 1.15 − 1.03 (m, 1H), | |
| | 0.87 − 0.73 (m, 6H). | |
| Ac-IQSR- | 1H NMR (399 MHz, dmso) δ 8.51 − 8.43 (m, 1H), 8.32 − | 662.5 |
| kbt, | 8.23 (m, 2H), 8.10 (d, J = 7.8 Hz, 1H), 7.99 − 7.86 (m, | |
| MM3177 | 1H), 7.81 (d, J = 7.4 Hz, 1H), 7.74 − 7.63 (m, 2H), 7.46 | |
| | (t, J = 5.8 Hz, 1H), 7.33 − 7.20 (m, 1H), 6.80 (s, 1H), | |
| | 5.52 (dd, J = 8.2, 3.9 Hz, 1H), 4.86 (s, 1H), 4.35 (p, J = | |
| | 5.6 Hz, 1H), 4.25 (td, J = 8.4, 5.3 Hz, 1H), 4.11 (t, J = 7.8 | |
| | Hz, 1H), 3.62 − 3.49 (m, 2H), 3.14 (q, J = 6.8 Hz, 2H), | |
| | 2.15 − 2.05 (m, 2H), 2.00 − 1.81 (m, 5H), 1.79 − 1.64 (m, | |
| | 3H), 1.60 (d, J = 6.2 Hz, 2H), 1.47 − 1.37 (m, 1H), 1.19 − | |
| | 1.03 (m, 1H), 0.85 − 0.73 (m, 6H). | |
| Ac-QFR- | 1H NMR (400 MHz, DMSO-d6) δ ppm 0.85 − 1.23 (m, | 609.5 |
| kbt, | 10 H), 1.23 − 1.49 (m, 4 H), 2.15 (dd, J = 13.89, 8.41 Hz, 1 | |
| MM3144, N- | H), 2.30 (dd, J = 13.69, 6.26 Hz, 1 H), 2.37 − 2.48 (m, 2 | |
| 0386 | H), 3.41 − 3.52 (m, 1 H), 3.79 − 3.89 (m, 1 H), 4.86 − 4.96 | |
| | (m, 1 H), 6.24 − 6.42 (m, 7 H), 6.84 (quin, J = 7.53 Hz, 3 | |
| | H), 7.33 (d, J = 7.43 Hz, 1 H), 7.37 − 7.45 (m, 3 H), 7.73 | |
| | (d, J = 7.04 Hz, 1 H). | |
| Ac-LQFR- | 1H NMR (400 MHz, MeOD) δ 8.75 (d, J = 6.7 Hz, 1H), | 722.4 |
| kbt, | 8.37 (d, J = 7.8 Hz, 1H), 8.32 (d, J = 6.1 Hz, 1H), 8.23 (d, | |
| MM3131 | J = 7.9 Hz, 1H), 8.17 − 8.08 (m, 2H), 7.72 − 7.58 (m, | |
| | 2H), 7.25 − 7.07 (m, 5H), 5.76 − 5.65 (m, 1H), 4.65 (q, | |
| | J = 7.6 Hz, 1H), 4.33 − 4.18 (m, 2H), 3.41 − 3.12 (m, 2H), | |
| | 2.97 (dd, J = 14.0, 9.5 Hz, 1H), 2.32 − 1.47 (m, 15H), | |
| | 0.97 (dd, J = 6.6, 2.0 Hz, 3H), 0.93 (dd, J = 6.6, 2.0 Hz, | |
| | 3H). | |
| Ac-MQFR- | ¹H NMR (400 MHz, dmso) δ 8.58 (d, J = 7.0 Hz, 1H), | 740.3 |
| kbt, | 8.28 (t, J = 8.4 Hz, 2H), 8.15 (dd, J = 14.5, 7.4 Hz, 2H), | |
| MM3130 | 7.96 (d, J = 8.2 Hz, 1H), 7.74 − 7.63 (m, 2H), 7.53 (d, J = | |
| | 5.5 Hz, 1H), 7.34 − 7.09 (m, 6H), 6.82 (s, 1H), 5.51 (q, | |
| | J = 6.0 Hz, 1H), 4.62 − 4.52 (m, 1H), 4.25 (q, J = 6.8 Hz, | |
| | 1H), 4.15 (q, J = 7.2 Hz, 1H), 3.16 (q, J = 6.7 Hz, 2H), | |
| | 3.07 − 2.98 (m, 1H), 2.77 (dd, J = 14.3, 10.0 Hz, 1H), | |
| | 2.46 − 2.38 (m, 2H), 2.09 − 1.57 (m, 16H). | |
| Ac-PQFR- | 1H NMR (400 MHz, DMSO-d6) δ ppm 0.87 − 1.54 (m, | 706.5 |
| kbt, | 19 H), 2.09-2.24 (m, 1 H), 2.37-2.49 (m, 3 H), | |
| MF1003, | 2.76-2.89 (m, 1 H), 2.96-3.09(m, 1 H), 3.35-3.57 (m, 2 H), | |
| MF1016, | 3.78-3.94 (m, 1 H), 4.90 (br. s., 1 H), 6.25-6.53 (m, 6 | |
| MF2003, | H), 6.76-6.90 (m, 2 H), 7.23 (d, J = 7.43 Hz, 1 H), 7.33 | |
| MM3123 | (d, J = 7.83 Hz, 1 H), 7.37-7.48 (m, 2 H), 8.44 (d, J = 5.48 | |
| | Hz, 1H) | |
| Ac-GQFR- | 1H NMR (400 MHz, DMSO-d6) δ ppm 1.51-1.69 (m, 3 | 666.5 |
| kbt, | H), 1.70-1.83(m, 2 H), 1.87 (s, 3 H), 1.91-2.11 (m, 3 | |
| MM3122-2 | H), 2.71-2.86 (m, 1 H), 2.98-3.08 (m, 1 H), 3.11-3.22 | |
| | (m, 2 H), 3.68 (d, J = 5.48 Hz, 2 H), 4.10-4.23 (m, 1 H), | |
| | 4.55 (br. s., 1 H), 5.50 (br. s., 1 H), 6.80 (br. s., 1 H), | |
| | 7.09-7.32 (m, 7 H), 7.53 (br. s., 1 H), 7.63-7.75 (m, 2 H), | |
| | 8.02 (d, J = 8.22 Hz, 1 H), 8.09-8.21 (m, 2 H), 8.23-8.34 | |
| | (m, 2 H), 8.52 (d, J = 6.26 Hz, 1 H). | |
| Ac-IQFR- | H NMR (400 MHz, DMSO-d6) δ ppm 0.70-0.84 (m, 7 | 722.6 |
| kbt, | H) 1.00-1.14 (m, 1 H) 1.31-1.48 (m, 1 H) 1.54-1.84 | |
| MM3116 | (m, 7 H) 1.84-2.13 (m, 7 H) 2.72-2.84 (m, 1 H) 2.95-3.07 | |
| | (m, 1 H) 3.10-3.21 (m, 2 H) 4.08 (t, J = 6.85 Hz, 1 | |
| | H) 4.12-4.21 (m, 1 H) 4.51-4.64 (m, 1 H) 5.45-5.55 | |
| | (m, 1 H) 6.81 (br. s., 1 H)7.10-7.31 (m, 6 H) 7.49-7.57 | |
| | (m, 1 H) 7.64-7.74 (m, 2 H) 7.95 (t, J = 9.00 Hz, 2 H) | |
| | 8.15 (d, J = 7.83 Hz, 1 H) 8.28 (t, J = 8.61 Hz, 2 H) 8.56 (d, | |
| | J = 5.87 Hz, 1 H). | |
| VD4162A | N.D. | 794.6 |
| VD4162B | 1H NMR (400 MHz, DMSO-d6) δ = 10.74 − 10.67 (m, 1 | 794.6 |
| | H), 8.57 − 8.47 (m, 1 H), 8.33 − 8.21 (m, 2 H), 8.15 − 7.90 | |
| | (m, 1 H), 7.69 (dd, J = 2.5, 4.5 Hz, 2 H), 7.55 − 7.42 (m, 1 | |
| | H), 7.33 − 6.68 (m, 2 H), 6.56 − 6.43 (m, 1 H), 5.48 (br s, 1 | |
| | H), 4.45 − 4.20 (m, 2 H), 4.08 − 3.95 (m, 1 H), 3.13 (d, J = | |
| | 5.9 Hz, 3 H), 2.96 − 2.82 (m, 2 H), 2.03 (s, 3 H), 1.95 − | |
| | 1.86 (m, 4 H), 1.60 (br s, 8 H) | |
| VD4158 | 1H NMR (400 MHz, DMSO-d6) δ = ppm 1.18 (br s, 2 | 794.7 |
| | H), 1,28 (d, J = 11.74 Hz, 1 H), 1.42 (br s, 1 H), | |
| | 1.50-1.64 (m, 3 H), 1.68 (br s, 1 H), 1.72-1.85 (m, 1 H), | |
| | 1.85-1.94 (s, 3 H), 1.95 (br s, 1 H), 2.52-2.62 (m, 1 H), | |
| | 2.80-2.95 (m, 2 H), 2.95-3.07 (m, 1 H), 3.13 (d, J = 5.87 Hz, 2 | |
| | H), 3.91-4.12 (m, 2 H), 4.17-4.47 (m, 3 H), 5.48 (d, J = | |
| | 5.48 Hz, 1 H), 6.47 (d, J = 5,48 Hz, 1 H), 6.67 (d, J = | |
| | 6.65 Hz, 1 H), 6.73 (d, J = 7.83 Hz, 2 H), 6.75-6.89 (m, | |
| | 2 H), 6.89-7.10 (m, 5 H), 7.10-7.23 (m, 2 H), 7.23-7.41 | |
| | (m, 2 H), 7.50 (br s, 1 H), 7.58-7.78 (m, 2 H), 8.01 | |
| | (d, J = 8.61 Hz, 1 H), 8.14-8.35 (m, 3 H), 8.50 (d, J = | |
| | 5.48 Hz, 1 H), 10.70 (br s, 1 H) | |
| VD4118A | N.D. | 696.6 |
| VD4111A | N.D. | 755.5 |
| VD4118B | 1H NMR (400 MHz, DMSO-d6) δ = 8.68 (d, J = 6.2 Hz, | 696.6 |
| | 1 H), 8.48 (d, J = 9.0 Hz, 1 H), 8.28 (dd, J = 8.0, 13.4 | |
| | Hz, 1 H), 8.13 (d, J = 9.3 Hz, 1 H), 7.83 (d, J = 7.4 Hz, 1 | |
| | H), 7.74 − 7.63 (m, 1 H), 7.50 (s, 1 H), 5.48 (d, J = 5.4 Hz, | |
| | 1 H), 4.82 − 4.72 (m, 1 H), 4.48 − 4.37 (m, 1 H), 4.36 − 4.20 | |
| | (m, 1 H), 3.20 − 3.07 (m, 2 H), 2.91 − 2.78 (m, 1 H), 1.98 | |
| | (br s, 1 H), 1.79 (s, 3 H), 1.62 (br s, 1 H), 1.51 − 1.38 (m, | |
| | 2 H), 1.36 − 1.21 (m, 2 H), 0.87 − 0.75 (m, 6 H). | |
| VD4111B | 1H NMR (400 MHz, DMSO-d6) δ = 8.60 (d, J = 5.9 Hz, | 755.5 |
| | 1 H), 8.32 − 8.21 (m, 2 H), 8.19 (d, J = 7.0 Hz, 1 H), 8.09 | |
| | (d, J = 9.4 Hz, 1 H), 7.74 − 7.63 (m, 2 H), 7.53 (br s, 1 H), | |
| | 7.07 (d, J = 6.3 Hz, 2 H), 6.97 (d, J = 6.3 Hz, 3 H), 6.71 | |
| | (d, J = 7.8 Hz, 1 H), 6.37 (d, J = 5.5 Hz, 1 H), 5.47 | |
| | (d, J = 5.5 Hz, 1 H), 4.44 − 4.25 (m, 2 H), 3.99 (br s, 2 H), | |
| | 3.16 (d, J = 6.7 Hz, 2 H), 2.93 − 2.83 (m, 2 H), 2.69 − 2.59 | |
| | (m, 4 H), 1.97 (d, J = 7.0 Hz, 2 H), 1.88 (s, 4 H), 1.81 − | |
| | 1.56 (m, 4 H), 1.45 (br s, 4 H), 1.37 − 1.15 (m, 4 H) | |
| VD4090 | 1H NMR (400 MHz, DMSO-d6) δ = 8.57 (d, J = 5.9 Hz, | 696.6 |
| | 1 H), 8.27 (dd, J = 7.8, 12.9 Hz, 1 H), 8.12 − 8.01 (m, 1 | |
| | H), 7.88 (d, J = 7.4 Hz, 1 H), 7.75 − 7.65 (m, 1 H), 7.50 | |
| | (br s, 1 H), 5.46 (br s, 1 H), 4.68 (br s, 1 H), 4.57 − 4.46 | |
| | (m, 1 H), 4.39 − 4.21 (m, 2 H), 3.13 (d, J = 5.5 Hz, 1 H), | |
| | 1.95 (s, 3 H), 1.88 − 1.82 (m, 2 H), 1.79 − 1.52 (m, 2 H), | |
| | 1.44 (dd, J = 6.3, 13.3 Hz, 2 H), 1.33 (br s, 1 H), 0.94 (br | |
| | s, 2 H), 0.80 (dd, J = 6.1, 18.2 Hz, 6 H) | |
| VD4072 | 1H NMR (400 MHz, DMSO-d6) δ = 8.46 (d, J = 6.3 Hz, | 721.5 |
| | 1 H), 8.25 (dd, J = 7.6, 17.0 Hz, 2 H), 8.13 (d, J = 7.0 | |
| | Hz, 1 H), 7.93 (d, J = 9.0 Hz, 1 H), 7.72 − 7.61 (m, 2 H), | |
| | 7.46 (br s, 1 H), 7.00 (d, J = 7.0 Hz, 2 H), 6.90 (d, J = 7.8 | |
| | Hz, 1 H), 6.76 (d, J = 7.8 Hz, 2 H), 5.37 (br s, 1 H), 4,49 | |
| | (d, J = 4.7 Hz, 1 H), 4.32 (d, J = 8.6 Hz, 1 H), 4.08 − 3.90 | |
| | (m, 1 H), 3.12 (d, J = 5.9 Hz, 3 H), 2.87 (d, J = 7.8 Hz, 2 | |
| | H), 1.98 − 1,90 (m, 2 H), 1.83 (s, 3 H), 1.70 (br s, 3 H), | |
| | 1.56 (br s, 2 H), 1.42 − 1.31 (m, 1 H), 1.29 − 1.12 (m, 5 H), | |
| | 0.77 − 0.70 (m, 10 H). | |
| VD4054 | 1H NMR (400 MHz, DMSO-d6) δ = 8.48 − 8.36 (m, 1 | 776.6 |
| | H), 8.34 − 8.23 (m, 2 H), 8.17 − 8.08 (m, 1 H), 7.69 (br s, 2 | |
| | H), 7.47 (br s, 1 H), 7.02 − 6.89 (m, 2 H), 6.78 − 6.68 (m, 1 | |
| | H), 5.68 (br s, 1 H), 5.49 (d, J = 18.0 Hz, 2 H), 5.03 (br s, | |
| | 1 H), 4.75 (d, J = 4.7 Hz, 1 H), 4.61 − 4.49 (m, 2 H), 4.38 − | |
| | 4.27 (m, 1 H), 3.80 (br s, 1 H), 3.13 (br s, 4 H), 1.96 (br | |
| | s, 2 H), 1.87 (s, 3 H), 1.75 (br s, 3 H), 1.66 − 1.45 (m, 4 | |
| | H), 1.37 (d, J = 6.3 Hz, 2 H), 0.87 − 0.73 (m, 11 H). | |
| VD4051 | 1H NMR (400 MHz, DMSO-d6) δ = 8.79 (d, J = 7.4 Hz, | 719.5 |
| | 1 H), 8.33 − 8.25 (m, 2 H), 8.20 (d, J = 9.0 Hz, 1 H), 8.13 | |
| | (d, J = 7.4 Hz, 1 H), 7.75 − 7.64 (m, 2 H), 7.53 (br s, 1 H), | |
| | 7.07 (d, J = 7.8 Hz, 2 H), 6.67 (d, J = 7.8 Hz, 1 H), 5.60 − | |
| | 5.36 (m, 2 H), 4.72 − 4.54 (m, 2 H), 4.37 − 4.25 (m, 1 H), | |
| | 4.17 (br s, 1 H), 3.16 (d, J = 6.3 Hz, 2 H), 2.98 (d, J = | |
| | 11.7 Hz, 3 H), 1.79 (s, 3 H), 1.64 (br s, 4 H), 1.41 − 1.32 | |
| | (m, 3 H), 1.24 − 1.14 (m, 4 H), 0.83 − 0.72 (m, 10 H) | |
| VD4018 | 1H NMR (400 MHz, DMSO-d6) δ = 8.63 (s, 1 H), 8.31 − | 790.5 |
| | 8.20 (m, 2 H), 7.91 (d, J = 7.8 Hz, 1 H), 7.68 (br s, 2 H), | |
| | 7.44 (br s, 2 H), 7.00 (d, J = 8.2 Hz, 2 H), 6.70 (d, J = 8.6 | |
| | Hz, 1 H), 5.47 (br s, 5 H), 4.57 (br s, 2 H), 4.47 (br s, 1 | |
| | H), 3.13 (br s, 7 H), 2.71 − 2.65 (m, 6 H), 2.33 (s, 1 H), | |
| | 1.85 (s, 3 H), 1.74 (s, 2 H), 1.60 (br s, 3 H), 0.88 − 0.75 | |
| | (m, 10 H) | |
| VD4010 | 1H NMR (400 MHz, DMSO-d6) δ = 8.54 (d, J = 5.5 Hz, | 719.5 |
| | 1 H), 8.26 (dd, J = 7.4, 14.5 Hz, 2 H), 8.07 (d, J = 7.0 | |
| | Hz, 1 H), 8.02 − 7.96 (m, 1 H), 7.67 (d, J = 5.1 Hz, 2 H), | |
| | 7.53 − 7.46 (m, 1 H), 7.17 (br s, 1 H), 6.97 (d, J = 7.4 Hz, | |
| | 2 H), 6.68 (d, J = 7.8 Hz, 2 H), 5.62 − 5.36 (m, 1 H), 4.61 | |
| | (d, J = 12.1 Hz, 1 H), 4.52 − 4.37 (m, 1 H), 4.09 − 4.00 (m, | |
| | 1 H), 3.13 (d, J = 6.3 Hz, 3 H), 2.88 − 2.79 (m, 2 H), 2.66 − | |
| | 2.58 (m, 3 H), 1.84 (s, 3 H), 1.65 − 1.55 (m, 2 H), 1.43 − | |
| | 1.34 (m, 2 H), 1.28 − 1.17 (m, 2 H), 0.78 − 0.71 (m, 10 H). | |
| VD3173 | ¹H NMR (400 MHz, DMSO-d6) d = 8.54 (d, J = 6.3 Hz, 1 | 690.4 |
| | H), 8.31 − 8.07 (m, 2 H), 7.93 (d, J = 8.2 Hz, 1 H), 7.68 | |
| | (br. s., 1 H), 7.49 (br. s., 1 H), 5.40 (br. s., 1 H), 4.51 (br. | |
| | s., 1 H), 4.22 (br. s., 1 H), 3.15 (br. s., 6 H), 2.00 (d, J = | |
| | 1.2 Hz, 4 H), 1.88 − 1.77 (s, 3 H), 1.58 (br. s., 3 H), 1.39 − | |
| | 1.03 (m, 16 H). | |
| VD3152 | ¹H NMR (400 MHz, DMSO-d₆) d = 9.19 (s, 1 H), 8.98 − | 687.5 |
| | 8.81 (m, 2 H), 8.78 − 8.67 (m, 1 H), 8.60 − 8.50 (m, 1 H), | |
| | 8.38 − 8.24 (m, 1 H), 8.24 − 8.13 (m, 1 H), 7.89 − 7.81 (m, | |
| | 1 H), 7.41 − 7.32 (m, 1 H), 7.29 − 7.11 (m, 1 H), 6.21 − | |
| | 5.98 (m, 2 H), 5.25 − 5.08 (m, 1 H), 4.94 − 4.82 (m, 1 H), | |
| | 4.05 (br. s., 2 H), 3.79 (t, J = 6.1 Hz, 6 H), 3.15 − 3.04 | |
| | (m, 6 H), 2.69 − 2.56 (m, 3 H), 2.44 − 2.32 (m, 3 H), 2.04 | |
| | (s, 3 H), 2.25 (br. s., 5 H). | |
| PK-1-45A1, | 1H NMR (400 MHz, METHANOL-d4) δ ppm 10.58 (br. | 669.5 |
| dWFR-kbt- | s., OH), 8.82 (s, 1H), 8.22-8.32 (m, 2H), 7.70 (dd, J = | |
| COOH | 8.02, 16.24 Hz, OH), 7.57-7.64 (m, 1H),7.37 (d, J = 6.26 | |
| | Hz, 1H), 7.22 (d, J = 13.30 Hz, OH), 7.11-7.18 (m, 2H), | |
| | 7.02-7.10 (m, 1H), 5.55-5.67 (m, 1H), 4.47-4.58 (m, | |
| | OH), 4.35 (dd, J = 5.09, 10.17 Hz, OH), 4.05-4.31 (m, | |
| | 2H), 3.37-3.55 (m, 1H), 3.07-3.27 (m, 3H), 2.65 (s, | |
| | 1H), 2.18 (dd, J = 6.06, 13.11 Hz,1H), 1.57-1.95 | |
| | (m,3H), 1.16-1.52 (m, 2H), 1.06 (td, J = 7.14, 13.89 Hz, | |
| | 1H), 0.91-1.00 (m, 2H), 0.65-0.81 (m, 6H) | |
| Ac-dWFR- | 1H NMR (399 MHz, DMSO-d6) δ ppm 1.56-2.07 (m, 6 | 667.5 |
| kbt, | H), 1.93-2.04 (m, 1 H), 2.52-2.81 (m, 4 H), 2.96-3.08 | |
| MM3158, | (m, 1 H), 4.39-4.50 (m, 1 H), 4.57-4.70 (m, 1 H), | |
| PK-1-89A1 | 5.46-5.58 (m, 1 H), 6.90-7.09 (m, 4 H), 7.09-7.36 (m, 8 | |
| | H), 7.44-7.59 (m, 2 H), 7.62-7.76 (m, 2 H), 7.99 (d, | |
| | J = 7.79 Hz, 1 H), 8.28 (t, J = 8.37 Hz, 2 H), 8.47 (d, J = 8.95 | |
| | Hz, 1 H), 8.63 (d, J = 6.62 Hz, 1 H), 10.74 (s, 1 H). | |
| Ac-dWLR- | 1H NMR (400 MHz, dmso) δ 10.77 (s, 1H), 8.91 (s, 1H), | 677.5 |
| kbt-COOH, | 8.66 (d, J = 6.5 Hz, 1H), 8.32 (d, J = 8.4 Hz, 1H), 8.21 − | |
| MM4123, | 8.11 (m, 1H), 8.02 (dd, J = 22.0, 8.2 Hz, 2H), 7.51 (d, J = | |
| PK-1-94A1 | 8.5 Hz, 3H), 7.30 (d, J = 8.1 Hz, 1H), 7.21 (q, J = 12.1 | |
| | Hz, 6H), 7.03 (d, J = 5.8 Hz, 3H), 6.97 (q, J = 8.6 Hz, | |
| | 2H), 5.47 (d, J = 7.2 Hz, 1H), 4.60 (s, 1H), 4.43 (s, 1H), | |
| | 3.14 (d, J = 7.1 Hz, 2H), 2.98 (dd, J = 33.9, 14.1 Hz, 2H), | |
| | 2.77 (dd, J = 26.3, 13.5 Hz, 2H), 1.98 (s, 2H), 1.73 (s, | |
| | 3H), 1.61 (d, J = 8.7 Hz, 2H). | |
| Ac-WLR- | 1H NMR (400 MHz, dmso) δ 10.79 (d, J = 2.4 Hz, 1H), | 677.5 |
| kbt-COOH, | 8.90 (d, J = 1.7 Hz, 1H), 8.59 (d, J = 6.3 Hz, 1H), 8.30 (d, | |
| MM4094, | J = 8.7 Hz, 1H), 8.17 (dd, J = 8.6, 1.7 Hz, 1H), 8.00 (dd, | |
| PK-1-105A1 | J = 8.1, 3.7 Hz, 2H), 7.57 (d, J = 7.8 Hz, 1H), 7.50 (t, J = | |
| | 5.8 Hz, 1H), 7.31 (d, J = 8.1 Hz, 2H), 7.11 (d, J = 2.4 Hz, | |
| | 1H), 7.05 (t, J = 7.5 Hz, 1H), 6.96 (t, J = 7.4 Hz, 2H), | |
| | 5.39 (q, J = 6.5 Hz, 1H), 4.57 − 4.44 (m, 1H), 4.37 (q, J = | |
| | 7.7 Hz, 1H), 3.15 (d, J = 6.3 Hz, 2H), 3.06 − 2.94 (m, | |
| | 1H), 2.87 − 2.73 (m, 1H), 1.99 − 1.88 (m, 1H), 1.76 (d, J = | |
| | 4.2 Hz, 3H), 1.69 − 1.51 (m, 3H), 1.40 (t, J = 7.3 Hz, | |
| | 2H), 0.81 (dd, J = 11.1, 6.5 Hz, 7H). | |
| Ac-dWFR- | 1H NMR (600 MHz, MeOD) δ 8.06 − 7.97 (m, 1H), 7.84 | 711.5 |
| kbt-COOH, | (d, J = 8.6 Hz, 1H), 7.29 − 7.04 (m, 8H), 7.04 − 6.94 (m, | |
| MM4058, | 1H), 6.90 − 6.76 (m, 3H), 4.61 − 4.48 (m, 1H), 3.89 − | |
| PK-1-93A1 | 3.69 (m, 2H), 3.28 − 3.23 (m, 2H), 3.22 − 3.17 (m, 2H), | |
| | 2.46 − 2.26 (m, 2H), 2.26 − 2.10 (m, 2H), 2.08 − 1.82 (m, | |
| | 4H), 1.79 (s, 4H), 1.72 − 1.53 (m, 2H), 1.40 − 1.25 (m, | |
| | 3H). | |
| Ac-WFR- | 1H NMR (400 MHz, dmso) δ 10.77 (s, 1H), 8.91 (s, 1H), | 711.5 |
| kbt-COOH, | 8.66 (d, J = 6.5 Hz, 1H), 8.32 (d, J = 8.4 Hz, 1H), 8.21 − 8.15 | |
| MM4095, | (m, 1H), 8.02 (dd, J = 22.0, 8.2 Hz, 2H), 7.51 (d, J = | |
| PK-1-103A1 | 8.5 Hz, 2H), 7.30 (d, J = 8.1 Hz, 2H), 7.24 − 7.12 (m, | |
| | 5H), 7.07 − 6.89 (m, 4H), 5.47 (d, J = 7.2 Hz, 1H), 4.60 | |
| | (s, 1H), 4.43 (s, 1H), 3.14 (d, J = 7.1 Hz, 2H), 2.98 (dd, | |
| | J = 33.9, 14.1 Hz, 2H), 2.78 (dt, J = 25.4, 11.4 Hz, 2H), | |
| | 1.98 (s, 1H), 1.73 (s, 3H), 1.61 (d, J = 8.7 Hz, 2H). | |
| Ac-WFR- | 1H NMR (399 MHz, DMSO-d6) δ ppm 1.48 (br. s., 1 H), | 667.5 |
| kbt, | 1.54-1.69 (m, 1 H), 1.74 (m, 4 H), 2.70-2.89 (m, 2 H), | |
| MN1066, | 2.90-3.22 (m, 4 H), 4.37-4.74 (m, 1 H), 5.41-5.58 (m, | |
| PK-1-102, | 1 H), 6.86-7.10 (m, 4 H), 7.11-7.36 (m, 8 H), 7.40-7.57 | |
| PK-1-102A1 | (m, 2 H), 7.62-7.76 (m, 2 H), 7.93-8.12 (m, 2 H), | |
| | 8.22-8.34 (m, 2 H), 8.60 (t, J = 7.59 Hz, 1 H), 10.76 (s, 1 | |
| | H). | |
| Ac-WLR- | ¹H NMR (400 MHz, d3-MeOD) δ ppm 0.48 (d, J = 5.09 | 633.5 |
| kbt, | Hz, 1 H) 0.53-0.67 (m, 2 H) 0.75-0.93 (m, 4 H) 1.06-1.27 | |
| MN1070, | (m, 1 H) 1.34 (br. s., 1 H) 1.50 (br. s., 1 H) 1.74 (br. | |
| PK-1-104, | s., 1 H) 1.87 (d, J = 11.35 Hz, 1 H) 1.94 (s, 1 H) 1.99 (d, | |
| PK-1-104A1 | J = 6.65 Hz, 2 H) 2.17 (br. s., 1 H) 3.01-3.20 (m, 2 H) | |
| | 4.05 (s, 1 H) 4.16 (s, 1 H) 4.39 (br. s., 1 H) 4.49 (br. s., 1 | |
| | H) 4.59 (br. s., 1 H) 4.67 (br. s., 1 H) 5.56 (s, 1 H) 5.62 | |
| | (s, 1 H) 5.68 (br. s., 1 H) 6.94-7.06 (m, 1 H) 7.08 (br. s., | |
| | 1 H) 7.11-7.20 (m, 1 H) 7.26-7.39 (m, 1 H) 7.48-7.73 | |
| | (m, 3 H) 8.01 (d, J = 6.65 Hz, 1 H) 8.07 (br. s., 1 H) 8.11 | |
| | (d, J = 7.04 Hz, 1 H) 8.16-8.28 (m, 1 H). | |
| Ac-dWLR- | ¹H NMR (400 MHz, METHANOL-d4) δ ppm 0.48 (d, | 633.5 |
| kbt, | J = 5.48 Hz, 1 H) 0.52-0.67 (m, 5 H) 1.06-1.26 (m, 1 H) | |
| MN1063, | 1.33 (ddd, J = 13.89, 9.39, 4.89 Hz, 1 H) 1.75 (tt, J = 13.06, | |
| PK-1-91, | 6.70 Hz, 2 H) 1.81-1.95 (m, 2 H) 1.99 (d, J = 6.65 Hz, 3 | |
| PK-1-91A1 | H) 2.15 (d, J = 8.22 Hz, 1 H) 3.08-3.29 (m, 5 H) 3.98-4.11 | |
| | (m, 1 H) 4.18 (d, J = 6.26 Hz, 1 H) 4.45-4.53 (m, 1 | |
| | H) 4.57 (dd, J = 9.19, 6.06 Hz, 1 H) 4.63-4.76 (m, 1 H) | |
| | 5.55 (dd, J = 9.78, 3.91 Hz, 1 H) 5.68 (dd, J = 9.98, 3.72 | |
| | Hz, 1 H) 6.95-7.06 (m, 1 H) 7.06-7.16 (m, 2 H) 7.33 | |
| | (dd, J = 8.02, 2.54 Hz, 1 H) 7.49-7.71 (m, 3 H) 8.04-8.16 | |
| | (m, 2 H) 8.19 (d, J = 8.22 Hz, 1 H) 8.25 (d, J = 7.83 | |
| | Hz, 1 H). | |
| JH1143-2, | ¹H NMR (399 MHz, DMSO-d6) δ ppm 0.69-0.87 (m, 6 | 888.8 |
| JH1190, | H) 0.98-1.14 (m, 3 H) 1.23 (br. s., 3 H) 1.34-1.55 (m, | |
| MM4032-2 | 6 H) 1.60 (d, J = 6.23 Hz, 3 H) 1.66 (br. s., 1 H) 1.71 (d, | |
| | J = 10.51 Hz, 1 H) 1.82 (s, 3 H) 1.96 (br. s., 2 H) | |
| | 2.88-3.07 (m, 4 H) 3.07-3.23 (m, 4 H) 4.13 (dd, J = 13.04, | |
| | 8.37 Hz, 2 H) 4.30-4.46 (m, 2 H) 4.47-4.57 (m, 2 H) | |
| | 4.59-4.74 (m, 1 H) 5.45 (d, J = 10.51 Hz, 2 H) 5.66 (s, 2 | |
| | H) 6.84 (s, 2 H) 7.02 (s, 2 H) 7.15 (br. s., 1 H) 7.18-7.42 | |
| | (m, 8 H) 7.58-7.78 (m, 4 H) 7.97 (d, J = 8.17 Hz, 1 H) | |
| | 8.06 (d, J = 7.40 Hz, 1 H) 8.16-8.30 (m, 2 H) 8.34 (d, | |
| | J = 8.56 Hz, 1 H) 8.79 (d, J = 6.62 Hz, 1 H) 9.11 (br. s., 2 | |
| | H). | |
| VD2173 | .1H NMR (400 MHz, DMSO-d6) δ ppm = 8.51 (d, J = 6.7 | 672.5 |
| | Hz, 1 H), 8.26 (dd, J = 8.0, 15.1 Hz, 1 H), 7.98 (d, J = 7.4 | |
| | Hz, 1 H), 7.93 − 7.83 (m, 2 H), 7.73 − 7.63 (m, 2 H), 7.53 | |
| | (br. s., 1 H), 5.44 − 5.33 (m, 1 H), 4.60 − 4.48 (m, 1 H), | |
| | 4.29 − 4.18 (m, 1 H), 3.42 (br. s., 4 H), 3.19 − 3.06 (m, 3 | |
| | H), 2.96 (br. s., 1 H), 1.84 (s, 3 H), 1.78 − 1.69 (m, 1 H), | |
| | 1.65 − 1.33 (m, 8 H), 1.23 − 1.07 (m, 2 H), 0.89 − 0.74 (m, | |
| | 7 H). | |
| AJS3200-2- | ¹H NMR (600 MHz, d3-MeOD) δ 8.23 − 8.17 (m, 1H), | 681.69 |
| 1, Ac-Ser-4- | 8.15 − 8.11 (m, 1H), 7.67 − 7.59 (m, 2H), 7.27 − 7.22 (m, | |
| AMPA-Leu- | 4H), 5.71 − 5.48 (m, 1H), 4.43 − 4.36 (m, 4H), 3.83 − 3.75 | |
| Arg-kbt, | (m, 2H), 3.57 − 3.51 (m, 2H), 3.20 − 3.12 (m, 2H), | |
| JH1125-2, | 2.20 − 2.10 (m, 1H), 2.02 (s, 2H), 2.00 (s, 1H), 1.85 − | |
| JH1196 | 1.50 (m, 6H), 0.91 (dd, J = 6.5, 4.5 Hz, 3H), 0.85 (dd, J = | |
| | 12.2, 6.4 Hz, 3H). | |
| PK-1-18A1, | 1H NMR (400 MHz, METHANOL-d4) δ ppm 8.21 (d, J = | 625.5 |
| dWFR kbt | 6.65 Hz, 1H), 8.11 (d, J = 7.43 Hz, 1H), 7.57-7.67 (m, | |
| | 4H), 7.12-7.36 (m, 10H), 7.01-7.08(m, 2H), 6.84 (s, | |
| | 1H), 3.48 (d, J = 1.96 Hz, 1H), 3.13 (d, J = 6.65 Hz, 4H), | |
| | 3.01-3.07 (m, 1H), 2.75-2.84 (m, 1H), 2.65 (s, 2H), | |
| | 2.03 (s, 5H), 1.41(d, J = 8.61 Hz, 1H) | |
| Ac-SKFdR- | N.D. | 646.5 |
| kt, | | |
| ZFH6095- | | |
| iso | | |
| Ac-SKFR- | 1H NMR (400 MHz, DEUTERIUM OXIDE) 1.25 (m, 2 | 646.5 |
| kt, ZFH6095 | H) 1.42-1.81 (m, 7 H) 1.86-2.11 (m, 4 H) 2.80-3.06 | |
| | (m, 4 H) 3.13 (m, 2 H) 3.64-3.86 (m, 2 H) 4.17-4.28 | |
| | (m, 1 H) 4.32 (t, J = 5.67 Hz, 1 H) 4.56 (t, J = 7.83 Hz, 1 H) | |
| | 5.38 (dd, J = 8.61, 4.30 Hz, 1 H) 7.00-7.36 (m, 5 H) 8.04 | |
| | (d, J = 2.74 Hz, 1 H) 8.07 (d, J = 2.74 Hz, 1 H). | |
| Ac-KQFdR- | N.D. | 687.5 |
| kt, | | |
| ZFH6101- | | |
| iso | | |
| Ac-KQFR- | 1H NMR (400 MHz, DEUTERIUM OXIDE) 1.35 (m, | 687.5 |
| kt, ZFH6101 | 2H) 1.45-1.77 (m, 7 H) 1.78-2.04 (m, 6 H) 2.06-2.29 | |
| | (m, 2 H) 2.92 (t, J = 7.63 Hz, 2 H) 3.00 (m, 2 H) 3.13 (m, | |
| | 2 H) 4.12-4.20 (m, 1 H) 4.21-4.29 (m, 1 H) 4.59 (t, | |
| | J = 7.63 Hz, 1 H) 5.30-5.45 (m, 1 H) 7.17 (m, 5 H) 8.04 | |
| | (br. s., 1 H) 8.07 (br. s., 1 H) | |
| Ac-SQLR- | 1H NMR (400 MHz, DEUTERIUM OXIDE) 0.85 (d, | 612.5 |
| kt, ZFH6138 | J = 5.87 Hz, 3 H) 0.90 (d, J = 5.87 Hz, 3 H) 1.48-1.76 (m, | |
| | 5 H) 1.77-1.88 (m, 1 H) 1.89-2.02 (m, 1 H) 2.02-2.20 | |
| | (m, 5 H) 2.26-2.47 (m, 2 H) 3.11-3.32 (m, 2 H) 3.84 | |
| | (tt, J = 11.44, 5.77 Hz, 2 H) 4.25-4.45 (m, 3 H) 5.47 (dd, | |
| | J = 9.39, 4.30 Hz, 1 H) 8.06 (d, J = 3.13 Hz, 1 H) 8.11 (d, | |
| | J = 3.13 Hz, 1 H). | |
| Ac-LLR-kt, | N.D. | N.D. |
| ZFH6201-1 | | |
| Ac-KQLR- | 1H NMR (400 MHz, CD3OD): δ = 0.88 (d, J = 6.26 Hz, | 703.6 |
| kbt, | 3 H) 0.92 (d, J = 6.26 Hz, 3 H) 1.36-1.90 (m, 12 H) | |
| ZFH7006 | 1.90-2.26 (m, 3 H) 2.02 (s, 3 H) 2.26-2.44 (m, 2 H) 2.87-2.97 | |
| | (m, 2 H) 3.20-3.40 (m, 2 H) 4.21-4.29 (m, 1 H) | |
| | 4.31-4.38 (m, 1 H) 4.39-4.48 (m, 1 H) 5.63-5.78 (m, 1 H) | |
| | 7.54-7.74 (m, 2 H) 8.08-8.16 (m, 1 H) 8.19-8.29 ppm | |
| | (m, 1 H); | |
| Ac-SKLR | 1H NMR (400 MHz, CD3OD): δ = 0.87 (d, J = 6.65 Hz, | 662.5 |
| kbt, | 3 H) 0.91 (d, J = 6.65 Hz, 3 H) 1.36-2.08 (m, 12 H) 2.01 | |
| ZFH7053 | (s, 3 H) 2.10-2.29 (m, 1 H) 2.82-3.04 (m, 2 H) 3.22-3.38 | |
| | (m, 2 H) 3.65-3.93 (m, 2 H) 4.24-4.53 (m, 3 H) | |
| | 5.58-5.74 (m, 1 H) 7.56-7.71 (m, 2 H) 8.08-8.16 (m, 1 H) | |
| | 8.18-8.25 ppm (m, 1 H) | |
| Ac-FLFR- | 1H NMR (400 MHz, CD3OD): δ = 0.85 (d, J = 6.26 Hz, | 741.6 |
| kbt, | 3 H) 0.90 (d, J = 6.26 Hz, 3 H) 1.36-1.63 (m, 3 H) | |
| ZFH7063 | 1.66-1.99 (m, 3 H) 1.91 (s, 3 H) 2.05-2.25 (m, 1 H) 2.80-2.91 | |
| | (m, 1 H) 2.92-3.02 (m, 1 H) 3.02-3.18 (m, 2 H) | |
| | 3.20-3.33 (m, 2 H) 4.19-4.39 (m, 1 H) 4.47-4.70 (m, 2 H) | |
| | 5.64-5.75 (m, 1 H) 7.01-7.33 (m, 10 H) 7.57-7.71 (m, | |
| | 2 H) 8.10-8.17 (m, 1 H) 8.19-8.25 ppm (m, 1 H) | |
| Ac-WLFR- | 1H NMR (400 MHz, CD3OD): δ = 0.79 (d, J = 6.26 Hz, | 780.6 |
| kbt, | 3 H) 0.84 (d, J = 6.26 Hz, 3 H) 1.32-1.50 (m, 3 H) 1.62-1.91 | |
| ZFH7064 | (m, 3 H) 1.95 (s, 3 H) 2.06-2.21 (m, 1 H) 2.88-3.00 | |
| | (m, 1 H) 3.05-3.29 (m, 5 H) 4.14-4.33 (m, 1 H) | |
| | 4.50-4.72 (m, 2 H) 5.61-5.77 (m, 1 H) 6.96-7.03 (m, 1 H) | |
| | 7.04-7.20 (m, 7 H) 7.29-7.37 (m, 1 H) 7.54-7.71 (m, | |
| | 3 H) 7.85-7.97 (m, 1 H) 8.10-8.16 (m, 1 H) 8.18-8.26 | |
| | ppm (m, 1 H) | |
| Ac-SKLR | 1H NMR (400 MHz, CD3OD): δ = 0.87 (d, J = 6.46 Hz, | 804.7 |
| kbt V- | 3 H) 0.91 (d, J = 6.46 Hz, 3 H) 1.06 (d, J = 2.54 Hz, 3 H) | |
| amide, | 1.07 (d, J = 2.54 Hz, 3 H) 1.37-1.97 (m, 12 H) 2.01 (s, | |
| VD2156, | 3 H) 2.13-2.28 (m, 2 H) 2.79-3.03 (m, 2 H) 3.22-3.38 | |
| VD3040, | (m, 2 H) 3.67-3.98 (m, 2 H) 4.23-4.54 (m, 4 H) 5.49-5.77 | |
| VD3182, | (m, 1 H) 8.07-8.12 (m, 1 H) 8.25-8.32 (m, 1 H) | |
| VD4132, | 8.61-8.66 ppm (m, 1 H) | |
| ZFH7116 | | |
| Fmoc-FR- | 1H NMR (400 MHz, CD3OD) δ ppm 8.04-8.25 (m, 2 | 661.5 |
| kbt, | H), 7.50-7.83 (m, 7 H), 7.04-7.45 (m, 9 H), 5.66 (br s, 1 | |
| MF1133, | H), 4.04-4.49 (m, 5 H), 3.46 (br s, 2 H), 2.80-3.24 (m, 4 | |
| ZFH7187-14 | H), 1.72 (br s, 2 H) | |
| Ac- | 1H NMR (400 MHz, DMSO): δ 8.70 (d, J = 7.1 Hz, 1H), | 813.3 |
| IdcGlu(OAII) | 8.46 (d, J = 7.8 Hz, 1H), 8.31 − 8.21 (m, 2H), 8.09 (d, J = | |
| F)Arg-kbt, M | 8.3 Hz, 1H), 8.00 (d, J = 8.1 Hz, 1H), 7.72 − 7.61 (m, | |
| MPM208 | 2H), 7.49 (t, J = 5.7 Hz, 1H), 7.21 (dd, J = 8.5, 5.7 Hz, | |
| | 3H), 7.11 − 6.96 (m, 5H), 6.83 (t, J = 7.4 Hz, 1H), 5.91 − 5.76 | |
| | (m, 1H), 5.50 − 5.41 (m, 1H), 5.22 (d, J = 16.2 Hz, | |
| | 1H), 5.13 (d, J = 1.6 Hz, 1H), 5.00 (d, J = 10.7 Hz, 1H), | |
| | 4.63 (q, J = 8.2 Hz, 1H), 4.50 − 4.44 (m, 2H), 4.22 (q, J = | |
| | 7.4 Hz, 1H), 3.50 (dd, J = 16.9, 11.1 Hz, 1H), 3.06 (q, | |
| | J = 6.6 Hz, 2H), 3.00 − 2.90 (m, 2H), 2.76 (dd, J = 13.8, | |
| | 9.1 Hz, 1H), 2.27 − 2.19 (m, 2H), 1.94 (s, 3H), | |
| | 1.90 − 1.68 (m, 3H), 1.63 − 1.40 (m, 3H). | |
| Ac- | 1H NMR (400 MHz, DMSO): δ 8.73 (d, J = 6.4 Hz, 1H), | 813.3 |
| IdcGlu(OAll | 8.41 (d, J = 7.9 Hz, 1H), 8.31 − 8.21 (m, 2H), 8.02 (t, J = | |
| )Phe(4- | 7.9 Hz, 1H), 7.72 − 7.61 (m, 2H), 7.53 (t, J = 5.6 Hz, | |
| F)dArg-kbt, | 1H), 7.21 (dd, J = 8.5, 5.7 Hz, 3H), 7.15 − 7.04 (m, 2H), | |
| MF1067, | 7.00 − 6.87 (m, 4H), 5.95 − 5.79 (m, 1H), 5.46 (q, J = 6.4 | |
| MPM2082A | Hz, 1H), 5.24 (d, J = 17.3 Hz, 1H), 5.16 (d, J = 9.0 Hz, | |
| | 1H), 5.00 (dd, J = 11.0, 3.1 Hz, 1H), 4.66 − 4.53 (m, 1H), | |
| | 4.49 (d, J = 5.4 Hz, 2H), 4.22 − 4.13 (m, 1H), 3.48 (dd, | |
| | J = 16.9, 11.1 Hz, 1H), 3.10 (q, J = 6.6 Hz, 2H), 3.03 − 2.88 | |
| | (m, 2H), 2.72 (dd, J = 14.1, 9.9 Hz, 1H), 2.28 − 2.15 | |
| | (m, 2H), 1.94 (s, 3H), 1.86 − 1.65 (m, 3H), 1.61 − 1.51 | |
| | (m, 2H). | |
| Ac-Ser-3- | N.D. | N.D. |
| AMPA-Leu- | | |
| dArg-kbt, | | |
| MM1132-1, | | |
| MM4035-1 | | |
| JH1169, | ¹H NMR (400 MHz, METHANOL-d4) δ ppm 0.79 (dd, | 720.5 |
| JH1189, Ac- | J = 6.46, 1.76 Hz, 6 H) 0.84 (dd, J = 6.26, 4.70 Hz, 2 H) | |
| Ser-D-Trp- | 0.89-0.97 (m, 1 H) 1.17-1.40 (m, 2 H) 1.52 (br. s., 1 | |
| Leu-Arg-kbt | H) 1.68 (br. s., 2 H) 1.97 (s, 3 H) 2.90 (s, 1 H) 3.10-3.16 | |
| | (m, 2 H) 3.24 (d, J = 7.83 Hz, 2 H) 3.45-3.49 (m, 2 H) | |
| | 3.49-3.60 (m, 2 H) 4.09-4.16 (m, 1 H) 4.16-4.21 (m, | |
| | 1 H) 4.23 (d, J = 3.52 Hz, 1 H) 6.96-7.05 (m, 2 H) 7.05-7.13 | |
| | (m, 3 H) 7.33 (d, J = 7.43 Hz, 2 H) 7.53-7.69 (m, 2 | |
| | H). | |
| JH1142-2, | ¹H NMR (400 MHz, METHANOL-d4) δ ppm 0.84 (d, | 718.5 |
| Ac- | J = 6.65 Hz, 6 H) 1.15-1.26 (m, 2 H) 1.41-1.53 (m, 1 H) | |
| His(BOM)- | 1.95 (s, 3 H) 2.99-3.17 (m, 1 H) 3.21-3.29 (m, 3 H) | |
| hLeu-Arg- | 4.22-4.38 (m, 1 H) 4.64 (d, J = 3.13 Hz, 2 H) 4.74 (t, | |
| kbt | J = 7.04 Hz, 1 H) 5.56-5.68 (m, 1 H) 5.69 (s, 2 H) 7.22-7.41 | |
| | (m, 6 H) 7.64 (quin, J = 7.43 Hz, 2 H) 8.13 (d, J = 7.83 | |
| | Hz, 1 H) 8.21 (d, J = 7.83 Hz, 1 H) 8.77 (s, 1 H). | |
| JH1141-2, | ¹H NMR (400 MHz, METHANOL-d4) δ ppm 0.84 (d, | 631.5 |
| Ac-hArg- | J = 4.70 Hz, 3 H) 0.82 (d, J = 4.30 Hz, 3 H) 0.90 (dd, | |
| hLeu-Arg- | J = 6.65, 2.74 Hz, 3 H) 1.10-1.27 (m, 3 H) 1.35-1.51 | |
| kbt | (m, 5 H) 1.51-1.73 (m, 11 H) 1.73-1.86 (m, 5 H) 1.94-2.02 | |
| | (m, 5 H) 2.16 (s, 4 H) 2.17-2.25 (m, 1 H) 3.17 (t, | |
| | J = 6.85 Hz, 4 H) 3.24-3.29 (m, 2 H) 3.44-3.65 (m, 2 H) | |
| | 4.23-4.35 (m, 2 H) 4.80 (d, J = 4.30 Hz, 1 H) 5.66 (dd, | |
| | J = 9.19, 4.11 Hz, 1 H) 7.58-7.71 (m, 2 H) 8.14 (d, | |
| | J = 7.83 Hz, 1 H) 8.21 (d, J = 7.83 Hz, 1 H). | |
| JH1140-2, | ¹H NMR (400 MHz, METHANOL-d4) δ ppm 0.77-0.84 | 817.7 |
| Ac-D-Trp- | (m, 7 H) 0.85 (d, J = 2.74 Hz, 2 H) 0.87-0.91 (m, 1 H) | |
| hArg-hLeu- | 1.31-1.39 (m, 2 H) 1.69-1.84 (m, 3 H) 1.84-1.96 (m, | |
| Arg-kbt | 1 H) 1.98 (s, 3 H) 2.66 (s, 1 H) 2.99 (t, J = 7.24 Hz, 2 H) | |
| | 3.08-3.18 (m, 1 H) 3.18-3.27 (m, 2 H) 4.07 (dd, | |
| | J = 9.19, 5.28 Hz, 1 H) 4.19 (dd, J = 8.02, 6.46 Hz, 1 H) | |
| | 4.43-4.55 (m, 1 H) 5.63 (dd, J = 8.80, 3.72 Hz, 1 H) | |
| | 6.96-7.06 (m, 1 H) 7.06-7.16 (m, 2 H) 7.34 (d, J = 8.61 Hz, 1 | |
| | H) 7.58 (d, J = 7.83 Hz, 1 H) 7.60-7.70 (m, 2 H) 8.13 (d, | |
| | J = 7.43 Hz, 1 H) 8.21 (d, J = 7.83 Hz, 1 H) | |
| Ac-Ser-3- | ¹H NMR (400 MHz, DMSO-d6) δ ppm 0.73-0.86 (m, 6 | 680.83 |
| AMPA-Leu- | H) 1.32-1.47 (m, 2 H) 1.47-1.68 (m, 3 H) 1.68-1.82 | |
| Arg-kbt, | (m, 1 H) 1.87 (d, J = 1.57 Hz, 3 H) 1.90-2.05 (m, 1 H) | |
| JH1126-2, | 3.13 (d, J = 5.48 Hz, 2 H) 3.34 (br. s., 1 H) 3.55-3.65 (m, | |
| MM1132-2, | 2 H) 4.25 (d, J = 5.09 Hz, 2 H) 4.27-4.33 (m, 1 H) 4.33-4.43 | |
| MM4035-2 | (m, 1 H) 5.27-5.48 (m, 1 H) 7.10 (br. s., 3 H) 7.16-7.28 | |
| | (m, 1 H) 7.53 (br. s., 1 H) 7.59-7.75 (m, 2 H) | |
| | 7.92 (d, J = 7.83 Hz, 1 H) 8.12-8.31 (m, 3 H) 8.35 (t, | |
| | J = 5.09 Hz, 1 H) 8.64 (d, J = 5.48 Hz, 1 H). | |
| MM1180, | ¹H NMR (400 MHz, DMSO-d6) δ ppm 0.84 (d, J = 6.26 | 868.94 |
| Ac-Ser- | Hz, 3 H) 0.81 (d, J = 5.87 Hz, 3 H) 0.95 (d, J = 6.65 Hz, 6 | |
| Phe(p- | H) 1.41 (t, J = 6.46 Hz, 2 H) 1.59 (d, J = 18.00 Hz, 3 H) | |
| NO2)-Leu- | 1.82 (s, 4 H) 1.97 (br. s., 1 H) 2.06-2.23 (m, 1 H) 2.78-2.98 | |
| Arg-kbt V | (m, 1 H) 3.16 (d, J = 6.65 Hz, 3 H) 3.42-3.53 (m, 3 | |
| amide | H) 4.21 (q, J = 6.13 Hz, 1 H) 4.27-4.43 (m, 2 H) 4.45-4.63 | |
| | (m, 1 H) 5.30-5.48 (m, 1 H) 7.11 (br. s., 1 H) 7.41-7.59 | |
| | (m, 4 H) 7.92 (d, J = 7.43 Hz, 1 H) 8.03 (d, J = 7.83 | |
| | Hz, 2 H) 8.06-8.18 (m, 3 H) 8.29 (d, J = 8.22 Hz, 1 H) | |
| | 8.41 (d, J = 8.22 Hz, 1 H) 8.66 (d, J = 5.48 Hz, 1 H) 8.81 (s, | |
| | 1 H). | |
| JH1114, | 1H NMR (400 MHz, DMSO-d6) δ ppm 0.57-0.80 (m, | 863.32 |
| MM1189, | 8 H) 0.80-0.89 (m, 2 H) 0.95 (d, J = 6.26 Hz, 10 H) 1.28-1.41 | |
| Ac-Ser-D- | (m, 2 H) 1.51-1.73 (m, 2 H) 1.73-1.90 (m, 5 H) | |
| Trp-Leu- | 1.90-2.05 (m, 1 H) 2.05-2.10 (m, 2 H) 2.10-2.23 (m, | |
| Arg-kbt V | 1 H) 2.52-2.57 (m, 1 H) 2.83-3.06 (m, 2 H) 3.06-3.22 | |
| amide | (m, 4 H) 3.43 (d, J = 5.48 Hz, 3 H) 4.11-4.27 (m, 1 H) | |
| | 4.32 (t, J = 7.83 Hz, 2 H) 4.40-4.57 (m, 1 H) 5.29-5.51 | |
| | (m, 1 H) 6.95 (t, J = 7.43 Hz, 1 H) 7.04 (t, J = 7.63 Hz, 1 H) | |
| | 7.07-7.23 (m, 3 H) 7.30 (d, J = 8.22 Hz, 1 H) 7.43-7.65 | |
| | (m, 4 H) 7.85 - 7.97 (m, 1 H) 8.06 (d, J = 7.43 Hz, 2 H) | |
| | 8.13 (d, J = 8.22 Hz, 1 H) 8.29 (d, J = 8.61 Hz, 1 H) 8.42 (d, | |
| | J = 8.61 Hz, 1 H) 8.57 (d, J = 6.26 Hz, 1 H) 8.81 (s, 1 H) | |
| | 10.83 (br. s., 1 H). | |

### Example 3: Compound Fluorogenic Assay Data

TABLE 9
Compound Fluorogenic Assay Data

| | TMPRSS2 | Matriptase | Hepsin | HGFA | Thrombin | Factor Xa |
|---|---|---|---|---|---|---|
| Name | IC₅₀ (nM) | IC₅₀ (nM) | IC₅₀ (nM) | IC₅₀ (nM) | IC₅₀ (nM) | IC₅₀ (nM) |
| MF1064 (YK) | 0.004 | 1.230 | 0.831 | N.D. | N.D. | N.D. |
| PK-1-102A1 | 0.388 | 5.700 | 7.600 | 329.000 | >20,000 | 21.600 |
| (MN1066) | | | | | | |
| MF1140 | 0.001 | 2.888 | 10.980 | N.D. | N.D. | N.D. |
| MF1068 (MPM) | 0.083 | 0.375 | 0.308 | N.D. | N.D. | N.D. |
| PK-1-93 (VB) | | 136.000 | 2.000 | 95.500 | 19900.000 | 30.500 |
| MF1065 (MLS) | 0.062 | 3.490 | 1.760 | N.D. | N.D. | N.D. |
| MF1125 | 0.085 | 3.046 | 3.500 | N.D. | N.D. | N.D. |
| MM3122 | 0.030 | 0.314 | 0.194 | 32.500 | >20,000 | 700.000 |
| PK-1-103 | 0.223 | 49.100 | 2.080 | 235.000 | N.D. | N.D. |
| (MM4095) | | | | | | |
| VD4010 | 1.209 | 3.400 | 0.170 | 53.000 | >20,000 | 122.000 |
| ZFH7064 | 0.092 | 12.000 | 0.690 | 266.000 | >20,000 | 2.000 |
| VD2173 | 2.138 | 2.600 | 19.000 | 8520.000 | 8140.000 | 2050.000 |
| VD4090 | 4.188 | 39.000 | 14.000 | 3077.000 | >20,000 | 4294.000 |
| VD4072 | 9.792 | 32.000 | 0.910 | 40.000 | >20,000 | 2646.000 |
| MF1142 | 0.040 | 2805.000 | 2392.000 | N.D. | N.D. | N.D. |
| MM4009-2 | 0.067 | 2.170 | 1.050 | 126.000 | N.D. | N.D. |
| PK-1-89A1 | 0.156 | 2.600 | 1.100 | 27.000 | >20,000 | 98.000 |
| (MM3158) | | | | | | |
| MF1101 | 1.020 | 2.887 | 0.800 | N.D. | N.D. | N.D. |
| VD5076B | 1.251 | 180.000 | 7.540 | 250.000 | N.D. | N.D. |
| PK-1-91 (MN1063) | 0.026 | 39.000 | 1.600 | 104.000 | 9370.000 | 1440.000 |
| MM3131 | 0.031 | 1.800 | 0.247 | 98.200 | >20,000 | 219.000 |
| MM3180-2 | 0.031 | 0.761 | 0.106 | 799.000 | N.D. | N.D. |
| MM4038 | 0.040 | 25.000 | 12.200 | 66.200 | N.D. | N.D. |
| MM3123 | 0.060 | 0.316 | 0.135 | 75.000 | >20,000 | 199.000 |
| (MF1016/MF1003) | | | | | | |
| MM4028-2 | 0.064 | 4.710 | 0.863 | 182.000 | N.D. | N.D. |
| AJS4016 | 0.067 | 10.270 | 21.758 | N.D. | N.D. | N.D. |
| MF1105 | 0.070 | 1.476 | 0.310 | N.D. | N.D. | N.D. |
| CA1043 | 0.081 | 0.880 | 0.110 | 8.450 | >20,000 | 1590.000 |
| (MF1017) | | | | | | |
| MM4037-2 | 0.109 | 2.420 | 0.350 | 276.000 | N.D. | N.D. |
| MF1099 (N-0385) | 0.130 | 2.733 | 1.030 | N.D. | N.D. | N.D. |
| MM3130 | 0.142 | 0.552 | 0.134 | 36.400 | >20,000 | 342.000 |
| MF1163 | 0.167 | N.D. | N.D. | N.D. | N.D. | N.D. |
| MM3186 | 0.173 | 15.500 | 0.980 | 18400.000 | >20,000 | >20,000 |
| MF1067B | 0.200 | N.D. | N.D. | N.D. | N.D. | N.D. |
| (MPM2082B) | | | | | | |
| MF1177 | 0.211 | 37.560 | 6.069 | N.D. | N.D. | N.D. |
| MM3178 | 0.227 | 0.100 | 1.250 | >20,000 | >20,000 | 170.000 |
| MF1168 | 0.264 | >20000 | >20000 | N.D. | N.D. | N.D. |
| MF1104 | 0.273 | 5.592 | 1.070 | N.D. | N.D. | N.D. |
| MM3194A | 0.286 | 0.293 | 2.430 | >20,000 | N.D. | N.D. |
| MM3144 (N-0386) | 0.300 | 0.130 | 0.080 | 14.000 | >20,000 | 4.500 |
| MF2008 | 0.303 | 75.325 | N.D. | N.D. | N.D. | N.D. |
| CA1033-2 | 0.316 | 2.810 | 0.300 | 48.500 | 10200.000 | 1060.000 |
| AJS3200 | 0.376 | 9.520 | 2.813 | N.D. | N.D. | N.D. |
| (JH1125-2/ | | | | | | |
| JH1196) | | | | | | |
| MM4037-1 | 0.385 | N.D. | N.D. | N.D. | N.D. | N.D. |
| VD3173 | 0.401 | 1.000 | 5.900 | 3240.000 | 18500.000 | 1390.000 |
| CA1018 | 0.407 | 0.990 | 0.650 | 75.200 | >20,000 | 212.000 |
| ZFH9141 | 0.418 | 1.016 | 1.252 | N.D. | N.D. | N.D. |
| MF1067A | 0.431 | N.D. | N.D. | N.D. | N.D. | N.D. |
| (MPM2082A) | | | | | | |
| MF1184 | 0.466 | 58.725 | 118.200 | N.D. | N.D. | N.D. |
| CA1041-2 | 0.860 | 3.500 | 1.890 | 609.000 | >20,000 | 3300.000 |
| MF1165 | 0.864 | 31.100 | 0.100 | N.D. | N.D. | N.D. |
| VD5027 | 0.978 | 5.680 | 0.693 | 4.580 | 19900.000 | 1360.000 |
| CA1022-2 | 0.993 | 2.310 | 0.200 | 50.400 | >20,000 | 40.000 |
| PK-1-104 | 1.300 | 25.700 | 0.930 | 76.300 | 17000.000 | 890.000 |
| (MN1070) | | | | | | |
| MM4028-1 | 1.418 | N.D. | N.D. | N.D. | N.D. | N.D. |
| PK-1-105 | 1.453 | 144.000 | 0.362 | 98.300 | N.D. | N.D. |
| (MM4094) | | | | | | |
| ZFH6138 | 1.540 | 18.000 | 0.680 | 364.000 | N.D. | N.D. |
| MM3116 | 1.730 | 0.920 | 0.140 | 30.000 | >20,000 | 792.000 |
| VD5123A | 1.780 | 478.000 | 146.000 | 17300.000 | N.D. | N.D. |
| PK-1-94 | 1.906 | N.D. | N.D. | N.D. | N.D. | N.D. |
| (MM4123) | | | | | | |
| PK-1-45A1 | 1.940 | N.D. | N.D. | N.D. | N.D. | N.D. |
| MM3187 | 2.189 | 1.230 | 0.319 | 1450.000 | >20,000 | 129.000 |
| CA1022-1 | 2.353 | 8.800 | 0.830 | 1300.000 | >20,000 | 442.000 |
| VD4018 | 2.477 | 9.800 | 6.300 | 6500.000 | >20,000 | 12400.000 |
| ZFH7116 | 2.500 | 14.000 | 1.000 | 23.000 | 7530.000 | 514.000 |
| MM3180-1 | 2.886 | 22.900 | 1.720 | 14600.000 | >20,000 | >20,000 |
| MM4027-2 | 2.958 | 0.510 | 1.306 | N.D. | N.D. | N.D. |
| ZFH7063 (ZFH) | 3.000 | 7.200 | 2.900 | 228.000 | >20,000 | 26.000 |
| MM3177 | 4.550 | 1.300 | 2.420 | 19900.000 | >20,000 | 1728.000 |
| VD4162B | 4.745 | 2.880 | 0.538 | 3.350 | 14600.000 | 1110.000 |
| MM4027-1 | 5.630 | 16.100 | 3.160 | 234.000 | N.D. | N.D. |
| MF1134 | 6.300 | >20,000 | 1629.000 | N.D. | N.D. | N.D. |
| MM3122-1 | 6.456 | N.D. | N.D. | N.D. | N.D. | N.D. |
| JH1143-1, | 7.540 | 3.020 | 1.920 | 543.000 | 4890.000 | 4800.000 |
| MM4032-1 | | | | | | |
| MM4031-1 | 7.542 | N.D. | N.D. | N.D. | N.D. | N.D. |
| (JH1144) | | | | | | |
| ZFH6095 | 7.890 | 6.100 | 17.000 | 114.000 | >20,000 | 1060.000 |
| CA1046-2 | 8.225 | 41.400 | 0.370 | 704.000 | >20,000 | 2070.000 |
| MM4031-2 | 9.391 | N.D. | N.D. | N.D. | N.D. | N.D. |
| (JH1144-2) | | | | | | |
| VD5064B | 11.032 | 90.000 | 0.168 | 61.400 | >20,000 | 955.000 |
| MF1141 | 12.800 | 1136.000 | 346.300 | N.D. | N.D. | N.D. |
| MF1131 | 14.000 | 966.650 | 1128.000 | N.D. | N.D. | N.D. |
| VD4051 | 14.170 | 14.000 | 20.000 | >20,000 | >20,000 | 8810.000 |
| VD4118B | 14.861 | 91.200 | 137.000 | 10900.000 | >20,000 | 9540.000 |
| VD5123B | 15.200 | 140.000 | 36.900 | 3980.000 | N.D. | N.D. |
| VD4158 | 15.831 | 56.800 | 2.840 | 85.200 | >20,000 | 9530.000 |
| MF1143 | 16.100 | 97.740 | 228.100 | N.D. | N.D. | N.D. |
| VD4111B | 16.415 | 17.000 | 0.330 | 12.000 | >20,000 | 8697.000 |
| CA1033-1 | 16.640 | 77.900 | 9.430 | 2870.000 | >20,000 | 16800.000 |
| ZFH7006 | 18.100 | 1.100 | 0.170 | 60.000 | >20,000 | 258.000 |
| CA1041-1 | 18.450 | 144.000 | 123.000 | >20,000 | >20,000 | 7010.000 |
| MF1150 | 19.250 | 5.440 | 29.460 | N.D. | N.D. | N.D. |
| MF1133 | 19.700 | 2564.000 | 2680.000 | 1927.000 | >20,000 | 28.400 |
| (ZFH7187-14) | | | | | | |
| VD4054 | 21.555 | 44.000 | 0.990 | >20,000 | >20,000 | 3185.000 |
| VD3152 | 24.310 | 7.700 | 22.000 | 16100.000 | >20,000 | >20,000 |
| PK-1-18A1 | 25.805 | N.D. | N.D. | N.D. | N.D. | N.D. |
| MF1132 | 28.600 | 143.645 | 231.100 | N.D. | N.D. | N.D. |
| ZFH6101 | 28.900 | 1.400 | 1.200 | 116.000 | >20,000 | 158.000 |
| ZFH7053 | 39.200 | 6.100 | 0.320 | 66.000 | >20,000 | 3800.000 |
| ZFH6201-1 | 44.180 | 56.000 | 4.600 | 506.000 | NA | NA |
| MF1158 | 68.730 | 39.915 | >20000 | N.D. | N.D. | N.D. |
| CA1046-1 | 69.220 | 1780.000 | 105.000 | >20,000 | >20,000 | 12200.000 |
| MF1169 | 74.980 | 290.400 | >20000 | N.D. | N.D. | N.D. |
| MM4036-1 | 102.000 | 139.000 | 1.470 | 1550.000 | N.D. | N.D. |
| MM4146 | 115.400 | 97.400 | 15.800 | 543.000 | 15900.000 | 3980.000 |
| MF2012 | 187.400 | >20000 | N.D. | N.D. | N.D. | N.D. |
| MF1152 | 199.800 | 47.445 | >20000 | N.D. | N.D. | N.D. |
| VD5064A | 260.750 | 680.000 | 2.600 | 550.000 | N.D. | N.D. |
| MF1151 | 283.300 | 11.095 | N.D. | N.D. | N.D. | N.D. |
| MF1164 | 457.600 | N.D. | N.D. | N.D. | N.D. | N.D. |
| VD5076A | 488.950 | 2370.000 | 55.300 | 2900.000 | N.D. | N.D. |
| MF2009 | 613.900 | >20000 | N.D. | N.D. | N.D. | N.D. |
| MF2011 | 895.300 | 0.455 | N.D. | N.D. | N.D. | N.D. |
| MF1149 | >20000 | 18.030 | 2311.000 | N.D. | N.D. | N.D. |
| MF1144 | >20000 | >20,000 | 1411.000 | N.D. | N.D. | N.D. |
| JH1140-2 | N.D. | 0.460 | 0.450 | 4.840 | >20,000 | 9.120 |
| JH1143-2, JH1190, | N.D. | 0.862 | 0.728 | 43.900 | 1630.000 | 177.000 |
| MM4032-2 | | | | | | |
| JH1141-2 | N.D. | 1.060 | 1.150 | 10.500 | >20,000 | 121.000 |
| JH1142-2 | N.D. | 1.160 | 0.930 | 131.000 | N.D. | N.D. |
| MM3194C | N.D. | 5.720 | 24.900 | >20,000 | N.D. | N.D. |
| MM4009-1 | N.D. | 18.400 | 2.080 | 1340.000 | N.D. | N.D. |
| VD4118A | N.D. | 19.600 | 117.000 | 15600.000 | >20,000 | 1930.000 |
| VD4162A | N.D. | 30.800 | 3.050 | 52.000 | >20,000 | 7470.000 |
| MM1132-2, | N.D. | 31.000 | 0.326 | 209.000 | 9440.000 | 1890.000 |
| MM4035-2 | | | | | | |
| MM4036-2 | N.D. | 77.700 | 2.810 | 1070.000 | N.D. | N.D. |
| MM1180 | N.D. | 133.000 | 3.100 | 231.000 | 134.000 | 64.200 |
| JH1114, MM1189 | N.D. | 231.000 | 1.800 | 57.400 | 3880.000 | 1280.000 |
| VD4111A | N.D. | 330.000 | 12.700 | 3280.000 | N.D. | N.D. |
| MM1132-1, | N.D. | 419.000 | 1.910 | 3250.000 | N.D. | N.D. |
| MM4035-1 | | | | | | |

Fluorogenic Kinetic Enzyme Inhibitor Assays of HGFA, matriptase, TMPRSS2, and hepsin. Inhibitors (11-pt serial dilutions, concentrations ranged from 2 μM to 0.15 pM final concentration in reaction) were serially diluted in DMSO (2% DMSO final concentration) and then mixed with recombinant HGFA (1514-SE-010, R&D Systems), matriptase (3946-SEB-010, R&D Systems), TMPRSS2 (CSB-YP023924HU, Cusabio Technology) or activated hepsin (see below) in black 384 well plates (Corning #3575). The final assay concentration for HGFA, matriptase, TMPRSS2 and hepsin are 7.5 nM, 0.2 nM, 50 nM, and 0.3 nM, respectively in TNC buffer (25 mM Tris, 150 mM NaCl, 5 mM CaCl₂, 0.01% Triton X-100, pH 8). After 30 min incubation at room temperature, Boc-QLR-AMC (Vivitude, MQR-3135-v) substrate (Kₘ=37 μM) was added to the HGFA assays and Boc-QAR-AMC substrate was added to the matriptase (Kₘ=93 μM), TMPRSS2 (Kₘ=87 μM), and hepsin (Kₘ=156 μM) assays. The final substrate concentrations for all assays were at the Kₘ for the respective enzymes. Changes in fluorescence (excitation at 380 nm and emission at 460 nm) were measured at room temperature over time in a Biotek Synergy 2 plate reader (Winnoski, VT). Using GraphPad Prism version 6.04 software program, (GraphPad Software, San Diego, CA), a four-parameter curve fit was used to determine the inhibitor IC₅₀s from a plot of the mean reaction velocity versus the inhibitor concentration. The IC₅₀ values represent the average of three separate experimental determinations.

Hepsin Activation: Recombinant Hepsin (4776-SE-010, R&D Systems; g, 0.44 mg/mL) was diluted to 2.4 μM in TNC buffer (25 mM Tris, 150 mM NaCl, 5 mM CaCl₂, 0.01% Triton X-100, pH 8) and incubated at 37° C. After 24 h, the hepsin was diluted in glycerol to 5000. This stock hepsin (1.2 μM) was stored in a −20° C. freezer and diluted in TNC buffer for use in assays.

### Example 4: Serine Protease PS-SCL and Hycosul Screening Data

TABLE 10
P4 Position Various Amino Acids Substrate Activity for TMPRSS2,
Matriptase, Hepsin, and HGFA (percent substrate proteolysis/cleavage)

| Residue | TMPRSS2 | matriptase | hepsin | HGFA |
|---|---|---|---|---|
| L-His(3-Bom) | 58.42 | 13 | 38.40 | 100.00 |
| L-Agp | 16.48 | 50 | 98.08 | 80.01 |
| L-Lys(2-ClZ) | 98.03 | 61 | 41.91 | 47.56 |
| dhLeu | 99.65 | 56 | 52.44 | 42.50 |
| L-Idc | 96.99 | 23 | 0.00 | 32.64 |
| L-Chg | 98.44 | 38 | 46.64 | 25.12 |
| dhAbu | 85.03 | 36 | 44.51 | 24.45 |
| L-Arg | 27.99 | 100 | 80.32 | 23.62 |
| D-Arg | 24.43 | 18 | 44.37 | 23.20 |
| L-hArg | 44.91 | 86 | 56.24 | 22.07 |
| L-Phe(guan) | 13.71 | 10 | 35.43 | 20.91 |
| L-Orn | 12.33 | 82 | 84.52 | 20.23 |
| L-Lys | 14.11 | 75 | 75.43 | 18.29 |
| D-Lys | 16.31 | 14 | 49.84 | 17.66 |
| L-Nle(O-Bzl) | 63.90 | 49 | 91.83 | 16.90 |
| L-Cys(Bzl) | 81.95 | 19 | 23.44 | 16.89 |
| L-Oic | 100.00 | 42 | 53.72 | 16.76 |
| L-Dab | 12.90 | 32 | 47.53 | 16.76 |
| L-Dab(Z) | 65.55 | 44 | 96.34 | 16.55 |
| L-Tyr | 31.87 | 14 | 22.39 | 16.47 |
| L-Arg(Z)₂ | 31.09 | 62 | 61.73 | 16.44 |
| L-hLeu | 53.91 | 27 | 41.61 | 14.86 |
| L-Ser(Bzl) | 60.92 | 14 | 21.57 | 14.49 |
| L-Ile | 63.74 | 32 | 47.02 | 14.09 |
| L-Glu(O-Bzl) | 45.51 | 21 | 60.31 | 13.89 |
| L-Cys(4- | 52.23 | 13 | 12.84 | 13.88 |
| MeOBzl) | | | | |
| L-hTyr | 84.46 | 52 | 31.56 | 13.69 |
| L-Phe(4-Br) | 39.55 | 11 | 5.08 | 13.65 |
| L-Cys(MeBzl) | 57.94 | 16 | 15.53 | 13.63 |
| L-Cha | 43.75 | 13 | 25.44 | 13.42 |
| L-Dap | 16.80 | 16 | 35.65 | 13.31 |
| L-Thr(Bzl) | 51.24 | 11 | 40.13 | 13.14 |
| L-hCha | 49.87 | 23 | 5.44 | 12.92 |
| L-Bpa | 35.19 | 11 | 0.86 | 12.77 |
| L-Tyr(2-Br-Z) | 39.47 | 10 | 22.51 | 12.67 |
| L-Thz | 36.87 | 42 | 45.46 | 12.65 |
| L-Phe(4-Cl) | 32.02 | 11 | 26.01 | 12.60 |
| L-Phe(F₅) | 63.75 | 4 | 26.60 | 12.25 |
| L-2-Aoc | 71.41 | 22 | 19.98 | 12.15 |
| L-hSer(Bzl) | N.D. | 15 | 40.00 | 12.03 |
| L-Phe(4-Me) | 41.95 | 11 | 15.51 | 11.98 |
| L-Abu(Bth) | 34.33 | 30 | 38.68 | 11.84 |
| L-Nva | 54.45 | 30 | 41.99 | 11.78 |
| L-Nle | 56.06 | 34 | 40.93 | 11.76 |
| L-2-Nal | 31.99 | 8 | 1.34 | 11.72 |
| L-Val | 34.19 | 29 | 43.08 | 11.66 |
| L-Lys(TFA) | 30.34 | 17 | 50.71 | 11.66 |
| L-Agb | 61.09 | 38 | 37.96 | 11.59 |
| L-Phg | 42.95 | 24 | 34.01 | 11.24 |
| L-Phe(4-F) | 21.80 | 8 | 27.78 | 11.22 |
| L-Glu(O- | 69.67 | 12 | 45.93 | 11.19 |
| CHx) | | | | |
| L-Ala(2-th) | 40.98 | 13 | 35.30 | 11.16 |
| L-hPhe | 59.36 | 26 | 44.79 | 11.14 |
| L-Phe(2-Cl) | 31.27 | 7 | 14.92 | 10.91 |
| Gly | 39.79 | 30 | 29.19 | 10.84 |
| L-Tic | 44.15 | 27 | 13.31 | 10.84 |
| L-Hyp | 33.28 | 50 | 37.81 | 10.66 |
| L-hTyr(Me) | 44.34 | 19 | 36.30 | 10.62 |
| L-Igl | N.D. | 10 | 25.47 | 10.62 |
| D-Leu | 28.04 | 9 | 24.76 | 10.56 |
| L-hCit | 34.34 | 28 | 40.51 | 10.53 |
| L-Phe(2-F) | 29.40 | 11 | 24.33 | 10.49 |
| L-Asp(O- | 45.02 | 8 | 27.32 | 10.34 |
| CHx) | | | | |
| L-Pro | 40.11 | 40 | 56.00 | 10.30 |
| D-Pro | 19.76 | 9 | 20.45 | 10.30 |
| L-Ala(Bth) | 85.70 | 11 | 14.64 | 10.26 |
| L-Phe(3-Cl) | 28.04 | 7 | 9.26 | 10.25 |
| L-Phe(3-F) | 28.28 | 8 | 24.03 | 10.21 |
| L-Dht | 26.15 | 7 | 1.56 | 10.19 |
| L-Leu | 30.24 | 17 | 33.10 | 10.18 |
| D-Tyr | 33.78 | 7 | 12.66 | 10.09 |
| B-Ala | 45.65 | 18 | 30.74 | 10.07 |
| L-Tyr(2,6-Cl₂- | 26.89 | 13 | 0.08 | 9.94 |
| Bz1) | | | | |
| L-Phe(3,4-Cl₂) | 23.13 | 7 | 2.40 | 9.92 |
| L-Abu | 40.45 | 21 | 38.68 | 9.87 |
| L-Bta | N.D. | 7 | 13.63 | 9.79 |
| L-Tle | 32.65 | 20 | 38.05 | 9.71 |
| L-Asp(O-Bzl) | 26.92 | 6 | 19.34 | 9.60 |
| L-Tyr(Me) | 42.43 | 7 | 31.38 | 9.54 |
| L-Phe | 31.64 | 14 | 30.80 | 9.42 |
| L-Tyr(Bzl) | 36.68 | 14 | 3.10 | 9.39 |
| L-Asp(O-Me) | 26.57 | 5 | 18.55 | 9.31 |
| L-4-Pal | 24.78 | 7 | 33.68 | 9.27 |
| L-Pip | 15.31 | 11 | 34.68 | 9.25 |
| L-Glu(O-Me) | 26.42 | 12 | 36.79 | 9.13 |
| L-Phe(NH₂) | 25.99 | 11 | 24.64 | 9.11 |
| D-Phg | 27.54 | 9 | 22.48 | 9.06 |
| L-Glu | 9.81 | 18 | 29.82 | 9.01 |
| L-Hyp(Bzl) | 76.11 | 28 | 33.33 | 8.91 |
| L-His | 24.98 | 10 | 25.35 | 8.90 |
| L-Phe(3,4-F₂) | 22.33 | 6 | 19.65 | 8.79 |
| L-Trp | 24.62 | 9 | 6.82 | 8.78 |
| L-Lys(Ac) | 28.24 | 17 | 35.02 | 8.71 |
| D-Thr | 22.57 | 9 | 25.74 | 8.61 |
| L-NptGly | 30.47 | 9 | 27.66 | 8.34 |
| L-Arg(NO2) | N.D. | 21 | 82.11 | 8.33 |
| D-Val | 17.70 | 7 | 22.58 | 8.25 |
| L-Asn | 21.89 | 10 | 25.25 | 8.20 |
| D-Phe | 24.14 | 6 | 13.15 | 8.13 |
| D-Gln | 20.76 | 7 | 20.13 | 8.03 |
| D-Ser | 31.72 | 10 | 21.84 | 7.94 |
| L-Hnv | N.D. | 17 | 33.75 | 7.92 |
| D-His | 24.51 | 7 | 19.33 | 7.79 |
| L-3-Pal | 25.89 | 7 | 31.27 | 7.69 |
| L-Thr | 21.84 | 14 | 29.29 | 7.67 |
| D-Trp | 22.02 | 5 | 1.48 | 7.65 |
| L-Ser(Ac) | 24.05 | 13 | 37.96 | 7.56 |
| D-Asn | 19.65 | 7 | 18.48 | 7.38 |
| L-hSer | 17.28 | 9 | 26.55 | 7.34 |
| D-Ala | 27.00 | 6 | 18.44 | 7.11 |
| L-Ser | 20.12 | 19 | 31.63 | 7.04 |
| L-Ala | 26.30 | 31 | 41.30 | 6.68 |
| L-Met(O) | 24.40 | 10 | 22.86 | 6.64 |
| L-Cit | 33.20 | 18 | 36.68 | 6.44 |
| L-1-Nal | 15.22 | 4 | 5.18 | 6.36 |
| D-Glu | 23.68 | 1 | 2.98 | 2.62 |
| L-Trp(Me) | 16.80 | 6 | 0.00 | 2.49 |
| D-Asp | 18.22 | 1 | 1.98 | 2.25 |
| L-Gln | 26.44 | 0 | 0.76 | 1.96 |
| L-Asp | 7.77 | 1 | 0.22 | 1.90 |
| 4-Abz | 97.13 | N.D. | N.D. | N.D. |
| 2-Abz | 76.14 | N.D. | N.D. | N.D. |
| 3-Abz | 59.19 | N.D. | N.D. | N.D. |
| L-Phe(4-I) | 53.62 | N.D. | N.D. | N.D. |
| TmbGly | 50.69 | N.D. | N.D. | N.D. |
| L-hSer(Bzl) | 48.27 | N.D. | N.D. | N.D. |
| L-Bip | 46.26 | N.D. | N.D. | N.D. |
| L-Met | 38.95 | N.D. | N.D. | N.D. |
| L-Glu(All) | 33.68 | N.D. | N.D. | N.D. |
| L-Phe(3-I) | 31.28 | N.D. | N.D. | N.D. |
| L-His(Bzl) | 28.17 | N.D. | N.D. | N.D. |
| L-Aze | 26.46 | N.D. | N.D. | N.D. |
| L-Asp(OAll) | 25.00 | N.D. | N.D. | N.D. |
| D-hPhe | 24.66 | N.D. | N.D. | N.D. |
| L-Met(O)₂ | 19.75 | 10 | N.D. | N.D. |
| AC5C | 17.02 | N.D. | N.D. | N.D. |
| L-Aad | 12.85 | N.D. | N.D. | N.D. |

TABLE 11
P3 Position Various Amino Acids Substrate Activity for TMPRSS2,
Matriptase, Hepsin, and HGFA (percent substrate cleavage/proteolysis)

| Residue | TMPRSS2 | matriptase | hepsin | HGFA |
|---|---|---|---|---|
| L-hArg | 18.34 | 44 | 47.00 | 100.00 |
| D-Trp | 31.94 | 5 | 24.90 | 87.59 |
| L-Agp | 35.32 | 100 | 97.35 | 74.18 |
| L-hCha | 83.89 | 33 | 0.13 | 73.74 |
| L-hTyr | 51.36 | 28 | 7.46 | 73.59 |
| L-hPhe | 60.91 | 18 | 10.63 | 61.77 |
| L-Dht | 47.44 | 12 | 0.00 | 58.65 |
| L-Orn | 22.76 | 74 | 79.70 | 54.04 |
| L-Igl | N.D. | 9 | 0.00 | 52.15 |
| L-Lys | 27.86 | 81 | 80.71 | 44.63 |
| L-Arg | 27.44 | 70 | 70.75 | 43.98 |
| L-Phe(guan) | 4.15 | 24 | 9.10 | 42.11 |
| D-Phg | 24.55 | 7 | 57.07 | 40.01 |
| L-Glu(O-Bzl) | 86.34 | 37 | 50.15 | 33.35 |
| L-Arg(Z)₂ | 30.07 | 58 | 50.93 | 33.31 |
| L-2-Aoc | 74.43 | 27 | 12.74 | 33.06 |
| L-Glu(O-Me) | 67.49 | 39 | 64.70 | 32.95 |
| L-Nle(O-Bzl) | 71.88 | 75 | 82.17 | 30.67 |
| L-Glu(O- | 100.00 | 30 | 43.91 | 30.08 |
| CHx) | | | | |
| L-Dab | 36.73 | 66 | 48.55 | 29.40 |
| D-Arg | 0.00 | 8 | 66.08 | 29.10 |
| L-hTyr(Me) | 28.43 | 17 | 10.00 | 25.48 |
| L-Cys(Bzl) | 42.21 | 16 | 3.06 | 25.14 |
| L-Tic | 3.59 | 3 | 8.10 | 25.13 |
| L-Dap | 43.61 | 52 | 48.71 | 24.27 |
| L-Bta | N.D. | 10 | 0.13 | 24.11 |
| L-Ala(Bth) | 65.54 | 20 | 20.97 | 23.90 |
| L-hLeu | 35.41 | 25 | 13.84 | 23.44 |
| L-Phg | 39.25 | 26 | 28.27 | 23.25 |
| L-1-Nal | 15.37 | 7 | 0.00 | 23.07 |
| L-Cha | 32.05 | 29 | 3.64 | 22.61 |
| L-Asp(O- | 64.95 | 12 | 19.99 | 22.46 |
| CHx) | | | | |
| L-Phe(F₅) | 41.33 | 46 | 5.13 | 22.36 |
| L-Thr(Bzl) | 20.76 | 11 | 16.36 | 21.72 |
| L-Ser(Ac) | 80.78 | 43 | 42.09 | 21.69 |
| L-Abu(Bth) | 49.43 | 13 | 4.21 | 21.63 |
| L-Phe(3-Cl) | 24.33 | 16 | 0.35 | 21.45 |
| L-Dab(Z) | 55.06 | 37 | 31.58 | 21.38 |
| D-Lys | 0.00 | 15 | 64.56 | 21.11 |
| L-Lys(TFA) | 34.26 | 22 | 35.84 | 21.11 |
| L-Ala(2-th) | 26.44 | 16 | 11.54 | 20.51 |
| L-hSer(Bzl) | N.D. | 13 | 21.24 | 20.03 |
| L-hCit | 30.16 | 19 | 27.31 | 19.71 |
| L-Agb | 59.07 | 37 | 26.80 | 19.62 |
| L-Ser(Bzl) | 33.53 | 14 | 11.56 | 19.45 |
| L-Nle | 38.11 | 34 | 24.09 | 19.15 |
| L-Chg | 22.27 | 13 | 6.60 | 18.69 |
| L-Trp(Me) | 21.50 | 7 | 0.00 | 18.14 |
| L-Nva | 38.62 | 25 | 25.85 | 17.60 |
| L-Trp | 22.96 | 11 | 0.00 | 17.50 |
| L-Tyr(Me) | 20.50 | 14 | 4.55 | 17.41 |
| L-Phe(NH₂) | 19.02 | 15 | 11.65 | 17.32 |
| D-Tyr | 4.49 | 6 | 29.94 | 17.32 |
| L-Cys(MeBzl) | 32.14 | 11 | 0.87 | 17.21 |
| L-Tyr(2-Br-Z) | 19.56 | 13 | 8.05 | 17.09 |
| L-Phe(4-Me) | 16.55 | 12 | 2.39 | 16.82 |
| L-Cys(4- | 33.53 | 12 | 0.10 | 16.76 |
| MeOBzl) | | | | |
| L-Met(O) | 57.41 | 52 | 68.16 | 16.63 |
| L-hSer | 31.39 | 24 | 33.48 | 16.59 |
| L-Phe(3,4-Cl₂) | 17.27 | 12 | 0.00 | 16.15 |
| L-Lys(Ac) | 23.11 | 20 | 29.15 | 16.02 |
| L-Arg(NO2) | N.D. | 26 | 41.82 | 16.01 |
| L-Glu | 56.51 | 44 | 58.69 | 15.98 |
| B-Ala | 14.98 | 7 | 22.78 | 15.95 |
| L-Abu | 29.64 | 29 | 28.98 | 15.31 |
| D-Phe | 3.80 | 5 | 24.92 | 15.22 |
| D-Asn | 0.00 | 2 | 38.75 | 15.16 |
| L-Phe(3-F) | 20.84 | 18 | 10.43 | 15.12 |
| L-Hnv | N.D. | 17 | 33.40 | 15.07 |
| L-Phe(3,4-F₂) | 17.51 | 16 | 0.71 | 15.06 |
| L-Phe(4-F) | 14.82 | 11 | 5.16 | 15.01 |
| L-Ser | 35.95 | 33 | 38.70 | 14.57 |
| L-4-Pal | 15.18 | 14 | 21.91 | 14.57 |
| L-Thr | 39.80 | 31 | 35.34 | 14.51 |
| L-Lys(2-ClZ) | 57.03 | 40 | 2.55 | 14.21 |
| L-Tyr(Bzl) | 19.02 | 9 | 0.00 | 14.03 |
| L-Phe(4-Br) | 12.65 | 9 | 0.00 | 13.96 |
| D-Gln | 10.18 | 7 | 97.80 | 13.84 |
| L-Leu | 18.81 | 22 | 19.51 | 13.71 |
| L-Phe | 14.74 | 19 | 2.91 | 13.51 |
| L-Cit | 32.80 | 21 | 29.53 | 13.41 |
| L-Phe(2-F) | 30.50 | 14 | 7.80 | 13.39 |
| L-Phe(4-Cl) | 12.09 | 11 | 0.31 | 13.32 |
| L-His(3-Bom) | 34.99 | 11 | 15.18 | 13.20 |
| L-NptGly | 13.50 | 8 | 9.92 | 12.95 |
| L-2-Nal | 18.53 | 8 | 0.00 | 12.75 |
| L-His | 26.27 | 17 | 43.05 | 11.93 |
| L-Tyr | 16.18 | 17 | 7.75 | 11.59 |
| D-His | 6.35 | 3 | 40.09 | 11.09 |
| dhLeu | 11.11 | 10 | 5.85 | 11.02 |
| L-3-Pal | 17.98 | 15 | 28.37 | 11.00 |
| D-Ser | 0.00 | 2 | 54.84 | 10.86 |
| L-Phe(2-Cl) | 16.46 | 9 | 0.98 | 10.78 |
| L-Asp(O-Me) | 22.71 | 3 | 13.81 | 10.52 |
| Gly | 10.34 | 10 | 26.14 | 10.42 |
| D-Ala | 0.00 | 2 | 48.09 | 9.84 |
| L-Val | 19.38 | 18 | 16.77 | 9.51 |
| dhAbu | 7.83 | 4 | 7.29 | 9.44 |
| L-Ile | 13.26 | 13 | 11.37 | 9.06 |
| L-Ala | 31.84 | 34 | 28.07 | 8.97 |
| L-Bpa | 13.74 | 6 | 0.00 | 8.16 |
| L-Thz | 4.04 | 4 | 9.93 | 7.76 |
| D-Leu | 0.00 | 4 | 20.14 | 7.08 |
| L-Asp(O-Bzl) | 26.70 | 2 | 7.29 | 6.92 |
| L-Tle | 9.54 | 7 | 7.48 | 6.70 |
| D-Thr | 0.00 | 2 | 36.69 | 6.45 |
| L-Idc | 8.11 | 10 | 0.00 | 6.43 |
| L-Asn | 19.47 | 14 | 24.47 | 6.12 |
| D-Val | 0.00 | 1 | 27.04 | 5.76 |
| D-Asp | 20.28 | 0 | 17.18 | 5.37 |
| D-Pro | 0.00 | 0 | 38.88 | 4.96 |
| L-Tyr(2,6-Cl₂- | 8.35 | 6 | 0.09 | 4.47 |
| Bz1) | | | | |
| L-Hyp(Bzl) | 2.56 | 2 | 1.29 | 4.34 |
| D-Glu | 12.50 | 2 | 34.55 | 3.61 |
| L-Gln | 52.35 | 18 | 16.72 | 3.47 |
| L-Asp | 25.70 | 2 | 3.89 | 2.81 |
| L-Oic | 0.00 | 0 | 2.92 | 2.78 |
| L-Hyp | 2.19 | 0 | 4.70 | 2.18 |
| L-Pro | 0.00 | 2 | 2.09 | 2.04 |
| L-Pip | 0.00 | 0 | 1.64 | 0.83 |
| L-Glu(All) | 99.00 | N.D. | N.D. | N.D. |
| L-Met(O)₂ | 85.25 | 0 | N.D. | N.D. |
| L-Met | 45.55 | 41 | N.D. | N.D. |
| L-hSer(Bzl) | 41.71 | N.D. | N.D. | N.D. |
| L-Aad | 38.26 | N.D. | N.D. | N.D. |
| L-His(Bzl) | 30.78 | N.D. | N.D. | N.D. |
| L-Phe(3-I) | 30.56 | N.D. | N.D. | N.D. |
| L-Asp(OAll) | 29.33 | N.D. | N.D. | N.D. |
| D-hPhe | 17.56 | N.D. | N.D. | N.D. |
| TmbGly | 15.61 | N.D. | N.D. | N.D. |
| L-Phe(4-I) | 12.46 | N.D. | N.D. | N.D. |
| L-Bip | 12.34 | N.D. | N.D. | N.D. |
| 3-Abz | 11.57 | N.D. | N.D. | N.D. |
| 4-Abz | 9.87 | N.D. | N.D. | N.D. |
| 2-Abz | 5.50 | N.D. | N.D. | N.D. |
| L-Aze | 1.98 | N.D. | N.D. | N.D. |
| AC5C | 0.00 | N.D. | N.D. | N.D. |

TABLE 12
P2 Position Various Amino Acids Substrate Activity for TMPRSS2,
Matriptase, Hepsin, and HGFA (percent substrate cleavage/proteolysis)

| Residue | TMPRSS2 | matriptase | hepsin | HGFA |
|---|---|---|---|---|
| L-Leu | 5.90 | 12 | 100.00 | 100.00 |
| L-hLeu | 8.15 | 5 | 14.44 | 72.69 |
| L-NptGly | 9.35 | 0 | 37.46 | 53.86 |
| L-Nle | 11.80 | 13 | 62.04 | 45.52 |
| L-hTyr | 6.91 | 9 | 0.00 | 42.92 |
| L-Nva | 13.88 | 11 | 58.40 | 39.80 |
| L-hPhe | 5.66 | 23 | 4.71 | 32.14 |
| L-Lys(2-ClZ) | 21.71 | 28 | 21.20 | 29.53 |
| L-Chg | 2.44 | 1 | 10.88 | 27.27 |
| L-hTyr(Me) | 3.80 | 9 | 0.00 | 23.13 |
| L-Oic | 17.92 | 16 | 0.67 | 20.71 |
| L-Dht | 25.64 | 15 | 13.91 | 20.10 |
| L-Igl | N.D. | 100 | 2.20 | 19.79 |
| L-hSer(Bzl) | N.D. | 25 | 46.09 | 17.39 |
| L-Phe | 95.60 | 39 | 25.22 | 16.60 |
| L-2-Aoc | 8.46 | 19 | 8.90 | 14.37 |
| L-Abu(Bth) | 1.84 | 15 | 0.64 | 14.29 |
| L-Phg | 12.16 | 3 | 48.33 | 14.18 |
| dhAbu | 0.00 | 5 | 7.72 | 14.12 |
| L-Phe(3-Cl) | 91.89 | 69 | 33.86 | 12.49 |
| L-Phe(2-Cl) | 20.05 | 9 | 2.71 | 11.87 |
| L-Agp | 0.00 | 15 | 35.36 | 11.83 |
| L-Phe(guan) | 0.00 | 20 | 34.36 | 11.74 |
| L-Phe(3-F) | 88.34 | 50 | 30.55 | 11.72 |
| L-Ala(2-th) | 37.95 | 15 | 10.54 | 11.48 |
| L-Cys(4- | 8.88 | 17 | 7.07 | 9.44 |
| MeOBzl) | | | | |
| L-Thr | 22.02 | 15 | 70.75 | 8.87 |
| L-Val | 28.86 | 8 | 51.85 | 8.33 |
| L-hCit | 9.47 | 3 | 36.25 | 7.59 |
| L-Cys(Bzl) | 6.13 | 11 | 1.50 | 7.46 |
| L-Lys(Ac) | 1.18 | 13 | 23.04 | 7.36 |
| L-Thr(Bzl) | 10.27 | 3 | 36.14 | 7.19 |
| L-Glu(O-Bzl) | 62.01 | 57 | 36.33 | 6.88 |
| L-Glu(O-Me) | 5.57 | 6 | 13.49 | 6.65 |
| L-Glu(O- | 27.32 | 28 | 14.72 | 6.42 |
| CHx) | | | | |
| L-Cit | 0.00 | 1 | 32.27 | 6.32 |
| L-Cys(MeBzl) | 8.87 | 20 | 3.49 | 6.24 |
| L-Cha | 11.26 | 20 | 74.49 | 6.03 |
| L-Phe(2-F) | 25.11 | 14 | 1.78 | 5.98 |
| L-hArg | 13.07 | 16 | 57.96 | 5.83 |
| L-Hnv | N.D. | 3 | 34.62 | 5.49 |
| L-Ser(Bzl) | 10.15 | 21 | 20.43 | 5.27 |
| L-Lys(TFA) | 2.37 | 3 | 38.29 | 5.16 |
| L-2-Nal | 1.91 | 2 | 0.00 | 5.10 |
| L-Ile | 7.29 | 3 | 45.68 | 4.94 |
| L-Arg(Z)₂ | 1.83 | 4 | 52.91 | 4.93 |
| L-Phe(4-F) | 100.00 | 65 | 32.86 | 4.84 |
| L-Dab(Z) | 4.13 | 8 | 20.86 | 4.84 |
| L-Asn | 9.49 | 12 | 63.72 | 4.79 |
| D-Phg | 0.00 | 0 | 13.53 | 4.40 |
| L-Hyp(Bzl) | 9.94 | 4 | 32.68 | 4.39 |
| L-4-Pal | 11.02 | 15 | 0.11 | 4.34 |
| L-3-Pal | 18.33 | 6 | 17.32 | 4.29 |
| L-1-Nal | 24.40 | 29 | 3.00 | 4.19 |
| L-Trp | 33.05 | 32 | 6.00 | 4.04 |
| L-His | 15.38 | 30 | 31.24 | 4.00 |
| L-Hyp | 10.38 | 2 | 38.67 | 3.95 |
| L-Phe(3,4-F₂) | 96.35 | 76 | 38.16 | 3.73 |
| L-Tyr(Bzl) | 12.41 | 7 | 0.28 | 3.71 |
| L-Bta | N.D. | 32 | 0.00 | 3.71 |
| L-Tic | 9.79 | 2 | 2.45 | 3.58 |
| L-Glu | 6.08 | 6 | 12.19 | 3.35 |
| L-Ser | 19.86 | 28 | 18.40 | 3.24 |
| L-Tyr | 31.93 | 12 | 22.79 | 3.17 |
| L-hCha | 1.22 | 36 | 0.00 | 2.90 |
| L-Tyr(2,6-Cl₂- | 2.73 | 4 | 0.00 | 2.83 |
| Bzl) | | | | |
| L-Thz | 12.45 | 5 | 6.16 | 2.80 |
| L-Arg(NO2) | N.D. | 1 | 17.44 | 2.76 |
| L-Phe(NH₂) | 4.84 | 3 | 30.51 | 2.45 |
| L-Phe(4-Br) | 0.00 | 1 | 2.42 | 2.41 |
| L-Tyr(2-Br-Z) | 32.50 | 10 | 7.72 | 2.39 |
| L-Ala | 25.14 | 28 | 17.85 | 2.37 |
| L-Phe(3,4-Cl₂) | 3.02 | 27 | 0.00 | 2.22 |
| L-Pro | 16.87 | 22 | 13.10 | 2.21 |
| L-Asp(O- | 5.14 | 2 | 0.79 | 2.16 |
| CHx) | | | | |
| L-Ala(Bth) | 0.00 | 4 | 6.52 | 2.16 |
| L-hSer | 15.64 | 15 | 31.76 | 2.12 |
| L-Arg | 5.65 | 0 | 57.23 | 2.08 |
| L-Abu | 30.74 | 0 | 0.00 | 1.81 |
| L-Tyr(Me) | 2.43 | 1 | 10.89 | 1.79 |
| L-Pip | 24.73 | 10 | 10.66 | 1.75 |
| L-Phe(4-Cl) | 2.51 | 5 | 2.85 | 1.74 |
| L-Met(O) | 0.00 | 1 | 11.40 | 1.68 |
| L-Ser(Ac) | 5.46 | 9 | 6.73 | 1.67 |
| L-Bpa | 2.21 | 1 | 0.00 | 1.57 |
| L-Phe(F₅) | 0.92 | 14 | 0.36 | 1.55 |
| L-Dap | 4.32 | 9 | 10.01 | 1.49 |
| L-Asp(O-Bzl) | 1.84 | 1 | 0.00 | 1.35 |
| L-Asp | 9.72 | 1 | 0.33 | 1.21 |
| Gly | 8.64 | 23 | 0.00 | 1.14 |
| L-Trp(Me) | 32.33 | 1 | 7.81 | 1.12 |
| L-Agb | 28.63 | 1 | 3.53 | 1.05 |
| L-Lys | 3.79 | 6 | 46.70 | 1.04 |
| L-Phe(4-Me) | 0.00 | 1 | 6.44 | 0.97 |
| L-Idc | 2.07 | 0 | 0.00 | 0.96 |
| L-Gln | 6.03 | 1 | 0.00 | 0.89 |
| L-Asp(O-Me) | 2.62 | 1 | 0.00 | 0.75 |
| L-Dab | 0.00 | 0 | 13.49 | 0.50 |
| dhLeu | 0.00 | 0 | 0.00 | 0.48 |
| L-His(3-Bom) | 0.00 | 0 | 0.00 | 0.43 |
| B-Ala | 0.00 | 0 | 0.00 | 0.24 |
| L-Orn | 0.00 | 4 | 78.48 | 0.00 |
| D-Ser | 0.00 | 0 | 11.55 | 0.00 |
| L-Nle(O-Bzl) | 52.14 | 0 | 0.00 | 0.00 |
| L-Tle | 2.45 | 0 | 0.00 | 0.00 |
| D-Ala | 0.00 | 0 | 0.00 | 0.00 |
| D-Arg | 0.00 | 0 | 0.00 | 0.00 |
| D-Asn | 0.00 | 0 | 0.00 | 0.00 |
| D-Asp | 0.00 | 0 | 0.00 | 0.00 |
| D-Gln | 0.00 | 0 | 0.00 | 0.00 |
| D-Glu | 0.00 | 0 | 0.00 | 0.00 |
| D-His | 0.00 | 0 | 0.00 | 0.00 |
| D-Leu | 0.00 | 0 | 0.00 | 0.00 |
| D-Lys | 0.00 | 0 | 0.00 | 0.00 |
| D-Phe | 0.00 | 0 | 0.00 | 0.00 |
| D-Pro | 0.00 | 0 | 0.00 | 0.00 |
| D-Thr | 0.00 | 0 | 0.00 | 0.00 |
| D-Trp | 0.00 | 0 | 0.00 | 0.00 |
| D-Tyr | 0.00 | 0 | 0.00 | 0.00 |
| D-Val | 0.00 | 0 | 0.00 | 0.00 |
| L-His(Bzl) | 88.77 | N.D. | N.D. | N.D. |
| L-hSer(Bzl) | 24.71 | N.D. | N.D. | N.D. |
| L-Phe(3-I) | 20.16 | N.D. | N.D. | N.D. |
| L-Aze | 11.87 | N.D. | N.D. | N.D. |
| L-Met | 8.64 | 5 | N.D. | N.D. |
| L-Glu(All) | 8.38 | N.D. | N.D. | N.D. |
| L-Aad | 3.19 | N.D. | N.D. | N.D. |
| 4-Abz | 3.14 | N.D. | N.D. | N.D. |
| 3-Abz | 2.89 | N.D. | N.D. | N.D. |
| 2-Abz | 2.55 | N.D. | N.D. | N.D. |
| L-Asp(OAll) | 1.65 | N.D. | N.D. | N.D. |
| L-Met(O)₂ | 0.00 | 0 | N.D. | N.D. |
| D-hPhe | 0.00 | N.D. | N.D. | N.D. |
| AC5C | 0.00 | N.D. | N.D. | N.D. |
| L-Phe(4-I) | 0.00 | N.D. | N.D. | N.D. |
| TmbGly | N.D. | N.D. | N.D. | N.D. |
| L-Bip | N.D. | N.D. | N.D. | N.D. |

TABLE 13
P4 Position Top Ten Amino Acids Substrate Activity for TMPRSS2,
Matriptase, Hepsin, and HGFA (percent substrate cleavage/proteolysis)

| Residue | TMPRSS2 | matriptase | hepsin | HGFA |
|---|---|---|---|---|
| L-Oic | 100.00 | 42.50 | 53.72 | 16.76 |
| dhLeu | 99.65 | 55.61 | 52.44 | 42.50 |
| L-Chg | 98.44 | 37.58 | 46.64 | 25.12 |
| L-Lys(2- | 98.03 | 61.43 | 41.91 | 47.56 |
| ClZ) | | | | |
| 4-Abz | 97.13 | N.D. | N.D. | N.D. |
| L-Idc | 96.99 | 22.95 | 0.00 | 32.64 |
| L-Ala(Bth) | 85.70 | 10.86 | 14.64 | 10.26 |
| dhAbu | 85.03 | 36.16 | 44.51 | 24.45 |
| L-hTyr | 84.46 | 52.36 | 31.56 | 13.69 |
| L-Cys(Bzl) | 81.95 | 19.34 | 23.44 | 16.89 |
| L-Arg | 27.99 | 100.00 | 80.32 | 23.62 |
| L-hArg | 44.91 | 76.43 | 56.24 | 22.07 |
| L-Orn | 12.33 | 82.42 | 84.52 | 20.23 |
| L-Lys | 14.11 | 75.49 | 75.43 | 18.29 |
| L-Arg(Z)₂ | 31.09 | 62.39 | 61.73 | 16.44 |
| L-Lys(2- | 98.03 | 61.43 | 41.91 | 47.56 |
| ClZ) | | | | |
| dhLeu | 99.65 | 55.61 | 52.44 | 42.50 |
| L-hTyr | 84.46 | 52.36 | 31.56 | 13.69 |
| L-Hyp | 33.28 | 50.16 | 37.81 | 10.66 |
| L-Agp | 16.48 | 50.11 | 98.08 | 80.01 |
| L-Agp | 16.48 | 50.11 | 98.08 | 80.01 |
| L-Dab(Z) | 65.55 | 76.43 | 96.34 | 16.55 |
| L-Nle(O- | 63.90 | 48.54 | 91.83 | 16.90 |
| Bzl) | | | | |
| L-Orn | 12.33 | 82.42 | 84.52 | 20.23 |
| L- | N.D. | 62.39 | 82.11 | 8.33 |
| Arg(NO2) | | | | |
| L-Arg | 27.99 | 100.00 | 80.32 | 23.62 |
| L-Lys | 14.11 | 75.49 | 75.43 | 18.29 |
| L-Arg(Z)₂ | 31.09 | 62.39 | 61.73 | 16.44 |
| L-Glu(O- | 45.51 | 20.84 | 60.31 | 13.89 |
| Bzl) | | | | |
| L-hArg | 44.91 | 86.17 | 56.24 | 22.07 |
| L-His(3- | 58.42 | 12.94 | 38.40 | 100.00 |
| Bom) | | | | |
| L-Agp | 16.48 | 76.43 | 98.08 | 80.01 |
| L-Lys(2- | 98.03 | 61.43 | 41.91 | 47.56 |
| CIZ) | | | | |
| dhLeu | 99.65 | 55.61 | 52.44 | 42.50 |
| L-Idc | 96.99 | 62.39 | 0.00 | 32.64 |
| L-Chg | 98.44 | 37.58 | 46.64 | 25.12 |
| dhAbu | 85.03 | 36.16 | 44.51 | 24.45 |
| L-Arg | 27.99 | 100.00 | 80.32 | 23.62 |
| D-Arg | 24.43 | 17.66 | 44.37 | 23.20 |
| L-hArg | 44.91 | 86.17 | 56.24 | 22.07 |

TABLE 14
P3 Position Top Ten Amino Acids Substrate Activity for TMPRSS2,
Matriptase, Hepsin, and HGFA (percent substrate cleavage/proteolysis)

| Residue | TMPRSS2 | matriptase | hepsin | HGFA |
|---|---|---|---|---|
| L-Glu(O- | 100.00 | 30.20 | 43.91 | 30.08 |
| CHx) | | | | |
| L-Glu(All) | 99.00 | N.D. | N.D. | N.D. |
| L-Glu(O- | 86.34 | 36.77 | 50.15 | 33.35 |
| Bzl) | | | | |
| L-Met(O)₂ | 85.25 | 0.00 | N.D. | N.D. |
| L-hCha | 83.89 | 33.17 | 0.13 | 73.74 |
| L-Ser(Ac) | 80.78 | 42.92 | 42.09 | 21.69 |
| L-2-Aoc | 74.43 | 27.32 | 12.74 | 33.06 |
| L-Nle(O- | 71.88 | 75.40 | 82.17 | 30.67 |
| Bzl) | | | | |
| L-Glu(O- | 67.49 | 38.80 | 64.70 | 32.95 |
| Me) | | | | |
| L-Ala(Bth) | 65.54 | 20.27 | 20.97 | 23.90 |
| L-Agp | 35.32 | 100.00 | 97.35 | 74.18 |
| L-Lys | 27.86 | 76.43 | 80.71 | 44.63 |
| L-Nle(O- | 71.88 | 75.40 | 82.17 | 30.67 |
| Bzl) | | | | |
| L-Orn | 22.76 | 73.75 | 79.70 | 54.04 |
| L-Arg | 27.44 | 62.39 | 70.75 | 43.98 |
| L-Dab | 36.73 | 66.02 | 48.55 | 29.40 |
| L-Arg(Z)₂ | 30.07 | 57.98 | 50.93 | 33.31 |
| L-Dap | 43.61 | 51.80 | 48.71 | 24.27 |
| L-Met(O) | 57.41 | 51.68 | 68.16 | 16.63 |
| L-Phe(F₅) | 41.33 | 46.32 | 5.13 | 22.36 |
| D-Gln | 10.18 | 7.43 | 97.80 | 13.84 |
| L-Agp | 35.32 | 76.43 | 97.35 | 74.18 |
| L-Nle(O- | 71.88 | 75.40 | 82.17 | 30.67 |
| Bzl) | | | | |
| L-Lys | 27.86 | 80.67 | 80.71 | 44.63 |
| L-Orn | 22.76 | 62.39 | 79.70 | 54.04 |
| L-Arg | 27.44 | 70.35 | 70.75 | 43.98 |
| L-Met(O) | 57.41 | 51.68 | 68.16 | 16.63 |
| D-Arg | 0.00 | 7.82 | 66.08 | 29.10 |
| L-Glu(O- | 67.49 | 38.80 | 64.70 | 32.95 |
| Me) | | | | |
| D-Lys | 0.00 | 14.70 | 64.56 | 21.11 |
| L-hArg | 18.34 | 44.42 | 47.00 | 100.00 |
| D-Trp | 31.94 | 76.43 | 24.90 | 87.59 |
| L-Agp | 35.32 | 100.00 | 97.35 | 74.18 |
| L-hCha | 83.89 | 33.17 | 0.13 | 73.74 |
| L-hTyr | 51.36 | 62.39 | 7.46 | 73.59 |
| L-hPhe | 60.91 | 18.00 | 10.63 | 61.77 |
| L-Dht | 47.44 | 12.40 | 0.00 | 58.65 |
| L-Orn | 22.76 | 73.75 | 79.70 | 54.04 |
| L-Igl | N.D. | 8.98 | 0.00 | 52.15 |
| L-Lys | 27.86 | 80.67 | 80.71 | 44.63 |

TABLE 15
P2 Position Top Ten Amino Acids Substrate Activity for TMPRSS2,
Matriptase, Hepsin, and HGFA (percent substrate cleavage/proteolysis)

| Residue | TMPRSS2 | matriptase | hepsin | HGFA |
|---|---|---|---|---|
| L-Phe(4-F) | 100.00 | 64.79 | 32.86 | 4.84 |
| L-Phe(3,4- | 96.35 | 76.43 | 38.16 | 3.73 |
| F₂) | | | | |
| L-Phe | 95.60 | 38.80 | 25.22 | 16.60 |
| L-Phe(3-Cl) | 91.89 | 68.78 | 33.86 | 12.49 |
| L-His(Bzl) | 88.77 | | | |
| L-Phe(3-F) | 88.34 | 50.15 | 30.55 | 11.72 |
| L-Glu(O- | 62.01 | 56.79 | 36.33 | 6.88 |
| Bzl) | | | | |
| L-Nle(O-Bzl) | 52.14 | 0.00 | 0.00 | 0.00 |
| L-Ala(2-th) | 37.95 | 14.60 | 10.54 | 11.48 |
| L-Trp | 33.05 | 31.98 | 6.00 | 4.04 |
| L-Igl | N.D. | 100.00 | 2.20 | 19.79 |
| L-Phe(3,4- | 96.35 | 76.43 | 38.16 | 3.73 |
| F₂) | | | | |
| L-Phe(3-Cl) | 91.89 | 68.78 | 33.86 | 12.49 |
| L-Phe(4-F) | 100.00 | 64.79 | 32.86 | 4.84 |
| L-Glu(O- | 62.01 | 62.39 | 36.33 | 6.88 |
| Bzl) | | | | |
| L-Phe(3-F) | 88.34 | 50.15 | 30.55 | 11.72 |
| L-Phe | 95.60 | 38.80 | 25.22 | 16.60 |
| L-hCha | 1.22 | 36.48 | 0.00 | 2.90 |
| L-Bta | N.D. | 32.12 | 0.00 | 3.71 |
| L-Trp | 33.05 | 31.98 | 6.00 | 4.04 |
| L-Leu | 5.90 | 11.90 | 100.00 | 100.00 |
| L-Orn | 0.00 | 76.43 | 78.48 | 0.00 |
| L-Cha | 11.26 | 19.99 | 74.49 | 6.03 |
| L-Thr | 22.02 | 14.81 | 70.75 | 8.87 |
| L-Asn | 9.49 | 62.39 | 63.72 | 4.79 |
| L-Nle | 11.80 | 13.13 | 62.04 | 45.52 |
| L-Nva | 13.88 | 10.85 | 58.40 | 39.80 |
| L-hArg | 13.07 | 15.51 | 57.96 | 5.83 |
| L-Arg | 5.65 | 0.46 | 57.23 | 2.08 |
| L-Arg(Z)₂ | 1.83 | 3.79 | 52.91 | 4.93 |
| L-Leu | 5.90 | 11.90 | 100.00 | 100.00 |
| L-hLeu | 8.15 | 76.43 | 14.44 | 72.69 |
| L-NptGly | 9.35 | 0.00 | 37.46 | 53.86 |
| L-Nle | 11.80 | 13.13 | 62.04 | 45.52 |
| L-hTyr | 6.91 | 62.39 | 0.00 | 42.92 |
| L-Nva | 13.88 | 10.85 | 58.40 | 39.80 |
| L-hPhe | 5.66 | 22.58 | 4.71 | 32.14 |
| L-Lys(2-ClZ) | 21.71 | 28.17 | 21.20 | 29.53 |
| L-Chg | 2.44 | 0.84 | 10.88 | 27.27 |
| L-hTyr(Me) | 3.80 | 8.51 | 0.00 | 23.13 |

The following are the preferred amino acids for the cap, positions P4, P3, P2, and P1.

For the cap, they are Ac, Bn, Bz, PhCH₂CH₂, Cbz, Fmoc, MeSO₂, PhSO₂, CypSO₂, BnSO₂, Piperidine, dimethyl, PhCH₂CO, and Boc.

For P4, they are 2-Abz, 3-Abz, 4-Abz, D-Arg, dhAbu, dhLeu, D-Lys, Gly, L-2-Aoc, L-Agb, L-Agp, L-Ala(Bth), L-Arg, L-Arg(NO₂), L-Arg(Z)₂, L-Chg, L-Cys(4-MeOBzl), L-Cys(Bzl), L-Cys(MeBzl), L-Dab(Z), L-Glu(O-Bzl), L-Glu(O-CHx), L-hArg, L-hCha, L-His(3-Bom), L-hLeu, L-hPhe, L-hTyr, L-Hyp, L-Hyp(Bzl), L-Idc, L-Ile, L-Leu, L-Lys, L-Lys(2-ClZ), L-Lys(TFA), L-Met, L-Nle, L-Nle(O-Bzl), L-Nva, L-Oic, L-Orn, L-Phe(4-I), L-Phe(F₅), L-Pro, L-Ser, L-Ser(Bzl), L-Thr(Bzl), and TmbGly.

For P3, they are 3-AMPA, 4-AMBA, 4-AMPA, D-Arg, D-Gln, D-Lys, D-Phg, D-Ser, D-Trp, L-2-Aoc, L-Abu(Bth), L-Agb, L-Agp, L-Ala(Bth), L-Arg, L-Arg(Z)₂, L-Asp(O-CHx), L-Dab, L-Dab(Z), L-Dap, L-Dht, L-Gln, L-Glu, L-Glu(All), L-Glu(Bzl), L-Glu(O-Bzl), L-Glu(O-CHx), L-Glu(O-Me), L-hArg, L-hCha, L-His(3-Bom), L-hPhe, L-hTyr, L-Igl, L-Lys, L-Lys(2-ClZ), L-Met, L-Met(O), L-Met(O)₂, L-Nle(O-Bzl), L-Orn, L-Ph(4-NO₂), L-Phe(F₅), L-Ser(Ac), and L-Thr.

For P2, they are 2-Nal, L-Ala, L-Ala(2-th), L-Arg, L-Arg(Z)₂, L-Asn, L-Bta, L-Cha, L-Chg, L-Glu(O-Bzl), L-hArg, L-hCha, L-His(Bzl), L-hLeu, L-hPhe, L-hTyr, L-hTyr(Me), L-Igl, L-Leu, L-Lys(2-ClZ), L-Nle, L-Nle(O-Bzl), L-NptGly, L-Nva, L-Orn, L-Phe, L-Phe(3,4-F₂), L-Phe(3-Cl), L-Phe(3-F), L-Phe(4-F), L-Phg, L-Ser, L-Thr, L-Trp, and L-Val.

For P1, then are L-Arg and D-Arg.

### PS-SCL Protease Substrate Specificity Profiling of TMPRSS2

To augment the rational design of more potent and selective TMPRSS2 inhibitors, existing data on the peptide substrate preferences of TMPRSS2, HGFA, matriptase and hepsin was analyzed. When comparing the positional scanning of substrate combinatorial libraries (PS-SCL) data of TMPRSS2 with that of matriptase, hepsin and HGFA, it became apparent that there was significant overlap in their preferences for substrates. This data reveals TMPRSS2 is tolerant of many different P2 sidechains but prefers Phe and Ala/Thr like matriptase which can also tolerate both large and small groups (prefers Ser/Ala) but hepsin and HGFA both prefer Leu. For P3 TMPRSS2 prefers Gln/Glu and Met whereas it is Lys/Gln for hepsin and matriptase and Lys/Arg for HGFA. The clearest distinction is in the P4 position where HGFA, matriptase and hepsin all prefer basic residues like Lys/Arg while TMPRSS2 prefers Ile/Gly and Pro, which is shared attribute with hepsin.

### Characterization of Matriptase, Hepsin and HGFA P4-P2 Substrate Specificity Using HyCoSuL.

To elucidate substrate specificities of matriptase, hepsin, and HGFA proteases at P4-P2 positions, we employed the P1-Arg HyCoSuL library. Each of the P4, P3, and P2 sublibraries was screened at a 100 μM concentration with either matriptase, hepsin, or HGFA in a 100 μL final volume per well (99 μL of protease in buffer+1 μL of 10 mM substrates mixture). The total time of the assay was 30 min, however only linear portions of the progress curve (5-15 min) were used for velocity (RFU/s) calculations. Each sublibrary screening was repeated at least 3 times and the average value calculated from each measurement was used to create the substrate specificity matrix—the best recognized amino acid in each position was set to 100%, and other amino acids were adjusted accordingly. Hepsin. Recombinant human hepsin (10 μg in 20 μL, 4776-SE-010, R&D Systems) was added to the 100 μL of activation buffer (0.1 M Tris, 0.15 M NaCl, 0.01 M CaCl₂, 0.05% Triton-X, pH=8.0) and incubated at 37° C. for 24 hours (hepsin concentration in activation buffer 2 μM). Once activated hepsin was diluted in assay buffer (0.05 M Tris, pH=9.0) to the final concentration of 4 nM and incubated at 37° C. for 15 min before added to a substrate. HGFA. HFGA enzyme was diluted in the assay buffer (0.15 M NaCl, 0.025 M Tris, 0.005 M CaCl₂, pH=8.0) to the final concentration of 10-20 nM (depending on sublibrary) and incubated at 25° C. for 30 min before added to a substrate.

### Example 5: SARS-CoV-2, H1N1, and MERS Assays

TABLE 16A
SARS-CoV-2 and MERS Infection and Host Cell Entry
Assays using human lung epithelial Calu-3 cells

| | SARS- | SARS- | SARS- | | |
|---|---|---|---|---|---|
| | CoV-2 | CoV-2 | CoV-2 | VSV SARS | VSV |
| | Calu-3 | Calu-3 | Calu-3- | CoV-2 Cell | MERS |
| | titer | titer | titer | Entry EC₅₀ | Cell Entry |
| Name | (1 uM) | (0.1 uM) | (0.01 uM) | (nM) | EC₅₀ (nM) |
| MF1064 (YK) | 434443 | 217302 | 14 | N.D. | N.D. |
| PK-1-102A1 | 434443 | 218365 | 7 | 1,377 | N.D. |
| (MN1066) | | | | | |
| MF1140 | 421488 | 217258 | 25 | N.D. | N.D. |
| MF1068 | 300324 | 10577 | 11 | N.D. | N.D. |
| (MPM) | | | | | |
| PK-1-93 | 218016 | 4174 | 69 | 141.3 | N.D. |
| (VB) | | | | | |
| MF1065 | 217670 | 108 | 14 | N.D. | N.D. |
| (MLS) | | | | | |
| MF1125 | 160676 | 128 | 43 | N.D. | N.D. |
| MM3122 | 150498 | 11357 | 72 | 0.43 | 0.87 |
| PK-1-103 | 32985 | 3092 | 39 | 115.9 | N.D. |
| (MM4095) | | | | | |
| VD4010 | 5924 | 105 | 23 | N.D. | N.D. |
| ZFH7064 | 531 | 56 | 44 | N.D. | N.D. |
| VD2173 | 57 | 17 | 11 | 197 | 273 |
| VD4090 | 50 | 14 | 7 | N.D. | N.D. |
| VD4072 | 22 | 7 | 5 | N.D. | N.D. |
| MF1142 | 18 | 12 | 13 | N.D. | N.D. |
| MM4009-2 | N.D. | N.D. | N.D. | 40.3 | N.D. |
| PK-1-89A1 | N.D. | N.D. | N.D. | 139.7 | N.D. |
| (MM3158) | | | | | |
| MF1101 | N.D. | 190 | 1 | N.D. | N.D. |
| VD5076B | N.D. | N.D. | N.D. | N.D. | N.D. |
| MM3131 | N.D. | 11 | 1 | 10.7 | N.D. |
| MM3180-2 | N.D. | 266 | 7 | 12 | N.D. |
| MM4038 | N.D. | 3 | 3 | 225.8 | N.D. |
| MM3123 | N.D. | 19 | 2 | 0.86 | 2.9 |
| (MF1016/ | | | | | |
| MF1003) | | | | | |
| MM4028-2 | N.D. | 12 | 1 | 103.4 | N.D. |
| MF1105 | N.D. | 340 | 1 | N.D. | N.D. |
| CA1043 | N.D. | 357 | 0 | N.D. | N.D. |
| (MF1017) | | | | | |
| MM4037-2 | N.D. | 29 | 3 | 23.95 | N.D. |
| MF1099 | N.D. | 99 | 6 | N.D. | N.D. |
| (N-0385) | | | | | |
| MM3130 | N.D. | 12 | 4 | 5.6/47.8 | N.D. |
| MM3186 | N.D. | 3 | 2 | 18.5 | N.D. |
| MM3178 | N.D. | 14 | 1 | 10.8 | N.D. |
| MF1104 | N.D. | 81 | 2 | N.D. | N.D. |
| MM3194A | N.D. | N.D. | N.D. | 612.6 | N.D. |
| MM3144 | N.D. | 7580 | 7 | 0.53 | 0.55 |
| (N-0386) | | | | | |
| CA1033-2 | N.D. | 6 | 3 | N.D. | N.D. |
| CA1018 | N.D. | 356 | 2 | N.D. | N.D. |
| ZFH9141 | N.D. | 4890 | 16 | N.D. | N.D. |
| CA1041-2 | N.D. | 0 | 1 | N.D. | N.D. |
| CA1022-2 | N.D. | 9 | 1 | N.D. | N.D. |
| PK-1-105 | N.D. | N.D. | N.D. | 721.1 | N.D. |
| (MM4094) | | | | | |
| ZFH6138 | N.D. | N.D. | N.D. | 1,320 | N.D. |
| MM3116 | N.D. | 18 | 0 | 3.6 | 2.3 |
| PK-1-45A1 | N.D. | N.D. | N.D. | 105 | N.D. |
| MM3187 | N.D. | 101 | 2 | 22 | N.D. |
| ZFH7116 | N.D. | N.D. | N.D. | 489 | N.D. |
| MM3180-1 | N.D. | N.D. | N.D. | 5.3 | N.D. |
| MM4027-2 | N.D. | 1 | 0 | N.D. | N.D. |
| ZFH7063 | N.D. | N.D. | N.D. | 101 | N.D. |
| (ZFH) | | | | | |
| MM3177 | N.D. | 3 | 2 | 36 | N.D. |
| VD4162B | N.D. | N.D. | N.D. | 2,058 | N.D. |
| MM4027-1 | N.D. | N.D. | N.D. | 92.3 | N.D. |
| ZFH6095 | N.D. | N.D. | N.D. | 1,157 | N.D. |
| VD5064B | N.D. | N.D. | N.D. | 8,374 | N.D. |
| ZFH7006 | N.D. | N.D. | N.D. | 572/652 | N.D. |
| PK-1-18A1 | N.D. | N.D. | N.D. | 356 | N.D. |
| ZFH6101 | N.D. | N.D. | N.D. | 262 | N.D. |
| ZFH7053 | N.D. | N.D. | N.D. | 4,272 | N.D. |
| ZFH6201-1 | N.D. | N.D. | N.D. | 1,838 | N.D. |

TABLE 16B
H1N1 Infection Assays

| | H1N1 IAV | H1N1 IAV | H1N1 IAV | H1N1 IAV |
|---|---|---|---|---|
| | Calu-3 - | Calu-3 - | Calu-3 - | Calu-3 - |
| | Infection | Infection | Infection | Infection |
| | assay | assay | assay | assay |
| Name | (10 uM) | (1 uM) | (0.1 uM) | (0.01 uM) |
| MF1064 (YK) | 8199 | 8199 | 11 | 1.007 |
| PK-1-102A1 | 209 | 59 | 3 | 1.112 |
| (MN1066) | | | | |
| MF1140 | 8199 | 2274 | 13 | 0.248 |
| MF1068 (MPM) | 526 | 104 | 2 | 4.241 |
| PK-1-93 (VB) | N.D. | N.D. | N.D. | N.D. |
| MF1065 (MLS) | N.D. | N.D. | N.D. | N.D. |
| MF1125 | N.D. | N.D. | N.D. | N.D. |
| MM3122 | 29014 | 41013 | 182 | 5.183 |
| PK-1-103 | 269 | 33 | 4 | 2.258 |
| (MM4095) | | | | |
| VD4010 | N.D. | N.D. | N.D. | N.D. |
| ZFH7064 | N.D. | N.D. | N.D. | N.D. |
| VD2173 | N.D. | N.D. | N.D. | N.D. |
| VD4090 | N.D. | N.D. | N.D. | N.D. |
| VD4072 | N.D. | N.D. | N.D. | N.D. |
| MF1142 | N.D. | N.D. | N.D. | N.D. |
| MM4009-2 | 2355.623 | 4.197 | 1.116 | 1.680 |
| PK-1-89A1 | 199.986 | 35.431 | 3.651 | 1.412 |
| (MM3158) | | | | |
| MF1101 | 140.622 | 155.023 | 8.396 | 0.714 |
| VD5076B | 2.095 | 1.346 | 1.766 | 1.016 |
| MM3131 | N.D. | N.D. | N.D. | N.D. |
| MM3180-2 | N.D. | N.D. | N.D. | N.D. |
| MM4038 | N.D. | N.D. | N.D. | N.D. |
| MM3123 | N.D. | N.D. | N.D. | N.D. |
| (MF1016/ | | | | |
| MF1003) | | | | |
| MM4028-2 | N.D. | N.D. | N.D. | N.D. |
| MF1105 | N.D. | N.D. | N.D. | N.D. |
| CA1043 | N.D. | N.D. | N.D. | N.D. |
| (MF1017) | | | | |
| MM4037-2 | N.D. | N.D. | N.D. | N.D. |
| MF1099 | N.D. | N.D. | N.D. | N.D. |
| (N-0385) | | | | |
| MM3130 | N.D. | N.D. | N.D. | N.D. |
| MM3186 | N.D. | N.D. | N.D. | N.D. |
| MM3178 | N.D. | N.D. | N.D. | N.D. |
| MF1104 | N.D. | N.D. | N.D. | N.D. |
| MM3194A | N.D. | N.D. | N.D. | N.D. |
| MM3144 | N.D. | N.D. | N.D. | N.D. |
| (N-0386) | | | | |
| CA1033-2 | N.D. | N.D. | N.D. | N.D. |
| CA1018 | N.D. | N.D. | N.D. | N.D. |
| ZFH9141 | N.D. | N.D. | N.D. | N.D. |
| CA1041-2 | N.D. | N.D. | N.D. | N.D. |
| CA1022-2 | N.D. | N.D. | N.D. | N.D. |
| PK-1-105 | N.D. | N.D. | N.D. | N.D. |
| (MM4094) | | | | |
| ZFH6138 | N.D. | N.D. | N.D. | N.D. |
| MM3116 | N.D. | N.D. | N.D. | N.D. |
| PK-1-45A1 | N.D. | N.D. | N.D. | N.D. |
| MM3187 | N.D. | N.D. | N.D. | N.D. |
| ZFH7116 | N.D. | N.D. | N.D. | N.D. |
| MM3180-1 | N.D. | N.D. | N.D. | N.D. |
| MM4027-2 | N.D. | N.D. | N.D. | N.D. |
| ZFH7063 (ZFH) | N.D. | N.D. | N.D. | N.D. |
| MM3177 | N.D. | N.D. | N.D. | N.D. |
| VD4162B | N.D. | N.D. | N.D. | N.D. |
| MM4027-1 | N.D. | N.D. | N.D. | N.D. |
| ZFH6095 | N.D. | N.D. | N.D. | N.D. |
| VD5064B | N.D. | N.D. | N.D. | N.D. |
| ZFH7006 | N.D. | N.D. | N.D. | N.D. |
| PK-1-18A1 | N.D. | N.D. | N.D. | N.D. |
| ZFH6101 | N.D. | N.D. | N.D. | N.D. |
| ZFH7053 | N.D. | N.D. | N.D. | N.D. |
| ZFH6201-1 | N.D. | N.D. | N.D. | N.D. |

### SARS-CoV-2 Assays

### Host Cell Entry Assay (VSV SARS-CoV-2 and MERS Chimeras):

VSV-eGFP, a recombinant VSV expressing a GFP reporter (depends on the VSV glycoprotein G for entry), has been previously described. VSV-SARS-CoV-2, a replication competent infectious VSV chimera which employs the SARS-CoV-2 Spike (S) protein for viral entry in place of VSV G and expresses eGFP, has also been previously described. VSV-MERS was created in the same manner as VSV-SARS-CoV-2, except the MERS Spike missing the terminal 21 amino acids (HCoV-EMC/2012 strain) was inserted in place of VSV G.

Human Calu-3 lung epithelial cells or Vero cells (African green monkey kidney) were seeded in a 96-well black plate in DMEM containing 10% FBS for 24 hours (37° C. and 5% CO₂). The next day, cells were pretreated for 2 hours with inhibitor or vehicle control (DMSO) in 50 μl serum-free DMEM, and subsequently infected with VSV-SARS-CoV2, VSV-MERS, or VSV-eGFP at a multiplicity of infection (MOI) of 0.5. At 7 hours post infection (single round of infection), cells were fixed in 2% formaldehyde and nuclei stained with 10 μg/ml Hoechst 33342 (Invitrogen) for 30 minutes at room temperature. Cells were washed once and stored in PBS after fixation, and automated microscopy performed using an InCell 2000 Analyzer (GE Healthcare) in the DAPI and FITC channels (10× objective, 9 fields per well, covering the entire well). Images were analyzed using the Multi Target Analysis Module of the InCell Analyzer 1000 Workstation Software (GE Healthcare) to identify the percentage of GFP-positive cells in each well (top-hat segmentation). The percentage GFP-positive cells for experimental conditions was normalized against the control (DMSO-treated) cells and expressed as relative infectivity. GraphPad Prism (version 8.4.2) was used to calculate the EC₅₀ of each drug. Statistics (comparison of VSV-SARS-2 to VSV-eGFP with each drug; Student's t-test) were performed in Microsoft Excel. Three biological replicates were performed.

SARS-CoV-2 in vitro inhibition assay. Calu-3 cells (2.5×10⁵ cells/well) were seeded in 24-well culture plates in infection medium (DMEM+1.0 mM Sodium pyruvate, NEAA, 100 U/mL of penicillin, 100 μg/mL streptomycin, 2.0 mM L-glutamine, 10 mM HEPES, and 2% FBS) and incubated overnight at 37° C. and 5% CO₂. After 24 h, media was removed and fresh 250 μL media was added to each well containing inhibitor, starting at 20 μM concentration and diluted 10-fold to 0.002 μM. Next, the cells were transferred to the BSL3 laboratory and 250 μL of media containing 4,000 PFU of SARS-CoV-2 was added for 1 h at 37° C. and 5% CO₂. After 1 h, the virus inoculum was removed, the cells were washed twice with infection media and fresh infection media containing inhibitor (10 μM to 0.001 μM) was added to each well. At 48 h post-infection, culture supernatant is collected and used to quantify virus titers by plaque assay as described below.

SARS-CoV-2 Virus titration assay. Plaque assays were performed on Vero-hACE2-hTRMPSS2 cells in 24-well plates. Tissue culture supernatant was diluted serially by 10-fold, starting at 1:10, in cell infection medium (DMEM+100 U/mL of penicillin, 100 μg/mL streptomycin, and 2% FBS). Two hundred and fifty microliters of the diluted virus were added to a single well per dilution per sample. After 1 h at 37° C., the inoculum was aspirated, the cells were washed with PBS, and a 1% methylcellulose overlay in MEM supplemented with 2% FBS was added. Seventy-two to ninety-six hours after virus inoculation, dependent on the virus strain, the cells were fixed with 4% formalin and the monolayer was stained with crystal violet (0.5% w/v in 25% methanol in water) for 30 min at 20° C. The number of plaques were counted and used to calculate the plaque forming units/mL (PFU/mL).

### Influenza Virus Assays

H1N1 in vitro inhibition assay. Calu-3 cells (2.5×10⁵ cells/well) were seeded in 24-well culture plates in infection medium (DMEM+1.0 mM Sodium pyruvate, NEAA, 100 U/mL of penicillin, 100 μg/mL streptomycin, 2.0 mM L-glutamine, 10 mM HEPES, and 2% FBS) and incubated overnight at 37° C. and 5% CO₂. After 24 h, media was removed, and the cells were washed one with PBS. Next, the cells were infected with of media containing 250 PFU of A/California/04/2009 was added for 1 h at 37° C. and 5% CO₂. After 1 h, the virus inoculum was removed, the cells were washed twice with PBS and 1.0 mL of fresh infection media containing inhibitor (10 μM to 0.001 μM) was added to each well. At 48 h post-infection, culture supernatant is collected and used to quantify virus titers by infectious virus assay as described below.

H1N1 Virus titration assay. Focus forming assays were performed on MDCK cells in 96-well plates. Tissue culture supernatant was diluted serially by 10-fold, starting at 1:10, in cell infection medium (MEM+100 U/mL of penicillin, 100 μg/mL streptomycin, and 0.1% BSA). One hundred microliters of the diluted supernatant were added to two wells per dilution. After 1 h at 37° C., the inoculum was aspirated, the cells were washed with PBS, and a 1% methylcellulose overlay in MEM supplemented with 0.1% BSA and 1 μg/ml of TPCK trypsin was added to each well. Twenty-four to forty-eight hours later, the cells were fixed with 4% formalin, the cells were washed and sequentially incubated with an anti-influenza HA monoclonal antibody (CR9114) and HRP-conjugated anti-human IgG (Sigma Cat #A6092) in PBS supplemented with 0.1% saponin and 0.1% bovine serum albumin. Influenza virus-infected cell foci were visualized using TrueBlue peroxidase substrate (KPL) and quantitated on an ImmunoSpot microanalyzer (Cellular Technologies).

### Example 6: Synthesis of Tetrapeptides

### Design and Synthesis

Four initial, potent tetrapeptide kbt inhibitors of TMPRSS2 were previously identified based on PS-SCL data, including Ac-IQFR-kbt (MM3116)-1, Ac-GQFR-kbt (MM3122)-2, and Ac-PQFR-kbt (MM3123)-3. PS-SCL studies revealed the sidechain preference of TMPRSS2 tetrapeptide substrates to be I in the P4, Q in the P3, and F in the P2 position of the peptide. A P1 Arg is preferred like all other trypsin-like serine proteases. While this information is integral to substrate and inhibitor design, it should be noted that PS-SCL only records individual preferences for one protease subpocket (S) binding relative to the control peptide and does not account for any cooperativity between the different protease subpockets and peptide sidechains. Thus, it is imperative to perform SAR studies evaluating several of the top amino acid sidechains and not just the best one identified from PS-SCL. In the case of TMPRSS2, it was found that it preferred I, G, P, M, and L in the P4 position, Q, E, M, S, and T in the P3 position and F, W, A, V, T, and S in the P2 position. Therefore, 3 focused libraries of peptide ketobenzothiazoles keeping two of the amino acids constant based on the top preferred amino acid for those positions while changing the third position were synthesized. The physiologically relevant peptide Ac-PSKR-kbt (MM3194-17) was also synthesized based on the SARS-CoV-2 sequence which gets cleaved by TMPRSS2.

### Tetrapeptides

P4 X-Q-F-R-kbt, X=L, M (I, G and P in PNAS)
P4,P3 Ac-WLFR-kbt
P3 I-X-F-R-kbt, X=dW, E, T, M, S (Q in PNAS)
P2 I-Q-X-R-kbt, X=W, T, V, A, S (F in PNAS)
SARS CoV-2 Spike Sequence PSKR-kbt

In previous studies, a shorter tripeptide, Ac-QFR-kbt (MM3144)-18 and another group reported on MeSO₂-QFR-kbt (N-0385)-26 were identified, which both had equipotent activity relative to any of the tetrapeptides, but both are not selective over Factor Xa, the coagulation protease which is essential to get selectivity over as well as thrombin. One reason for the required selectivity is to prevent any blood thinning in infected patients in which the marketed Factor Xa inhibitor apixaban is used for. The second reason is that Factor Xa has been shown to proteolytically cleave the Spike protein of SARS-CoV-2 into and form that is incapable of binding the ACE2 receptor. Here, different N-terminal capping groups of MM3144-17 in place of the acetyl were explored to find improved inhibitors with better potency and selectivity over Factor Xa. These included substitution on the amine with alkyl, acyl, and sulfonamide groups.

### Capping of Q-F-R-kbt

Amines, PhEt (CA1033), Me2 (CA1041), Pip (CA1046)
Acetamides, Bz (CA1043), PhAc (CA1018), Bn (CA1022) and carbamate MFXXXX
Sulfonamides, Me (MF1099), cyclopropyl (MF1104), Ph (MF1101), Bn (MF1105)

### Detailed Materials and Methods

### General Synthesis, Purification, and Analytical Chemistry Procedures.

Starting materials, reagents, and solvents were purchased from commercial vendors unless otherwise noted. 1H NMR spectra were measured on a Varian 400 MHz or Bruker Avance III 600 MHz NMR instrument. The chemical shifts were reported as 6 ppm relative to TMS using residual solvent peak as the reference unless otherwise noted. The following abbreviations were used to express the multiplicities: s=singlet; d=doublet; t=triplet; q=quartet; m=multiplet. High-performed liquid chromatography (HPLC) was carried out on GILSON GX-281 using Waters C18 5 μM, 4.6×50 mm and Waters Prep C18 5 μM, 19×150 mm reverse phase columns, eluted with a gradient system of 5:95 to 95:5 acetonitrile:water with a buffer consisting of 0.05% TFA. Mass spectra (MS) were performed on HPLC/MSD using electrospray ionization (ESI) for detection. All reactions were monitored by thin layer chromatography (TLC) carried out on Merck silica gel plates (0.25 mm thick, 60F254), visualized by using UV (254 nm) or dyes such as KMnO₄, p-Anisaldehyde, and CAMA (Cerium Ammonium Molybdate or Hanessian's Stain). Silica gel chromatography was carried out on a Teledyne ISCO CombiFlash purification system using pre-packed silica gel columns (12 g to 330 g sizes). All compounds used for biological assays are greater than 95% purity based on NMR and HPLC by absorbance at 220 nm and 254 nm wavelengths.

Compounds MM3116 (1), MM3122 (2), MM3123 (3), ZFH7064 (6), MM3144 (18) and MF1099 (26) were prepared as described earlier (Mahoney, M., et al., Proceedings of the National Academy of Sciences 2021, 118 (43), e2108728118; Shapira, T., et al. Nature 2022, 605 (7909), 340-348). General procedure for synthesis of peptides (Han, Z., et al., Chemmedchem 2016, 11 (6), 585-599.)

Solid phase peptide coupling and deprotection: Into a reaction vial (with a fritted glass filter) under nitrogen containing 2-chlorotrityl resin (0.5 mmol) modified with corresponding amino acid (Phe, Trp, Thr, Val, Ala, Ser or Lys) was added DMF/CH₂Cl₂ (15/15 ml). The mixture was shaken at RT for 30 min and then filtered. The resin was washed with DMF (10 ml) 2 times. A mixture of Fmoc-AA-OH (2.5 mmol) in DMF (20 ml), HBTU (0.853 g, 2.25 mmol), and iPr₂NEt (0.87 ml, 5 mmol) was stirred at RT for 10 min and then added to the resin. The resultant heterogeneous mixture was shaken at RT overnight and then filtered. The resin was washed with DMF (4×20 ml), dried, and then piperidine/DMF (20% v/v, 30 ml) was added. The mixture was shaken for 1-4 h at RT, then filtered and washed with DMF (4×10 ml). Following the Fmoc deprotection, the peptide is carried on to the next step, or coupling of another Fmoc-AA-OH was performed identically as described above, and then subsequently a final Fmoc deprotection to the tripeptide.

Acetyl capping and cleavage from resin: The peptide-containing resin was suspended in 30 ml of 0.5M Ac₂O and 1M iPr₂NEt in DMF and shaken at RT for 1 h. The reaction was filtered, and the resin was washed with DMF (4×10 ml) followed by CH₂Cl₂ (4×10 ml). The resin was then suspended in 30 ml of 25% v/v HFIP/CH₂Cl₂ and shaken for 1 h. The reaction was filtered, and the filtrate was concentrated and dried in a vacuum.

Coupling of Arg-(Pbf)-kbt:HCl and final deprotection: To crude peptide acid (400 mg, 1.0 mmol) dissolved in dry DMF (10 ml) under a nitrogen atmosphere at 0° C. was added HATU (456 mg, 1.20 mmol) followed by stirring for 15 min. Next, Arg-(Pbf)-kbt:HCl (638 mg, 1.1 mmol) and iPr₂NEt (0.87 ml, 5.0 mmol) were added to the reaction at 0° C. The reaction was allowed to reach room temperature and then stirred for an additional 2-3 h. DMF was removed under vacuum and water (250 ml) was added to the residue. The precipitate was filtered and washed with water (2×50 ml) and then dried under vacuum. The precipitate was suspended in 10 ml of TFA/thioanisole/water (92/4/4 v/v/v) and stirred for 2 h at RT. The solvent was removed, and cold diethyl ether (100 ml) was added. The resulting precipitate was collected by centrifugation and the crude product was purified by HPLC (C18 5 μM, 19×150 mm column; eluent: acetonitrile/water (0.05% TFA)) to give the final compound.

General procedure for the synthesis of H₂N-Q(Trt)-F—COOMe dipeptide: Into a round bottom flask containing Boc-Gln(Trt)-OH (5 g, 10 mmol) and HATU (4.57 g, 12 mmol) in DMF (20 ml) was added Phe-OMexHCl (2.37 g, 11 mmol) and iPr₂NEt (9 ml, 50 mmol) at 0° C. and stirred overnight under argon atmosphere. The reaction mixture was poured into 300 ml of water and the precipitated peptide was collected by filtration and dried in a vacuum. The Boc protecting group was removed by adding 10 ml of 4M HCl solution in dioxane and 5 ml of dioxane to peptide stirring for 2 h under argon. The reaction mixture was concentrated and dried in a vacuum to get crude amine H₂N-Q(Trt)-F—COOMe.

General procedure for the synthesis of RSO₂—H₂N-Q-R-kbt tripeptide: Into a round bottom flask containing Boc-Gln(Trt)-OH (5 g, 10 mmol) and HATU (4.57 g, 12 mmol) in DMF (20 ml) was added Phe-OMexHCl (2.37 g, 11 mmol) and iPr₂NEt (9 ml, 50 mmol) at 0° C. and stirred overnight under argon atmosphere. The reaction mixture was poured into 300 ml of water and the precipitated peptide was collected by filtration and dried in a vacuum. To the residue (5.3 g, 8.2 mmol) 50 ml of MeOH was added, followed by 50 ml of water, and 20 ml of THF. LiOH (364 mg, 15.1 mmol) was added to the reaction mixture and stirred for 36 h. Solvents were removed by evaporation and 40 ml of water was added to the residue. Then, 1M HCl solution was added dropwise to pH 3 at 0° C. The reaction mixture was incubated for 30 min at 0° C., the formed precipitate was collected by filtration, washed with water, and dried under a vacuum. The crude peptide acid residue (581 mg, 0.9 mmol) was dissolved in DMF (5 ml). To the solution under argon atmosphere at 0° C. was added HATU (410 mg, 1.08 mmol) and stirred for 5 min. Next, Arg(Pbf)-kbtxHCl (530 mg, 0.94 mmol) and iPr₂NEt (323 μl, 1.8 mmol) were added to the reaction and stirred overnight. The reaction mixture was poured into 100 ml of ice-cold water. The precipitate was collected by filtration, washed with water, and dried in a vacuum. The crude peptide was purified on a Silica column using Ethyl acetate/hexane gradient as an eluent. To the tripeptide (600 mg, 0.52 mmol) 10 ml of 4M HCl solution in dioxane was added and stirred for 2 h under Argon. Solvents were removed under reduced pressure. Free amine (100 mg, 0.092 mmol) was dissolved in DCM at 0° C. Then Et₃N (52 μl, 0.36 mmol) was added to the reaction mixture, followed by corresponding sulfochloride (0.18 mmol, 2 eq). The reaction mixture was stirred 1 h at rt under Argon. Then, the reaction mixture was diluted with DCM, the organic layer was washed with NaHCO₃ sat., dried over sodium sulfate and the solvent was removed under vacuum. To the residue, 10 ml of TFA/thioanisole/water mixture (92/4/4 v/v/v) was added and stirred for 30 min. The solvent was evaporated, and the residue was dried in a vacuum. The crude product was purified by HPLC to give the final compound.

### Synthesis of Tetrapeptidyl Ketobenzothiazoles, 1-18

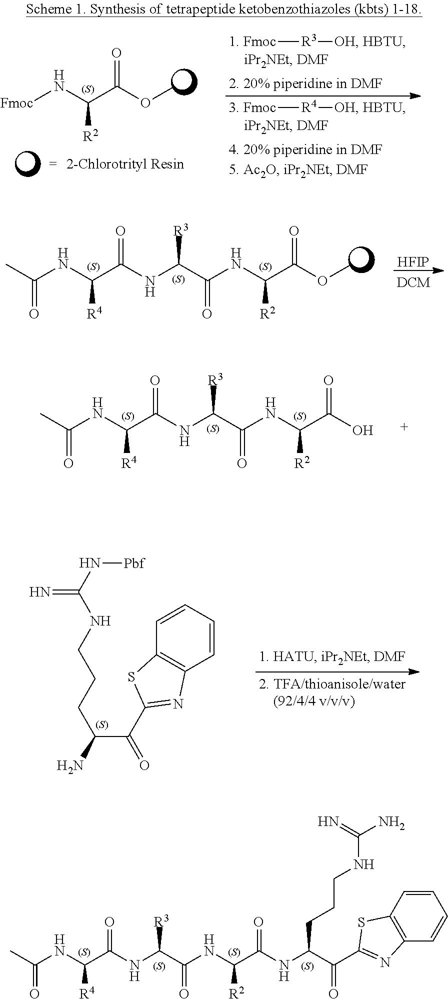

The methodology for the synthesis of the kbt peptide inhibitors has been previously published by the inventors (Han, Z., et al., Chemmedchem 2016, 11 (6), 585-599.). As shown in Scheme 1, the tripeptide was first constructed on the 2-chlorotrityl resin using standard Fmoc-solid phase peptide synthesis protocols including HBTU for amide bond coupling and 20% piperidine for Fmoc deprotection steps. The peptide was then capped with an acetyl group using acetic anhydride. Cleavage of the 2-chlorotrityl resin without removal of the protecting groups is accomplished using HFIP, then H-Arg(Pbf)-kbt is installed with HATU or EDC/HOBt in DMF. Final deprotection of the amino acid sidechains using TFA:water:thioanisole (95:2.5:2.5% v/v) generates the target compounds which are purified by reverse-phase prep HPLC.

### Synthesis of Capped Tripeptidyl Ketobenzothiazoles, 19-25

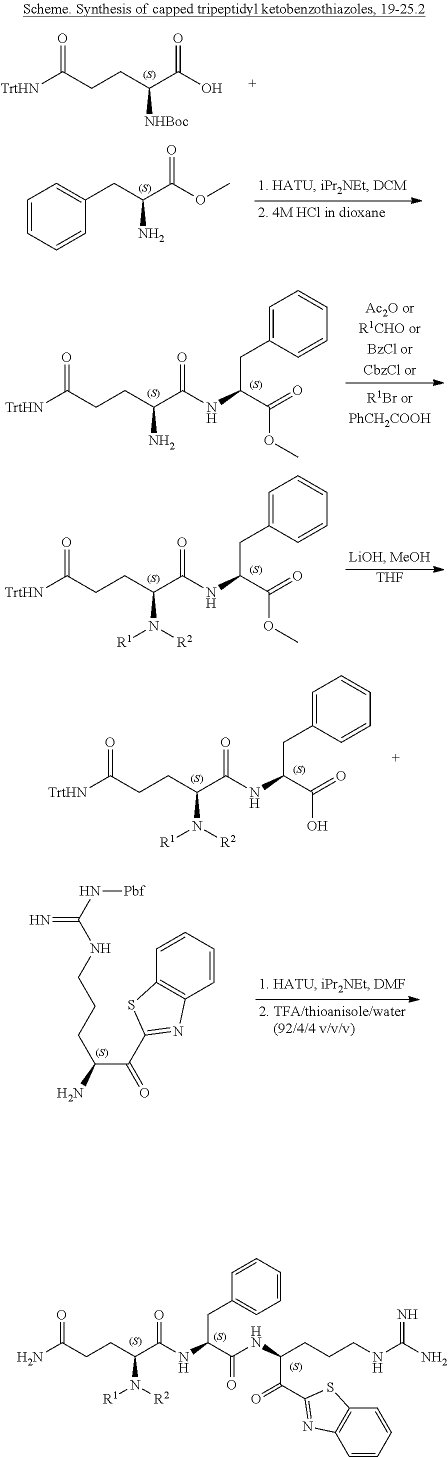

As shown in Scheme 2, the H₂N-Q(Trt)-F—COOMe peptide was first constructed using standard coupling protocols including HATU for amide bond coupling and 4M HCl in dioxane solution for Boc deprotection. The peptide was then capped with the corresponding acyl or alkyl group. Next, deprotection of methyl ester was performed by lithium hydroxide followed by H-Arg(Pbf)-kbt introduced with HBTU in DMF. Final deprotection of the amino acid sidechains using TFA:water:thioanisole (95:2.5:2.5% v/v) generates the target compounds which were purified by reverse-phase prep HPLC.

See, for example, the below exemplary synthesis. Other similar compounds can be made similarly.

### CA1041 (19) or Dimethyl-QFR-kbt

### N—[(S)-1-{N—(S)-1-[(1,3-benzothiazol-2-yl)carbonyl]-4-guanidinobutylcarbamoyl}-2-phenylethyl](S)-2-(dimethylamino)glutaramide

Into a round bottom flask containing H₂N-Q(Trt)-F—COOMe (300 mg, 0.54 mmol) and formaldehyde (0.5 ml) dissolved in acetonitrile (4 ml) was added sodium cyanoborohydride (303 mg, 4.8 mmol) and stirred for 2 h. The solvent was removed and dried in a vacuum. EtOAc and water were added to the residue, the organic layer was washed with brine, dried with MgSO₄, filtered, and concentrated. To the residue, 2M LiOH solution in water (0.312 ml, 0.63 mmol), 1.5 ml of MeOH, and 1.5 ml of THF were added. The flask was stirred for 3 h under argon and the solvents were removed under reduced pressure. The crude peptide acid residue (270 mg, 0.48 mmol) was dissolved in DMF (8 ml). To the solution under argon atmosphere at 0° C. was added HBTU (200 mg, 0.52 mmol) and stirred for 5 min. Next, Arg(Pbf)-kbtxHCl (260 mg, 0.48 mmol) and iPr₂NEt (0.17 ml, 1.0 mmol) were added to the reaction and stirred overnight. The solvent was removed in a vacuum. To the residue, 10 ml of TFA/thioanisole/water mixture (92/4/4 v/v/v) was added and stirred for 3 h. The solvent was evaporated, and the residue was dried in a vacuum. The crude product was purified by HPLC to give the final compound.

### Synthesis of Sulfonyl-Capped QFR-Kbt Peptides, 27-29

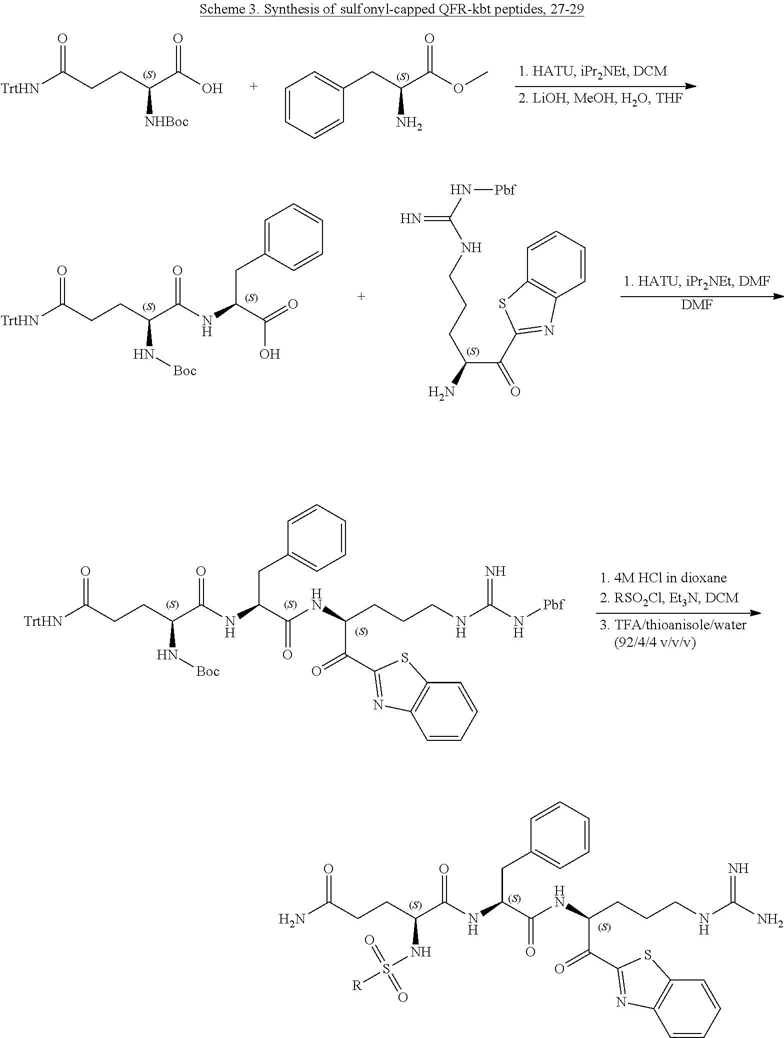

As shown in Scheme 3, the Boc-H₂N-Q(Trt)-F-Arg(pbf)-kbt peptide was first constructed using standard coupling protocols including HATU for amide bond coupling, LiOH in water/methanol/THF mixture to ester hydrolysis, and 4M HCl in dioxane solution for Boc deprotection. The peptide was then capped with the corresponding sulfonyl chloride, followed by final deprotection of the amino acid sidechains using TFA:water:thioanisole (95:2.5:2.5% v/v) to give the target compounds which are purified by reverse-phase prep HPLC.

### Example 7: Unnatural Amino Acid Syntheses

### General Synthesis, Purification, and Analytical Chemistry Procedures.

Starting materials, reagents, and solvents were purchased from commercial vendors unless otherwise noted. ¹H NMR spectra were measured on a Varian 400 MHz NMR instrument equipped with an autosampler. The chemical shifts were reported as 6 ppm relative to TMS using residual solvent peak as the reference unless otherwise noted. The following abbreviations were used to express the multiplicities: s=singlet; d=doublet; dd=doublet of doublets; t=triplet; q=quartet; m=multiplet; br=broad. High-performance liquid chromatography (HPLC) purifications were carried out on GILSON GX-281 using Waters C18 5 μM, 4.6*50 mm and Waters Prep C18 5 μM, 19*150 mm reverse phase columns, eluted with a gradient system of 5:95 to 95:5 acetonitrile:water with a buffer consisting of 0.05-0.1% TFA. Mass spectra (MS) were obtained on and Agilent or Waters HPLC/MSD using electrospray ionization (ESI) for detection. All reactions were monitored by thin layer chromatography (TLC) carried out on Merck silica gel plates (0.25 mm thick, 60F254), visualized by using UV (254 nm) or dyes such as KMnO₄, p-Anisaldehyde and CAMA (Cerium Ammonium Molybdate or Hanessian's Stain). Silica gel chromatography was carried out on a Teledyne ISCO CombiFlash MPLC purification system using pre-packed silica gel columns (12 g to 330 g sizes). All compounds used for biological assays are greater than 95% purity based on ¹H NMR and HPLC by absorbance at 220 nm and 254 nm wavelengths.

General procedure for Fmoc solid phase peptide synthesis (SPPS) of dipeptide and tripeptide intermediates. Leu-2-chlorotrityl resin (or hLeu-2-chlorotrityl resin) was swelled in methylene chloride prior to use. The resin was then treated with 2 eq of P2 Fmoc protected amino acid, 2 eq of HBTU and 4 eq DIEA in DMF for several hours. The reagents were drained by filtration, and the resin washed 4× with DMF. The resin was subsequently treated 2× with 25% piperidine/DMF and then washed 4× with DMF. Next, the resin was treated with 2 eq of P3 Fmoc protected amino acid, 2 eq of HBTU and 4 eq DIEA in DMF for several hours. The reaction mixture was filtered, and the resin washed 4× with DMF. The resin was then treated 2× with 25% piperidine/DMF and finally washed 4× with DMF. [For tetrapeptides] The resin was treated with 2 eq of P4 Fmoc protected amino acid, 2 eq. of HBTU and 4 eq. DIEA in DMF for several hours. The reagents were drained by filtration, and the resin washed 4× with DMF. The resin was then treated 2× with 25% piperidine/DMF and washed 4× with DMF. Acetylation: The free amino dipeptide or tripeptide was treated with acetic anhydride (4 eq.) and DIPEA (6 eq.) in DMF. The reaction was filtered, and the resin washed with DMF. Cleavage from Resin: The dry resin was washed 3× with methylene chloride prior to cleavage using 25% HFIP in methylene chloride for 1 h. The resin was filtered and washed with methylene chloride, then the combined filtrates were concentrated in vacuo to give the following N-acetyl dipeptide and tripeptide carboxylic acids:

See, for example, the below exemplary synthesis. Other similar compounds can be made similarly.

MN1063/PK-1-91 Ac-dTrp-Leu-Arg-kbt (4). To a solution of Ac-dTrp(Boc)Leu-OH (0.208 g, 0.453 mmol), HBTU (0.206 g, 0.544 mol) and H-Arg (Pbf)-kbt hydrochloride³³ (0.271 g, 0.498 mmol) in 10 mL of DMF cooled in an ice bath, was added DIPEA (0.162 mL, 0.929 mmol). The reaction was stirred for 4 h, The DMF was concentrated in vacuo and then water was added and the precipitate was filtered, washed with water and then dried under high vacuum to give intermediate Ac-dTrp(Boc)-Leu-Arg(Pbf)-kbt, 0.48 g (100%) as a white solid. LC/MS: ESI, calcd for C₅₀H₆₄N₈O₉S₂ 984.42 (M) found 985.70 (M+H⁺). To the intermediate was added a TFA:anisole:water (9.2 mL, 0.5 mL, 0.3 mL) mixture and was stirred for 1 h at room temperature. The solvent was concentrated in vacuo and the crude product was purified by reversed phase C18 prep HPLC (acetonitrile/water, 0.05% TFA) to give Ac-dTrp-Leu-Arg-kbt (0.11 g, 38%).

### Example 8: Tripeptide, Tetrapeptide, and Cyclic Peptide Syntheses

### Synthesis of Peptidyl Ketobenzothiazoles (Kbts), 4-7

The methodology for the synthesis of the kbt peptide inhibitors (Han, Z., Harris, P. K., Karmakar, P., Kim, T., Owusu, B. Y., Wildman, S. A., Klampfer, L., and Janetka, J. W. (2016) alpha-Ketobenzothiazole Serine Protease Inhibitors of Aberrant HGF/c-MET and MSP/RON Kinase Pathway Signaling in Cancer, ChemMedChem 11, 585-599.) has previously been published by the inventors. As shown in Scheme 4, the tripeptide (e.g. 6) or tetrapeptide (e.g. 4) is first constructed on the 2-chlorotrityl resin using standard Fmoc-solid phase peptide synthesis protocols including HBTU for amide bond coupling and 20% piperidine for Fmoc deprotection steps. The peptide is then capped with an acetyl group using acetic anhydride. Cleavage of the 2-chlorotrityl resin without removal of the protecting groups is accomplished using HFIP, then H-Arg(Mtr)-kbt is installed with HATU or EDC/HOBt in DMF. Final deprotection of the amino acid sidechains using TFA:water:thioanisole (95:2.5:2.5% v/v) generates the target compounds which are purified by reverse phase prep HPLC.

Cyclic peptide VD2173 (2) was synthesized as described in another publication (Voss, J., et al. (2021) Cancers (Basel).) but follows the generic procedure outlined in Scheme 5 in which is also used for the construction of 19 and 20. Synthesis of the tripeptide cyclization precursors are synthesized on Wang resin using standard Fmoc solid phase peptide synthesis (SPPS) using 2HBTU for coupling steps and 20% piperidine for Fmoc removal steps. After the final Fmoc deprotection, the tripeptide is acetylated and then the Asp and Lys protecting groups are removed with 4N HCl. Cyclization is performed using EDCI and HOBt on the resin followed by TFA cleavage, then a final HATU coupling with H-Arg-(Pbf)-kbt followed by Arg deprotection to give the cyclic peptides which are purified by reverse phase prep HPLC.

Compound 21 was prepared from Fmoc-allylglycine as shown in Scheme 7 below. N-Deprotection followed by esterification and acetylation gives Ac-allylglycine which is then coupled to H-Leu-OMe using EDC/HOBt. The resulting dipeptide ester is hydrolyzed with LiOH and then coupled to H-Tyr(Oallyl)-OMe once again with EDC/HOBt to yield the cyclization precursor. Olefin metathesis cyclization is accomplished with Grubbs 2^{nd} generation catalyst to give key aryl allyl ether cyclic peptide intermediate. Ester hydrolysis followed by amide coupling to H-Arg(Pbf)-kbt and final deprotection with TFA as before gives 21 which is purified by prep HPLC.

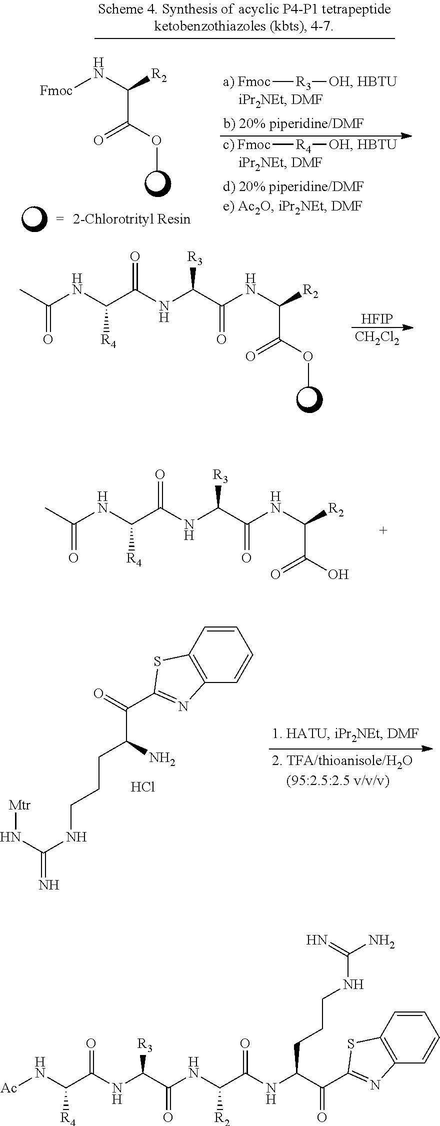

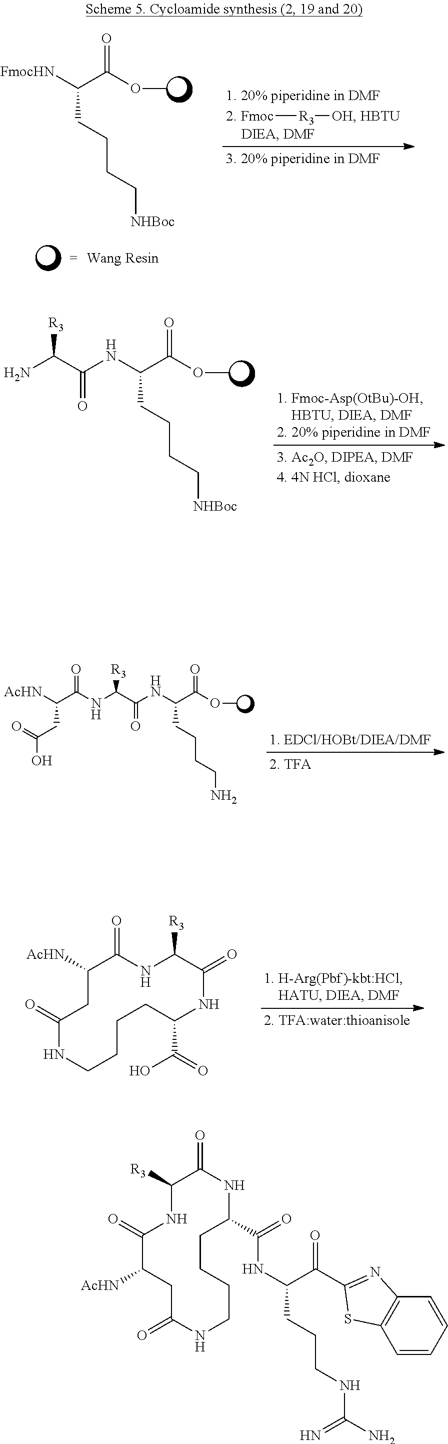

General synthesis, purification, and analytical chemistry procedures. Starting materials, reagents, and solvents were purchased from commercial vendors unless otherwise noted. 1H NMR spectra were measured on a Varian 400 MHZ NMR instrument. The chemical shifts were reported as 8 ppm relative to TMS using residual solvent peak as the reference unless otherwise noted. The following abbreviations were used to express the multiplicities: s=singlet; d=doublet; t=triplet; q=quartet; m=multiplet; br=broad. High-performed liquid chromatography (HPLC) was carried out on GILSON GX-281 using Waters C18 5 μM, 4.6*50 mm and Waters Prep C18 5 μM, 19*150 mm reverse phase columns, eluted with a gradient system of 5:95 to 95:5 acetonitrile:water with a buffer consisting of 0.05% TFA. Mass spectra (MS) were performed on HPLC/MSD using electrospray ionization (ESI) for detection. All reactions were monitored by thin layer chromatography (TLC) carried out on Merck silica gel plates (0.25 mm thick, 60F254), visualized by using UV (254 nm) or dyes such as KMnO₄, p-Anisaldehyde and CAMA (Cerium Ammonium Molybdate or Hanessian's Stain). Silica gel chromatography was carried out on a Teledyne ISCO CombiFlash purification system using pre-packed silica gel columns (12 g to 330 g sizes). All compounds used for biological assays are greater than 95% purity based on NMR and HPLC by absorbance at 220 nm and 254 nm wavelengths.

General procedure for synthesis of acyclic peptides (Han, Z., et al. (2016) ChemMedChem 11, 585-599.)

Solid phase peptide coupling and deprotection: Into a reaction vial (with a fritted glass filter) under nitrogen containing H-Leu-2-Cl trityl or H-Phe-2-Cl trityl resin (0.714 g, 0.5 mmol) was added DMF/CH₂Cl₂ (15/15 mL). The mixture was shaken at RT for 30 min and then filtered. The resin was washed with DMF (10 mL) 2 times. A mixture of Fmoc-AA-OH (2.5 mmol) in DMF (20 mL), HBTU (0.853 g, 2.25 mmol) and iPr₂NEt (0.87 mL, 5 mmol) was stirred at RT for 10 min and then added to the resin. The resultant heterogeneous mixture was shaken at RT overnight and then filtered. The resin was washed with DMF (20 mL×4), dried and then piperidine/DMF (20% v/v, 30 mL) was added. The mixture was shaken for 1-4 h at RT, then filtered and was washed with DMF (10 mL×4). Following the Fmoc deprotection of the dipeptide, the dipeptide is carried on to the next step or coupling of another Fmoc-AA-OH is performed in an identical fashion as described above and then subsequently a final Fmoc deprotection to the tripeptide.

Acetyl capping and cleavage from resin: The peptide-containing resin was suspended in 30 mL of 0.5 M Ac₂O and 1 M iPr₂NEt in DMF and shaken at RT for 1 h. The reaction was filtered, and resin washed with DMF (10 mL×4) followed by CH₂Cl₂ (10 mL×4). The resin was then suspended in 30 ml of 25% v/v HFIP/CH₂Cl₂ and shaken for 1 h. The reaction was filtered, and the filtrate was concentrated and dried in vacuo.

Coupling of Arg (Pbf)-kbt:HCl and final deprotection. To crude peptide acid (400 mg, 1.0 mmol) dissolved in dry DMF (10 mL) under a nitrogen atmosphere at 0° C. was added HATU (456 mg, 1.20 mmol) followed by stirring for 15 min. Next, Arg(Pbf)-kbt:HCl (638 mg; 1.10 mmol) and iPr₂NEt (0.87 mL, 5.0 mmol) were added to the reaction at 0° C. The reaction was allowed to reach room temperature and then stirred for an additional 2-3 h. DMF was removed under vacuum and water (250 mL) was added to the residue. The precipitate formed was filtered and washed with water (2×50 mL) then dried under vacuum. The precipitate was suspended in 10 mL TFA/thioanisole/water (95:2.5:2.5 v/v/v) and stirred for 2 h at RT. The solvent was removed, and cold ether (100 mL) was added. The resulting precipitate was collected by centrifugation and the crude product was purified by HPLC (Cis, 15×150 mm column; eluent: acetonitrile/water (0.05% TFA) to give the final compound.

See, for example, the below exemplary synthesis. Other similar compounds can be made similarly.

H-dWFR-kbt (14): Boc-dWFR(Mtr)-kbt (85 mg, crude product from previous step) was taken in 5 mL TFA:thioanisole:H₂O (95:2.5:2.5) and the reaction mixture was stirred for 6 hours at 25° C. The completion of the reaction was confirmed by LC-MS monitoring. On completion, the reaction mixture was concentrated under reduced pressure and triturated with diethyl ether to obtain the crude product as brown solid. The crude product was then subjected to reverse phase semi-preparative HPLC (Stationary phase: C18 column, mobile phase: H₂O-Acetonitrile with 0.1% TFA in each, 15-65% Acetonitrile in H₂O gradient for 20 minutes) to obtain the pure title product

Ac-Cyclo(DLK)—R-ketobenzothiazole (2, VD2173). Into a reaction vessel (with fritted glass for resin support) containing Fmoc-L-Lys(Boc) Wang resin (5 g, 1.7 mmol), DCM (40 mL) was added. The mixture was shaken at RT for 15 min and then filtered. To the dry resin was added piperidine/DMF (20% v/v, 40 mL) and the mixture was shaken for 30 min at RT, then filtered. The resin was washed with DMF (2×30 mL) and DCM (2×30 mL). Fmoc-Leu-OH (1.8 g, 5.1 mmol), HBTU (2.25 g, 5.95 mmol), iPr₂NEt (1.31 g, 10.2 mmol), and DMF (50 mL) were added to the vessel and shaken for 12 h, then filtered. The resin was washed with DCM (2×40 mL) and DMF (2×40 mL), then piperidine/DMF (20% v/v, 40 mL) was added and the reaction was shaken for 30 min at RT, then filtered. The resin washed with DCM (2×30 mL) and DMF (2×30 mL). Fmoc-Asp(OtBu)-OH (2.10 g, 5.1 mmol), HBTU (2.25 g, 5.95 mmol), iPr₂NEt (1.31 g, 10.2 mmol), and DMF (50 mL) were added to the vessel and shaken for 12 h, then filtered. The resin was washed with DCM (2×40 mL) and DMF (2×40 mL). The peptide resin was then suspended in a solution of Ac₂O (1.04 g, 10.2 mmol), and iPr₂NEt (3.07 g, 23.8 mmol) in 40 mL of DMF. The mixture was shaken at RT for 1-2 h, filtered and resin washed with DCM (2×40 mL) followed by DMF (2×40 mL). To the resin was added 40 mL of dry 4M HCl in 1, 4-dioxane followed by shaking for 30-40 min. at RT. The reaction was filtered, and the resin washed with DCM (2×40 mL) followed by DMF (2×40 mL). EDCI (0.98 g, 5.1 mmol), HOBt (0.78 g, 5.1 mmol), iPr₂NEt (1.1 g, 8.5 mmol), and DMF (80 mL) were added to the resin and the resulting mixture was shaken for overnight at RT. The mixture was filtered and the resin and washed with DCM (2×40 mL) followed by DMF (2×40 mL). To the acetyl capped peptide resin was added TFA (2×35 mL) and shaken for 30 min. The mixture was filtered, and the resin washed with DCM (2×40 mL). The filtrate was concentrated, and cold ether was added to the residue yielding the crude product as a precipitate which was purified by flash chromatography to give an off-white sold (400 mg).

The macrocyclic tripeptide acid (400 mg, 1.0 mmol) was dissolved in dry DMF (10 mL) under a nitrogen atmosphere at 0° C. and HATU (456 mg, 1.20 mmol) was added followed by stirring for 15 min, and then the addition of Arg(Pbf)-kbt:HCl (638 mg; 1.10 mmol) and iPr₂NEt (0.87 mL, 5.0 mmol) at 0° C. The reaction is allowed to reach RT and then stirred for 2-3 h. The DMF was removed in vacuo and water (250 mL) was added to the resulting residue. The precipitate formed was filtered and washed with water (2×50 mL) and dried. To this precipitate was added 10 mL of TFA/thioanisole/water (95:2.5:2.5 v/v/v) and the mixture was stirred for 2 h at RT. The solvent was removed, and then cold ether (100 mL) was added. The crude product was collected by centrifugation. The crude product was purified by HPLC (Cis, 15×150 mm column; eluent: acetonitrile/water (0.05% TFA) to give the title compound as a white solid.

Ac-Cyclo(Allyl-Y)—R-ketobenzothiazole, 21. Fmoc-(L)-glycine (3.5 g, 10 mmol) stirred in 20% piperidine in DMF (20 mL) for 1 hr. Solvent was removed under reduced pressure, product triturated with DCM and hexanes (1:3), filtered the product and washed with hexanes, dried and used in the next reaction. Above material was dissolved in methanol (10 mL) and cooled the reaction to 0° C. followed by added thionyl chloride (2 mL) dropwise and stirred for 10 min and ice bath was replaced by a water bath, and the reaction mixture heated to −50° C. for 3 hr while stirring. Removal of the solvent left a white residue which was washed with diethyl ether (100 mL) and collected by vacuum filtration to yield the amino acid methyl ester hydrochloride as a solid (1.7 g). Above ester (500 mg; 3.02 mmol) was taken in DCM (10 mL) and added DIEA (1.58 mL; 9.06 mmol) and Ac₂O (0.86 mL; 9.06 mmol) at RT and stirred for 3 hrs. Solvent was removed under reduced pressure and crude was purified by flash chromatography using EtOAc and Hexanes (1:9). A solution of ester (395 mg; 2.5 mmol) in THF (3 mL) was treated with 1M aqueous LiOH (3 mL) and the reaction mixture was stirred for 3 h at RT, and the absence of starting material was monitored by TLC. After the solvent was evaporated off, the residue was diluted with water and the pH was adjusted to ˜3.0 using 5% aq. HCl. The product was extracted with ethyl acetate (2×50 mL) and the combined organic layer washed with brine (20 mL), dried over anhydrous Na₂SO₄, filtered off and concentrated, which is used in the next step without further purification. N-acetyl allyl glycine acid (167 mg; 1.06 mmol) in DMF (5 mL) was stirred with peptide coupling reagent EDCI/HOBt or HATU (1.3 eq) for 30 min. The reaction was cooled to 0-5° C. and charged with amino acid methyl ester hydrochloride (1.1 eq.) followed by diisopropylethylamine (3.0 eq.). After 15 min, allowed the reaction was brought to RT and stirred overnight. Solvent was removed under reduced pressure and the residue partitioned between EtOAc and 5% aq. HCl. The separated organic layer was washed with aq. 5% HCl, saturated NaHCO₃ solution (2×) and brine (1×) then dried over anhydrous Na₂SO₄. The crude product was purified by silica gel column chromatography using EtOAc and Hexanes (2:8). A solution of the ester (343 mg; 1.2 mmol) in THF (4 mL) was treated with 1M aqueous LiOH (4 mL). The reaction mixture was stirred for 3 h at RT, and the absence of starting material was monitored by TLC. After the solvent was evaporated off, the residue was diluted with water and the pH was adjusted to ˜3.0 using 5% aq. HCl. The crude product was extracted into ethyl acetate (3×100 mL). The combined organic layers were washed with brine (25 mL) and dried over anhydrous Na₂SO₄. N-acetyl dipeptide acid (135 mg; 0.5 mmol) was stirred with EDCI (1.3 eq) and HOBt (1.3 eq) in DMF (3 mL) for 30 min. The reaction was cooled to 0-5° C. and H-L-O-allyl Tyr-OMe. HCl (130 mg: 0.55 mmol) followed by DIEA (3.0 eq). After 15 min, allowed the reaction to RT and stirred overnight. Solvent was removed under reduced pressure and the residue partitioned between EtOAc and 5% aq. HCl. The separated organic layer was washed with aq. 5% HCl, saturated NaHCO₃ solution (2×) and brine (1×) then dried over anhydrous Na₂SO₄. The crude product was purified by silica gel column chromatography using EtOAc and Hexanes (3:7).

A solution of acyclic diene precursor (150 mg, 0.3076 mmol) in DCM (280 mL, 0.2 Mol.) degassed for 30 min by purging nitrogen gas and then Grubbs 2^{nd} generation catalyst (26 mg, 10 mol %) was added. The reaction was refluxed for 30 min and then additional Grubbs 2^{nd} generation (13 mg, 5 mol %) was added. The reaction was refluxed for 18 h under nitrogen atmosphere. After depletion of the starting material as monitored by TLC and LCMS, the reaction was cooled to RT and quenched by adding activated charcoal (100 mg) followed by stirring for 1 h. The mixture was filtered through celite bed and washed generously with DCM. The filtrate was concentrated in vacuo and the crude product was purified by silica gel chromatography to yield an off-white solid. A solution of the macrocyclic ester (50 mg: 0.10 mmol) in MeOH (2 mL) was treated with 1M aq. LiOH (2 mL) at RT for 3 hrs, and the absence of starting material was monitored by TLC. After the solvent was evaporated off, the residue was diluted with water and the pH was adjusted to ˜3.0 using 5% aq. HCl. The product was extracted with ethyl acetate (3×50 mL) and the combined organic layer washed with brine (20 mL), dried over anhydrous Na₂SO₄, filtered off and concentrated, which is used in the next step without further purification.

The macrocyclic acid (45 mg, 0.101 mmol) was dissolved in dry DMF (3 mL) under a nitrogen atmosphere at 0° C. and HATU (50 mg, 1.30 mmol) was added followed by stirring for 15 min, and then the addition of Arg(Pbf)-kbt:HCl (65 mg; 0.111 mmol) and iPr₂NEt (70 uL, 0.404 mmol) at 0° C. The reaction is allowed to reach RT and then stirred for 2-3 h. The DMF was removed in vacuo and water (250 mL) was added to the resulting residue. The precipitate formed was filtered and washed with water (2×50 mL) and dried. To this precipitate was added 2.5 mL of TFA/thioanisole/water (95:2.5:2.5 v/v/v) and the mixture was stirred for 2 h at RT. The solvent was removed, and then cold ether (35 mL) was added. The crude product was collected by centrifugation. The crude product was purified by HPLC (Cis, 15×150 mm column; eluent: acetonitrile/water (0.05% TFA) to give the title compound as a white solid.

The synthesis of 9, 10, 11, and 18 have been previously reported (Han, Z., et al. (2014) ACS Med Chem Lett 5, 1219-1224.).

The synthesis of 1, 3, 12, 16, and 17 are as previously described (Han, Z., et al. (2016) ChemMedChem 11, 585-599.).

### Example 9: Macrocycle Syntheses

Rational Design and Synthesis of Sidechain Cyclized Peptides with Alkenyl Phenyl Ether or Alkyl Phenyl Ether Linkers

In this approach to develop and study newly designed macrocyclic peptides, it was desired to introduce increased conformational restriction relative to the simple, flexible cycloamides previously reported. In this new set of analogs, alternate linkers were utilized to investigate and understand how this modification would change the potency, protease selectivity as well as the physical and ADME properties of the macrocyclic inhibitor. In the rational design strategy, the idea of sidechain cyclization is based on using the X-ray structure of Ac-KQLR-cmk, an irreversible inhibitor bound to HGFA (PBD: 2WUC) which shows there is a 6.1 Å distance from the P4 Lys and P2 Leu sidechains of the inhibitor and which are pointing in the same direction. By connecting the sidechains into different macrocyclic ring systems with various linkers such as a simple cycloamide from a P4 Lys and P2 Asp (green) or a phenyl ether via a P4 Tyr and P2 AllylGly results in conformationally restricted macrocyclic peptides as j-sheet mimetics. These cyclic tripeptides were modeled to the HGFA active site where the cyclic linker fully encompasses the S4-S2 pockets.

Shown in Schemes 7 and 8, the 10-atom phenyl-alkenyl and alkenyl-phenyl sidechain linker macrocycles were constructed from acyclic peptides with the P2 and P4 residues being either an allylglycine or Tyr(O-allyl). The synthesis involved multistep solution phase peptide couplings and deprotection reactions from the commercially available amino acid derivatives Fmoc-allylGly-OH and Boc-Tyr(O-allyl)-OH. The synthesis begins with Fmoc removal of Fmoc-allylGly-OH followed by formation of the methyl ester, acetylation and then methyl ester hydrolysis to yield intermediate amino acid 26 (Scheme 7). Coupling to H-Leu-OMe using standard peptide coupling conditions (EDC/HOBt/DIEA/DMF), hydrolysis of ester 27, followed by another coupling of resultant acid 28 to Tyr(Oallyl)-OMe gives key intermediate 29. In a similar fashion starting with Boc-Tyr(Oallyl)-OH and allylGly-OMe, intermediate 44 was obtained (Scheme 8). The final target alkenyl phenyl ether macrocycles 3 and 4 were obtained via a ring-closing metathesis reaction of intermediate tripeptides 29 (Scheme 7) and 44 (Scheme 8) using Grubbs second generation catalyst to give 30 and 48 followed by hydrolysis of the methyl ester and then coupling to Arg(Pbf)-kbt and final deprotection to give 3 and 4, respectively. When tested against TMPRSS2, HGFA, matriptase, and hepsin, compound 3 (P4-P2, alkenyl-ether-phenyl), there was no inhibition of HGFA but excellent inhibition of TMPRSS2 and matriptase (IC₅₀=14 nM) and hepsin (IC₅₀=20 nM). Surprisingly, for compound 4 which has the reversed orientation of P4 Tyr and P2 allyl ether group (Scheme 8), potent HGFA inhibition was found with an IC₅₀=53 nM, while matriptase potency is retained with an IC₅₀=3.4 nM, and TMPRSS2 (IC₅₀=1.2 nM) was increased over 10-fold and hepsin activity was enhanced 65-fold (IC₅₀=0.17 nM). Noticeably was also the potency seen against factor Xa with an IC₅₀ of 122 nM. It appears that the conformation of macrocycles where a phenyl ring is in the P4 position allows for significantly improved binding to HGFA in the S4 pocket, presumably from the twisting of the ring out of the plane. Docking studies of 3 and 4 to HGFA performed to explain these results were not conclusive but clearly the experimental values indicate this conclusion.

In order to investigate the effect of less conformational restriction in the linker, the reduced version of alkenyl macrocycle 4 was synthesized as shown in Scheme 8. To obtain this compound 5 first the alkene of methyl ester 48 was reduced to alkyl 52 using catalytic hydrogenation, followed by ester hydrolysis. Then coupling of acid 52 to Arg(Pbf)-kbt and subsequent deprotection of the Arg Pbf group gave target compound 5. When compared to the alkene linker of 4, the alkyl linker of 5, the HGFA potency improves slightly (IC₅₀=40 nM) but TMPRSS2 (IC₅₀=9.8 nM), matriptase (IC₅₀=32 nM) and hepsin (IC₅₀=0.91 nM) inhibition drops 9 and 5-fold, respectively and with a >20-fold drop in factor Xa (IC₅₀=2646 nM). Based on this new and promising SAR and knowledge of preferred residues in the P3 position for HGFA, the central P3 residue of this macrocyclic tripeptide series was modified from Leu to Phe, Trp, D-Trp (Scheme 8). These new analogs showed a substantial improvement of HGFA inhibition. Compound 6, with a Phe group at the P3 position displayed improved inhibition for HGFA, matriptase and hepsin but less potency against TMPRSS2 (IC₅₀=16.4 nM), while compound 7 with a P3 Trp has lower HGFA (IC₅₀=85 nM), matriptase (IC₅₀=57 nM) and hepsin (IC₅₀=2.8 nM) inhibition. Both 6 and 7 have excellent selectivity over factor Xa and thrombin. Excitingly, compound 8b with a D-Trp at P3 had significantly improved potency for all four target enzymes TMPRSS2 (IC₅₀=4.7 nM), HGFA (IC₅₀=3.3 nM), matriptase (IC₅₀=2.9 nM), and hepsin (IC₅₀=0.54 nM). The corresponding alkenyl linker analog with the central D-Trp residue, 8a showed equivalent inhibition of all target proteases.

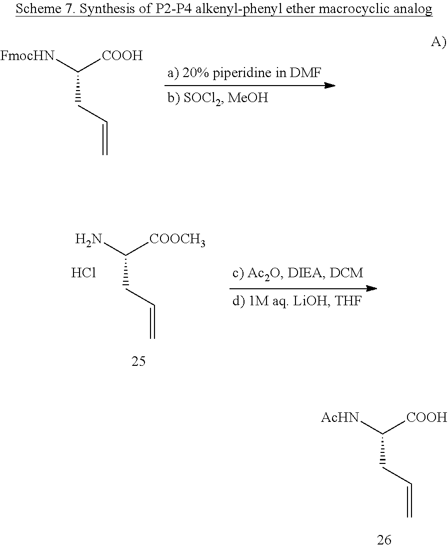

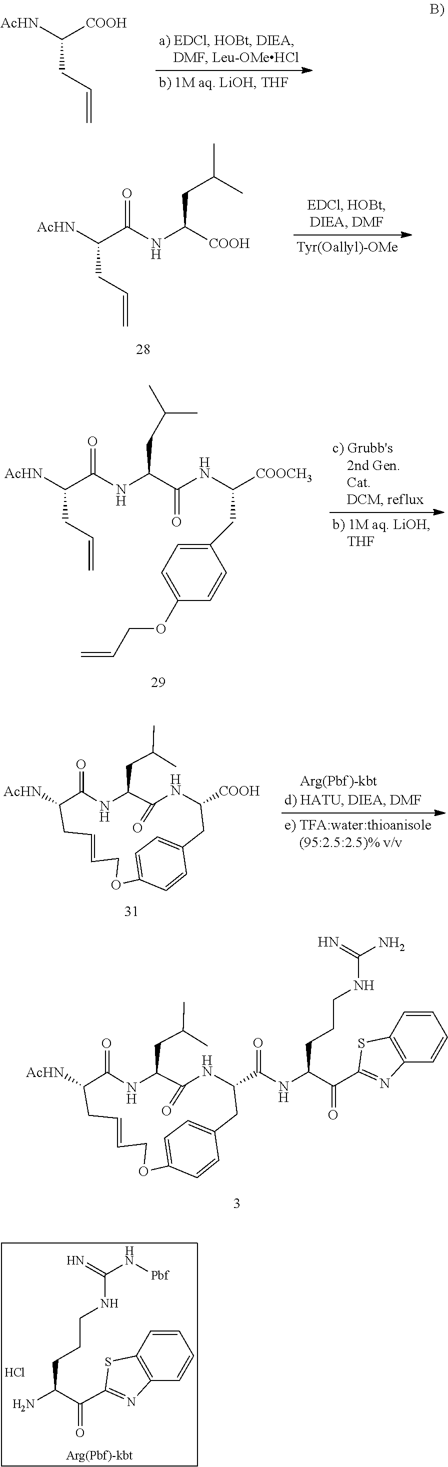

### Development of Tyr-Alkenyl-Amide P4-P2 and P2-P4 Cyclized Macrocycles

Building on previous success and learning with the phenyl ether alkenyl and alkyl macrocycles, it was next desired to study the effect on size of the macrocycle in relation to potency and selectivity. To accomplish this aim, an Asp or Glu as one sidechain was retained, allowing us to vary the size of the ring (Scheme 9) like in the simple cycloamide series, but now have incorporated a Tyr (Phe) as the other sidechain, where the two sidechains are further linked through an alkene attached to Tyr as an ether and to the Asp or Glu as an amide. So, in a sense these are larger hybrids of the phenyl ether macrocycles and cycloamides but with larger rings having a 13-atom and 14-atom linker to the peptide backbone, respectively. Shown in Scheme 9, the synthesis begins with dipeptide acid 36 of Scheme 8 which was coupled with allyl amide intermediates (23 or 24) to give Boc-protected tripeptides 15 and 16, respectively. Intermediates 23 and 24 were synthesized via EDC coupling of either the sidechain carboxylic acid of Boc-Asp-OMe or Boc-Glu-OMe with allylamine followed by Boc-deprotection with 4 N HCl in dioxane. Boc deprotection followed by N-acetyl capping produced the key cyclization substrates 17 and 18. Intramolecular reaction of these acyclic dienes was affected using a ring-closing metathesis reaction employing Grubbs catalyst as before, to furnish macrocyclic esters 19 and 20. The esters were hydrolyzed, and the resultant acids 21 and 22 were coupled with Arg(Pbf)-kbt using HATU to give the final target macrocyclic compounds 1 and 2 after Pbf deprotection with TFA/water/thioanisole.

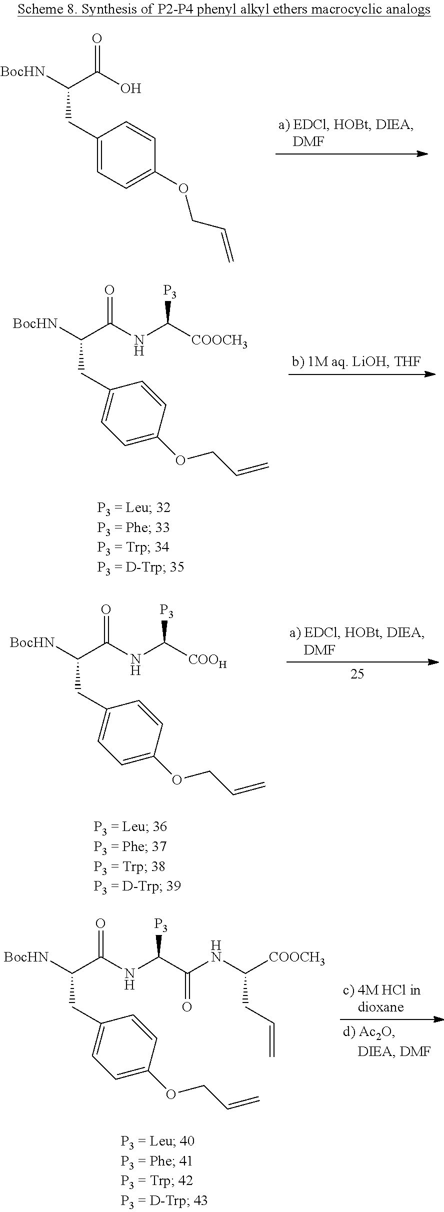

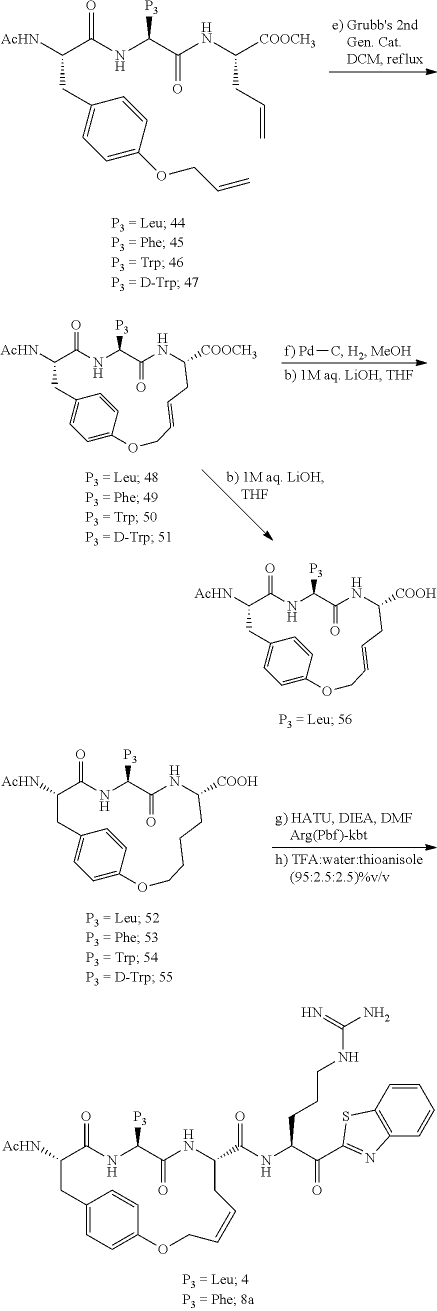

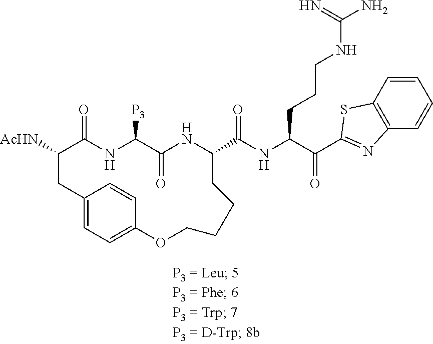

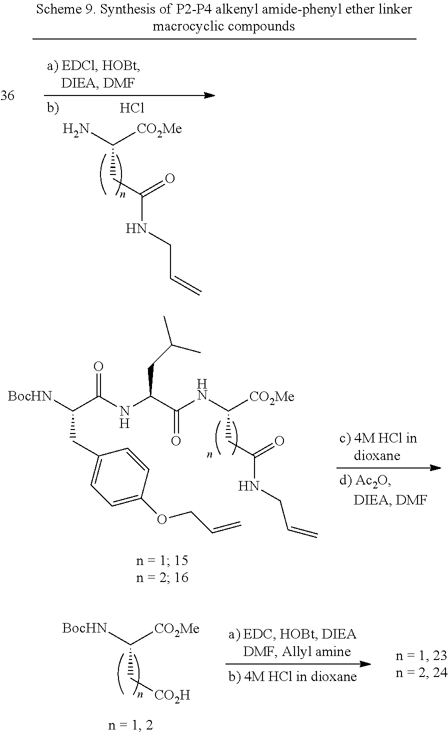

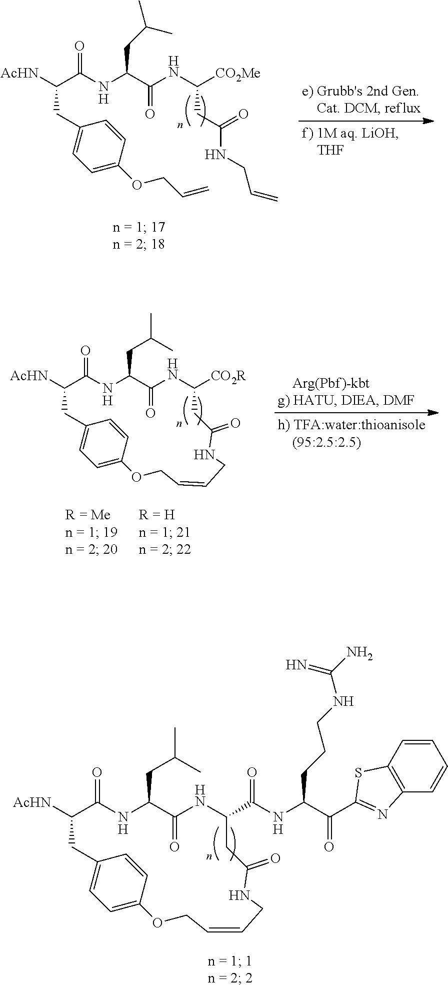

### Triazole Linked Macrocycles

Next was exploration of analogs where the phenyl group is replaced with a smaller 5-membered ring aryl heterocycle beginning with a triazole, in part due to its synthetic feasibility from an azide and alkyne. This change would create macrocycles as well as allow for different conformational ensembles with a different overall more constrained ring size of an 8-atom or 9-atom linker to the peptide backbone. Shown in Scheme 10, a matched pair of compounds were synthesized with one having the triazole in the P4 position (S4 pocket) and the other having it at P2 (S2). The synthesis utilizes standard amide couplings as previously described, protection and deprotection reactions for peptide synthesis starting from commercially available propargyl glycine and azido lysine derivatives to make the acetylated tripeptide cyclization precursors 60 and 66. Macrocyclization of 60 and 66 was performed using standard copper-mediated triazole formation from “Click” reaction of the alkyne and azide sidechains to give macrocycles 61 and 67. Ester hydrolysis followed by addition of the Arg(Pbf)-kbt and subsequent deprotection, gave the two final target compounds 9 and 10. When tested for their inhibitory potency against the six serine proteases as before, the P4 triazole linker 9 mimics the P4 phenyl linker (3) for its effect against all proteases except is 5-fold less potent for hepsin but in contrast when P2 triazole (10) is compared to P2 phenyl (4), surprisingly HGFA potency is significantly decreased to 3.1 μM and the IC₅₀ for matriptase is 10-fold less (39 nM) while the hepsin IC₅₀ is also reduced 100-fold (117 nM). Both of the triazole linked macrocycles have excellent potency against TMPRSS2. It should be pointed out that the cyclic triazole linkers are connected via an alkyl group and compound 3 has an alkenyl-phenyl linker while 4 has a phenyl-alkyl linker. No P2 alkyl to P4 phenyl linked macrocycles were synthesized. In any case, the reason for this outcome of reduced inhibition of the triazoles when compared to the phenyl paired compounds is currently not clear but is most likely due to the different overall ring sizes (triazole 15-membered (8-atom linker); phenyl 17-membered (10-atom linker)) and the differences in their conformational flexibility.

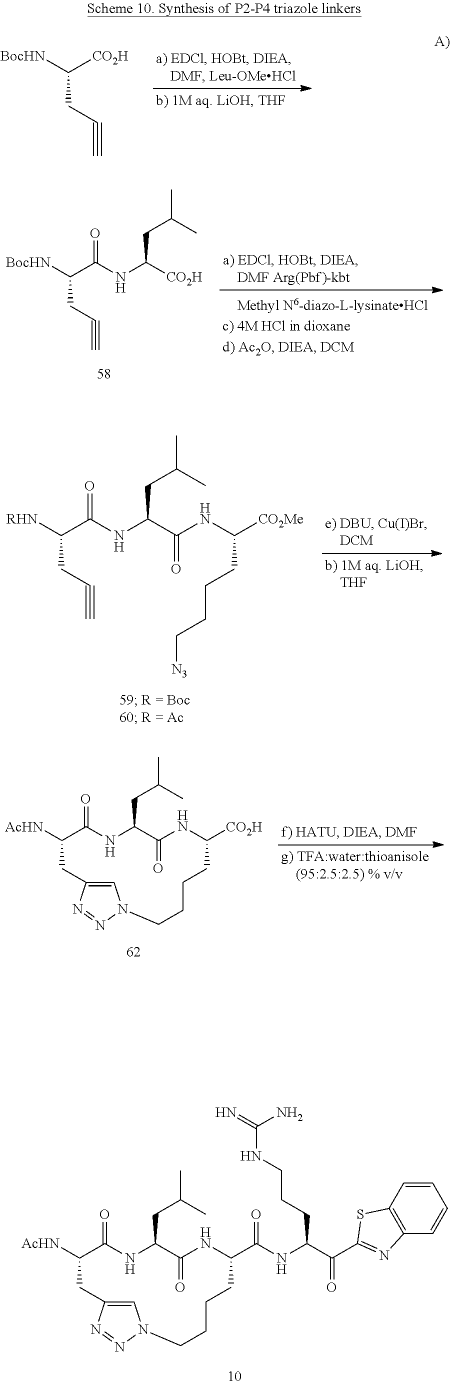

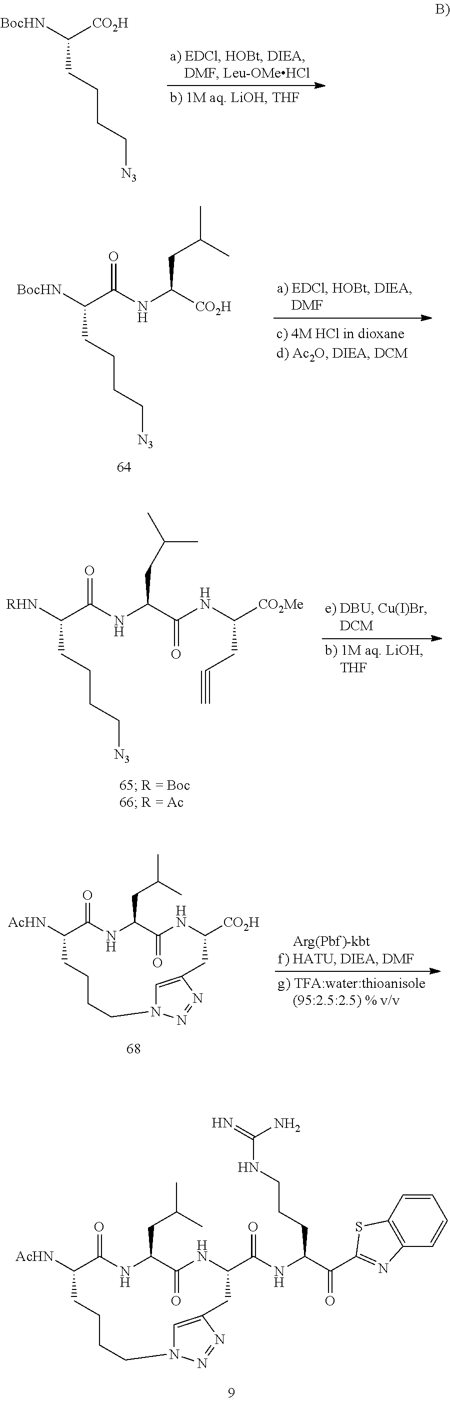

### Cyclic Biphenyl Ether Linked Macrocycles

To explore a more conformationally restricted macrocycle, the natural products K-13 and OF4949-IV were turned to as was done by the inventors in the development of HIV-protease inhibitors back in 1997. Shown in Schemes 11-13, the para-meta (11), para-para (12), and meta-para (13) isomers of the biphenyl ether branching the P2 and P4 positions via a Tyr and Phe sidechain were constructed. In all three syntheses, an aryl boronic acid derivative of Phe was created and employed, which readily reacts with phenols (in this case Tyr sidechain hydroxyl) in the presence of copper acetate and in this case a ring closing Evans-Chan-Lam reaction. To be consistent with other analogs, the central amino acid of the macrocyclic peptide was kept as a Leu. The synthesis begins as shown in Scheme 11 from Boc-protected para-bromo phenylalanine which is converted into the aryl boronate 69, which was coupled to Leu-OMe to give boronate ester 70, which was hydrolyzed under basic conditions to give dipeptide acid 71. Coupling to meta-tyrosine methyl ester gives 72 and hydrolysis of the boronate ester to the boronic acid then yields the cyclization intermediate 73. Intramolecular Chan-Lam-Evans reaction produces macrocycle 74, which was Boc deprotected to give 75, acetylated providing 76 which was coupled to Arg-kbt as described earlier giving para-meta cyclic biphenyl ether tripeptide 76. The final target compound 11 is obtained after Arg deprotection under standard conditions. The synthesis of 12, shown in Scheme 12, begins with intermediate 71 as in Scheme 11 but is coupled to tyrosine methyl ester to give intermediate tripeptide 77 which after boronate ester hydrolysis was carried forward to the macrocyclic para-para cyclic biphenyl ether 12 in an identical manner to that of 11. Shown in Scheme 13, the synthesis of para-para cyclic biphenyl ether macrocycle 13 started with Boc-meta-tyrosine, leucine methyl ester, and para-boronic acid phenylalanine methyl ester, to construct the tripeptide cyclization precursor 87 which was subsequently converted into compound 13 following the same steps used for 11 and 12.

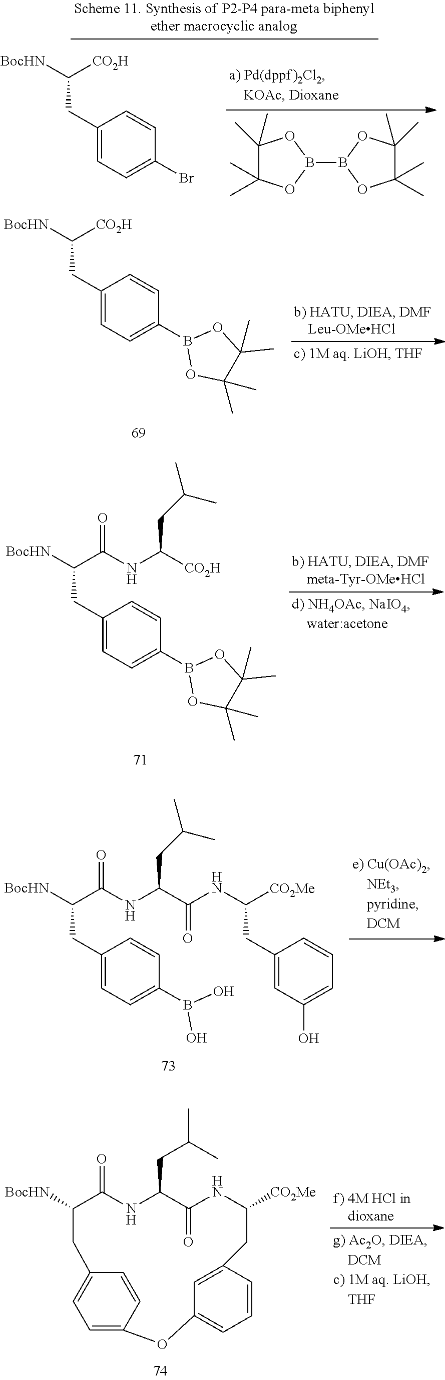

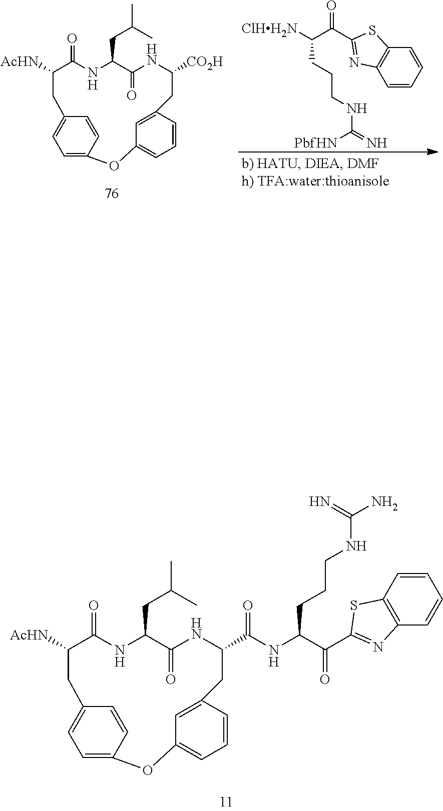

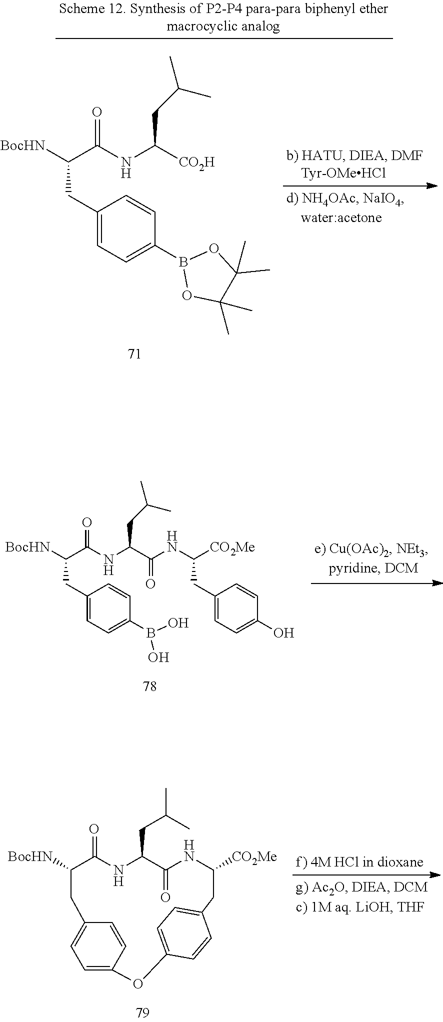 [SMILES: CC(C)C[C@H](NC(=O)[C@H](Cc1ccc(B2OC(C)(C)C(C)(C)O2)cc1)NC(=O)OC(C)(C)C)C(=O)O ; COC(=O)[C@H](Cc1ccc(O)cc1)NC(=O)[C@H](CC(C)C)NC(=O)[C@H](Cc1ccc(B(O)O)cc1)NC(=O)OC(C)(C)C ; COC(=O)[C@@H]1Cc2ccc(cc2)Oc2ccc(cc2)C[C@H](NC(=O)OC(C)(C)C)C(=O)N[C@@H](CC(C)C)C(=O)N1]

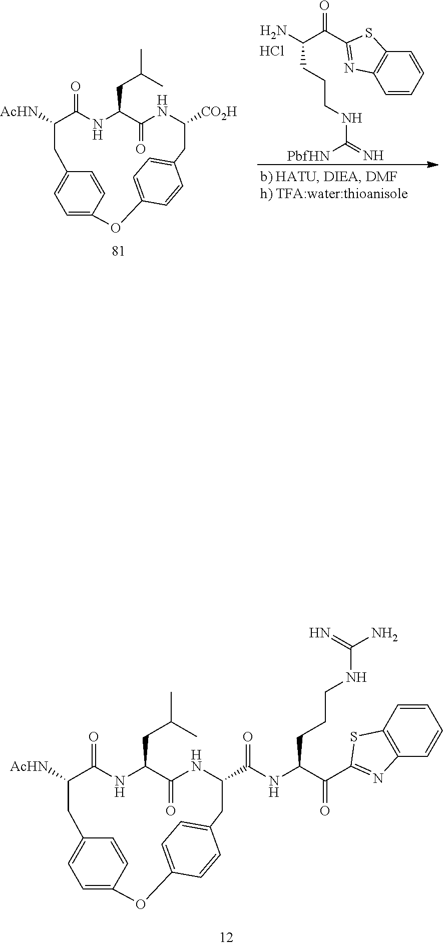

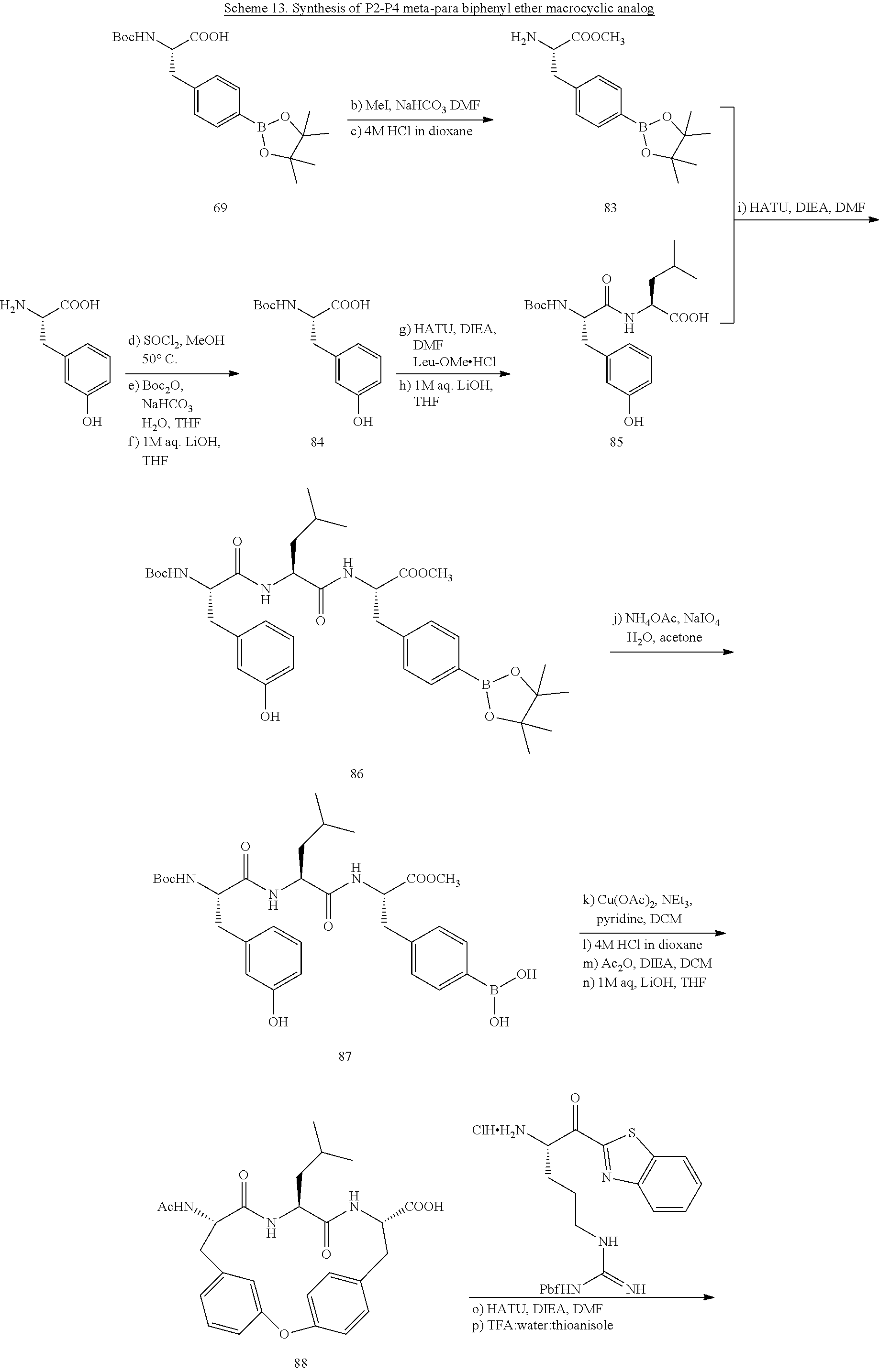

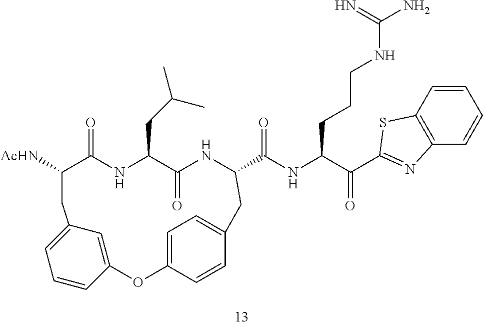 [SMILES: CC(=O)N[C@H]1Cc2cccc(c2)Oc2ccc(cc2)C[C@@H](C(=O)N[C@@H](CCCNC(=N)N)C(=O)c2nc3ccccc3s2)NC(=O)[C@H](CC(C)C)NC1=O]

### EXPERIMENTAL

General synthesis, purification, and analytical chemistry procedures. Starting materials, reagents, and solvents were purchased from commercial vendors unless otherwise noted. ¹H NMR spectra were obtained on a Varian 400 MHz NMR instrument equipped with an autosampler. The chemical shifts (6) are reported as δ ppm relative to TMS using residual solvent peak as the reference unless otherwise noted. The following abbreviations are used to express the multiplicities: s=singlet; d=doublet; dd=doublet of doublets; t=triplet; q=quartet; m=multiplet; br=broad. High-performance liquid chromatography (HPLC) was carried out on GILSON GX-281 using Waters C18 5 μM, 4.6*50 mm and Waters Prep C18 5 μm, 19*150 mm reverse phase column, eluted with a gradient system of 5:95 to 95:5 acetonitrile:water with a buffer consisting of 0.05% TFA. Mass spectra (MS) were generated on an Agilent or Waters HPLC/MSD using electrospray ionization (ESI) for detection. All reactions were monitored by thin layer chromatography (TLC) which were conducted on Merck silica gel plates (0.25 mm thick, 60F254), visualized by using UV (254 nm) or dyes such as KMnO₄, p-anisaldehyde and CAM (Cerium Ammonium Molybdate or Hanessian's Stain). Silica gel chromatography was performed on a Teledyne ISCO CombiFlash purification system using pre-packed silica gel columns (12 g˜330 g sizes). All compounds tested in biological assays are greater than 95% purity based on ¹H NMR peak integration and HPLC by absorbance at 220 nm and 254 nm wavelengths and AUC of the MS peak.

See, for example, the below exemplary synthesis. Other similar compounds can be made similarly.

Methyl ((S)-3-(4-(allyloxy)phenyl)-2-((tert-butoxycarbonyl)amino)propanoyl)-L-leucinate (32). Boc-O-allyl-L-Tyr-OH 12 (330 mg, 1.25 mmol) was treated with EDCI (311 mg, 1.3 eq.), HOBt (249 mg, 1.3 eq.) in DMF for 30 min. The reaction was cooled to 0-5° C. and Leu-OMe (250 mg, 1.375 mmol) was added followed by iPr₂NEt (0.87 mL, 3.0 eq.) at 0-5° C. and the stirring was continued for 15 min. The reaction was allowed to reach room temperature and then stirred overnight. The reaction was monitored by TLC/LCMS. The solvent was removed under reduced pressure and the resulting residue partitioned between EtOAc and 5% aq. HCl. The separated organic layer was washed with aq. 5% HCl, saturated NaHCO₃ solution (2×) and brine (1×). The organic layer was then dried over anhydrous Na₂SO₄, filtered, and concentrated. The crude product was purified by silica gel column chromatography (EtOAc/hexanes gradient) to give a solid.

Synthesis of Macrocyclic Compound. Ring Closing Metathesis (RCM)

See, for example, the below exemplary synthesis. Other similar compounds can be made similarly.

Methyl (7S,10S,13S,E)-7-acetamido-10-isobutyl-8,11-dioxo-2,9-dioxa-12-aza-1(1,4)-benzenacyclotetradecaphan-4-ene-13-carboxylate (30). Acyclic precursor compound 29 (150 mg, 0.512 mmol) was dissolved in DCM (279 mL) and the stirred mixture was degassed for 30 min by purging with nitrogen gas and then Grubbs 2^{nd} generation catalyst lot-1 (26 mg, 10 mol %) was added followed by heating the reaction to reflux temperature. The reaction was refluxed for 30 min and then more Grubbs 2^{nd} generation catalyst lot-2 (13 mg, 5 mol %) was added and the reaction was continued for 18 h under nitrogen atmosphere. The reaction was monitored by TLC or LCMS and after disappearance of the starting material, the reaction was cooled to room temperature and quenched with the addition of activated charcoal (100 mg) and stirring for 1 h. The reaction was filtered through a celite bed and washed 3× with DCM. The filtrate was concentrated, and crude product was purified by silica column (EtOAc/hexanes). Off-white solid yielded as a solid product.

### Ring Closing Metathesis (RCM)

See, for example, the below exemplary synthesis. Other similar compounds can be made similarly.

Ac-cyclo-(L) Tyr-Leu-allyl-OMe (48). Acyclic diene 44 (500 mg, 1.025 mmol) was dissolved in DCM (800 mL) which was degassed for 30 min by purging nitrogen gas and then Grubbs 2^{nd} generation catalyst lot-1 (130 mg, 15 mol %) was added, followed by heating the reaction to reflux temperature for 18 h under a nitrogen atmosphere. The reaction was monitored by TLC or LCMS. After disappearance of the starting material, the reaction was cooled to room temperature and quenched by adding activated charcoal (100 mg) and stirring for 1 h. The reaction was filtered through a celite bed and washed 3× with DCM, the filtrate was concentrated, and resultant residue was purified by silica gel column (EtOAc/hexanes). Off-white solid was obtained as a product. Yield (300 mg, 65%).

### Reduction of Alkene

See, for example, the below exemplary synthesis. Other similar compounds can be made similarly.

Ac-cyclo-(L) Tyr-Leu-alkyl-OMe (52a). The macrocyclic alkene compound 48 (250 mg, 0.544 mmol) was dissolved in MeOH (10 mL) and 10% Pd—C (˜50 mg) was carefully added under an argon atmosphere, and the reaction was equipped with a hydrogen balloon and stirred overnight at RT. After completion of reaction as indicated by the disappearance of starting material using TLC, the reaction mixture was filtered through a celite bed and washed with MeOH several times. The solvent was evaporated, and the residue was purified by silica chromatography (EtOAc/hexanes). White solid, yield (200 mg, 85%).

### Intramolecular Click Chemistry.

See, for example, the below exemplary synthesis. Other similar compounds can be made similarly.

Methyl (3S,6S,9S,Z)-3-acetamido-6-isobutyl-4,7-dioxo-11H-5,8-diaza-1(4,1)-triazolacyclotridecaphane-9-carboxylate (61). To a solution of acyclic azido alkyne 60 (500 mg, 1.01 mmol) in anhydrous DCM (845 mL) was added DBU (0.454 mL, 3.03 mmol) at RT under argon atmosphere while stirring, and after 15 min Cu(I)Br (145 mg, 1.01 mmol) was added and the reaction was then stirred overnight (˜18 h) at RT. Progress of the reaction was monitored by TLC/LCMS. The reaction was quenched by adding 3 N HCl (100 mL). The layers were separated, and the aqueous layer was extracted with DCM (2×). The combined organic layers were washed with water, brine and dried over anhydrous Na₂SO₄, filtered, and concentrated to yield a crude product, which was purified by silica chromatography (EtOAc/hexanes).

1. A compound of Formula (IA), (IB), or (IC), a pharmaceutically acceptable salt thereof, or a stereoisomer thereof: 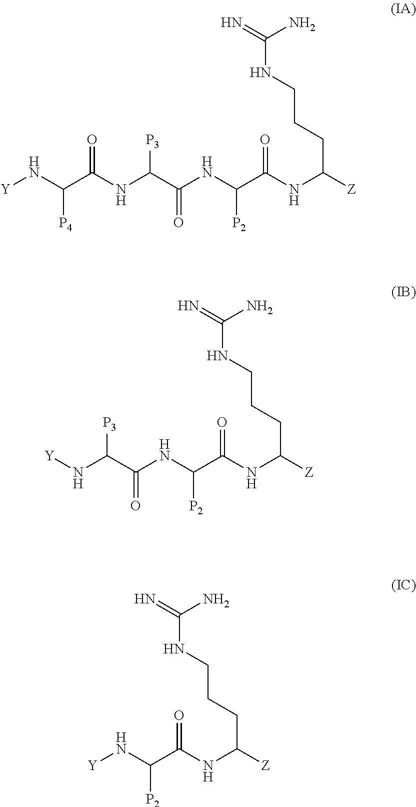
wherein:
each P2 is independently a side chain of 1-Nal, 2-Nal, Gly(2-th), D-Ala, L-Ala, L-Ala(2-th), L-Arg, L-Arg(Z)₂, L-Asn, L-Bla, L-Cha, L-Chg, L-Glu(OBzl), L-hArg, L-hCha, L-His(Bzl), L-hLeu, L-hPhe, L-hTyr, L-hTyr(Me), L-Igl, L-Leu, L-Lys, L-Lys(2-ClZ), L-Nle, L-Nle(OBzl), L-NptGly, L-Nva, L-Orn, L-Phe, L-Phe(3,4-F2), L-Phe(3-Cl), L-Phe(3-F), L-Phe(4-F), L-phg, L-Ser, L-Thr, L-Trp, or L-Val;
each P3 is independently a side chain of 3-AMPA, 4-AMBA, 4-AMPA, D-Arg, D-Gln, D-Lys, D-Phg, D-Ser, D-Trp, L-2-Aoc, L-Abu(Bth), L-Agb, L-Agp, L-Ala(Bth), L-Arg, L-Arg(Z)₂, L-Asp(OCHx), L-Dab, L-Dap, L-Dht, L-Gln, L-Glu, L-Glu(All), L-Glu(Bzl), L-Glu(Obzl), L-Glu(OCHx), L-Glu(OMe), L-hArg, L-hCha, L-His(3-Bom), L-hPhe, L-hTyr, L-Igl, L-Leu, L-Lys, L-Lys(2-ClZ), L-Met, L-Met(O), L-Met(O)₂, L-Nle(OBzl), L-Orn, L-Phe(4-NO₂), L-Phe(F5), L-Ser, L-Ser(Ac), L-Thr, or L-Trp;
each P4 is independently a side chain of 2-Abz, 3-Abz, 4-Abz, D-Arg, dhAbu, dhLeu, D-Lys, D-Trp, Gly, L-2-Aoc, L-Agb, L-Agp, L-Ala(Bth), L-Arg, L-Arg(NO₂), L-Arg(Z)₂, L-Chg, L-Cys(4-MeOBzl), L-Cys(Bzl), L-Cys(MeBzl), L-DAB(Z), L-Glu(OBzl), L-Glu(OCHx), L-hArg, L-hCha, L-His(3-Bom), L-hLeu, L-hPhe, L-hTyr, L-Hyp, L-Hyp(Bzl), L-Idc, L-Ile, L-Leu, L-Lys, L-Lys(2-Cl-Z), L-Lys(TFA), L-Met, L-Nle, L-Nle(OBzl), L-Nva, L-Oic, L-Orn, L-Phe, L-Phe(4-I), L-Phe(F5), L-Pro, L-Ser, L-Ser(Bzl), L-Thr(Bzl), L-Trp, or TmbGly;
Y is H, acetyl, tert-butyloxycarbonyl (Boc), benzyloxycarbonyl (Cbz), fluorenylmethyloxycarbonyl (Fmoc), benzyl, —C(O)R, —SOOR, —COOR, —C(O)NHR, —COCH(NHC(O)CH₃)—(CH₂)ₓ—NHC(O)—(CH₂)ₓ-p-halo-phenyl, substituted or unsubstituted —(CH₂)ₓaryl, substituted or unsubstituted —(CH₂)ₓheteroaryl, substituted or unsubstituted —(CH₂)ₓcycloalkyl, or substituted or unsubstituted —(CH₂)ₓheterocycle;
each x is independently an integer of 0, 1, 2, 3, or 4;
each R is independently C₁ to C₆ alkyl, C₃ to C₆ cycloalkyl, heterocycle, alkylheterocycle, aralkyl, or aryl;
each Z is independently 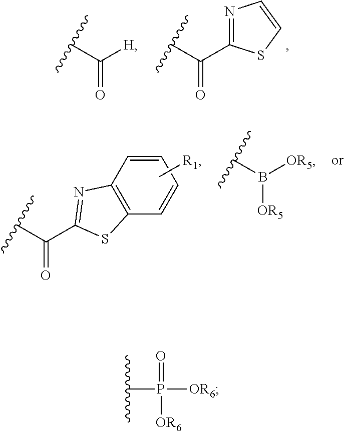
R₁ is hydrogen, 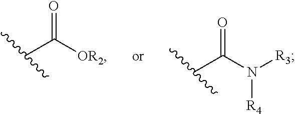
R₂ and R₃ are each independently hydrogen, substituted or unsubstituted alkyl, substituted or unsubstituted cycloalkyl, substituted or unsubstituted aryl, substituted or unsubstituted aralkyl, substituted or unsubstituted heteroaryl, or substituted or unsubstituted heteroarylalkyl;
R₄ is hydrogen, substituted or unsubstituted alkyl, or a residue of an amino acid, or R₃ and R₄ can form a ring;
each R₅ is independently hydrogen, substituted or unsubstituted alkyl, or the R₅ moieties can form a ring; and
each R₆ is substituted or unsubstituted aryl.

2.-8. (canceled)

9. A compound of Formula (IA), (IB), or (IC), a pharmaceutically acceptable salt thereof, or a stereoisomer thereof: 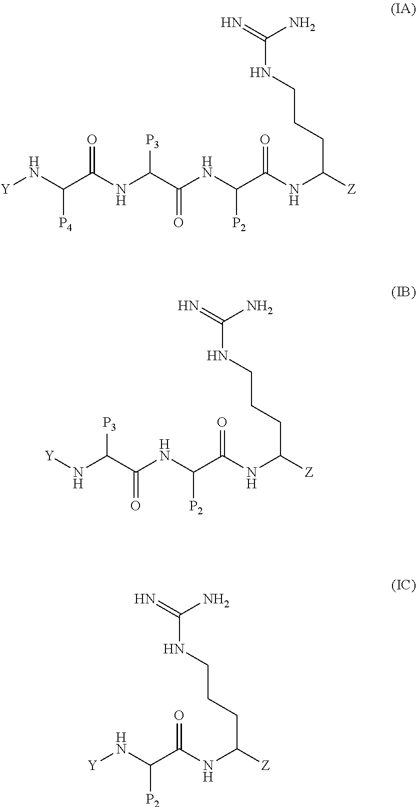
wherein:
each P2 is independently a side chain of L-Ala(2-th), L-Arg(Z)₂, L-Asn, L-Bta, L-Cha, L-Chg, L-Glu(OBzl), L-hArg, L-hCha, L-His(Bzl), L-hLeu, L-hPhe, L-hTyr(Me), L-hTyr, L-Igl, L-Leu, L-Lys(2-ClZ), L-Nle(OBzl), L-Nle, L-NptGly, L-Nva, L-Orn, L-Phe(3,4-F2), L-Phe(3-Cl), L-Phe(3-F), L-Phe(4-F), L-Phe, L-Thr, or L-Trp;
each P3 is independently a side chain of D-Arg, D-Gln, D-Lys, D-Trp, L-2-Aoc, L-Agp, L-Ala(Bth), L-Arg(Z)₂, L-Arg, L-Dab, L-Dap, L-Dht, L-Glu(All), L-Glu(OBzl), L-Glu(OCHx), L-Glu(OMe), L-hArg, L-hCha, L-hPhe, L-hTyr, L-Igl, L-Lys, L-Met(O), L-Met(O)₂, L-Nle(OBzl), L-Orn, L-Phe(F₅), or L-Ser(Ac);
each P4 is independently a side chain of 4-Abz, chAbu, D-Arg, dhAbu, dhLeu, L-Agp, L-Ala(Bth), L-Arg(NO₂), L-Arg(Z)₂, L-Arg, L-Chg, L-Cys(Bzl), L-Dab(Z), L-Glu(OBzl), L-hArg, L-His(3-BOM), L-hTyr, L-Hyp, L-Idc, L-Lys(2-ClZ), L-Lys, L-Nle(OBzl), or L-Oic, L-Orn;
Y is H, acetyl, tert-butyloxycarbonyl (Boc), benzyloxycarbonyl (Cbz), fluorenylmethyloxycarbonyl (Fmoc), benzyl, —C(O)R, —SOOR, —COOR, —C(O)NHR, —COCH(NHC(O)CH₃)—(CH₂)ₓ—NHC(O)—(CH₂)ₓ-p-halo-phenyl, substituted or unsubstituted —(CH₂)ₓaryl, substituted or unsubstituted —(CH₂)ₓheteroaryl, substituted or unsubstituted —(CH₂)ₓcycloalkyl, or substituted or unsubstituted —(CH₂)ₓheterocycle;
each x is independently an integer of 0, 1, 2, 3, or 4;
each R is independently C₁ to C₆ alkyl, C₃ to C₆ cycloalkyl, heterocycle, alkylheterocycle, aralkyl, or aryl;
each Z is independently 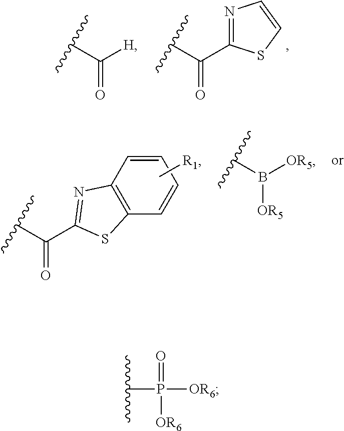
R₁ is hydrogen, 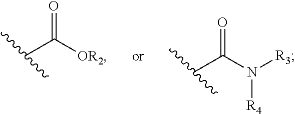
R₂ and R₃ are each independently hydrogen, substituted or unsubstituted alkyl, substituted or unsubstituted cycloalkyl, substituted or unsubstituted aryl, substituted or unsubstituted aralkyl, substituted or unsubstituted heteroaryl, or substituted or unsubstituted heteroarylalkyl;
R₄ is hydrogen, substituted or unsubstituted alkyl, or a residue of an amino acid, or R₃ and R₄ can form a ring;
each R₅ is independently hydrogen, substituted or unsubstituted alkyl, or the R₅ moieties can form a ring; and
each R₆ is substituted or unsubstituted aryl.

10. (canceled)

11. The compound of claim 9, wherein each P2 is independently a side chain of L-Phe(4-F), L-Ph3(3,4-F2), L-Phe, L-Phe(3-Cl), or L-His(Bzl);
each P3 is independently a side chain of L-Glu(OCHx), L-Glu(All), L-Glu(OBzl), L-Met(O)₂, or L-hCha; and/or
each P4 is independently a side chain of L-Oic, dhLeu, L-Chg, L-Lys(2-ClZ), or 4-Abz.

12. (canceled)

13. The compound of claim 9, wherein each P2 is independently a side chain of L-Igl, L-Phe(3,4-F2), L-Phe(3-Cl), L-Phe(4-F), or L-Glu(OBzl);
each P3 is independently a side chain of L-Agp, L-Lys, L-Nle(OBzl), L-Orn, or L-Arg; and/or
each P4 is independently a side chain of L-Arg, L-hArg, L-Orn, L-Lys, or L-Arg(Z)₂.

14. (canceled)

15. The compound of claim 9, wherein each P2 is independently a side chain of L-Leu, L-Orn, L-Cha, L-Thr, or L-Asn;
each P3 is independently a side chain of D-Gln, L-Agp, L-Nle(OBzl), L-Lys, or L-Orn; and/or
each P4 is independently a side chain of L-Agp, L-Dab(Z), L-Nle(OBzl), L-Orn, or L-Arg(NO₂).

16. (canceled)

17. The compound of claim 9, wherein each P2 is independently a side chain of L-Leu, L-hLeu, L-NptGly, L-Nle, or L-hTyr;
each P3 is independently a side chain of L-hArg, D-Trp, L-Agp, L-hCha, or L-hTyr; and/or
each P4 is independently a side chain of L-His(3-BOM), L-Agp, L-Lys(2-ClZ), dhLeu, or L-Idc.

18-26. (canceled)

27. The compound of claim 1, having the following structure:

| | |
|---|---|
| PK-1-91 | Ac-dWLR-kbt |
| (MN1063) | |
| AJS4016 | Ac-Ser-4-AMBA-Leu-Arg-kbt |
| MF1163 | Fmoc-hTyr-Arg-kbt |
| MF1067B | Ac-IdcGlu(OAll)Phe(4-F)Arg-kbt |
| (MPM2082B) | |
| MF1177 | Fmoc-Gly(2-th)-Arg-kbt |
| MF1168 | Cbz-Bta-Arg-kbt |
| MM3194A | Ac-PSKR-kbt |
| MF2008 | Cbz-Igl-Arg-kbt |
| MM4037-1 | Ac-IEFdR-kbt |
| MF1184 | Fmoc-Ala(2-th)-Arg-kbt |
| MF1165 | Fmoc-Nle(OBzl)-Arg-kbt |
| PK-1-104 | Ac-WLR-kbt |
| (MN1070) | |
| PK-1-105 | Ac-WLR-kbt-COOH |
| (MM4094) | |
| PK-1-94 | Ac-dWLR-kbt-COOH. |
| (MM4123) | |

28. A compound of Formula II, a pharmaceutically acceptable salt thereof, or a stereoisomer thereof: 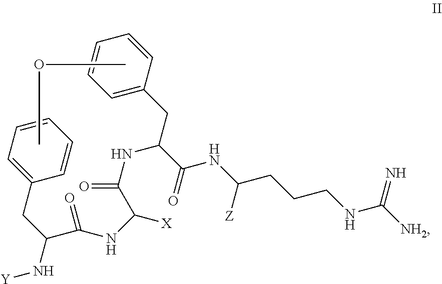
wherein
X is a side chain of a natural or unnatural amino acid;
Y is H, acetyl, tert-butyloxycarbonyl (Boc), benzyloxycarbonyl (Cbz), fluorenylmethyloxycarbonyl (Fmoc), benzyl, —C(O)R, —SOOR, —COOR, —C(O)NHR, —COCH(NHC(O)CH₃)—(CH₂)ₓ—NHC(O)—(CH₂)ₓ-p-halo-phenyl, substituted or unsubstituted —(CH₂)ₓaryl, substituted or unsubstituted —(CH₂)ₓheteroaryl, substituted or unsubstituted —(CH₂)ₓcycloalkyl, or substituted or unsubstituted —(CH₂)ₓheterocycle;
each R is independently C₁ to C₆ alkyl, C₃ to C₆ cycloalkyl, heterocycle, alkylheterocycle, aralkyl, or aryl; and
Z is independently 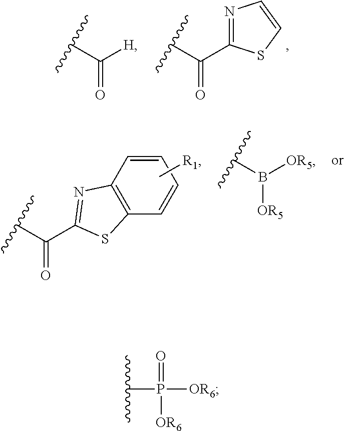
R₁ is hydrogen, 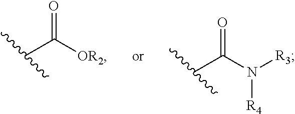
R₂ and R₃ are each independently hydrogen, substituted or unsubstituted alkyl, substituted or unsubstituted cycloalkyl, substituted or unsubstituted aryl, substituted or unsubstituted aralkyl, substituted or unsubstituted heteroaryl, or substituted or unsubstituted heteroarylalkyl;
R₄ is hydrogen, substituted or unsubstituted alkyl, or a residue of an amino acid, or R₃ and R₄ can form a ring;
R₅ is independently hydrogen, substituted or unsubstituted alkyl, or the R₅ moieties can form a ring; and
R₆ is substituted or unsubstituted aryl.

29.-32. (canceled)

33. The compound of claim 28, wherein X is a side chain of dhLeu, Gly, L-Idc, L-Ile, L-Leu, L-Met, L-Oic, or L-Pro.

34.-35. (canceled)

36. The compound of claim 28, wherein X is a side chain of 4-Abz, chAbu, D-Arg, dhAbu, dhLeu, L-Agp, L-Ala(Bth), L-Arg(NO₂), L-Arg(Z)₂, L-Arg, L-Chg, L-Cys(Bzl), L-Dab(Z), L-Glu(OBzl), L-hArg, L-His(3-BOM), L-hTyr, L-Hyp, L-Idc, L-Lys(2-ClZ), L-Lys, L-Nle(OBzl), or L-Oic, L-Orn.

37.-44. (canceled)

45. The compound of claim 28, having the following structure:

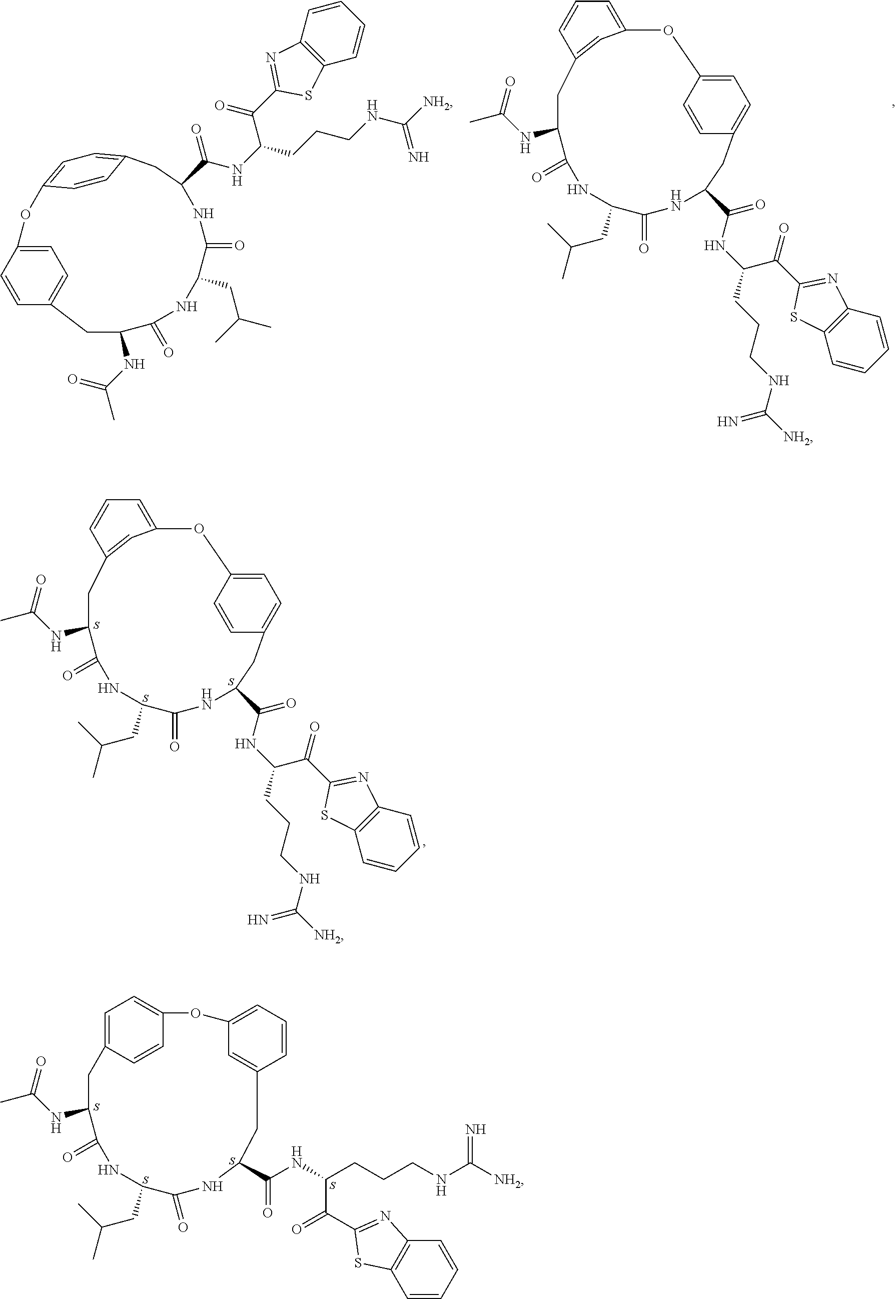

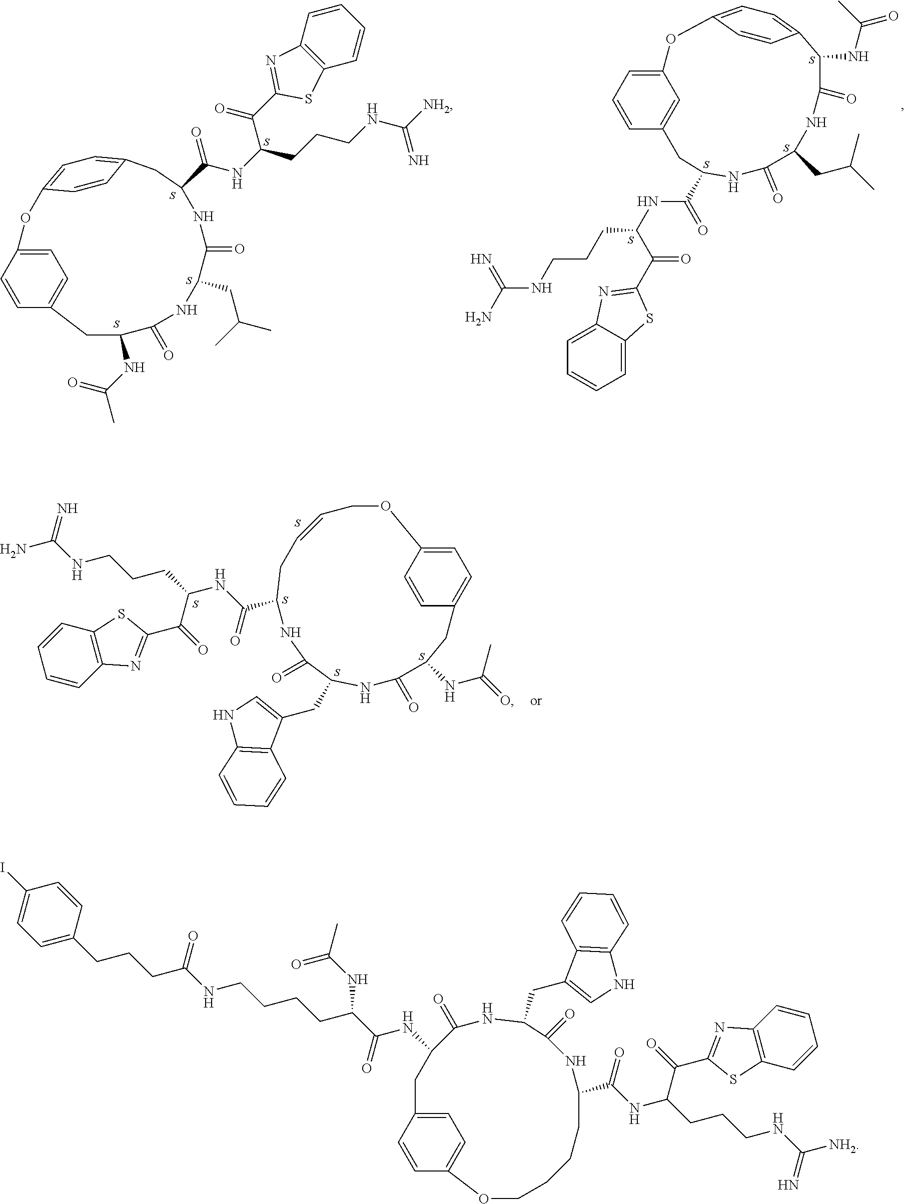

46. A method of inhibiting matriptase, hepsin, TMPRSS2, or hepatocyte growth factor activator (HGFA) comprising administering to an organism a composition comprising an effective amount of at least one compound of claim 1.

47. A method of overcoming and preventing resistance to anticancer drugs including targeted therapies, immunotherapy, radiation, and chemotherapy comprising administering to a subject in need thereof a pharmaceutical composition comprising a therapeutically effective amount of at least one compound of claim 1.

48. A method of overcoming and preventing resistance to a kinase small molecule or antibody inhibitor including those targeting EGFR and MET by blocking HGF and MSP production or activation comprising administering to a subject in need thereof a pharmaceutical composition comprising a therapeutically effective amount of at least one compound of claim 1.

49. A method of overcoming and preventing resistance to a DNA-damaging agent including gemcitabine comprising administering to a subject in need thereof a pharmaceutical composition comprising a therapeutically effective amount of at least one compound of claim 1.

50. A method of overcoming and preventing resistance to an immunotherapy agent including a PD-1 antagonist comprising administering to a subject in need thereof a pharmaceutical composition comprising a therapeutically effective amount of at least one compound of claim 1.

51. A method of inhibiting tumor progression and metastasis comprising administering to the subject in need thereof a pharmaceutical composition comprising a therapeutically effective amount of at least one compound of claim 1.

52.-55. (canceled)

56. A pharmaceutical composition comprising a therapeutically effective amount of at least one compound of claim 1 or a salt thereof and a pharmaceutically acceptable excipient.

57. A method of treating or preventing a viral infection in a subject comprising administering to the subject in need thereof a pharmaceutical composition comprising a therapeutically effective amount of a compound of claim 1.

58.-63. (canceled)

64. A method of inhibiting TMPRSS2 and/or matriptase in an organism comprising administering to the organism a composition comprising an effective amount of a compound of claim 1.
